(19) United States
(12) Patent Application Publication
(10) Pub. No.: US 2018/0179593 A1
(43) Pub. Date: Jun. 28, 2018

(54) MARKERS FOR MATURE BETA-CELLS AND METHODS OF USING THE SAME

(71) Applicant: President and Fellows of Harvard College, Cambridge, MA (US)

(72) Inventors: Douglas A. Melton, Lexington, MA (US); Sinisa Hrvatin, Cambridge, MA (US)

(21) Appl. No.: 15/696,140

(22) Filed: Sep. 5, 2017

Related U.S. Application Data
(63) Continuation of application No. 14/228,959, filed on Mar. 28, 2014, now abandoned.
(60) Provisional application No. 61/806,371, filed on Mar. 28, 2013.

(51) Int. Cl. C12Q 1/6881

(52) U.S. Cl. CPC C12Q 1/6881; C12Q 2600/158

(57) ABSTRACT

Markers for mature β-cells and methods of using these markers are disclosed.

### RELATED APPLICATIONS

This application is a continuation of U.S. patent Ser. No. 14/228,959, filed Mar. 28, 2014, which claims the benefit of U.S. Provisional Application No. 61/806,371, filed Mar. 28, 2013, the entire teachings of all of which are herein incorporated by reference.

### GOVERNMENT SUPPORT

This invention was made with government support under 2 U01 DK072473-07 awarded by the National Institutes of Health. The government has certain rights in the invention.

### BACKGROUND OF THE INVENTION

The directed differentiation of stem cells has the potential to produce β-cells for administration to individuals suffering from diseases associated with β-cell abnormality (e.g., diabetes). However, existing in vitro differentiation protocols often produce “β-like” cells, which do not have the same functional properties as mature β-cells. In addition, the complete set of signals and mechanisms governing β-cell maturation remains unknown.

### SUMMARY OF THE INVENTION

The present invention provides solutions to one or more of the problems outlined above. In particular, the present invention provides markers for identifying mature β-cells and methods of using the markers for identifying mature β-cells, methods of identifying agents that modulate maturity of β-cells (e.g., agents that induce functional β-cell maturation in vitro, or agents that induce β-cell maturation in vivo), methods of modulating disorders associated with β-cell deficiency, and related compositions and methods.

In some aspects, the invention provides a method of detecting β-cells, the method comprising: (a) obtaining a putative β-cell or a population of putative β-cells to be assessed; (b) measuring expression of a plurality of genes in the putative β-cell or the population of putative β-cells to produce a transcriptome of the putative β-cell or the population of putative β-cells; (c) comparing the transcriptome of the putative β-cell or the population of putative β-cells to a reference mature β-cell transcriptome exhibiting a pattern of expression depicted in FIG. 12; and (d) detecting β-cells, wherein the putative β-cell or a population of putative β-cells are mature β-cells if the transcriptome of the putative β-cell or the population of putative β-cells exhibits a pattern of expression similar to the reference mature β-cell transcriptome.

In some aspects, the invention provides a method of distinguishing β-cells and non-β-cells in a sample comprising pancreatic cells, the method comprising: (a) obtaining a sample comprising pancreatic cells; (b) measuring expression of a plurality of genes in the sample to produce a transcriptome of the pancreatic cells; (c) comparing the transcriptome of the pancreatic cells to a reference β-cell transcriptome exhibiting a pattern of expression depicted in FIG. 12 and/or a reference non-β-cell transcriptome exhibiting a pattern of expression depicted in FIG. 13; and (d) distinguishing β-cells and non β-cells, wherein (i) the pancreatic cells are β-cells if the transcriptome of pancreatic cells exhibits a pattern of expression similar to the reference β-cell transcriptome, or (ii) the pancreatic cells are non-β-cells if the transcriptome of pancreatic cells exhibits a pattern of expression similar to the reference non-β-cell transcriptome.

In some aspects, the present invention provides a method of identifying the functional maturity of β-cells, the method comprising: (a) obtaining a putative β-cell or a population of putative β-cells; and (b) detecting an expression level in the putative β-cell or the population of putative β-cells of one or more genes listed in Table 2, Table 3 or Table 4, wherein: (i) an elevated level of expression of one or more genes listed in Table 1 or Table 2 in the putative β-cell or the population of putative β-cells indicates that the putative β-cell or the population of putative β-cells are functionally mature β-cells; and (ii) an elevated level of expression of one or more genes listed in Table 3 or Table 4 in the putative β-cell or the population of putative (3-cells indicates that the putative β-cell or the population of putative β-cells are functionally immature β-cells.

In some aspects, the present invention provides a method of identifying mature β-cells, the method comprising: (a) obtaining a putative β-cell or a population of putative β-cells; and (b) detecting an expression level in the putative β-cell or the population of putative β-cells of one or more genes listed in Table 1 or Table 3, wherein: (i) an elevated level of expression of one or more genes listed in Table 1 or Table 2 in the putative β-cell or the population of putative β-cells indicates that the putative β-cell or the population of putative β-cells are mature β-cells.

In some aspects, the present invention provides a method of identifying fetal β-cells, comprising: (a) obtaining a putative β-cell or a population of putative β-cells; and

(b) detecting an expression level in the putative β-cell or the population of putative (3 cells of one or more genes listed in Table 3, wherein: (i) an elevated level of expression of one or more genes listed in Table 3 in the putative β-cell or the population of putative β-cells indicates that the putative β-cell or the population of putative β-cells are fetal β-cells.

In some aspects, the present invention provides a method of identifying in vitro-differentiated insulin-positive β-like cells, comprising: (a) obtaining a putative β-cell or a population of putative β-cells; and (b) detecting an expression level in the putative β-cell or the population of putative β-cells of one or more genes listed in Table 4, wherein: (i) an elevated level of expression of one or more genes listed in Table 4 in the putative β-cell or the population of putative β-cells indicates that the putative β-cell or the population of putative β-cells are in vitro-differentiated insulin-positive β-like cells.

In some aspects, the present invention provides a method of distinguishing mature and immature β-cells, comprising: (a) obtaining a putative β-cell or a population of putative β-cells; (b) measuring expression of a group of genes in the putative β-cell or the population of putative β-cells to produce an expression profile of the β-cell or the population of β-cells; (c) comparing the expression profile of the putative β-cell or the population of putative β-cells to any or all of: (i) a reference mature β-cell expression profile selected from the group consisting of a first group of genes having higher expression levels in mature β-cells compared to fetal β-cells, wherein the first group of genes is selected from the group consisting of STAT4, NPAS2, STAT3, NPAS2, STAT3, PBX3, NR3C2, DDIT3, SIX4, ETV5, SIX2, TP53, BCL6, MESP1, HOPX, BHLHB3, EPAS1, KLF9, KCNK3, GPI, CHGB, ALDOA, MAFA, SYT7, IAPP, WNT4, PDK3, KCNK1, SLC2A2, ESR1, G6PC2, and a second group of genes having higher expression levels in mature β-cells compared to insulin-positive β-like cells, wherein the second group of genes is selected from the group consisting of XBP1, NFIA, PURA, PDX1, NR3C2, MNX1, GLIS3, EPAS1, HSF4, TSHZ3, MAFA, NKX6-1, HOPX, RORC, NFIX, PEG3, CEBPD, KLF9, STX1A, KCNMA1, PDX1, CHGB, MNX1, PCSK2, NKX6.1, GLIS3, KCNK12, KCNK3, GCGR, KCNK1, SLC30A8, PCSK1, MAFA, ESR1, SLC2A2, IAPP, G6PC2, STXBP1, KCNH2, KCNMB2, UCN3, WNT4; (ii) a reference immature fetal β-cell expression profile selected from the group consisting of a third group of genes having higher expression levels in fetal β-cells compared to mature β-cells, wherein said third group of genes is selected from the group consisting of LZTS1, EVI1, MYCN, FOS, EGR1, RCOR2, TCF3, ASCL2, NOTCH1, LMO4, PAX4, NFIB, ISX, SOX11, LHX4, ZNF423, SOX8, RFX1, PROX1, HHEX, CSRNP3, LZTR1, SOX4, NKX6.2, COL1A1, PAX4, KCNH6, RIMS3, PROX1, SOX4, ACSS1, GHRL, NOTCH1, KCNN3, GCK, PYY, HCN3, KCNJ4, and a fourth group of genes having higher expression levels in insulin-positive β-like cells compared to mature β-cells, wherein said fourth group of genes is selected from the group consisting of FOXA1, HHEX, NR2F1, FEV, IRX2, SOX11, PAX4, ONECUT2, LMO4, AEBP1, HES6, TGIF2, LZTS1, TCF3, GATA4, ARX, EGR1, RCOR2, CEBPA, ELF4, HNF4G, PBX2, ISX, ZNF217, NTS, GAST, RIMS3, CACNA1E, PYY, SCT, FOXA1, GATA4, KCNH6, ARX, DLL3, NOTCH1, IRX2, DPP4, PAX4, ACOX2, KCNB1, PROX1, GHRL, SLC2A1, ONECUT2, and SLC2A3; and (d) distinguishing mature and immature β-cells, wherein the putative β-cell or the population of putative β-cells are: (i) mature β-cells if the expression profile of the putative β-cell or the population of putative β-cells exhibit a pattern of expression similar to either reference mature β-cell expression profile; or (ii) immature β-cells if the expression profile of the putative β-cell or the population of putative β-cells exhibit a pattern of expression similar to either reference immature β-cell expression profile expression profile.

In some aspects, the present invention provides a method of distinguishing mature and immature β-cells, comprising: (a) obtaining a putative β-cell or a population of putative β-cells; (b) assessing enrichment of a signaling pathway to produce a signaling pathway enrichment plot of the putative β-cell or the population of putative β-cells, wherein the signaling pathway is selected from the group consisting of an unfolded protein response signaling pathway, an insulin synthesis and secretion signaling pathway, and a metal ion SLC transporters signaling pathway; and (c) distinguishing mature and immature β-cells, wherein the putative β-cell or the population of putative β-cells are: (i) mature β-cells if the signaling pathway enrichment plot of the β-cell or the population of β-cells indicates that at least one of the unfolded protein response signaling pathway, the insulin synthesis and secretion signaling pathway, and the metal ion SLC transporters signaling pathway are enriched in the putative β-cell or the population of putative β-cells; or (ii) immature β-cells if the signaling pathway enrichment plot of the β-cell or the population of β-cells indicates that none of the unfolded protein response signaling pathway, the insulin synthesis and secretion signaling pathway, and the metal ion SLC transporters signaling pathway are enriched in the putative β-cell or the population of putative β-cells.

In some aspects, the present invention provides a method of distinguishing mature and immature β-cells, comprising: (a) obtaining a putative β-cell or a population of putative β-cells; (b) assessing enrichment of a biological process to produce a biological process enrichment list of the putative β-cell or the population of putative β-cells, wherein the biological process is selected from the group consisting of: (i) generation of precursor metabolites and energy, (ii) oxidation reduction, (iii) vesicle-mediated transport, (iv) electron transport chain, (v) monosaccharide metabolic process, (vi) cell morphogenesis, (vii) cellular component morphogenesis, (viii) cell projection organization, (ix) Wnt receptor signaling pathway, (x) cell projection morphogenesis, (xi) cytoskeleton organization, (xii) sterol biosynthetic process, (xiii) cholesterol biosynthetic process, (xiv) actin filament-based process, (xv) actin cytoskeleton organization, (xvi) sterol metabolic process, and (xvii) neuron projection development; (c) distinguishing mature and immature β-cells, wherein the putative β-cell or the population of putative β-cells are: (i) mature β-cells if the biological process enrichment list of the putative β-cell or the population of putative β-cells indicates that at least one of the biological processes selected from the group consisting of (i), (ii), (iii), (iv), and (v) is enriched in the β-cell or the population of β-cells; or (ii) immature β-cells if the biological process enrichment list of the putative β-cell or the population of putative β-cells indicates that at least one of the biological processes selected from the group consisting of (vi), (vii), (viii), (ix), (x), (xi), (xii), (xiii), (xiv), (xv), (xvi), and (xvii) is enriched in the putative β-cell or the population of putative β-cells.

In some aspects, the present invention provides a method of identifying a candidate agent that modulates the functional maturity of β-cells, comprising: (a) contacting a β-cell or a population of β-cells with a test agent; (b) monitoring expression of a group of genes in the β-cell or the population of β-cells, in the presence of the test agent, to produce an expression profile of the β-cell or the population of β-cells; (c) comparing the expression profile of the β-cell or the population of β-cells to: (i) a reference mature β-cell expression profile selected from the group consisting of a first group of genes having higher expression levels in mature β-cells compared to fetal β-cells, wherein the first group of genes is selected from the group consisting of KCNK3, GPI, CHGB, ALDOA, MAFA, SYT7, IAPP, WNT4, PDK3, KCNK1, SLC2A2, ESR1, G6PC2, and a second group of genes having higher expression levels in mature β-cells compared to insulin-positive β-like cells, wherein the second group of genes is selected from the group consisting of STX1A, KCNMA1, PDX1, CHGB, MNX1, PCSK2, NKX6.1, GLIS3, KCNK12, KCNK3, GCGR, KCNK1, SLC30A8, PCSK1, MAFA, ESR1, SLC2A2, IAPP, G6PC2, STXBP1, KCNH2, KCNMB2, UCN3, and WNT4; (ii) a reference immature β-cell expression profile selected from the group consisting of a third group of genes having higher expression levels in fetal β-cells compared to mature β-cells, wherein said third group of genes is selected from the group consisting of NKX6.2, COL1A1, PAX4, KCNH6, RIMS3, PROX1, SOX4, ACSS1, GHRL, NOTCH1, KCNN3, GCK, PYY, HCN3, and KCNJ4, and a fourth group of genes having higher expression levels in insulin-positive β-like cells compared to mature β-cells, wherein said fourth group of genes is selected from the group consisting of NTS, GAST, RIMS3, CACNA1E, PYY, SCT, FOXA1, GATA4, KCNH6, ARX, DLL3, NOTCH1, IRX2, DPP4, PAX4, ACOX2, KCNB1, PROX1, GHRL, SLC2A1, ONECUT2, and SLC2A3; and (d) identifying the test agent as a candidate agent that modulates the functional maturity of β-cells, wherein: (i) the test agent is a candidate agent that induces β-cells to become functionally mature if the β-cell or the population of β-cells exhibit a pattern of expression similar to the either reference mature β-cell expression profile in the presence of the test agent; (ii) the test agent is a candidate agent that induces β-cells to become functionally immature if the β-cell or the population of β-cells exhibit a pattern of expression similar to either reference immature β-cell expression profile.

In some aspects, the present invention provides a method of identifying a candidate agent that modulates the functional maturity of β-cells, comprising: (a) contacting a β-cell or a population of β-cells with a test agent; (b) assessing enrichment of a signaling pathway in the presence of the test agent to produce a signaling pathway enrichment plot of the β-cell or the population of β-cells, wherein the signaling pathway is selected from the group consisting of an unfolded protein response signaling pathway, an insulin synthesis and secretion signaling pathway, and a metal ion SLC transporters signaling pathway; (c) identifying the test agent as a candidate agent that modulates the functional maturity of β-cells, wherein: (i) the test agent is a candidate agent that induces β-cells to become functionally mature if the signaling pathway enrichment plot of the β-cell or the population of β-cells indicates that at least one of the unfolded protein response signaling pathway, the insulin synthesis and secretion signaling pathway, and the metal ion SLC transporters signaling pathway are enriched in the β-cell or the population of β-cells; or (ii) the test agent is a candidate agent that induces β-cells to become functionally immature if the signaling pathway enrichment plot of the β-cell or the population of β-cells indicates that none of the unfolded protein response signaling pathway, the insulin synthesis and secretion signaling pathway, and the metal ion SLC transporters signaling pathway are enriched in the β-cell or the population of β-cells.

In some aspects, the present invention provides a method of identifying a candidate agent that modulates the functional maturity of β-cells, comprising: (a) contacting a β-cell or a population of β-cells with a test agent; (b) assessing enrichment of a biological process in the presence of the test agent to produce a biological process enrichment list of the β-cell or the population of β-cells, wherein the biological process is selected from the group consisting of (i) generation of precursor metabolites and energy, (ii) oxidation reduction, (iii) vesicle-mediated transport, (iv) electron transport chain, (v) monosaccharide metabolic process, (vi) cell morphogenesis, (vii) cellular component morphogenesis, (viii) cell projection organization, (ix) Wnt receptor signaling pathway, (x) cell projection morphogenesis, (xi) cytoskeleton organization, (xii) sterol biosynthetic process, (xiii) cholesterol biosynthetic process, (xiv) actin filament-based process, (xv) actin cytoskeleton organization, (xvi) sterol metabolic process, and (xvii) neuron projection development; and (c) identifying the test agent as a candidate agent that modulates the functional maturity of β-cells, wherein: (i) the test agent is a candidate agent that induces β-cells to become functionally mature if the biological process enrichment list of the β-cell or the population of β-cells indicates that at least one biological process selected from the group consisting of (i), (ii), (iii), (iv), and (v) is enriched in the β-cell or the population of β-cells; or (ii) the test agent is a candidate agent that induces β-cells to become functionally immature if the biological process enrichment list of the β-cell or the population of β-cells indicates that at least one biological process selected from the group consisting of (vi), (vii), (viii), (ix), (x), (xi), (xii), (xiii), (xiv), (xv), (xvi), and (xvii) is enriched in the β-cell or the population of β-cells.

In some embodiments, the β-cell or the population of β-cells are obtained from an in vitro source. In some embodiments, the putative β-cell or the population of putative β-cells are obtained from an in vitro source.

In some embodiments, the in vitro source is a culture of differentiating stem cells.

In some embodiments, the stem cells are selected from the group consisting of human embryonic stem cells (hESCs), induced pluripotent stem cells (iPSCs), and combinations thereof. In some embodiments, the in vitro source is selected from the group consisting of a cell bank, cell line, cell culture, cell population, and combinations thereof. In some embodiments, the in vitro source is an artificial tissue or organ.

In some embodiments, the cell or population of cells are obtained from an in vivo source. In some embodiments, β-cell or the population of β-cells are obtained from an in vivo source. In some embodiments, the putative β-cell or the population of putative β-cells are obtained from an in vivo source.

In some embodiments, the in vivo source is an individual that has received an administration of β-cells. In some embodiments, the in vivo source is an individual suffering from a β-cell disorder selected from the group consisting of a disorder associated with immature β-cells, a disorder associated with destruction of β-cells, a disorder associated with dysfunctional β-cells, and a disorder associated with an insufficient number of β-cells. In some embodiments, the in vivo source is an individual suspected of being in need of functionally mature β-cells. In some embodiments, the in vivo source is a tissue or organ obtained from a donor individual.

In some embodiments,
the individual is a human or animal individual.

In some embodiments, measuring expression comprises utilizing a technique selected from the group consisting of a microarray analysis, RNA-seq, RT-PCR, and q-RT-PCR.

In some embodiments, detecting the expression level comprises conducting at least one binding assay to determine the expression level of the one or more genes.

In some embodiments, the methods further comprise sorting immature and mature β-cells. In some embodiments, sorting the immature and mature β-cells fluorescence-activated cell sorting (FACS). In some embodiments, the FACS comprises staining at least one antibody specific for a putative β-cell surface marker selected from the group consisting of ABCA3, CD79B, FXYD2, KCNB2, NLGN1, PTPRU, SLC6A9, ABCC8, CD8A, GCGR, KCNF1, NPR2, ROBO1, SORL1, ABCG1, CDH2, GPR120, KCNG3, NRCAM, RTN4, SVOP, ACSL1, CDH22, GPR19, KCNH2, PCDHA1, SEMA5A, TGFBR3, ATP1B2, CHRNA5, GRIA2, KCNMA1, PCDHA3, SERP2, TRPM2, CACNA1H, CYB561, KCNH1, KCNQ2, PIGU, SLC17A6, TRPM5, CADM1, EFNB3, IGSF11, MADD, PLXNA2, SLC43A2, TSPAN13, CASR, FFAR1, IL17RB, NEO1, PRRG2, SLC6A6, and UNC5A.

In some embodiments, the methods further comprise quantifying the sorted β-cells.

In some embodiments, the methods further comprise preserving the sorted β-cells.

In some embodiments, the methods further comprise conducting a GSIS assay on the β-cell or population of β-cells.

In some embodiments, the mature β-cell is a human β-cell.

In some embodiments, the fetal β-cell is a human β-cell.

In some embodiments, the insulin-positive β-like cell is derived from human cells selected from the group consisting of human embryonic stem cells, reprogrammed human somatic cells, and induced human pluripotent stem cells.

In some aspects, the invention provides a method of delivering a molecule of interest to a β-cell or a population of β-cells, comprising: contacting the β-cell or the population of β-cells with a composition comprising the molecule of interest conjugated to an antibody that binds to a putative β-cell surface marker selected from the group consisting of ABCA3, CD79B, FXYD2, KCNB2, NLGN1, PTPRU, SLC6A9, ABCC8, CD8A, GCGR, KCNF1, NPR2, ROBO1, SORL1, ABCG1, CDH2, GPR120, KCNG3, NRCAM, RTN4, SVOP, ACSL1, CDH22, GPR19, KCNH2, PCDHA1, SEMA5A, TGFBR3, ATP1B2, CHRNA5, GRIA2, KCNMA1, PCDHA3, SERP2, TRPM2, CACNA1H, CYB561, KCNH1, KCNQ2, PIGU, SLC17A6, TRPM5, CADM1, EFNB3, IGSF11, MADD, PLXNA2, SLC43A2, TSPAN13, CASR, FFAR1, IL17RB, NEO1, PRRG2, SLC6A6, and UNC5A.

In some aspects, the present invention provides a method of identifying a candidate agent that modulates differentiation of β-cells, comprising: contacting a cell, population of cells, cell line or cell culture with a test agent; and monitoring the cell, population of cells, cell line, or cell culture for expression of one or more β-cell specific transcription factors in the presence of the test agent, wherein the β-cell specific transcription factors are selected from the group consisting of ASCL2, NR0B1, SIX4, CHD7, TOX, OLIG1, TSHZ3, DACH1, TSNAX, DACH2, MYT1L, PEG3, ZNF10, NDN, ZNF395, ETV5, ZNF540, HOPX, RXRG and ZNF672; and identifying the test agent as a candidate agent that modulates differentiation of β-cells if the cell, population of cells, cell line, or cell culture expresses one or more of the β-cell specific transcription factors in the presence of the test agent.

In some aspects, the present invention provides a composition for differentiating a precursor cell to a pancreatic β-cell, comprising one or more β-cell specific transcription factors selected from the group consisting of ASCL2, NR0B1, SIX4, CHD7, TOX, OLIG1, TSHZ3, DACH1, TSNAX, DACH2, MYT1L, PEG3, ZNF10, NDN, ZNF395, ETV5, ZNF540, HOPX, RXRG and ZNF672.

In some aspects, the present invention provides a method for differentiating a precursor cell to a pancreatic β-cell, comprising causing the precursor cell to upregulate expression of one or more β-cell specific transcription factors selected from the group consisting of ASCL2, NR0B1, SIX4, CHD7, TOX, OLIG1, TSHZ3, DACH1, TSNAX, DACH2, MYT1L, PEG3, ZNF10, NDN, ZNF395, ETV5, ZNF540, HOPX, RXRG and ZNF672.

In some aspects, the present invention provides a method of detecting mature β-cells comprising conducting at least one binding assay for at least one marker of β-cell functional maturity in a cell or a population of cells, wherein the presence of the at least one marker of β-cell functional maturity in the cell or a population of cells indicates that the cell or population of cells are mature β-cells.

In some embodiments, the at least one marker of β-cell functional maturity is selected from the group consisting of the genes listed in Table 1 (Table 1A, Table 1B, or both Tables 1A and 1B) or Table 2 (Table 2A, Table 2B, or both Tables 2A and 2B).

In some embodiments, the at least one marker of β-cell functional maturity comprises a protein marker of β-cell functional maturity.

In some embodiments, the at least one marker of β-cell functional maturity comprises an mRNA marker of β-cell functional maturity.

In some aspects, the present invention provides a method of detecting immature β-cells comprising conducting at least one binding assay for at least one marker of β-cell functional immaturity in a cell or population of cells, wherein the presence of the at least one marker of β-cell functional immaturity in the cell or a population of cells indicates that the cell or population of cells are immature β-cells.

In some embodiments, the at least one marker of β-cell functional immaturity is selected from the group consisting of the genes listed in Table 3 (Table 3A, Table 3B, or both Tables 3A and 3B) or Table 4 ((Table 4A, Table 4B, or both Tables 4A and 4B).

In some embodiments, the at least one marker of β-cell functional immaturity comprises a protein marker of β-cell functional immaturity.

In some embodiments, the at least one marker of β-cell functional immaturity comprises an mRNA marker of β-cell functional immaturity.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIGS. 1A-1B depict an exemplary six stage directed differentiation protocol. Cell types shown in FIG. 1A include: ES=embryonic stem cell; DE=definitive endoderm; PP=pancreatic progenitors; EP=endocrine progenitors; EN=endocrine; Media components: Alk5i=a TGFβ signaling inhibitor that acts via inhibition of activin receptor-like kinase 5; FAF-BSA=fatty acid-free bovine serum albumin; KGF=keratinocyte growth factor, also known as fibroblast growth factor 7; ITS-X=insulin, transferrin, selenium; noggin=a BMP signaling antagonist that acts via binding of BMP-7; PDBu=phorbol 12,13-dibutyrate, a protein kinase C agonist; RA=retinoic acid; SANT-1=a sonic hedgehog pathway antagonist that acts via inhibition of the receptor smoothened. FIG. 1B depicts the stepwise differentiation from hESCs to pancreatic endocrine cells. The Table shown in FIG. 1B contains reagents used during each stage of directed differentiation.

FIGS. 2A-2C demonstrate that human β-cells display a molecular signature distinct from other islet cells. FIG. 2A is a Table showing the results of a global analysis for probe sets differentially expressed (514 up, 889 down) in human islet populations enriched for β-cells compared to those depleted of β-cells. Human islet isolations likely contain contaminating non-islet tissues (e.g. exocrine cells, mesenchyme), contributing to the heterogeneity of the pool. Expression in unsorted islets is shown for comparison. Left, listed are the top ten gene ontology terms for non-β islet cell-specific genes and all overrepresented terms for β-cell-specific genes (q≤0.05). Right, row-normalized heatmap of probes shown with samples (columns) in the order of human β-cells, other human islet cells, hESC-derived stage 6 cells, and insulin+ stage 6 cells. From this list of probes, array signals are displayed for select established markers of non-β pancreatic cell types (FIG. 2B) and select known transcription factors preferentially expressed in β-cells (FIG. 2C).

FIGS. 3A-3D demonstrate the results of protein analysis of sorted islet populations. FIG. 3A is a bar graph showing results obtained using the Qproteome FFPE tissue kit; the inventors extracted a mean 104 pg protein per cell, as determined by the micro BCA assay, from fixed human islets and hESC-derived pancreatic cultures. Error bars are ±SEM. FIG. 3B is a schematic showing that Pdx1 protein of expected molecular weight was resolved by SDS-PAGE and Western blot using lysate from hESC-derived stage 6 cells that were paraformaldehyde-fixed and saponin-permeabilized and from cells that were fresh/unfixed. FIG. 3C is a FACS plot depicting human islets dispersed, fixed, and stained for intracytoplasmic insulin and glucagon, resolving by FACS clear populations of β- and α-cells. FIG. 3D are schematics depicting Western blots of proteins isolated from sorted β- and α-cells. Pdx1 was detected in β-cells but not α-cells, while glucokinase and the housekeeping protein Ran were equally found in β- and α-cells.

FIGS. 4A-4D demonstrate global expression (GO) analysis of hESC-derived cells and β-cells. FIGS. 4A-4D show gene ontology biological process annotation clustering for genes up-regulated in S6 insulin+ cells compared to all unsorted S6 cells (2.6% of probes) (FIG. 4A), unsorted S6 cells compared to the S6 insulin+ population (2.3%) (FIG. 4B), β-cells compared to S6 insulin+ cells (4.0%) (FIG. 4C), and S6 insulin+ cells compared to β-cells (4.9%) (FIG. 4D). Shown are the broadest enriched terms that are each representative of a cluster. Numbers indicate genes in each group with a given annotation.

FIGS. 5A-5C illustrate the relative expression of genes associated with endocrine pancreas development or function in β-cells and hESC-derived cells. FIG. 5A is a heatmap showing normalized expression values of 71 established endocrine lineage genes in human β-cells, hESC-derived stage 6 (S6) cells fresh and unsorted, or S6 insulin+ cells. Of these genes, 40 were significantly differentially expressed between the S6 insulin+ cells and the unsorted population (FIG. 5B), and 35 were differentially expressed between S6 insulin+ cells and human β-cells (FIG. 5C). Genes are listed by descending bio-weight.

FIGS. 6A-6D depict human β cell maturation. FIG. 6A shows FACS plots of human adult islets and human fetal pancreata sorted for INS⁺ cells (APC⁺). FIG. 6B shows differentially expressed transcription factors between adult and fetal β cells. FIG. 6C is a bar graph showing the relative expression of UCN3 in mouse and human fetal and adult β cells. Expression normalized to fetal levels in each species. FIG. 6D is a Table listing the top 5 most significant (Benjamini q value) Gene ontology biological processes relatively enriched in either adult or fetal β cells.

FIGS. 7A-7D show the results of differential expression between human β cells and hPSC-derived insulin expressing cells. FIG. 7A is a Table listing 152 pancreatic lineage genes colored for genes that are differentially over-expressed in adult β cells (red) or hPSC-INS⁺ cells (blue). Differential gene expression was calculated based on microarray data between human adult β cells and hPSC-INS⁺ cells (fold change >3, p<0.05) and confirmed by RNAseq between HUES8-INS⁺ cells and adult β cells (fold change >3). Asterisk indicates genes that are also differentially expressed between fetal and adult β cells. FIG. 7B shows immunofluorescence staining of hPSC-INS⁺ cells for PDX1, NKX6-1 and MAFA. Scale 100 um. FIG. 7C is alist of 42 transcription factors that were differentially expressed based on microarray data between human adult β cells and hPSC-derived INS⁺ cells (fold change >3, p<0.05) and confirmed by RNAseq between HUES8-INS⁺ cells and adult β cells (fold change >3). FIG. 7D is a Table listing the top 10 most significant (Benjamini q value) gene ontology biological processes enriched hPSC-INS⁺ cells over adult β cells.

FIGS. 8A-8C demonstrate a dysregulated ER stress response in hESC-derived insulin+ cells. FIG. 8A shows results of gene set enrichment analysis using curated Reactome gene sets identified three canonical pathways significantly underexpressed in hESC-derived stage 6 (S6) insulin+ cells compared to adult β-cells (FDR q-value<0.05). Enrichment plots are ordered from left to right by increasing q-value. FIG. 8B shows an Ingenuity Pathway Analysis (IPA) map of the endoplasmic reticulum stress pathway. All factors that overlapped with the Reactome unfolded protein response pathway are shown. Members are colored such that green indicates underexpression in S6 insulin+ cells compared to β-cells, and red indicates overexpression. Disease/function annotations of pathway members are noted in ovals when relevant to β-cell biology. FIG. 8C is a schematic depicting genes downstream of XBP1 comprised the most significantly inhibited transcription factororiginated gene set in S6 insulin+ cells relative to β-cells. 28 of 34 differentially expressed genes had expression direction consistent with XBP1 inhibition. The p-value corresponds to the overlap between all underexpressed genes and the genes regulated by XBP1, as determined in IPA. Asterisks indicate that a gene is represented in the microarray by multiple probes.

FIGS. 9A-9C depict the expression of β-cell signature genes in hESC-derived cells. The signature gene sets of adult β-cells (458 genes) and non-β pancreatic cells (775 genes) identified in this study were used to assess hESC-derived stage 6 (S6) cells. FIG. 9A shows that gene set enrichment analysis indicated significant enrichment of β-cell genes in S6 insulin+ cells compared to unsorted S6 cells (top panel), and of non-β pancreatic genes in unsorted S6 cells over the insulin+ fraction (middle panel). All genes were ranked by signal-to-noise ratio. p<0.001. FIG. 9B depicts hierarchical clustering based on Pearson correlation between centroids demonstrated that S6 insulin+ cells cluster more closely with β-cells than do unsorted S6 cells, but many differences between S6 insulin+ cells and β-cells remain. A heatmap is shown with row-normalized expression of all 1403 probes that comprise the signature sets. FIG. 9C is a graph showing results of statistical weighted voting using the 1403 probe signature classified unsorted S6 cells as non-β pancreatic cells with modest confidence, but S6 insulin+ cells could not be confidently classified as β-cells. A positive confidence index (maximum 1.0) corresponds to expression similarity with the β-cell signature compared to the non-β-cell signature. Bars are mean±SEM.

FIGS. 10A-10D depict β-cell signature transcription factor expression in hESC-derived insulin+ cells. FIG. 10A is a list of 49 transcription factors within the signature set which were differentially expressed in S6 insulin+ cells compared to β-cells. Of the 20 factors having higher expression levels in β-cells than S6 insulin+ cells, 18 are normally preferentially expressed in β-cells. Genes are ordered by bioweight and expression is displayed as fold change±SEM. FIGS. 10B-10D show the results of immunohistochemistry, which revealed no co-expression of Nkx6.1 in c-peptide+ (insulin-producing) cells (FIG. 10B), little co-expression of MafA (FIG. 10C), and full co-expression of Pdx1 (FIG. 10D).

FIGS. 11A-11C demonstrate that hPSC-derived insulin expressing cells resemble human fetal β cells. FIG. 11A is a bar graph showing glucose stimulated insulin secretion of dispersed cells. In contrast to adult β cells, fetal β cells and hPSC-INS⁺ cells both appear functionally immature as indicated by increased basal glucose secretion and lack of glucose stimulation. FIG. 11B shows hierarchical clustering based on microarray global gene expression across all genes indicated that hPSC-INS⁺ cells cluster closely with human fetal and not adult β cells. Numbers in parentheses indicate biological replicates. Lengths in the dendrogram represent correlation distances. FIG. 11C shows R² values based on microarray data across all genes are shown. Each row and column represents one sample. R² values between biological replicates of adult β cell samples (Adult_ins) are on average 0.89±0.04. R² values between sorted hPSC-derived insulin⁺ stage 6 cells and sorted fetal β cells are 0.88±0.02. The biological variation between adult β cells is not statistically smaller then the variation between fetal β cells and hPSC-INS⁺ stage 6 cells (p=0.49). This indicates that a high degree of similarity between hPSC-INS⁺ cells and human fetal β cells.

FIG. 12 shows a Table of genes with higher expression levels in sorted β cells relative to non β islet cells.

FIG. 13 shows a Table of genes with higher expression levels in non β islet cells relative to sorted β cells.

### DETAILED DESCRIPTION OF THE INVENTION

The present invention relates to novel markers for detecting β-cells (e.g., functionally mature β-cells), and methods of using those markers for identifying functionally mature β-cells and distinguishing between immature and mature β-cells (e.g., determining whether an in vitro-differentiated β-cell has matured). In particular, the work described herein provides for the first time the mature β-cell transcriptome. In addition, the work described herein provides genes which are differentially expressed between mature and immature β-cells, signaling pathways which are enriched in mature and immature β-cells, biological processes which are enriched in mature and immature β-cells, and putative β-cell cell-surface markers.

Accordingly, the present invention provides markers and methods for identifying the functional maturity of β-cells (e.g., distinguishing between immature and mature β-cells in a population), identifying agents that modulate the functional maturity of a β-cell, identifying agents that modulate disorders associated with immature β-cells, identifying individuals in need of functionally mature β-cells, selecting functionally mature in vitro-differentiated β-cells or β-like cells for administration to an individual (e.g., transplantation of mature β-cells into the individual, e.g., a human or animal), and identifying whether β-cells that have been administered to an individual are mature β-cells in vivo.

In one aspect, the present invention provides a method of detecting mature β-cells. An exemplary method of detecting mature β-cells comprises measuring expression of genes in a putative β-cell or a population of putative β-cells to produce a transcriptome of the β-cell or a population of β-cells, and comparing the transcriptome of the β-cell or the population of β-cells to a reference mature β-cell transcriptome, wherein the β-cell or the population of β-cells are mature β-cells if the transcriptome of the β-cell or the population of β-cells exhibit a pattern of expression similar to the reference mature β-cell transcriptome.

In some embodiments, a method of detecting mature β-cells comprises (a) obtaining a putative β-cell or a population of putative β-cells; (b) measuring expression of a plurality of genes in the β-cell or a population of β-cells to produce a transcriptome of the β-cell or the population of β-cells; (c) comparing the transcriptome of the β-cell or the population of β-cells to a reference mature β-cell transcriptome exhibiting a pattern of expression depicted in FIG. 12; and (d) detecting mature β-cells, wherein the β-cell or the population of β-cells are mature β-cells if the transcriptome of the β-cell or the population of β-cells exhibit a pattern of expression similar to the reference mature β-cell transcriptome.

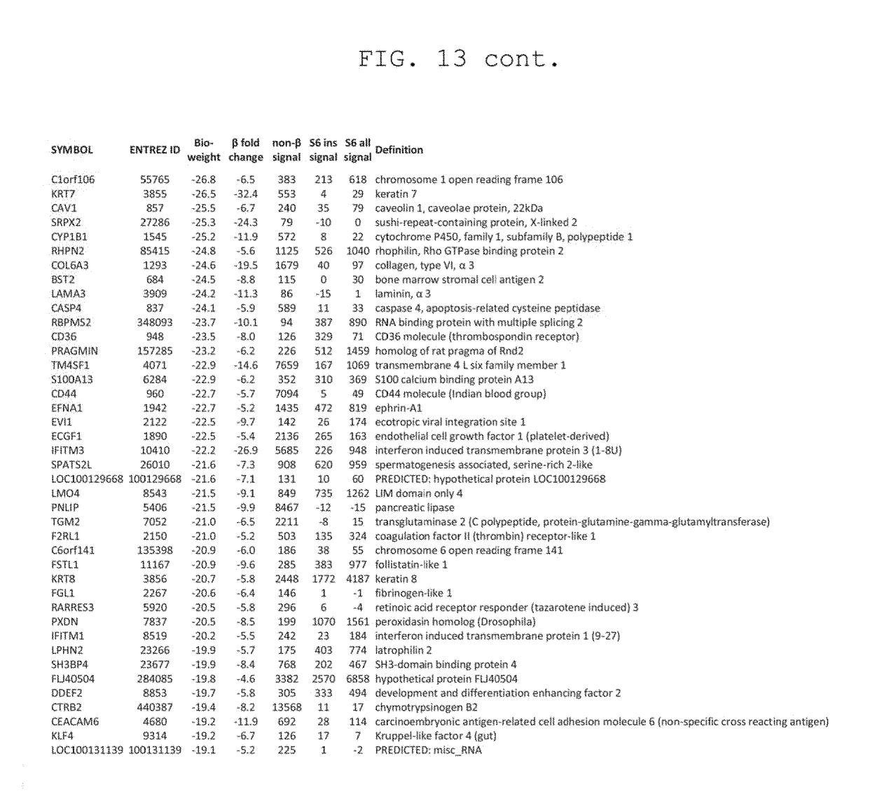

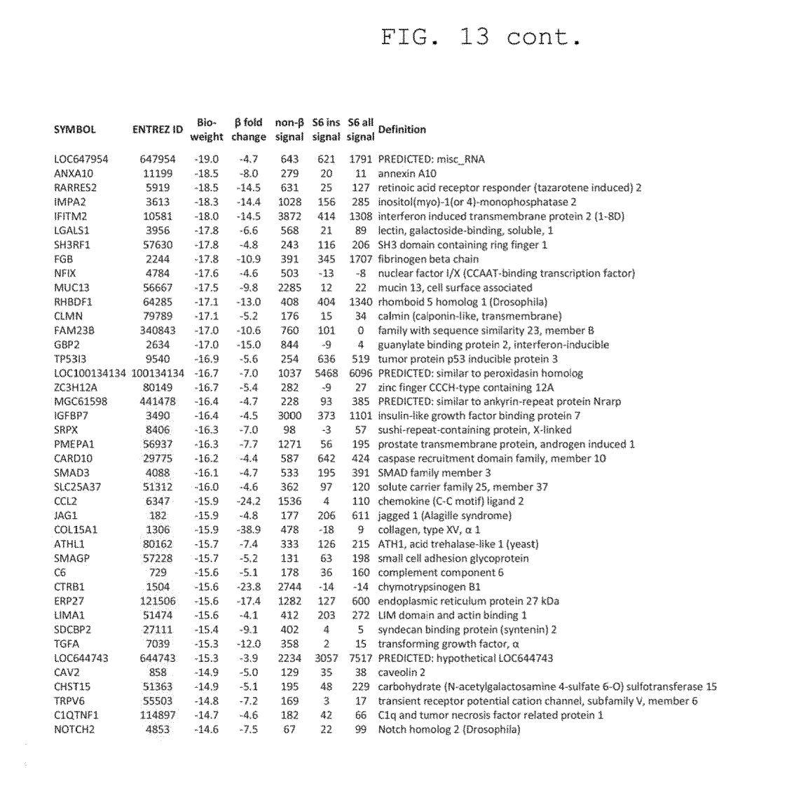

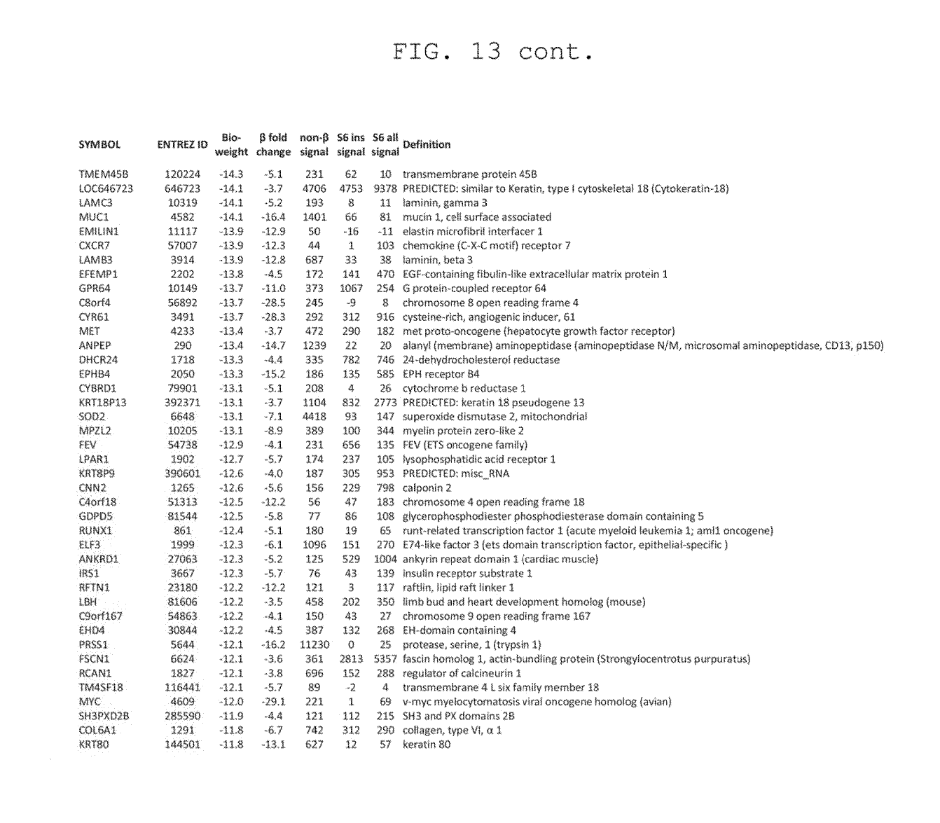

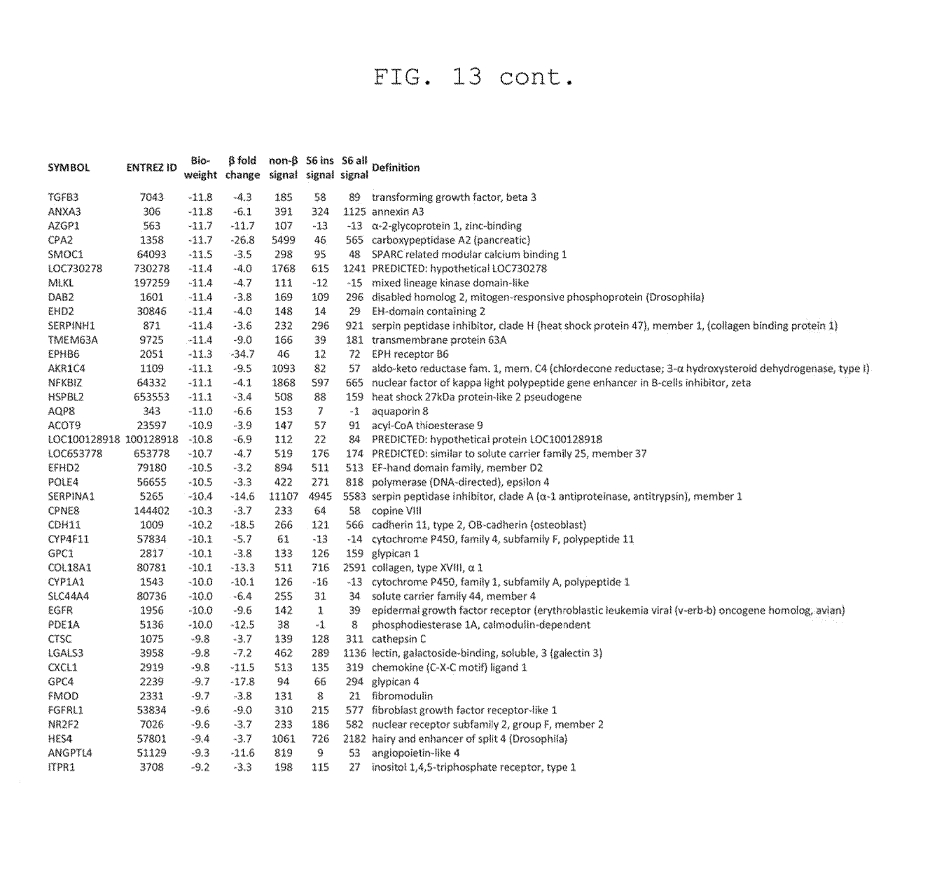

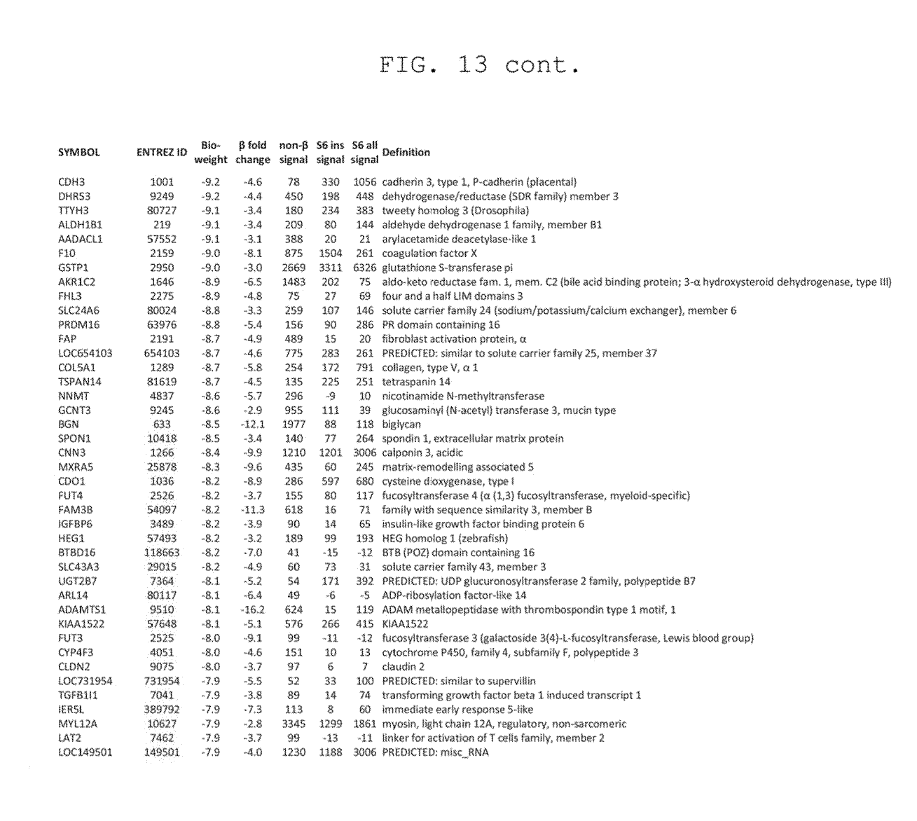

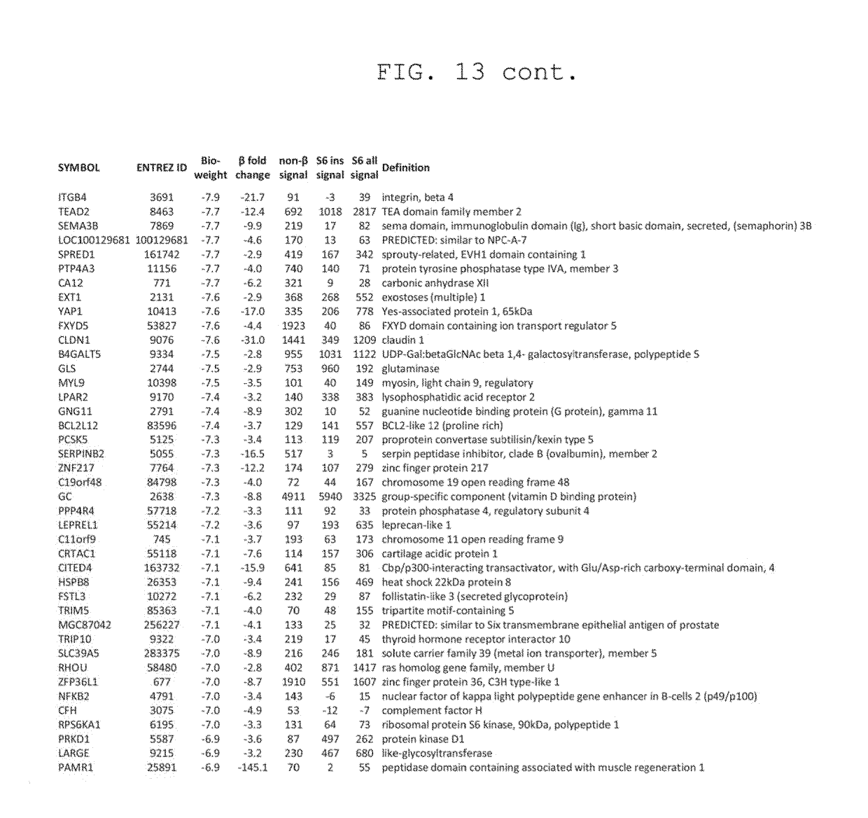

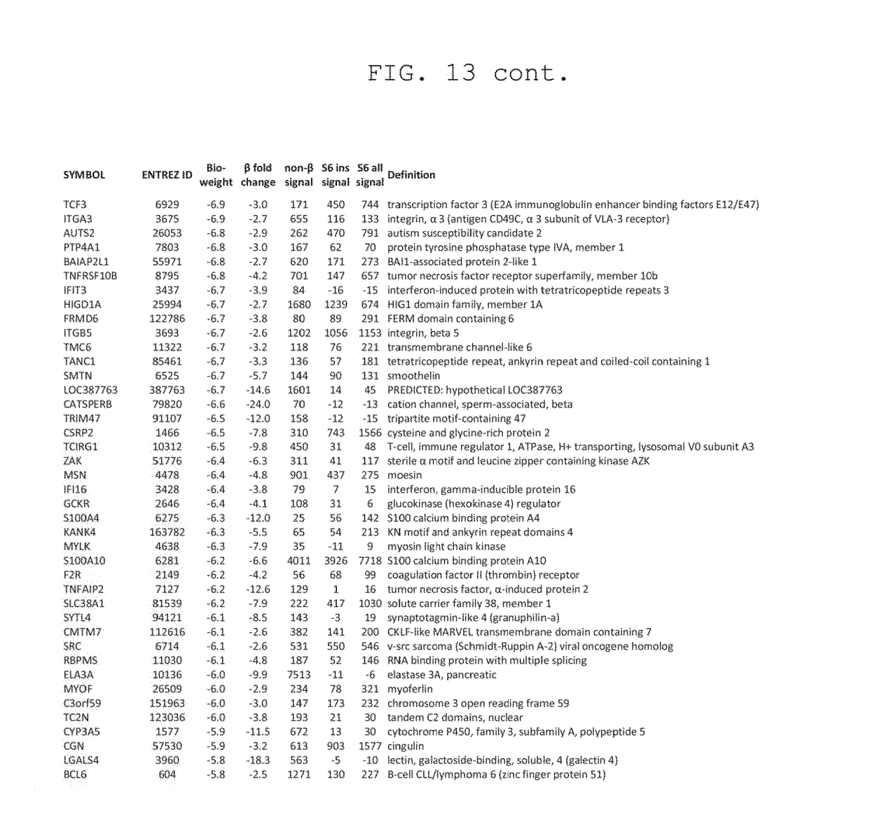

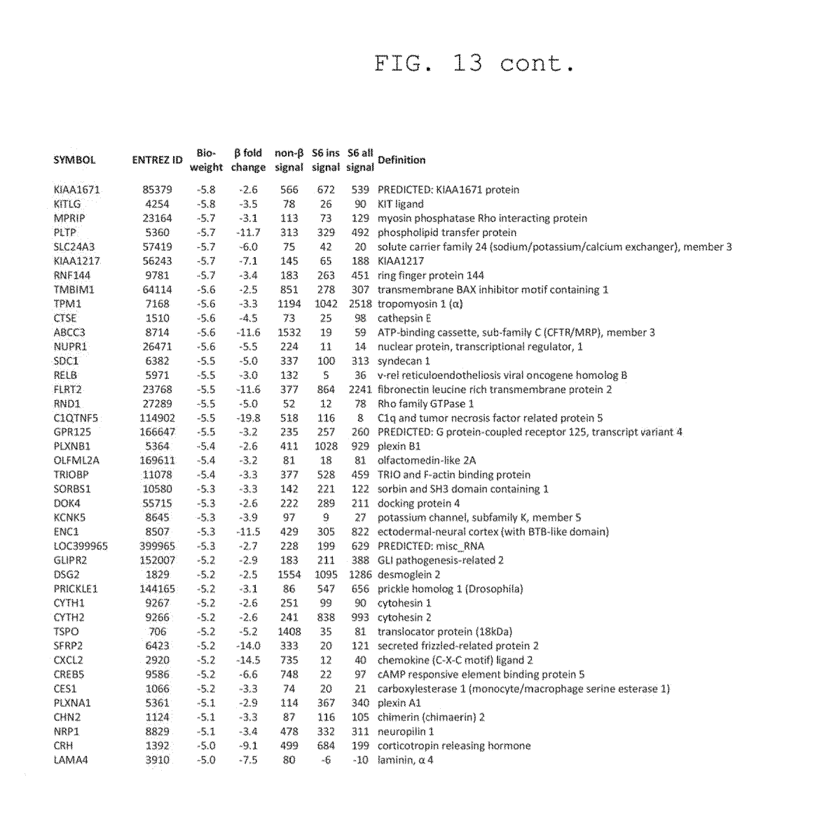

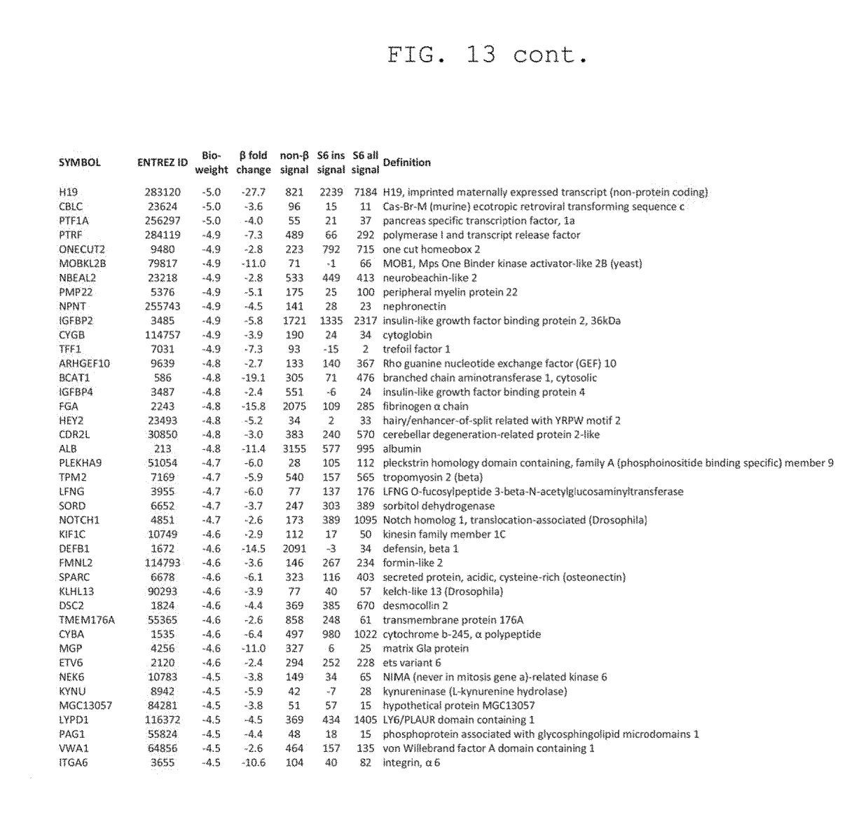

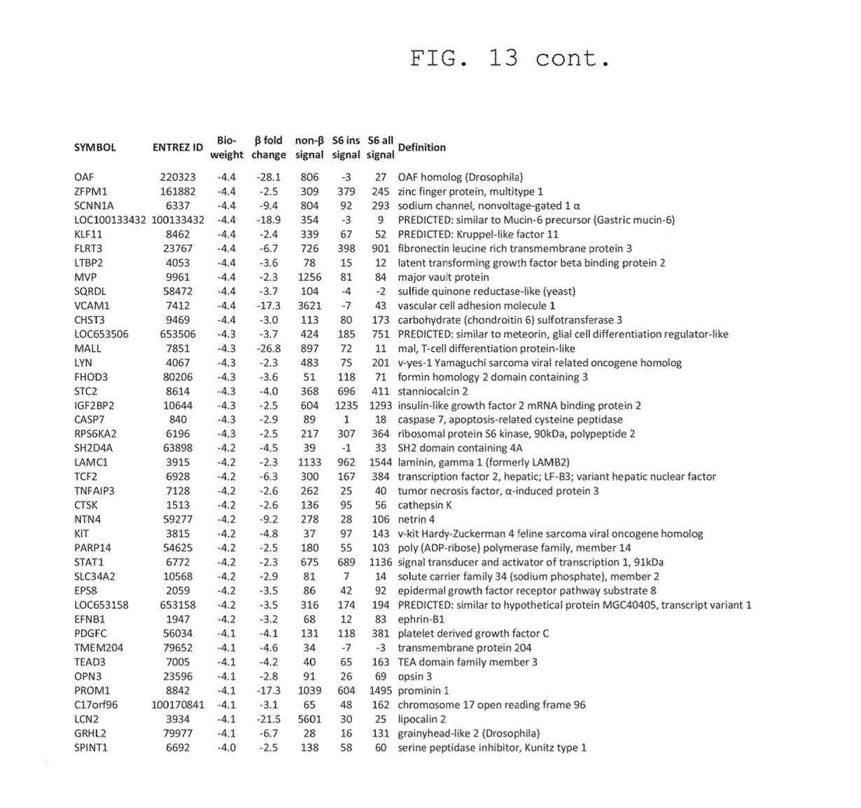

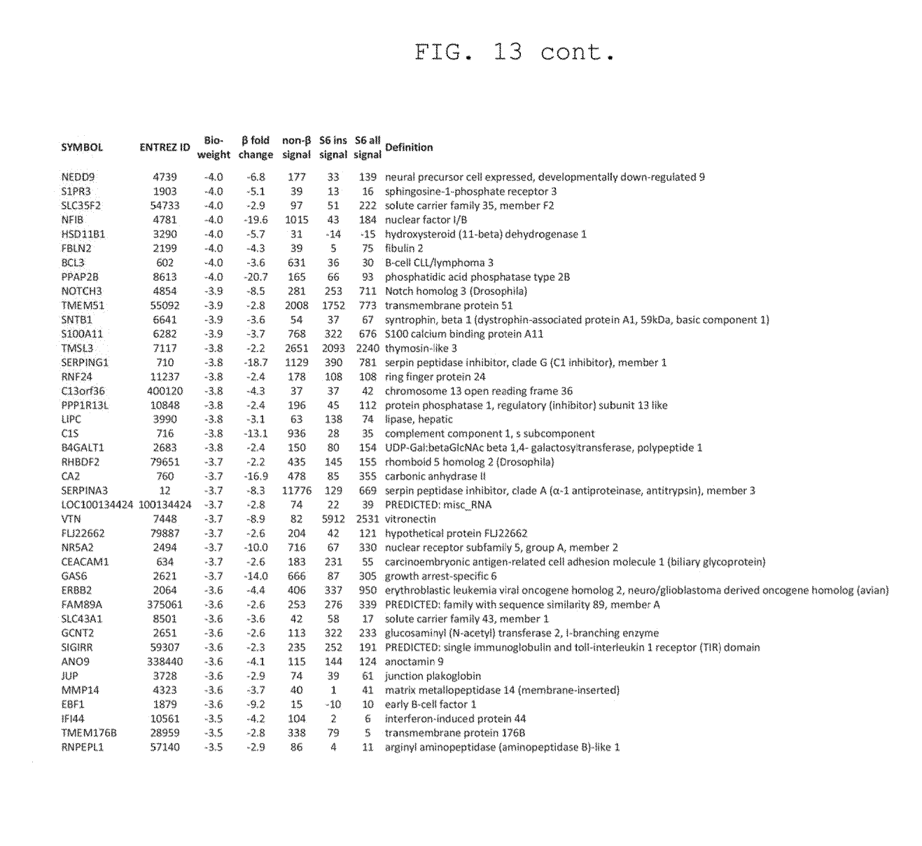

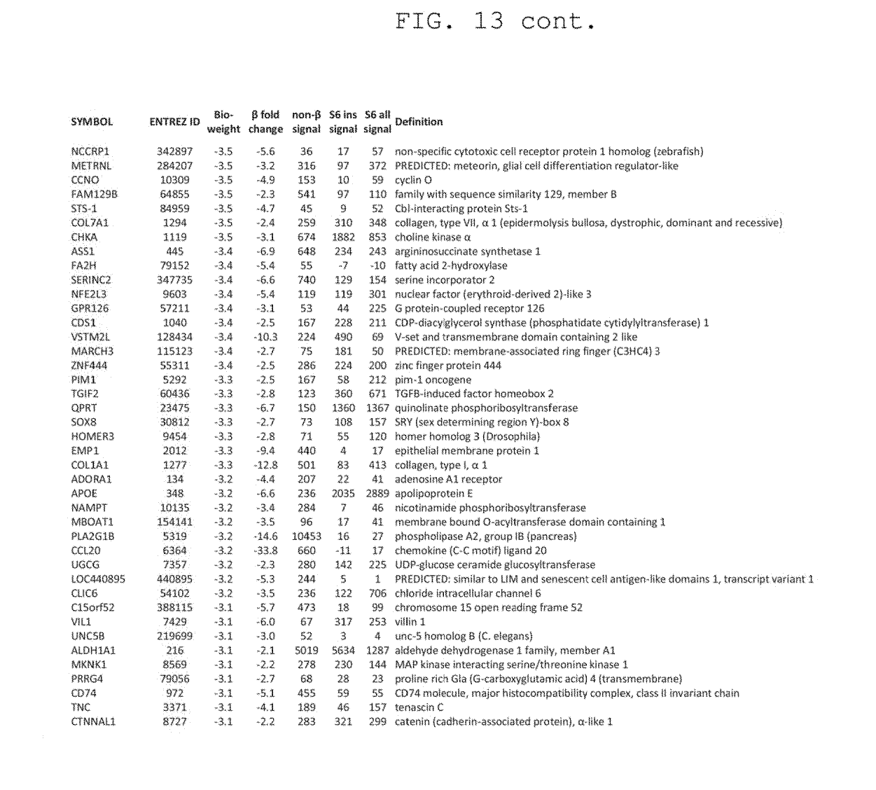

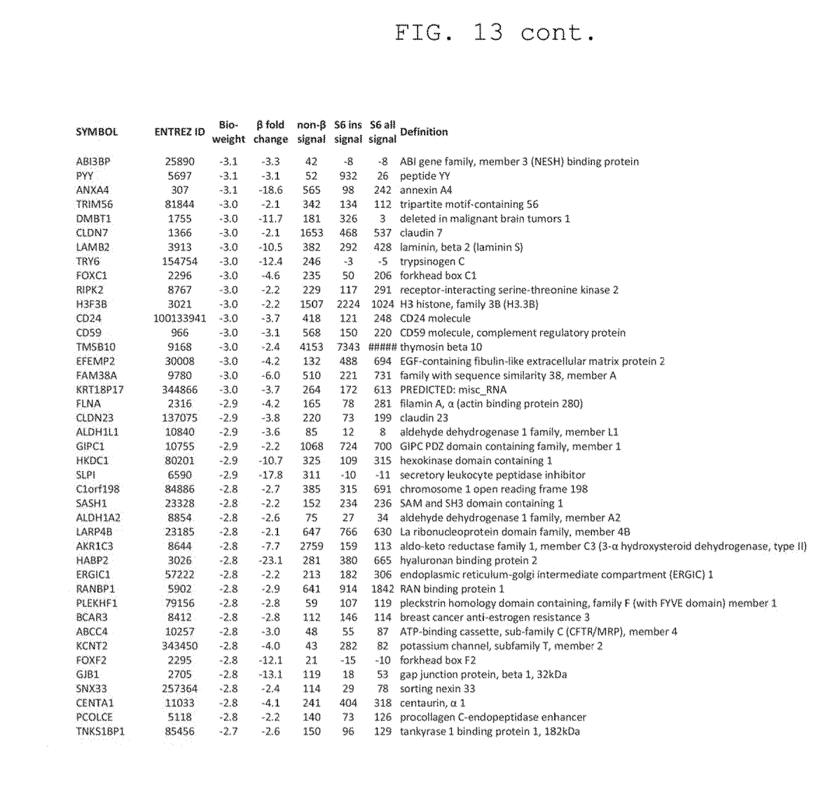

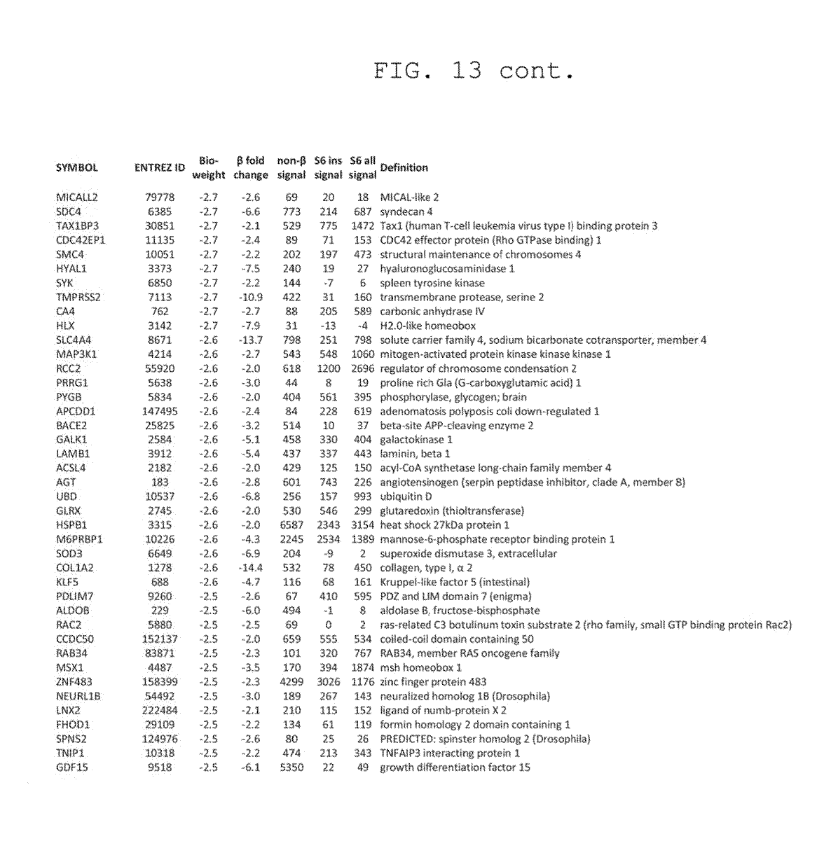

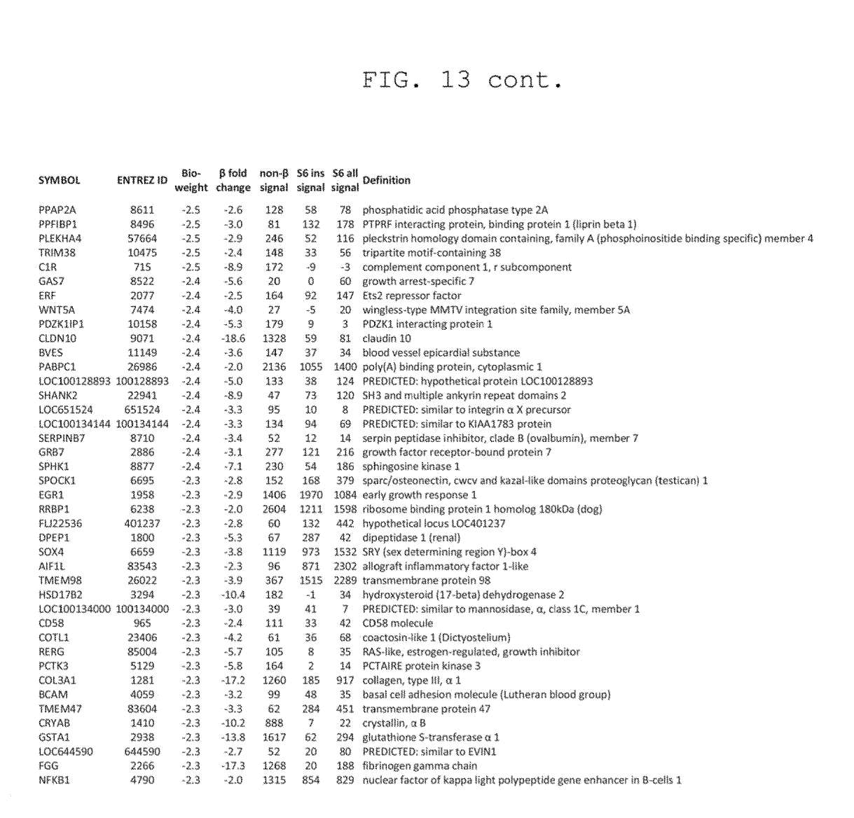

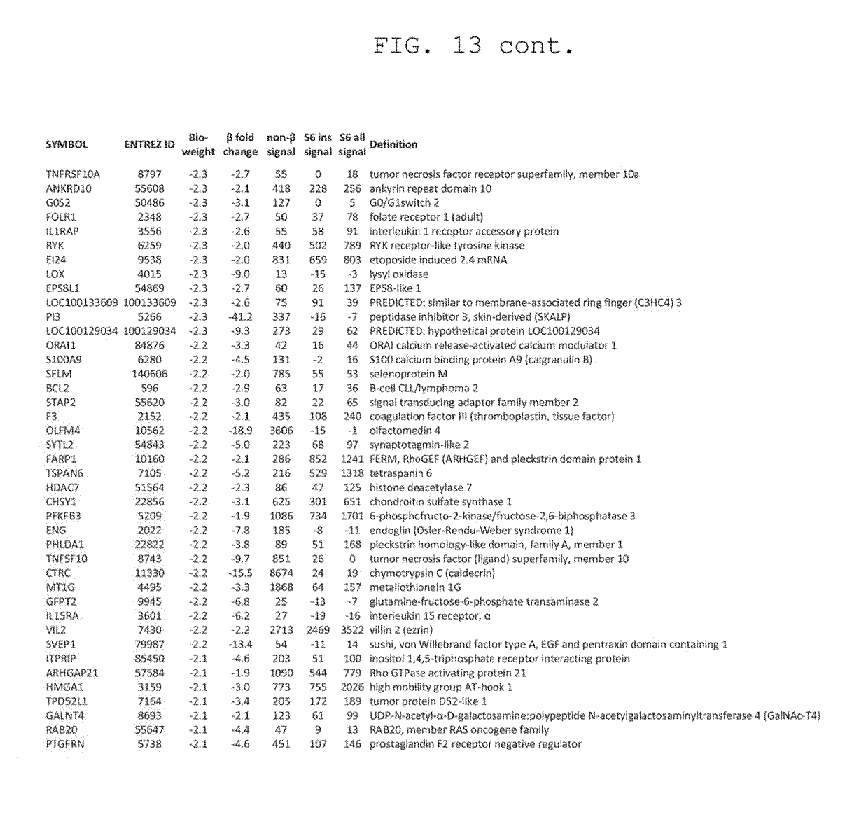

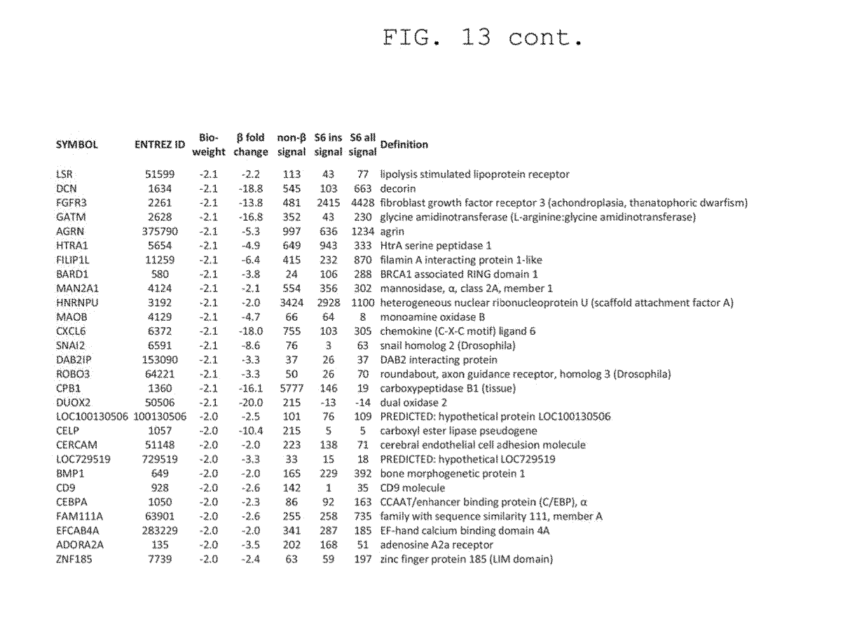

In some aspects, the invention provides a method of distinguishing β-cells and non-β-cells in a sample comprising pancreatic cells, the method comprising: (a) obtaining a sample comprising pancreatic cells; (b) measuring expression of a plurality of genes in the sample to produce a transcriptome of the pancreatic cells; (c) comparing the transcriptome of the pancreatic cells to a reference β-cell transcriptome exhibiting a pattern of expression depicted in FIG. 12 and/or a reference non-β-cell transcriptome exhibiting a pattern of expression depicted in FIG. 13; and (d) distinguishing β-cells and non β-cells, wherein (i) the pancreatic cells are β-cells if the transcriptome of pancreatic cells exhibits a pattern of expression similar to the reference β-cell transcriptome, or (ii) the pancreatic cells are non-β-cells if the transcriptome of pancreatic cells exhibits a pattern of expression similar to the reference non-β-cell transcriptome.

The present invention contemplates the use of any suitable method to measure the expression of the genes in a putative β-cell or a population of putative β-cells to produce a transcriptome. Other suitable methods will be apparent to those skilled in the art. In some embodiments of this and other aspects of the invention, measuring expression comprises utilizing a technique selected from the group consisting of a microarray analysis, RNA-seq, RT-PCR, and q-RT-PCR.

As used here, “transcriptome” refers to the collection of all gene transcripts in a and their appropriate relative abundances given cell (e.g., a β-cell), including both coding RNA (mRNAs) and non-coding RNAs (e.g., miRNA, tRNA, lincRNAs etc.).

In some embodiments the disclosure provides a mature β-cell transcriptome that may be used for a variety of purposes. In some embodiments the mature β-cell transcriptome comprises expression levels of the genes listed in FIG. 12 or counterparts thereof (e.g., orthologs in other organisms). In some embodiments measurement of expression levels of the genes or a subset thereof may be used to identify mature β-cells (e.g., as compared with immature β-cells, such as fetal β-cells or in vitro-differentiated insulin-positive β-like cells). In some embodiments a subset comprises at least 5, 10, 20, 50, 100, 200, 300, 400, or more genes listed in FIG. 12. Gene expression levels may be measured by measuring mRNA, protein or other gene product. Any suitable method may be used. In some embodiments gene expression may be measured using RNA-Seq, microarray analysis, or quantitative PCR. In some embodiments β-cells or populations of β-cells are classified based on the mature β-cell transcriptome. For example, whether the β-cell's or β-cell population's transcriptome more closely resembles a mature β-cell transcriptome or not may be determined. Heirarchical clustering or PCA analysis may be used, for example, to determine whether a particular β-cell population (e.g., colony, culture, cell line, etc.) clusters with mature β-cells as described herein or clusters with immature β-cells as described herein. Mature β-cells which substantially match the transcriptome of the mature β-cell transcriptome (e.g., that cluster with mature β-cells as described herein), may, for example, be suitable for administration in vivo. In some embodiments a mature β-cell transcriptome may be used in identifying compounds or conditions that promote formation of mature β-cells. For example, compounds or conditions may be used in a differentiation protocol and their effect on the transcriptome of pluripotent cells subjected to the differentiation protocol may be assessed. Compounds that promote a β-cell transcriptome resembling that of a mature β-cell transcriptome may be identified. Such compounds may be used in a differentiation protocol to generate β-cells (e.g., functionally mature β-cells). In some embodiments, an R2 correlation can be used to determine whether a cell is a β-cell. In such embodiments, a cell is a β-cell when compared to the β-cell transcriptome gene expression data the cells have a correlation coefficient (r) across all genes assessed r>0.9 or r>0.95 or r>0.98 or r>0.99.

As used herein, a “functionally immature β-cell” and “immature β-cell” are used interchangeably to refer to a cell that displays one or more markers of β-cell functional immaturity or does not display one or more markers of β-cell functional maturity, and lacks an appropriate GSIS response.

As used herein, “marker of β-cell maturity” and “marker of functional β-cell maturity” are used interchangeably and refer to a mature β-cell transcriptome, one or more genes having higher expression levels in mature β-cells compared to fetal β-cells or in-vitro-differentiated insulin-positive β-like cells, one or more signaling pathways which are enriched in mature β-cells compared to fetal β-cells or in vitro-differentiated insulin-positive β-like cells, or one or more biological processes which are enriched in mature β-cells compared to fetal β-cells or in-vitro-differentiated insulin-positive β-like cells.

As used herein, a “marker of β-cell immaturity” and “marker of functional β-cell immaturity” are used interchangeably to refer to one or more genes having higher expression levels in fetal β-cells or in vitro-differentiated insulin-positive β-like cells compared to mature β-cells, one or more signaling pathways which are enriched in fetal β-cells or in vitro-differentiated insulin-positive β-like cells compared to mature β-cells, or one or more biological processes which are enriched in fetal β-cells or in vitro-differentiated insulin-positive β-like cells compared to mature β-cells.

It is to be understood that the markers of β-cell maturity and markers of β-cell immaturity may be detectably expressed in the form of protein and or mRNA within a cell. Accordingly, as used herein, “protein marker of β-cell maturity” and “protein marker of β-cell functional maturity” are used interchangeably to refer to a protein encoded by a gene which is a marker of β-cell maturity. As used herein, “protein marker of β-cell immaturity” and “protein marker of β-cell functional immaturity” are used interchangeably to refer to a protein encoded by a gene which is a marker of β-cell immaturity. As used herein, “mRNA marker of β-cell maturity” and “mRNA marker of β-cell functional maturity” are used interchangeably to refer to mRNA encoded by a gene which is a marker of β-cell maturity. As used herein, “mRNA marker of β-cell immaturity” and “mRNA marker of β-cell functional immaturity” are used interchangeably to refer to mRNA encoded by a gene which is a marker of β-cell immaturity.

As used herein, a “functionally mature β-cell” and “mature β-cell” are used interchangeably to refer to a cell that displays one or more markers of β-cell functional maturity and exhibits an appropriate GSIS response.

The markers and methods of the present invention are capable of identifying the functional maturity of any β-cell, β-like cell, or putative β-cell. As used herein “(3-like cell” refers to a cell that displays at least two markers indicative of a pancreatic β-cell, such as, pancreas duodenum homeobox-1 (PDX-1), insulin, somatostatin, glucose transporter-2 (GLUT-2), glycogen, amylase, and neurogenin-3 (NGN-3), or genes having higher expression levels in mature β-cells. Markers indicative of pancreatic β-cells also include morphological characteristics (e.g., spherical shape). As used herein “insulin-positive β-like cell” refers to a cell that displays the at least two markers indicative of a pancreatic β-cell and also express insulin but lack an appropriate GSIS response. As used herein “in vitro-differentiated insulin-positive β-like cell,” “S6 insulin+ cells,” “hPSC-ins+ cells,” HUE28-ins+ cells,” and “hESC-derived stage 6 (s6) insulin+ cells” are used interchangeably to refer to β-like cells that are positive for insulin expression, and are derived from differentiation of stem cells.

The markers and methods of the present invention are capable of distinguishing mature and immature β-cells. Generally, distinguishing between mature and immature β-cells can be accomplished by analyzing putative β-cell for one or more markers of β-cell functional maturity or one or more markers of β-cell functional immaturity. On the one hand, if a putative β-cell or a population of putative β-cells displays one or more markers of β-cell functional maturity, then the β-cell or population of β-cells are likely mature β-cells. On the other hand, if a putative β-cell or a population of putative β-cells displays one or more markers of β-cell functional maturity, then the β-cell or population of β-cells are likely immature β-cells. Those skilled in the art will appreciate that confirmation of β-cell functional maturity or functional immaturity can be done by performing a GSIS assay to determine if the putative β-cell or the population of putative β-cells exhibits or lacks an appropriate GSIS response, respectively.

### Genes Differentially Expressed Between Mature and Immature β-Cells

The present invention contemplates distinguishing mature and immature β-cells by detecting the presence or absence of expression of one or more genes which are differentially expressed between mature and immature β-cells in a β-cell, population of β-cells, a putative β-cell, or a population of putative β-cells.

In some aspects, the present invention provides a method of detecting mature β-cells, the method comprising conducting at least one binding assay for at least one marker of β-cell functional maturity in a cell or a population of cells, wherein the presence of the at least one marker of β-cell functional maturity in the cell or a population of cells indicates that the cell or population of cells are mature β-cells.

The present invention contemplates detecting mature β-cells by detecting the presence of any marker of β-cell functional maturity in a cell or a population of cells. The cell or population of cells can be a cell or population of cells suspected of being β-cells (e.g., a culture differentiating stem cells). In some embodiments, the at least one marker of β-cell functional maturity is selected from the group consisting of the genes listed in Table 1 or Table 3. In some embodiments, the at least one marker of β-cell functional maturity comprises a protein marker of β-cell functional maturity, as described herein. In some embodiments, the at least one marker of β-cell functional maturity comprises an mRNA marker of β-cell functional maturity, as described herein.

In some aspects, the present invention provides a method of detecting immature β-cells, the method comprising conducting at least one binding assay for at least one marker of β-cell functional immaturity in a cell or population of cells, wherein the presence of the at least one marker of β-cell functional immaturity in the cell or a population of cells indicates that the cell or population of cells are immature β-cells.

The present invention contemplates detecting immature β-cells by detecting the presence of any marker of β-cell functional immaturity in a cell or a population of cells. The cell or population of cells can be a cell or population of cells suspected of being β-cells (e.g., a culture differentiating stem cells).

In some embodiments, the at least one marker of β-cell functional immaturity is selected from the group consisting of the genes listed in Table 3 or Table 4. In some embodiments, the at least one marker of β-cell functional immaturity comprises a protein marker of β-cell functional immaturity. In some embodiments, the at least one marker of β-cell functional immaturity comprises an mRNA marker of β-cell functional immaturity.

Generally, the presence of a protein marker of β-cell functional maturity in a β-cell or population of β-cells is indicative that the β-cell or population of β-cells is functionally mature, whereas the absence of the same protein marker in the β-cell or population of β-cells may be indicative that the β-cell or population of β-cells is functionally immature. Conversely, the presence of a protein marker of β-cell functional immaturity in a β-cell or population of β-cells is indicative that the β-cell or population of β-cells is functionally immature, whereas the absence of the same protein marker in the β-cell or population of β-cells may be indicative that the β-cell or population of β-cells is functionally mature.

The present invention contemplates detecting the presence or absence of protein markers of β-cell functional maturity or immaturity according to any technique available to the skilled artisan. In some embodiments of this and other aspects of the invention, detecting the presence or absence of protein markers of β-cell functional maturity or β-cell functional immaturity comprises immunostaining (e.g., Western blotting, immunohistochemistry, ELISA, etc). In such embodiments, anti-protein marker antibodies targeted to a particular protein marker of β-cell functional maturity or β-cell functional immaturity can be used to detect the presence or absence of the particular protein marker. For the purposes of the invention the immunostaining techniques described or mentioned herein are considered binding assays.

Such antibodies can include polyclonal antibodies, monoclonal antibodies, chimeric antibodies, single-chain antibodies, antibody fragments, humanized antibodies, multi-specific antibodies, and modified antibodies (e.g., fused to a protein to facilitate detection.) Suitable anti-marker protein antibodies can be generated according to routine protocols, or can be readily obtained from a variety of commercial sources (e.g., Sigma-Aldrich). Other suitable techniques for detecting the presence of proteins in β-cells are apparent to those skilled in the art.

Generally, the presence of elevated levels of mRNA markers of β-cell functional maturity in a β-cell or population of β-cells is indicative that the β-cell or population of β-cells is functionally mature, whereas the absence of the same mRNA markers (or relative lower levels of the same mRNA markers) in the β-cell or population of β-cells may be indicative that the β-cell or population of β-cells is functionally immature. Conversely, the presence of elevated levels of mRNA markers of β-cell functional immaturity in a β-cell or population of β-cells is indicative that the β-cell or population of β-cells is functionally immature, whereas the absence of the same mRNA markers (or relative lower levels of the same mRNA markers) in the β-cell or population of β-cells may be indicative that the β-cell or population of β-cells is functionally mature.

It is to be understood that the phrase “elevated levels of mRNA” refers to levels of mRNA in a mature β-cell relative to an immature β-cell or levels of mRNA in an immature β-cell relative to a mature β-cell. Elevated levels of mRNA may be represented as a fold-change in expression of the mRNA in the mature β-cell relative to the immature β-cell, and vice versa.

The present invention contemplates detecting the presence or absence of elevated levels of mRNA according to any technique available to the skilled artisan. In some embodiments of this and other aspects of the invention, detecting the presence or absence of elevated levels of mRNA markers of β-cell functional maturity or immaturity in a β-cell or population of β-cells comprises conducting one or more hybridization assays. In some embodiments of this and other aspects of the invention, the one or more hybridization assays comprise a microarray. In some embodiments of this and other aspects of the invention, the one or more hybridization assay comprises RNA-seq. In some embodiments of this and other aspects of the invention, the one or more hybridization assays comprises q-RT-PCR. For the purposes of the invention the hybridization assays described or mentioned herein are considered binding assays.

In some embodiments of this and other aspects of the invention, the presence of elevated levels of mRNA markers of β-cell functional maturity comprises at least a 2 fold increase, a 3 fold increase, a 4 fold increase, a 5 fold increase, or N-fold increase (where N is a positive integer) in the levels of the mRNA marker in a mature β-cell relative to the levels of the mRNA maker in immature β-cells.

In some embodiments of this and other aspects of the invention, the presence of elevated levels of mRNA markers of β-cell functional immaturity comprises at least a 2 fold increase, a 3 fold increase, a 4 fold increase, a 5 fold increase, or N-fold increase (where N is a positive integer) in the levels of the mRNA marker in an immature β-cell relative to the levels of the mRNA marker in mature β-cells.

In some embodiments of this and other aspects of the invention, the marker of β-cell functional immaturity does not include PDX1. In some embodiments of this and other aspects of the invention, the marker of β-cell functional immaturity does not include MAFB. In some embodiments of this and other aspects of the invention, the marker of β-cell functional immaturity does not include NKX6.1.

In some embodiments of this and other aspects of the invention, the marker of β-cell functional maturity does not include MAFA. In some embodiments of this and other aspects of the invention, the marker of β-cell functional maturity does not include NKX2.2. In some embodiments of this and other aspects of the invention, the marker of β-cell functional maturity does not include UCN3.

In some aspects, a marker of β-cell functional maturity includes one, any combination or sub-combination, or all genes having a higher expression level in mature β-cells compared to fetal β-cells or in vitro-differentiated insulin-positive β-like cells.

Exemplary genes having higher expression levels in mature β-cells compared to fetal β-cells are listed in Table 1 (Table 1 includes both Table 1A and 1B).

TABLE 1A
Genes with higher expression levels in mature
β-cells compared to fetal β-cells

CHGB, IAPP, ESR1, HES1, SOX9, ALDOA, ATP6V0E1, GPI, PDK3,
SYT4, G6PC2, SLC2A2, KCNK1, KCNK12, KCNK3, CDH2, WNT4,
KLF9, EPAS1, BHLHB3, HOPX, MESP1, BCL6, TP53, SIX2, ETV5,
SIX4, DDIT3, NR3C2, PBX3, STAT3, NPAS2, STAT4

TABLE 1B
Genes with higher expression levels in mature β-cells compared to fetal β-cells

AADACL1, AARS, ABHD5, ABHD6, ABLIM1, ACAA2, ACAT1, ACBD3,
ACSL1, ACTN1, ADCY9, ADIPOR1, ADM, ADSSL1, AFTPH, AGA, AGPAT9,
AIDA, AIG1, AK2, AKT3, AKTIP, ALCAM, ALDH1L2, ALDOA, ALG5,
ALKBH3, AMPH, AMZ2, ANGPTL2, ANKMY2, ANKRD12, ANKRD23,
ANKRD40, ANKRD43, ANKRD57, ANTXR1, ANXA7, APITD1, APOA1BP,
ARF4, ARHGEF3, ARL1, ARL8B, ARPC1A, ARX, AS3MT, ASNSD1, ATF1,
ATG3, ATL1, ATP1B2, ATP6V0B, ATP6V0E1, ATP6V1D, ATP6V1E1,
ATP6V1E2, ATP6V1G1, ATP6V1H, ATXN1, B2M, B3GNT6, B3GNT9, BAG3,
BCL2L13, BCL2L2, BCL6, BEX2, BEX5, BHLHB2, BHLHB3, BMI1, BMP5,
BOK, BOLA3, BPIL2, BRMS1, BRP44L, BTG3, BUD31, C10orf10, C10orf39,
C11orf1, C11orf10, C11orf52, C11orf75, C12orf57, C12orf62, C13orf1, C13orf27,
C14orf109, C14orf112, C14orf149, C14orf156, C14orf72, C15orf24, C15orf48,
C15orf59, C16orf42, C16orf45, C17orf58, C17orf71, C17orf79, C18orf8, C19orf10,
C19orf70, C1orf128, C1orf133, C1orf174, C1orf25, C1orf41, C1orf43, C1orf57,
C1orf66, C1orf97, C21orf124, C21orf63, C22orf25, C2CD4B, C2orf28, C2orf30,
C2orf32, C2orf42, C2orf7, C2orf76, C3orf14, C4orf34, C5orf32, C5orf53, C6orf64,
C6orf89, C7orf23, C7orf30, C7orf42, C7orf52, C8orf40, C8orf59, C8orf76,
C9orf103, C9orf119, C9orf9, C9orf91, CA5B, CAB39, CALCB, CAMK2N2,
CAMTA1, CAPN13, CARS, CBX7, CCDC104, CCDC109B, CCDC28A, CCKBR,
CCND3, CCNG1, CCPG1, CCT6B, CD2BP2, CD55, CD59, CD63, CD99, CD99L2,
CDC14B, CDCP1, CDH18, CDH2, CDK5R2, CDKN1B, CDR2, CEBPG, CGRRF1,
CHCHD10, CHCHD2, CHD5, CHDH, CHGB, CHPF, CHST1, CIB1, CKAP4,
CLDN12, CLGN, CLIP4, CLN5, CMAS, CMBL, CMC1, CNIH4, CNPY2, CNRIP1,
COPB1, COPS7A, COQ10A, COQ10B, COQ3, COX17, COX6B1, COX6C,
COX7A1, COX7A2, COX7B, CPE, CPLX1, CPNE8, CRB3, CRY1, CRYZ, CSTB,
CTBS, CTSL2, CUTC, CWC15, CXorf39, CXorf57, CYB561D1, CYB5R1,
CYB5R4, CYP27A1, DAZAP2, DBI, DBNDD2, DCLRE1A, DCUN1D4, DDIT3,
DEGS1, DGCR11, DGCR5, DHRS2, DHRS4, DHRS7B, DHX32, DIAPH1, DIRC2,
DISP2, DKFZp686O24166, DKK4, DLG2, DMKN, DNAI1, DNAJB9, DNAJC12,
DNAJC14, DNHD1, DNM3, DPM2, DPM3, DPYSL4, DRAM1, DUSP23,
DYNC1I1, DYRK4, ECE2, EDEM1, EFHD1, EHBP1, EIF1AY, EIF2B3, EIF3I,
EIF4E3, ELF1, ELMO1, ELMOD2, EMG1, ENAM, ENPP2, ENPP4, ENPP5,
ENTPD3, EPAS1, EPB41L4B, EPDR1, EPSTI1, ERO1L, ERRFI1, ESYT1, ETFA,
ETV5, EXOC5, EXOD1, F2RL1, FADS1, FAF1, FAM102A, FAM104A, FAM107A,
FAM119B, FAM159B, FAM167A, FAM174A, FAM176A, FAM181B, FAM190B,
FAM27A, FAM58A, FAM59A, FAM69A, FAM80A, FAM91A1, FAM96B, FAP,
FBXL14, FBXL16, FBXO34, FBXO6, FBXO8, FHL1, FICD, FKBP11, FKBP2,
FLJ20273, FLJ41603, FLJ43752, FLVCR2, FNDC3B, FOXN2, FRG1, FUCA1,
FVT1, FYCO1, G3BP2, G6PC2, GABARAPL2, GAD1, GAD2, GAPDH, GARS,
GAS2, GBE1, GC, GCNT3, GEM, GFPT1, GHDC, GHITM, GLO1, GLRB, GLRX,
GLRX2, GLS2, GLT25D2, GLT8D2, GLTP, GMPR, GNE, GNPDA1, GOLGA4,
GOLGA5, GOLPH3, GOLSYN, GOLT1A, GORASP2, GPI, GPM6A, GPR137B,
GPR177, GPR19, GPR3, GPX3, GPX4, GRAMD1C, GREM2, GRIA3, GSN,
GSTO1, GTF2E2, GTF2IRD1, GUCY1A3, GUCY1B3, GYG1, HABP4, HACL1,
HAPLN4, HAX1, HDDC3, HELQ, HERC5, HERC6, HEXB, HINT1, HIVEP2,
HLA-A29.1, HMGN4, HOPX, HPCAL4, HPRT1, HRASLS3, HS2ST1, HS6ST2,
HSD17B12, HSPA4L, HSPA9, HSPC157, HSPC171, HTATIP2, IAH1, IAPP,
ICAM2, IDI1, IER3, IER3IP1, IFI27L2, IFI35, IFI6, IFIH1, IFIT1, IFIT5, IFNAR2,
IFNGR1, IFNGR2, IGFBP4, IL13RA1, IL17RB, IL20RA, INA, ING2, INPP1,
INPP4B, INSIG2, IRAK2, IRAK4, IRS2, ISCU, ISG20L1, ITFG1, ITGA10, ITM2B,
ITPR3, JAGN1, JAZF1, JPH3, JTB, KBTBD10, KCNF1, KCNJ16, KCNJ8, KCNK1,
KCNK3, KCTD13, KCTD14, KCTD8, KDELR2, KIAA0247, KIAA0367,
KIAA0408, KIAA0494, KIAA0672, KIAA1107, KIAA1128, KIAA1324,
KIAA1644, KLF9, KLHDC8A, KLHL5, KPNA4, KPNA6, KRT222, LACTB2,
LAMP1, LAMP2, LAMP3, LAPTM4B, LCMT2, LDLR, LDLRAP1, LGALS8,
LGI2, LGI3, LHFPL4, LIMCH1, LIN7B, LIPL3, LMAN1, LMBRD1, LMNA,
LMTK2, LOC100128353, LOC100128731, LOC100129673, LOC100129759,
LOC100130633, LOC100131261, LOC100131531, LOC100131785,
LOC100131801, LOC100133273, LOC100134537, LOC100144604, LOC133993,
LOC145853, LOC147727, LOC154761, LOC203547, LOC283481, LOC284988,
LOC285412, LOC387882, LOC388789, LOC389293, LOC389791, LOC390530,
LOC391075, LOC391578, LOC399744, LOC400948, LOC440043, LOC440487,
LOC440737, LOC440957, LOC442454, LOC643310, LOC643320, LOC643740,
LOC644237, LOC644310, LOC644683, LOC644761, LOC645058, LOC645313,
LOC645586, LOC645609, LOC646135, LOC646723, LOC646900, LOC647037,
LOC647302, LOC647307, LOC648399, LOC648638, LOC648659, LOC649456,
LOC650200, LOC650215, LOC650254, LOC650392, LOC650803, LOC651143,
LOC652607, LOC652882, LOC653071, LOC653566, LOC727768, LOC728032,
LOC728178, LOC728431, LOC728635, LOC729314, LOC729317, LOC729768,
LOC731365, LOC732007, LOC732391, LOC88523, LPPR2, LRP10, LRP11,
LRRC36, LRRFIP2, LSAMP, LSM1, LSM10, LSM3, LSMD1, LYPLAL1, LYRM1,
LYSMD2, LZIC, MAD2L2, MAMDC2, MAN1A2, MAN1C1, MAN2B2, MANSC1,
MAP1LC3B, MAP2K1IP1, MAP3K6, MAP6D1, MAPK9, MAPRE2, MARK1,
MDH1, MDH2, ME1, MEA1, MEIS3, MEIS3P1, MESP1, METTL5, MFSD6,
MGAT4A, MGC16291, MGST2, MGST3, MID2, MIR129-2, MKKS, MKRN2,
MLF1, MMGT1, MMP1, MMP7, MOCS1, MOSC2, MPP5, MPV17, MR1, MRAP2,
MRPL14, MRPE18, MRPL20, MRPL33, MRPL34, MRPL36, MRPL40, MRPL53,
MRPS18C, MRPS23, MRPS28, MRPS33, MSI2, MST4, MT1X, MTCP1, MTHFD2,
MTMR11, MTP18, MVP, MX1, MYL12A, MYO1D, MYOM1, N6AMT2, NAB1,
NANS, NARS, NAT5, NBL1, NCALD, NCRNA00200, NDEL1, NDFIP1,
NDUFA1, NDUFAB1, NDUFAF1, NDUFAF2, NDUFB3, NDUFB9, NDUFC1,
NEBL, NEIL2, NEK1, NFE2L1, NFE2L2, NIPA1, NKIRAS1, NLF2, NLGN1,
NLGN4Y, NMB, NMD3, NNMT, NOL3, NOL7, NOSTRIN, NPAS2, NPC1, NPM2,
NPR2, NPTX2, NPY, NQO1, NQ02, NR0B1, NR2C2AP, NR3C2, NRD1, NUAK1,
NUCB2, NUDT18, NUDT22, NUDT5, NUDT7, NUP88, OAT, OBFC1, OCEL1,
OLFM1, OLIG1, OPTN, ORC3L, ORMDL2, OSBPL10, OSGIN2, OSTM1, OVOL2,
OXCT2, OXGR1, P4HA2, PAK3, PALLD, PAM, PAPSS2, PARM1, PBX3, PCBP1,
PCBP3, PCGF1, PCK1, PDE12, PDGFRL, PDK3, PDK4, PDLIM1, PDXK, PDZD8,
PEA15, PEBP1, PELI1, PELO, PERP, PGAM1, PGCP, PGM1, PGM3, PGRMC2,
PHF1, PHF11, PHF15, PHF17, PHF21B, PHLDA3, PIGH, PIGP, PIGT, PIGY, PIR,
PITPNC1, PKIB, PLA2G4C, PLCB1, PLCL2, PLK2, PLOD2, PLS1, PNLIPRP2,
PNMA2, PNPO, POLE3, POLR1D, PON3, PPA1, PPBP, PPCS, PPM1E, PPM1H,
PPP1R1A, PPP1R3D, PPP2CB, PPP2R2C, PPP2R5A, PPP3CC, PPT1, PRAMEF19,
PRDX1, PRDX4, PRICKLE4, PRKCA, PRNP, PROCR, PROS1, PROSC, PRR13,
PRRT1, PRSS23, PRUNE2, PSMA6, PSMB1, PSMB4, PSMB6, PSMC3, PSMD8,
PSMG1, PSMG2, PTGS2, PTPN11, PTPN3, PTPRN, PURA, PYROXD1, QPCT,
RAB11FIP5, RAB1A, RAB21, RAB24, RAB2A, RAB31, RAB39B, RAB7A,
RABAC1, RABGGTB, RABL3, RALYL, RAPH1, RASD1, RBBP8, RBM47,
RCAN2, RCBTB2, REEP1, RFC4, RFESD, RGS17, RGS22, RGS4, RHEB, RICH2,
RIN2, RIOK2, RIPPLY2, RLTPR, RNASEK, RNASEL, RNF130, RNF145,
RNF181, RNF187, RPA3, RPH3AL, RPL26, RPL36AL, RPL41, RPP25, RPS19BP1,
RPS27L, RQCD1, RSAD1, RSRC1, RTCD1, RTN4, SAMD3, SAMD4B, SAT2,
SBDS, SC5DL, SCG2, SCG3, SCG5, SCGB2A1, SDC2, SDC4, SDF2, SDF2L1,
SDHB, SDSL, SEC11C, SEC22A, SEC22B, SEC61B, SEC61G, SEL1L3, SELK,
SELM, SEMA5A, SEPHS2, SEPW1, SERF2, SERINC1, SERINC3, SERPINB1,
SERTAD4, SESN1, SEZ6L, SFMBT1, SFT2D1, SGCE, SGIP1, SGK3, SGMS2,
SH3GLB1, SIPA1L2, SIX2, SIX4, SLBP, SLC12A8, SLC17A6, SLC1A4,
SLC25A4, SLC2A10, SLC2A12, SLC2A13, SLC31A2, SLC35A5, SLC35D3,
SLC35F3, SLC39A1, SLC39A8, SLC41A1, SLC45A3, SLC46A3, SLC6A17,
SLC6A6, SLC7A1, SLC7A5, SLC8A2, SLC8A3, SMAP1, SMPDL3A, SNAP91,
SNCB, SNPH, SNRPG, SOD1, SORL1, SPATA18, SPCS1, SPCS2, SPCS3, SPG3A,
SPIRE1, SPPL2A, SPR, SPTBN4, SQSTM1, SRD5A1, SRP54, SRPRB, SRXN1,
STAMBPL1, STAT3, STAT4, STEAP2, STIM1, STXBP5, STXBP6, STYXL1,
SUCLG2, SUMO3, SURF1, SURF2, SUSD4, SYNGR3, SYNGR4, SYPL1, SYS1,
SYT13, SYT4, T1560, TACC1, TAF12, TANC2, TAP1, TARS, TAX1BP1,
TBC1D15, TBC1D22A, TBC1D8, TBC1D8B, TBCA, TCEAL2, TCEAL3,
TCEAL6, TCEB1, TDRD7, TGFBR3, THEM2, THOC7, TICAM2, TIMM10,
TIMP2, TM9SF2, TMBIM1, TMBIM6, TMCO3, TMCO4, TMED10P, TMED2,
TMED3, TMED7, TMEM106C, TMEM109, TMEM111, TMEM116, TMEM126B,
TMEM130, TMEM133, TMEM144, TMEM147, TMEM14A, TMEM14B,
TMEM14D, TMEM163, TMEM166, TMEM167A, TMEM167B, TMEM208,
TMEM219, TMEM27, TMEM30B, TMEM38B, TMEM4, TMEM54, TMEM59,
TMEM60, TMEM66, TMEM70, TMEM99, TMOD1, TncRNA, TNFRSF11A,
TNFRSF21, TOMM5, TOMM7, TP53, TRAM1, TRAPPC2P1, TRAPPC4, TSGA14,
TSHZ3, TSPAN13, TSTD1, TTC1, TTC39B, TTC39C, TUFT1, TXN, TXNDC11,
TXNDC17, TXNDC9, TXNL1, UAP1L1, UBA5, UBE2D3, UBE2D4, UBE2E2,
UBL3, UBTD1, UCHL5, UFM1, UFSP2, UGDH, UNC50, UNC5CL, UQCRB,
UQCRQ, USO1, UTP11L, VAMP2, VAMP4, VAV3, VCPIP1, VGF, VLDLR,
VPS37A, VPS37C, VTI1B, VWA5A, WAC, WASL, WBSCR27, WDR25, WDR5B,
WDYHV1, WNT4, WSB2, WWC1, XBP1, YARS, ZC3H12C, ZCWPW2, ZMYM6,
ZNF143, ZNF226, ZNF252, ZNF331, ZNF385D, ZNF395, ZNF540, ZNF83, ZNFX1

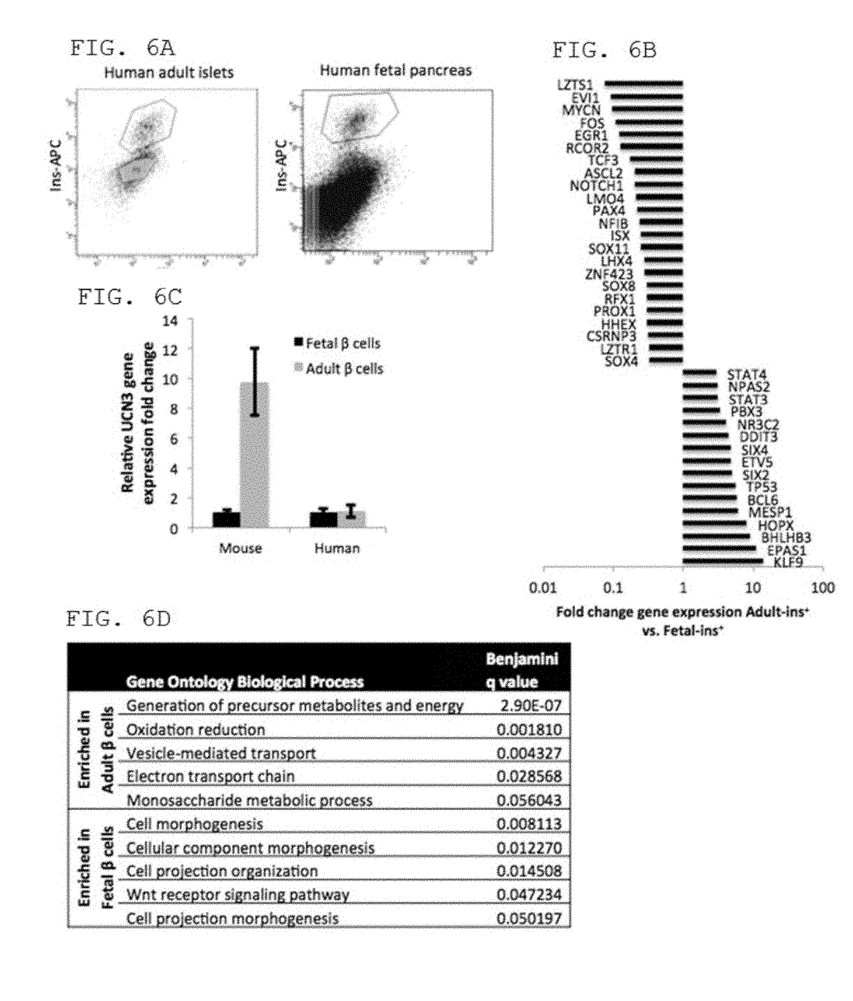

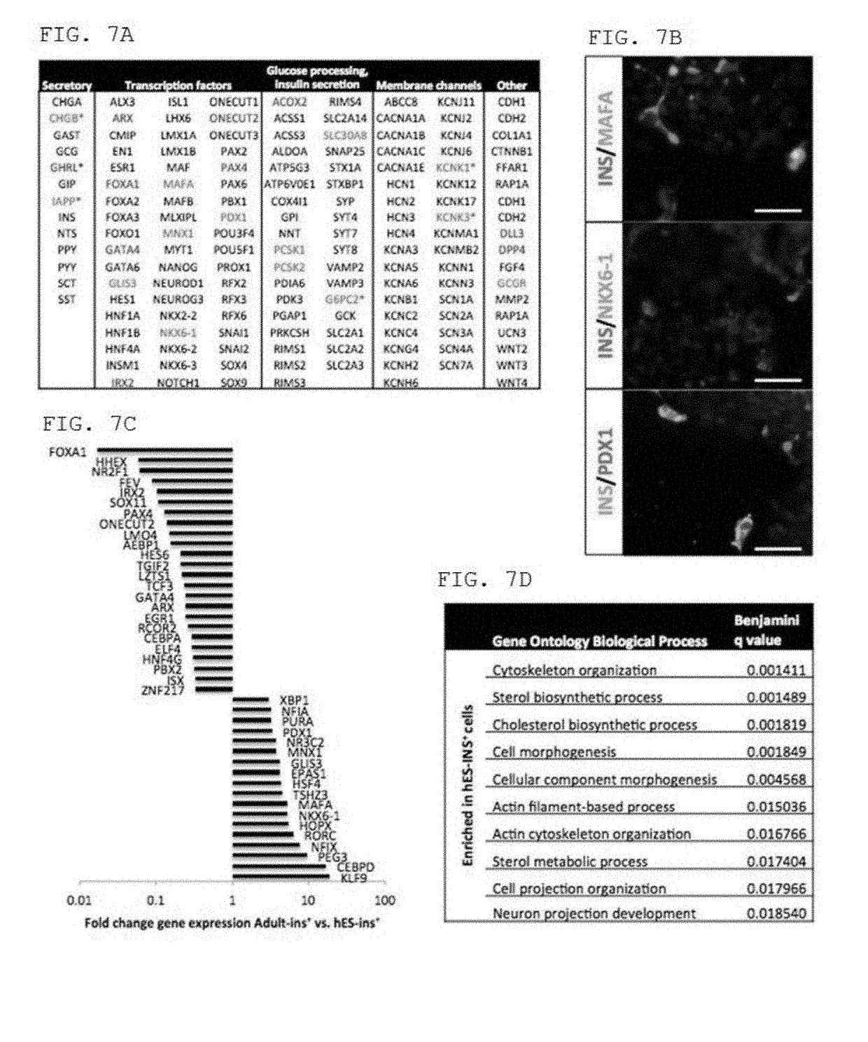

It is to be understood that the levels of expression of one or more of the genes listed in Table 1 are depicted in FIG. 6C as a fold change in expression of mature β-cells compared to fetal β-cells.

It should also be appreciated that any gene listed in Table 1 can be used as a marker for detecting mature β-cells by measuring the level of expression of the gene in a cell, culture, cell line, tissue, or population of cells (e.g., suspected of being β-cells), wherein if the level of expression of the gene in the cell, culture, cell line, tissue, or population of cells is elevated (for example, as depicted in FIG. 6C), the cell, culture, cell line, tissue, or population of cells comprises mature β-cells.

Those skilled in the art will also appreciate that any two or more of the genes listed in Table 1 can be used in combinations of up to N genes (where N is a positive integer greater than or equal to 2) as markers for detecting mature β-cells by measuring the levels of expression of the combination of genes in a cell, culture, cell line, tissue, or population of cells (e.g., suspected of being β-cells), wherein if the levels of expression of the combination of genes in the cell, culture, cell line, tissue, or population of cells is elevated (for example, as depicted in FIG. 6C), the cell, culture, cell line, tissue, or population of cells comprises mature β-cells.

Exemplary genes which have higher expression levels in mature β-cells compared to in vitro-differentiated insulin-positive β-like cells are listed in Table 2 (Table 2 includes both Tables 2A and 2B).

TABLE 2A
Genes with higher expression levels in mature β-cells compared
to in vitro-differentiated insulin-positive β-like cells

KLF9, CEBPD, PEG3, NFIX, RORC, HOPX, TSHZ3, HSF4,
EPAS1, GLIS3, NR3C2, SIX4, PURA, NFIA, XBP1, CHGB,
IAPP, ESR1, GLIS3, MAFA, MNX1, NKX6-1, PDX1, GPI, PCSK1,
PCSK2, SLC30A8, STX1A, STXBP1, SYT4, G6PC2, SLC2A2,
KCNA5, KCNH2, KCNK1, KCNK12, KCNK3, KCNMA1, GCGR,
UCN3, WNT4

TABLE 2B
Genes with higher expression levels in mature β-cells compared
to in vitro-differentiated insulin-positive β-like cells

A2LD1, A2M, A4GNT, AADAC, AADACL1, AAED1, AANAT, AARS, AARSD1,
AASDH, AB019439.68, AB019441.29, ABCA10, ABCA13, ABCA17P, ABCA5,
ABCA6, ABCA8, ABCA9, ABCA9-AS1, ABCB1, ABCB10P1, ABCB10P4,
ABCB11, ABCB7, ABCC12, ABCC2, ABCC3, ABCC9, ABCD2, ABCE1, ABCG1,
ABCG8, ABHD1, ABHD10, ABHD11-AS1, ABHD2, ABHD3, ABHD5, ABHD6,
ABI3, ABP1, ABRACL, AC000036.4, AC000110.1, AC000124.1, AC002056.3,
AC002056.5, AC002064.5, AC002128.4, AC002306.1, AC002310.10, AC002314.2,
AC002398.12, AC002398.9, AC002400.1, AC002429.5, AC002456.2, AC002467.5,
AC002486.2, AC002511.2, AC002511.4, AC002519.5, AC002519.6, AC002553.1,
AC003087.4, AC003092.2, AC003101.1, AC003104.1, AC003989.3, AC003991.3,
AC004014.4, AC004019.10, AC004041.2, AC004069.2, AC004074.3, AC004156.3,
AC004159.1, AC004240.2, AC004381.7, AC004383.3, AC004461.4, AC004471.9,
AC004485.3, AC004528.4, AC004562.1, AC004696.2, AC004744.3, AC004771.1,
AC004797.1, AC004832.1, AC004837.5, AC004840.8, AC004851.1, AC004854.4,
AC004854.5, AC004870.5, AC004876.1, AC004878.7, AC004899.3, AC004941.3,
AC004945.1, AC004988.1, AC005027.3, AC005037.1, AC005062.2, AC005076.5,
AC005077.8, AC005082.12, AC005102.1, AC005229.1, AC005255.1, AC005262.4,
AC005264.2, AC005280.1, AC005281.2, AC005307.1, AC005307.3, AC005336.2,
AC005336.5, AC005351.1, AC005355.1, AC005356.1, AC005391.2, AC005481.5,
AC005498.3, AC005517.3, AC005532.5, AC005534.9, AC005546.2, AC005616.2,
AC005682.7, AC005740.3, AC005779.2, AC005783.1, AC005884.1, AC005895.3,
AC006001.1, AC006019.3, AC006019.4, AC006026.10, AC006026.13, AC006026.9,
AC006033.22, AC006038.4, AC006042.8, AC006070.11, AC006116.12,
AC006116.13, AC006116.15, AC006116.17, AC006116.21, AC006116.24,
AC006116.26, AC006157.2, AC006227.1, AC006296.3, AC006355.3, AC006369.2,
AC006378.2, AC006378.3, AC006460.2, AC006465.3, AC006483.5, AC006486.1,
AC006509.7, AC006539.1, AC006946.12, AC007000.11, AC007009.1, AC007009.2,
AC007014.1, AC007041.2, AC007098.1, AC007099.2, AC007126.1, AC007204.2,
AC007229.3, AC007246.3, AC007249.3, AC007253.1, AC007255.7, AC007255.8,
AC007271.3, AC007272.3, AC007277.3, AC007279.2, AC007282.6, AC007283.4,
AC007283.5, AC007318.5, AC007319.1, AC007349.5, AC007362.1, AC007362.3,
AC007365.3, AC007386.3, AC007386.4, AC007389.4, AC007390.5, AC007390.6,
AC007392.3, AC007392.4, AC007405.2, AC007567.1, AC007618.3, AC007620.3,
AC007682.1, AC007731.1, AC007750.5, AC007787.2, AC007796.1, AC007842.2,
AC007879.2, AC007879.4, AC007879.5, AC007881.1, AC007919.18, AC007952.1,
AC007966.1, AC007970.1, AC008069.2, AC008132.12, AC008147.2, AC008155.1,
AC008269.2, AC008279.1, AC008280.3, AC008427.2, AC008537.2, AC008555.2,
AC008581.1, AC008592.3, AC008592.4, AC008592.5, AC008703.1, AC008746.5,
AC008937.2, AC008937.3, AC008984.7, AC008992.2, AC009093.1, AC009095.4,
AC009120.10, AC009120.4, AC009166.5, AC009223.1, AC009232.2, AC009236.1,
AC009236.2, AC009237.1, AC009237.11, AC009237.8, AC009274.6, AC009299.2,
AC009299.3, AC009302.2, AC009303.1, AC009475.2, AC009487.6, AC009495.3,
AC009495.4, AC009499.1, AC009501.4, AC009502.1, AC009506.1, AC009948.5,
AC009948.7, AC009950.2, AC009961.5, AC009963.6, AC009994.2, AC010095.5,
AC010127.4, AC010136.2, AC010149.4, AC010226.4, AC010240.2, AC010240.3,
AC010336.2, AC010468.1, AC010487.1, AC010525.4, AC010525.5, AC010525.7,
AC010547.9, AC010641.1, AC010733.4, AC010878.3, AC010890.1, AC010894.5,
AC010969.1, AC010982.1, AC011239.1, AC011242.6, AC011290.5, AC011298.2,
AC011322.1, AC011330.12, AC011330.5, AC011330.6, AC011385.1, AC011406.2,
AC011450.2, AC011477.1, AC011498.1, AC011499.1, AC011526.1, AC011537.1,
AC011754.1, AC011816.3, AC011891.5, AC011933.2, AC012065.5, AC012066.1,
AC012074.2, AC012087.2, AC012146.7, AC012309.5, AC012314.6, AC012317.1,
AC012354.6, AC012354.8, AC012360.4, AC012363.4, AC012442.5, AC012512.1,
AC013268.5, AC013269.3, AC013275.2, AC013468.1, AC013470.6, AC013717.3,
AC013733.3, AC015726.1, AC015849.14, AC015922.6, AC015922.7, AC015923.1,
AC015971.2, AC015987.1, AC015987.2, AC016292.3, AC016582.2, AC016586.1,
AC016644.1, AC016689.1, AC016700.3, AC016700.4, AC016700.6, AC016716.1,
AC016732.2, AC016745.1, AC016831.7, AC016909.1, AC016910.1, AC016912.3,
AC016995.3, AC017048.3, AC017048.4, AC017071.1, AC017074.1, AC017079.3,
AC017079.4, AC017083.2, AC017083.3, AC017101.10, AC017104.2, AC017104.4,
AC018462.2, AC018642.1, AC018696.4, AC018696.7, AC018720.10, AC018735.1,
AC018737.1, AC018737.4, AC018867.2, AC018890.6, AC019050.1, AC019097.7,
AC019117.2, AC019181.3, AC019186.1, AC019294.1, AC020571.3, AC020594.5,
AC020900.2, AC020907.1, AC022153.1, AC022182.3, AC022431.1, AC022819.3,
AC023085.1, AC023797.1, AC024082.3, AC024560.2, AC024592.9, AC024704.2,
AC025287.1, AC025335.1, AC025442.3, AC025750.6, AC025918.2, AC026150.6,
AC026150.9, AC026166.1, AC026202.3, AC026471.6, AC034110.1, AC034154.1,
AC034187.2, AC034228.4, AC034229.1, AC037459.4, AC044860.1, AC046130.1,
AC053503.4, AC055811.1, AC058791.2, AC060226.1, AC062028.1, AC062029.1,
AC063976.3, AC063976.6, AC064843.1, AC064843.2, AC064850.4, AC064871.3,
AC064872.1, AC066692.3, AC067945.4, AC067950.1, AC067961.1, AC068042.1,
AC068134.6, AC068134.8, AC068137.2, AC068279.3, AC068491.1, AC068491.3,
AC068492.1, AC068538.2, AC068754.1, AC069154.2, AC069155.1, AC069200.1,
AC069282.6, AC069292.6, AC069363.1, AC072031.1, AC073043.2, AC073072.5,
AC073109.2, AC073115.7, AC073128.10, AC073135.2, AC073254.1, AC073316.1,
AC073343.1, AC073343.11, AC073343.2, AC073410.1, AC073479.1, AC073626.2,
AC073635.5, AC073834.3, AC073842.18, AC073842.19, AC073934.6, AC074011.2,
AC074091.1, AC074091.13, AC074182.1, AC074183.4, AC074212.3, AC074391.1,
AC078819.1, AC078852.1, AC078852.2, AC078883.4, AC078899.1, AC079140.1,
AC079145.4, AC079250.1, AC079354.6, AC079466.1, AC079630.4, AC079741.2,
AC079922.3, AC083799.1, AC083843.3, AC083862.1, AC083863.7, AC083873.4,
AC083899.3, AC083900.1, AC084125.2, AC084149.1, AC084809.3, AC084859.1,
AC087163.2, AC087294.2, AC087501.1, AC087793.1, AC090044.1, AC090044.2,
AC090286.2, AC090286.4, AC090420.1, AC090519.7, AC090587.4, AC090602.1,
AC090617.1, AC090952.4, AC091167.3, AC091492.2, AC091633.2, AC091729.7,
AC091729.8, AC091969.1, AC092106.2, AC092117.1, AC092155.1, AC092165.4,
AC092170.1, AC092295.4, AC092574.1, AC092574.2, AC092594.1, AC092597.3,
AC092610.12, AC092646.2, AC092661.2, AC092664.1, AC092667.2, AC092687.5,
AC092755.4, AC092811.1, AC092835.2, AC093106.7, AC093142.2, AC093162.5,
AC093171.1, AC093390.1, AC093391.2, AC093510.1, AC093620.5, AC093627.12,
AC093662.6, AC093702.1, AC093899.3, AC094019.4, AC096579.1, AC096591.1,
AC096649.1, AC096649.2, AC096669.1, AC096669.3, AC096670.3, AC096753.1,
AC096753.2, AC097359.1, AC097381.1, AC097382.1, AC097467.2, AC097500.1,
AC097500.2, AC097523.1, AC097523.2, AC097635.5, AC097662.2, AC097711.1,
AC097721.2, AC097724.3, AC098592.6, AC098614.2, AC098820.3, AC098823.3,
AC098828.3, AC099048.1, AC099344.3, AC099535.4, AC099544.2, AC099552.4,
AC099850.1, AC100803.1, AC103681.1, AC103740.1, AC103801.2, AC104024.1,
AC104076.3, AC104134.2, AC104135.2, AC104297.1, AC104306.1, AC104306.2,
AC104306.4, AC104600.1, AC104650.2, AC104651.2, AC104695.3, AC104841.1,
AC104984.4, AC104986.1, AC105020.1, AC105052.2, AC105053.4, AC105247.1,
AC105396.3, AC105399.2, AC105402.1, AC105402.4, AC105461.1, AC106053.1,
AC106722.1, AC106827.2, AC107021.1, AC107072.2, AC107081.5, AC107982.4,
AC108039.1, AC108066.1, AC108448.2, AC108463.1, AC108463.2, AC108463.3,
AC108488.3, AC108676.1, AC108868.3, AC108868.4, AC108938.2, AC109309.4,
AC109309.5, AC109826.1, AC109826.2, AC110491.1, AC111200.2, AC112229.7,
AC113554.1, AC113607.1, AC113607.2, AC113607.3, AC114546.1, AC114730.2,
AC114730.3, AC114730.5, AC114752.2, AC114755.3, AC114772.1, AC114776.1,
AC114947.1, AC116050.1, AC116366.5, AC117490.2, AC117834.1, AC118344.1,
AC120194.1, AC121336.1, AC121336.2, AC123768.3, AC123886.2, AC124890.1,
AC126118.1, AC128709.1, AC129492.6, AC131011.1, AC131097.4, AC133485.1,
AC133644.3, AC135776.1, AC136289.1, AC137590.1, AC137932.6, AC138430.2,
AC138430.4, AC138655.4, AC138783.12, AC138972.1, AC139099.3, AC139099.5,
AC139099.6, AC139452.2, AC139712.2, AC140061.1, AC140061.11, AC140481.2,
AC140481.4, AC140481.7, AC140481.8, AC140481.9, AC144521.1, AC144835.1,
AC145124.2, AC145291.1, AC145343.2, AC145676.2, AC147651.4, AC147651.5,
AC156455.1, AC195454.1, AC226119.1, AC226119.4, ACA59, ACA64, ACACB,
ACAD11, ACADM, ACADSB, ACAP2, ACAT1, ACBD3, ACBD5, ACCSL, ACE2,
ACLY, ACMSD, ACN9, ACOT12, ACOT13, ACOT4, ACOT6, ACOXL, ACPP,
ACRBP, ACRC, ACRV1, ACSBG1, ACSBG2, ACSL1, ACSL3, ACSL4, ACSL5,
ACSL6, ACSM1, ACSM5, ACSS1, ACTA1, ACTBP1, ACTBP11, ACTBP7,
ACTBP9, ACTC1, ACTG1, ACTG1P10, ACTN2, ACTR10, ACTR3B, ACTR6,
ACYP1, AD000091.3, AD001527.5, ADAL, ADAM17, ADAM1B, ADAM20,
ADAM20P1, ADAM21, ADAM24P, ADAM28, ADAM29, ADAM9, ADAMDEC1,
ADAMTS4, ADAMTS5, ADAMTSL2, ADAP2, ADCY3, ADCYAP1, ADH1C,
ADH5P2, ADK, ADM, ADO, ADORA2BP, ADORA3, ADPRM, ADRA1D,
ADRA2A, ADRB2, ADSS, ADTRP, AE000661.37, AEBP2, AF001548.5,
AF011889.2, AF038458.4, AF064858.8, AF064860.7, AF124730.4, AF127577.1,
AF127577.10, AF127577.8, AF130249.5, AF131215.1, AF131215.5, AF131215.6,
AF186192.1, AF186192.5, AF186192.6, AF196972.4, AF196972.9, AF207550.1,
AF213884.2, AF228730.12, AF228730.7, AF230666.2, AFF1, AFF4, AFTPH,
AGAP10, AGBL3, AGGF1, AGMO, AGPAT9, AGPHD1, AGRP, AGTPBP1,
AGXT2L1, AHCTF1, AHCYL1, AHI1, AHNAK2, AHRR, AIDA, AIG1, AIG1P1,
AIMP1, AIRE, AK2, AK2P2, AKAP11, AKAP14, AKAP2, AKAP3, AKAP6,
AKAP7, AKNAD1, AKR1B10, AKR1C3, AKR1CL1, AKT3, AKT3-IT1,
AL022393.7, AL023807.1, AL031601.3, AL031602.1, AL031768.1, AL049757.3,
AL050327.1, AL078621.11, AL078621.13, AL078621.3, AL078633.1, AL109615.1,
AL109761.5, AL109767.1, AL109806.1, AL132709.1, AL132709.2, AL132709.5,
AL132709.8, AL135791.1, AL136419.1, AL136985.1, AL137127.1, AL137229.1,
AL137855.1, AL138479.3, AL138706.1, AL138795.1, AL138796.1, AL138968.1,
AL139099.1, AL139239.1, AL139333.1, AL139377.1, AL157788.1, AL159997.1,
AL160275.1, AL160314.1, AL161645.1, AL161645.2, AL161793.1, AL162389.1,
AL162431.1, AL353626.1, AL353626.2, AL353629.1, AL353662.3, AL353671.1,
AL353997.7, AL358175.1, AL358175.2, AL359218.1, AL359314.1, AL359392.1,
AL359473.1, AL359736.1, AL359955.1, AL360004.1, AL360297.1, AL390071.1,
AL390877.1, AL391152.1, AL441988.1, AL512652.1, AL512791.1, AL589743.1,
AL590113.1, AL590226.1, AL590431.1, AL590703.1, AL590762.6, AL590762.7,
AL590764.1, AL590787.1, AL591516.1, AL591516.2, AL591516.3, AL591516.4,
AL591704.5, AL591893.1, AL592494.1, AL592494.3, AL592494.5, AL645730.2,
AL672294.1, AL691479.1, AL773572.7, AL773604.8, ALAS2, ALCAM,
ALDH1A2, ALDH1A3, ALDH1L1-AS1, ALDH1L2, ALDH9A1, ALDOB, ALG10,
ALG10B, ALG11, ALG13-AS1, ALG14, ALG2, ALG5, ALG8, ALG9, ALKBH1,
ALKBH3, ALMS1, ALOX12P1, ALOX5, ALOXE3, ALS2CL, ALS2CR12,
ALS2CR8, ALX1, AMBN, AMD1, AMIGO3, AMMECR1, AMMECR1LP1,
AMN1, AMPD1, AMPD3, AMY2A, AMZ2, AMZ2P1, ANAPC10, ANAPC10P1,
ANAPC13, ANAPC16, ANAPC4, ANGEL1, ANGPT4, ANGPTL1, ANGPTL2,
ANGPTL3, ANGPTL4, ANGPTL5, ANGPTL6, ANGPTL7, ANKAR, ANKDD1B,
ANKH, ANKHD1, ANKMY2, ANKRD12, ANKRD13C, ANKRD16, ANKRD18EP,
ANKRD2, ANKRD20A1, ANKRD20A14P, ANKRD20A19P, ANKRD20A2,
ANKRD20A3, ANKRD20A5P, ANKRD22, ANKRD23, ANKRD26P1,
ANKRD26P4, ANKRD27, ANKRD29, ANKRD30A, ANKRD31, ANKRD32,
ANKRD33, ANKRD33B, ANKRD34C, ANKRD36BP1, ANKRD36BP2,
ANKRD37, ANKRD40, ANKRD42, ANKRD43, ANKRD45, ANKRD5, ANKRD6,
ANKRD62, ANKRD7, ANO5, ANP32BP1, ANP32C, ANP32E, ANTXR1,
ANXA11, ANXA2, ANXA2P2, ANXA2P3, ANXA5, ANXA7, AP000233.3,
AP000253.1, AP000282.2, AP000304.2, AP000354.2, AP000356.1, AP000432.2,
AP000435.1, AP000442.1, AP000462.1, AP000462.2, AP000473.5, AP000525.1,
AP000525.8, AP000552.1, AP000568.2, AP000580.1, AP000593.5, AP000593.6,
AP000593.7, AP000648.6, AP000662.9, AP000695.4, AP000695.6, AP000705.7,
AP000769.1, AP000790.1, AP000797.2, AP000797.4, AP000807.2, AP000925.2,
AP000936.2, AP000974.1, AP001007.1, AP001010.1, AP001042.1, AP001044.2,
AP001046.5, AP001052.9, AP001053.11, AP001059.5, AP001062.9, AP001065.2,
AP001187.9, AP001189.4, AP001205.1, AP001324.1, AP001372.2, AP001432.14,
AP001469.7, AP001596.6, AP001597.1, AP001607.1, AP001610.5, AP001619.2,
AP001625.4, AP001625.6, AP001626.1, AP001630.5, AP001793.1, AP003068.18,
AP003774.1, AP004550.1, AP006309.4, AP1AR, AP1G1, AP1S3, AP3B1, AP3M1,
AP4B1, AP5M1, APBB1IP, APBB2, APC, APCDD1L, APCDD1L-AS1, APCS,
API5, API5P1, APIP, APLF, APLP2, APOA5, APOBEC2, APOBEC3B-AS1,
APOBEC4, APOD, APOF, APOL1, APOL2, APOL3, APOL4, APOL6, APOO,
APOOL, APPL1, AQP12A, AQP2, AQP3, AQP4, AQP9, AQR, ARAP3, ARCN1,
ARF4, ARFGAP3, ARFGEF2, ARFIP1, ARG2, ARGFX, ARHGAP11A,
ARHGAP12, ARHGAP24, ARHGAP27, ARHGAP31, ARHGAP31-AS1,
ARHGAP42, ARHGAP44, ARHGAP5, ARHGDIB, ARHGEF12, ARHGEF15,
ARHGEF26-AS1, ARHGEF3, ARHGEF37, ARHGEF38, ARHGEF6, ARID4B-IT1,
ARID5A, ARID5B, ARIH1, ARIH2P1, ARL1, ARL11, ARL13A, ARL14,
ARL14EP, ARE17B, ARL2BP, ARL4C, ARL4D, ARL5AP3, ARL5C, ARL6IP1,
ARL6IP5, ARL8B, ARL9, ARMC1, ARMC10, ARMC10P1, ARMC2-AS1,
ARMCX2, ARMCX4, ARMCX5, ARNTL, ARPC3P2, ARPC3P5, ARPC5, ARPP19,
ARRDC2, ARRDC3, ARRDC5, ARSB, ARSH, ART4, ARV1, ASAH1, ASAH2,
ASAH2C, ASAP1, ASB11, ASB12, ASB14, ASB18, ASB2, ASB3, ASB4, ASB5,
ASB9, ASB9P1, ASCC3, ASF1A, ASNS, ASNSD1, ASPA, ASPG, ASPH, ASPN,
ASS1P2, ATAD3C, ATAD5, ATF1, ATF6, ATF7, ATG10, ATG12P2, ATG16L1,
ATG3, ATG4A, ATG4C, ATG5, ATL1, ATL2, ATL3, ATM, ATMIN, ATP10B,
ATP10D, ATP11A-AS1, ATP11B, ATP13A3, ATP1A1, ATP1B1, ATP1B3,
ATP1B4, ATP2C1, ATP5EP2, ATP5F1P6, ATP5H, ATP5HP1, ATP5JP1, ATP5L2,
ATP6AP1L, ATP6AP2, ATP6V0B, ATP6V0CP1, ATP6V0CP3, ATP6V0D2,
ATP6V0E1, ATP6V1A, ATP6V1C2, ATP6V1D, ATP6V1E1, ATP6V1E1P1,
ATP6V1E2, ATP7A, ATP8A1, ATP8A2P3, ATP8B4, ATP8B5P, ATPBD4,
ATRNL1, ATXN1, ATXN8OS, AURKAPS1, AVIL, AWAT1, AXUD1, AZGP1,
AZI2, AZIN1, B2M, B3GALT5, B3GNT2, B3GNT3, B3GNT5, B3GNT7, B3GNT9,
B4GALT4-AS1, B4GALT6, BAAT, BACE2, BACH1-IT1, BACH2, BAG2, BAG3,
BAG4, BAG5, BAG6, BAIAP3, BANF1P1, BANK1, BARHL1, BATF2, BATF3,
BBS10, BBS7, BBX, BCAN, BCAP29, BCAS2, BCCIP, BCDIN3D-AS1, BCL10,
BCL2A1, BCL2L10, BCL2L13, BCL2L14, BCL2L15, BCL2L2, BCL3, BCL6,
BCL6B, BCMO1, BCO2, BCORP1, BCRP8, BDNF-AS, BDP1P, BEND6, BEST3,
BET1, BET3L, BHLHA15, BHLHE40, BHLHE41, BHMT2, BIN2, BIN2P1, BIRC3,
BLOC1S2, BLOC1S5, BLZF1, BMI1, BMP2K, BMP5, BMP8A, BMP8B, BMPER,
BMPR1APS1, BMPR1APS2, BMPR2, BMX, BNIP3L, BNIPL, BOD1L2, BOK,
BOLA3, BORA, BP75, BPIFB1, BPIFB9P, BPIFC, BPIL2, BRAF, BRAFP1, BRAP,
BRCA1, BRCC3, BRD2, BRD7, BRD7P2, BRD7P4, BRD7P5, BRDT, BRI3BP,
BRIX1, BRMS1, BROX, BRP44L, BRWD1-IT1, BRWD1-IT2, BSN-AS2, BSND,
BST2, BSX, BTAF1, BTBD16, BTC, BTF3L4P1, BTF3L4P2, BTF3P12, BTF3P7,
BTG3, BTK, BTLA, BTN3A1, BTN3A3, BUB3, BX004987.3, BX004987.4,
BX004987.5, BX284650.2, BX470187.1, BX571672.1, BX571672.2, BX571672.3,
BX571672.5, BX649563.4, BX649597.1, BZW1, BZW1P2, C10orf10, C10orf104,
C10orf108, C10orf115, C10orf118, C10orf128, C10orf129, C10orf53, C10orf62,
C10orf67, C10orf85, C10orf88, C11orf1, C11orf10, C11orf20, C11orf34, C11orf52,
C11orf60, C11orf63, C11orf75, C11orf85, C11orf89, C11orf91, C11orf92, C11orf93,
C11orf96, C12orf23, C12orf29, C12orf4, C12orf40, C12orf5, C12orf56, C12orf60,
C12orf62, C12orf66, C12orf68, C12orf69, C12orf77, C13orf15, C13orf33, C13orf35,
C14orf106, C14orf142, C14orf164, C14orf182, C14orf183, C14orf28, C15orf26,
C15orf29, C15orf48, C15orf52, C15orf55, C15orf56, C15orf57, C15orf59, C16orf52,
C16orf70, C16orf72, C16orf88, C16orf95, C17orf104, C17orf105, C17orf51,
C17orf66, C17orf80, C17orf82, C17orf99, C18orf25, C18orf34, C18orf42, C18orf54,
C19orf10, C19orf2, C19orf38, C19orf67, C19orf77, C1D, C1DP1, C1DP4,
C1GALT1C1, C1orf101, C1orf114, C1orf127, C1orf128, C1orf131, C1orf132,
C1orf133, C1orf138, C1orf146, C1orf147, C1orf170, C1orf174, C1orf177, C1orf189,
C1orf192, C1orf195, C1orf203, C1orf204, C1orf227, C1orf27, C1orf41, C1orf43,
C1orf57, C1orf61, C1orf65, C1orf66, C1orf97, C1orf98, C1QA, C1QB, C1QC,
C1QTNF2, C1QTNF4, C1QTNF9B, C1QTNF9B-AS1, C1S, C20orf197, C20orf26,
C20orf45, C20orf72, C21orf119, C21orf128, C21orf63, C21orf7, C21orf77,
C21orf91, C21orf91-OT1, C22orf24, C22orf25, C22orf26, C22orf28, C22orf34,
C2CD4A, C2CD4B, C2orf18, C2orf27A, C2orf30, C2orf32, C2orf42, C2orf44,
C2orf47, C2orf50, C2orf61, C2orf65, C2orf69, C2orf70, C2orf71, C2orf73, C2orf76,
C3AR1, C3orf14, C3orf15, C3orf26, C3orf33, C3orf38, C3orf70, C4BPAP1, C4BPB,
C4orf22, C4orf27, C4orf32, C4orf33, C4orf34, C4orf45, C4orf47, C4orf51, C4orf52,
C5AR1, C5orf17, C5orf22, C5orf27, C5orf28, C5orf30, C5orf32, C5orf34, C5orf43,
C5orf50, C5orf51, C5orf53, C5orf54, C5orf58, C5orf60, C5orf63, C6orf115,
C6orf117, C6orf130, C6orf132, C6orf160, C6orf203, C6orf222, C6orf225, C6orf62,
C6orf89, C7orf10, C7orf23, C7orf25, C7orf30, C7orf31, C7orf42, C7orf52, C7orf57,
C7orf63, C8orf4, C8orf40, C8orf46, C8orf47, C8orf74, C9orf106, C9orf119,
C9orf135, C9orf139, C9orf152, C9orf153, C9orf24, C9orf3, C9orf40, C9orf41,
C9orf72, C9orf9, C9orf91, C9orf95, CA1, CA12, CA13, CA5A, CA5B, CA5B-AS1,
CA7, CA8, CAB39, CAB39L, CABLES1, CABP1, CABP4, CABP5, CABP7,
CACNA1C-AS3, CACNA1D, CACNA2D4, CACYBP, CADM1, CALM1,
CALM2P4, CALU, CAMK2D, CAMKK1, CAMLG, CAMTA1-IT1, CANX,
CAP2P1, CAPN11, CAPN13, CAPN7, CAPN8, CAPZA3, CARD16, CARD6,
CASC4, CASC4P1, CASP1, CASP10, CASP3P1, CASP4, CASP8, CASP8AP2,
CASQ1, CASQ2, CASR, CASS4, CAST, CATSPER4, CATSPERB, CATSPERD,
CBX1P4, CBX3P1, CBX7, CC2D2B, CCDC104, CCDC109B, CCDC110,
CCDC117, CCDC121, CCDC125, CCDC144A, CCDC144B, CCDC144C,
CCDC147, CCDC15, CCDC155, CCDC158, CCDC163P, CCDC28A, CCDC36,
CCDC47, CCDC48, CCDC53, CCDC58P1, CCDC59, CCDC6, CCDC66, CCDC67,
CCDC7, CCDC70, CCDC73, CCDC75, CCDC81, CCDC84, CCDC86, CCDC87,
CCDC89, CCDC90A, CCDC91, CCL11, CCL18, CCL2, CCL22, CCL24, CCL28,
CCL3, CCL3L1, CCL4, CCNA2, CCNC, CCND1, CCND2, CCND3, CCNG1,
CCNH, CCNI, CCNO, CCNT1, CCNYL2, CCNYL3, CCP110, CCPG1, CCR1,
CCR3, CCR5, CCR7, CCRL1, CCRL2, CCRN4L, CCSAP, CCT4, CCT4P2,
CCT5P2, CCT6P2, CCT6P4, CCT7P1, CCT8, CCT8L1P, CCT8P1, CD14, CD163,
CD163L1, CD180, CD1D, CD200R1, CD209, CD22, CD244, CD274, CD2AP,
CD300E, CD300LB, CD300LF, CD300LG, CD302, CD33, CD38, CD40, CD44,
CD46, CD48, CD5, CD53, CD55, CD58, CD59, CD68, CD72, CD79B, CD80, CD83,
CD84, CD86, CD9, CD93, CD99, CDADC1, CDC14A, CDC14B, CDC26,
CDC27P1, CDC37L1, CDC42BPA, CDC42EP5, CDC5L, CDC6, CDC7, CDC73,
CDCA2, CDH19, CDH22, CDH26, CDH5, CDHR4, CDK14, CDK2AP2P1,
CDK2AP2P2, CDK6, CDK7, CDK8, CDKL3, CDKL4, CDKN1A, CDKN1B,
CDKN2A, CDKN2AIPNLP1, CDKN2B, CDKN2B-AS1, CDKN2C, CDNF, CDR2,
CDRT1, CDV3, CEACAM21, CEACAM3, CEACAM6, CEACAM7, CEACAMP5,
CEBPB, CEBPD, CEBPE, CEBPG, CEBPZ, CECR7, CEL, CELA2A, CELA2B,
CELA3A, CELA3B, CELP, CENPC1, CENPC1P1, CENPQ, CENPW, CEP120,
CEP128, CEP152, CEP350, CEP41, CEP44, CEP57L1, CEP57L1P1, CEP63, CEP70,
CEP76, CEPT1, CERK, CERS3, CERS6, CES1P1, CETN3, CETN4P, CETP,
CFC1B, CFH, CFI, CFL1P4, CFL1P5, CFL2, CFLAR, CFLAR-AS1, CFTR, CGB7,
CGGBP1, CGI-148P, CGRRF1, CHAC2, CHAF1B, CHAMP1, CHCHD10,
CHCHD2, CHCHD2P2, CHCHD2P6, CHCHD4, CHCHD7, CHCHD9, CHD1,
CHD1L, CHD9, CHEK2P2, CHGB, CHI3L2, CHIT1, CHL1-AS1, CHM, CHMP1B,
CHMP2B, CHMP4BP1, CHMP5, CHN1, CHODL, CHORDC1, CHPF, CHPT1,
CHRDL2, CHRM1, CHRM4, CHRNA1, CHRNA3, CHRNA5, CHRNA9, CHRNG,
CHST1, CHST2, CHUK, CHURC1, CIB1, CICP3, CICP5, CICP6, CICP9, CIDEA,
CIR1P1, CIR1P2, CISD1, CISH, CITED2, CKAP4, CKLF, CLCA1, CLCA2,
CLCA3P, CLCA4, CLCC1, CLCN3, CLCN4, CLDN12, CLDN14, CLDN2, CLDN4,
CLDN5, CLDN9, CLEC10A, CLEC14A, CLEC17A, CLEC18A, CLEC18C,
CLEC1A, CLEC3A, CLEC4D, CLEC4G, CLEC4M, CLEC5A, CLEC7A, CLGN,
CLINT1, CLIP1, CLIP4, CLK1, CLK4, CLLU1OS, CLMN, CLN5, CLOCK,
CLRN2, CLTC, CLVS1, CMA1, CMAHP, CMBL, CMC1, CMKLR1, CMPK1,
CNBP, CNOT10-AS1, CNOT6L, CNOT6LP1, CNOT7, CNOT7P1, CNOT8, CNP,
CNR2, CNRIP1, CNST, CNTD1, CNTN1, CNTN4, CNTN4-AS2, CNTN5, COA5,
COG3, COG5, COG6, COL10A1, COL13A1, COL15A1, COL20A1, COL24A1,
COL28A1, COL4A3, COL5A3, COLEC10, COLEC12, COMMD3, COMMD6,
COMP, COPB1, COPB2, COPS2, COPS4, COPZ1, COPZ2, COQ10B, COQ3,
CORO6, CORO7, CORT, COX11P1, COX17, COX17P1, COX20, COX5AP2,
COX5BP2, COX6A2, COX6B1P1, COX6CP1, COX7A1, COX7A2P1, COX7B,
COX7CP1, CP, CPA1, CPA4, CPB1, CPD, CPE, CPEB3, CPEB4, CPLX4, CPNE3,
CPNE4, CPNE8, CPO, CPQ, CPSF2, CPSF4L, CPT1A, CPT2, CR2, CR381653.2,
CREB3L2, CREB5, CREBL2, CREBRF, CREG1, CREM, CRIP1, CRNKL1, CRP,
CRTAP, CRY1, CRYAB, CRYBG3, CRYZ, CSDE1, CSE1L, CSF2, CSF2RB,
CSGALNACT2, CSNK1A1P3, CSNK2A1P, CSRNP1, CST2, CST7, CTA-126B4.7,
CTA-14H9.5, CTA-204B4.6, CTA-211A9.5, CTA-212D2.2, CTA-215D11.4, CTA-
229A8.5, CTA-246H3.11, CTA-253N17.1, CTA-276O3.4, CTA-292E10.7, CTA-
299D3.8, CTA-313A17.2, CTA-360L10.1, CTA-363E6.6, CTA-440B3.1, CTA-
833B7.2, CTA-972D3.2, CTAGE1, CTAGE10P, CTAGE11P, CTAGE13P,
CTAGE3P, CTAGE5, CTAGE6P, CTAGE9, CTB-102L5.4, CTB-104F4.2, CTB-
109A12.1, CTB-113P19.3, CTB-118N6.1, CTB-118N6.2, CTB-118N6.3, CTB-
120L21.1, CTB-12O2.1, CTB-131B5.4, CTB-131B5.5, CTB-131K11.1, CTB-
133G6.1, CTB-134H23.3, CTB-13H5.1, CTB-140J7.2, CTB-157D17.1, CTB-
158E9.2, CTB-161M19.1, CTB-161M19.4, CTB-193M12.1, CTB-31C7.1, CTB-
31N19.2, CTB-35F21.1, CTB-35F21.2, CTB-35F21.4, CTB-36O1.3, CTB-3M24.3,
CTB-43P18.1, CTB-46B19.1, CTB-49A3.5, CTB-50L17.16, CTB-50L17.7, CTB-
51J22.1, CTB-54D4.1, CTB-55O6.10, CTB-55O6.4, CTB-75G16.1, CTBS, CTC-
209H22.3, CTC-228N24.1, CTC-228N24.3, CTC-250I14.1, CTC-251H24.1, CTC-
260E6.6, CTC-260F20.5, CTC-263F14.5, CTC-264K15.6, CTC-265F19.1, CTC-
265F19.2, CTC-281B15.1, CTC-297N7.10, CTC-303L1.1, CTC-308K20.1, CTC-
308K20.2, CTC-308K20.3, CTC-329H14.1, CTC-338M12.1, CTC-338M12.5, CTC-
339F2.2, CTC-347C20.1, CTC-422A18.1, CTC-428G20.1, CTC-431G16.2, CTC-
439O9.2, CTC-444D3.7, CTC-448D22.1, CTC-454I21.3, CTC-454M9.1, CTC-
457E21.1, CTC-457L16.2, CTC-459F4.1, CTC-459F4.3, CTC-459F4.6, CTC-
459F4.7, CTC-463N11.1, CTC-471C19.1, CTC-471J1.1, CTC-471J1.2, CTC-
493L21.1, CTC-498J12.3, CTC-499B15.4, CTC-499B15.7, CTC-499B15.8, CTC-
504A5.1, CTC-506B8.1, CTC-512J14.7, CTC-523E23.4, CTC-524C5.5, CTC-
534A2.2, CTC-534B23.1, CTC-542B22.2, CTC-543D15.3, CTC-548K16.6, CTC-
550B14.7, CTC-551A13.2, CTC-559E9.2, CTC-559E9.4, CTC-756D1.3, CTC-
774J1.2, CTC-782O7.2, CTC-788C1.1, CTD-2003C8.1, CTD-2005H7.1, CTD-
2005H7.2, CTD-2006C1.10, CTD-2006C1.12, CTD-2006C1.13, CTD-2006K23.1,
CTD-2007H13.1, CTD-2007H13.3, CTD-2008P7.1, CTD-2008P7.10, CTD-
2008P7.3, CTD-2008P7.9, CTD-2012K14.7, CTD-2013N17.1, CTD-2013N24.2,
CTD-2014B16.3, CTD-2015B23.2, CTD-2017C7.1, CTD-2022H16.3, CTD-
2024I7.13, CTD-2024P10.1, CTD-2026G6.1, CTD-2026G6.2, CTD-2026K11.2,
CTD-2026K11.3, CTD-2031P19.4, CTD-2031P19.5, CTD-2034I21.2, CTD-
2036P10.3, CTD-2047H16.2, CTD-2050B12.1, CTD-2050N2.1, CTD-2058B24.3,
CTD-2060C23.1, CTD-2061E19.1, CTD-2072I24.1, CTD-2085F10.1, CTD-
2085J24.3, CTD-2086L14.1, CTD-2086O20.3, CTD-2089O24.1, CTD-2095E4.1,
CTD-2104P17.1, CTD-2105E13.13, CTD-2116N17.1, CTD-2119F7.2, CTD-
2122P11.1, CTD-2130O13.2, CTD-2134P3.1, CTD-2138O14.1, CTD-2139B15.2,
CTD-2140G10.2, CTD-2144E22.10, CTD-2145A24.3, CTD-2154I11.1, CTD-
2154I11.2, CTD-2162K18.3, CTD-2162K18.5, CTD-2165H16.3, CTD-2165H16.4,
CTD-2175M1.1, CTD-2179L22.1, CTD-2184C24.2, CTD-2184D3.4, CTD-
2184D3.5, CTD-2189E23.2, CTD-2193P3.2, CTD-2195M18.1, CTD-2198K18.1,
CTD-2201E9.1, CTD-2201E9.2, CTD-2206G10.2, CTD-2207P18.1, CTD-
2210P15.2, CTD-2210P24.4, CTD-2213F21.2, CTD-2215E18.1, CTD-2215L10.1,
CTD-2224J9.3, CTD-2224J9.8, CTD-2228K2.1, CTD-2228K2.2, CTD-2240E14.4,
CTD-2240H23.2, CTD-2240J17.1, CTD-2245E15.3, CTD-2246P4.1, CTD-
2248H3.1, CTD-2256P15.1, CTD-2265D6.1, CTD-2265O21.3, CTD-2267D19.2,
CTD-2270L9.2, CTD-2272G21.2, CTD-2277K2.1, CTD-2281D19.1, CTD-
2281M20.1, CTD-2286N8.2, CTD-2287O16.1, CTD-2288F12.1, CTD-2288O8.1,
CTD-2292M16.8, CTD-2292P10.2, CTD-2292P10.4, CTD-2293H3.2, CTD-
2302E22.3, CTD-2302E22.4, CTD-2307P3.1, CTD-2308G16.1, CTD-2313J17.1,
CTD-2313J17.2, CTD-2313J17.5, CTD-2313N18.2, CTD-2314B22.3, CTD-
2319I12.1, CTD-2319I12.3, CTD-2319I12.4, CTD-2319I12.5, CTD-2323K18.1,
CTD-2323K18.3, CTD-2324F15.2, CTD-2329K10.1, CTD-2331D11.1, CTD-
2335A18.1, CTD-2337A12.1, CTD-2345M20.1, CTD-2349P21.1, CTD-2349P21.11,
CTD-2349P21.9, CTD-2366F13.1, CTD-2369P2.8, CTD-2373H9.5, CTD-2373H9.6,
CTD-2380F24.1, CTD-2382E5.1, CTD-2382E5.4, CTD-2501E16.1, CTD-
2503H21.2, CTD-2503O16.2, CTD-2509G16.4, CTD-2509G16.5, CTD-2510F5.4,
CTD-2514C3.1, CTD-2515C13.1, CTD-2516F10.4, CTD-2516K3.3, CTD-
2517M22.9, CTD-2521M24.4, CTD-2522E6.4, CTD-2526A2.2, CTD-2526L21.3,
CTD-2526M8.2, CTD-2528L19.3, CTD-2529O21.1, CTD-2531D15.4, CTD-
2537I9.13, CTD-2537I9.5, CTD-2537O9.1, CTD-2538C1.3, CTD-2540L5.3, CTD-
2540L5.6, CTD-2540M10.1, CTD-2541M15.1, CTD-2544N14.3, CTD-2545H1.1,
CTD-2553C6.1, CTD-2555C10.3, CTD-2555K7.1, CTD-2555O16.2, CTD-
2561B21.11, CTD-2574D22.4, CTD-2574D22.5, CTD-2574D22.6, CTD-2583P5.3,
CTD-2589O24.1, CTD-2595P9.1, CTD-2609K8.3, CTD-2630F21.1, CTD-
2631K10.1, CTD-2644I21.1, CTD-2647E9.1, CTD-2650P22.1, CTD-2653M23.1,
CTD-3010D24.3, CTD-3035D6.1, CTD-3035D6.2, CTD-3049M7.1, CTD-
3051D23.4, CTD-3057O21.1, CTD-3064C13.1, CTD-3064H18.6, CTD-3064M3.4,
CTD-3064M3.6, CTD-3065B20.2, CTD-3083F21.1, CTD-3083F21.5, CTD-
3092A11.1, CTD-3105H18.11, CTD-3105H18.13, CTD-3110H11.1, CTD-
3118D11.2, CTD-3157E16.1, CTD-3162L10.1, CTD-3162L10.3, CTD-3162L10.5,
CTD-3185P2.1, CTD-3193O13.1, CTD-3220F14.2, CTD-3234P18.2, CTD-
3239E11.2, CTD-3244O18.6, CTD-3247F14.2, CTDSPL2, CTF2P, CTGLF12P,
CTNNA3, CTNNAP1, CTR9, CTRB1, CTRB2, CTRC, CTSF, CTSL1, CTSL2,
CTSO, CTSS, CTSW, CUL3, CUL4B, CUL5, CUTC, CUZD1, CWC15, CWC22,
CWC27, CWF19L1, CWF19L2, CX3CR1, CXCL10, CXCL16, CXCL17, CXCL3,
CXCR3, CXCR6, CXorf21, CXorf22, CXorf24, CXorf26, CXorf36, CXorf57,
CXorf67, CYB5R1, CYB5R4, CYBRD1, CYCS, CYCSP55, CYLD, CYP17A1,
CYP17A1-AS1, CYP19A1, CYP1A1, CYP1B1, CYP2A6, CYP2A7, CYP2C18,
CYP2G1P, CYP2J2, CYP2U1, CYP3A43, CYP3A5, CYP3A7, CYP4F11, CYP4F12,
CYP4F24P, CYP4F25P, CYP4F3, CYP4F43P, CYP4V2, CYP51P2, CYS1,
CYSLTR1, CYSTM1, CYTIP, CYYR1, DAAM1, DACH2, DAD1, DAP, DAP3P1,
DAPK1-IT1, DAPK2, DAPL1, DAPP1, DARS, DARS2, DAXX, DAZAP2,
DAZAP2P1, DAZL, DBIL5P, DBNDD2, DBR1, DCAF10, DCAF13P3, DCAF17,
DCAF4L1, DCBLD1, DCDC1, DCDC2, DCLK3, DCLRE1A, DCT, DCTN4,
DCTN6, DCUN1D4, DDHD1, DDHD2, DDI2, DDO, DDX1, DDX10, DDX11-AS1,
DDX11L10, DDX11L2, DDX11L5, DDX18, DDX21, DDX24, DDX26B, DDX3Y,
DDX3YP2, DDX4, DDX43, DDX46, DDX50, DDX50P2, DDX55, DDX60,
DDX60L, DDX6P2, DEFB1, DEFB109P1, DEFB109P1B, DEFB135, DEGS1, DEK,
DENND2D, DENND4A, DEPTOR, DERL1, DERL2, DERL3, DES, DESI2,
DGCR11, DGCR5, DGCR6, DGKB, DGKD, DGKE, DHFRL1, DHRS7, DHRS7B,
DHRS9, DHX29, DHX32, DHX40, DHX58, DHX9P1, DIAPH1, DIEXF, DIO1,
DIO2, DIP2C, DIS3, DIXDC1, DKFZp686O24166, DKK4, DLAT, DLEU1, DLEU2,
DLEU2L, DLG1, DLG1-AS1, DLG2, DLG3-AS1, DLGAP1-AS1, DLGAP1-AS2,
DLX6, DMC1, DMGDH, DMP1, DMRTA1, DMRTA2, DMXL1, DNA2, DNAH3,
DNAH5, DNAH6, DNAJA1P3, DNAJA4, DNAJB1, DNAJB11, DNAJB14,
DNAJB3, DNAJB4, DNAJB8-AS1, DNAJB9, DNAJC12, DNAJC14, DNAJC15,
DNAJC22, DNAJC25, DNAJC25-GNG10, DNAJC3, DNAJC3-AS1, DNAJC5B,
DNASE2B, DNM1, DNM1P32, DNM3, DNM3-IT1, DNMT3L, DNTT, DNTTIP2,
DOCK10, DOCK11, DOCK3, DOCK9, DOK2, DOK7, DOM3Z, DPEP2, DPH3P1,
DPM1, DPM2, DPM3, DPPA3P1, DPRX, DPRXP4, DPT, DPY19L2P4, DPYS,
DR1, DRAM1, DRAM2, DRD5P1, DSCR4-IT1, DSCR8, DSTN, DSTNP1,
DSTNP2, DSTYK, DTWD1, DTWD2, DTX2, DTX2P1-UPK3BP1-PMS2P11,
DTX3L, DUOX2, DUOXA2, DUS4L, DUSP11, DUSP19, DUSP2, DUSP23,
DUSP26, DUSP5, DUSP5P, DUTP7, DYDC1, DYDC2, DYM, DYNC1I1,
DYNC2H1, DYNC2LI1, DYNLL2, DYNLT3, DYNLT3P1, DYNLT3P2, DYRK1A,
DYRK3, DYRK4, DYTN, DZIP1, EAF1, FAF1-AS1, EAF2, EAPP, EBAG9, EBI3,
ECEL1P2, ECI2, ECM1, EDA2R, EDEM1, EDEM3, EDIL3, EDN3, EDNRB,
EEA1, EED, EEF1A1P24, EEF1A1P29, EEF1A1P30, EEF1A1P33, EEF1A1P41,
EEF1B2, EEF1B2P2, EEF1B2P3, EEF1DP3, EEF1DP5, EEF1G, EEPD1, EFCAB10,
EFCAB13, EFCAB6-AS1, EFHA1, EFHC2, EFHD1, EFNA5, EFR3A, EFTUD1P1,
EGF, EGFLAM, EGLN1, EGOT, EGR3, EHBP1, EHBP1L1, EHF, EHHADH,
EHHADH-AS1, EI24, EID3, EIF1, EIF1AY, EIF2A, EIF2AK1, EIF2AK2,
EIF2AK3, EIF2B3, EIF2C2, EIF2S1, EIF2S2P3, EIF2S2P4, EIF2S3L, EIF3A,
EIF3E, EIF3H, EIF3J, EIF3M, EIF4A2, EIF4B, EIF4BP3, EIF4BP5, EIF4BP6,
EIF4BP7, EIF4E, EIF4E1B, EIF4E3, EIF5A2, EIF5AL1, EIF5AP2, EIF5AP3,
EIF5B, ELA3A, ELAVL4, ELK2BP, ELK4, ELL2P1, ELL3, ELMO1, ELMO3,
ELOVL3, ELOVL5, ELOVL7, ELP2, ELP4, ELTD1, EMB, EMBP1, EMC2, EMC3,
EMC7, EME1, EMG1, EML5, EMP1, EMP2, EMP3, EMR1, EMR2, EMR3, EMX1,
EN1, ENAM, ENDOD1, ENDOU, ENKUR, ENO1-IT1, ENOSF1, ENOX1-AS1,
ENOX2, ENPP2, ENPP3, ENPP4, ENPP5, ENTPD3, ENTPD3-AS1, EPAS1,
EPB41L2, EPB41L4B, EPCAM, EPDR1, EPHA1-AS1, EPHA2, EPHA5,
EPM2AIP1, EPRS, EPS15, ERAP2, ERAS, ERCC4, ERCC5, ERCC6, ERCC6L2,
ERCC8, EREG, ERG, ERGIC2, ERHP1, ERI1, ERLEC1, ERLIN1, ERLIN2, ERMN,
ERMP1, ERO1LB, ERP27, ERP29P1, ERP44, ERRFI1, ERV3-1, ERVFRD-1,
ERVH48-1, ESCO2, ESF1, ESM1, ESR1, ESR2, ESRG, ESRP1, ETF1, ETF1P2,
ETFDH, ETNK1, ETS1, ETS2, ETV5, ETV5-AS1, ETV7, EVI2B, EVI5, EXD1,
EXOC1, EXOC5, EXOC8, EXOSC3P1, EXOSC9, EXPH5, F11, F2RL3, F8, F8A1,
FABP1, FABP4, FABP5P3, FAF1, FAIM3, FAM102A, FAM103A2P, FAM104A,
FAM105A, FAM106A, FAM107A, FAM108A7P, FAM108C1, FAM109B,
FAM110C, FAM114A1, FAM115C, FAM115D, FAM117A, FAM120A, FAM122B,
FAM129A, FAM132B, FAM133A, FAM133B, FAM133CP, FAM133DP,
FAM134B, FAM135A, FAM138D, FAM13A, FAM13C, FAM149A, FAM149B1P1,
FAM150B, FAM151B, FAM153A, FAM153B, FAM153C, FAM155A-IT1,
FAM156A, FAM159B, FAM160A1, FAM160B1, FAM167A, FAM169A,
FAM169B, FAM175A, FAM175B, FAM176C, FAM177A1, FAM177B, FAM179B,
FAM180A, FAM182A, FAM182B, FAM183B, FAM186A, FAM18B, FAM18B1,
FAM18B2-CDRT4, FAM190A, FAM190B, FAM199X, FAM19A3, FAM19A4,
FAM200A, FAM205A, FAM206A, FAM208B, FAM209A, FAM209B, FAM216A,
FAM217A, FAM220A, FAM227B, FAM23B, FAM24B, FAM25G, FAM26E,
FAM26F, FAM27A, FAM27B, FAM27C, FAM27D1, FAM27E1, FAM27E3,
FAM35A, FAM35B2, FAM3B, FAM3C, FAM3C2, FAM40B, FAM45B, FAM46C,
FAM47C, FAM47E, FAM50B, FAM53A, FAM58A, FAM59A, FAM60A, FAM63B,
FAM66A, FAM70A, FAM71A, FAM71E2, FAM71F1, FAM72A, FAM72C,
FAM72D, FAM75C1, FAM82A1, FAM83C, FAM83C-AS1, FAM86DP, FAM87B,
FAM8A1, FAM90A25P, FAM90A2P, FAM92A1P1, FAM95B1, FAM98A, FAM9C,
FANCB, FANCF, FANCL, FAP, FAR1, FAR2, FARSB, FAS, FASTKD1,
FASTKD2, FAT1, FBXL17, FBXL3, FBXL5, FBXL8, FBXO22, FBXO25,
FBXO28, FBXO30, FBXO32, FBXO33, FBXO34, FBXO39, FBXO4, FBXO43,
FBXO45, FBXO47, FBXO48, FBXO7, FBXO8, FBXW10, FBXW11P1, FBXW7,
FCAMR, FCER1G, FCGBP, FCGR2A, FCGR2C, FCGR3A, FCHO2, FCN3,
FCRL6, FDPSP1, FDPSP4, FDPSP5, FEM1B, FFAR3, FGD2, FGF14-IT1, FGF19,
FGF20, FGF6, FGF7, FGFBP1, FGFR1OP2, FGL2, FGR, FHDC1, FHL5, FICD,
FIG.F, FIG.NL1, FITM2, FKBP11, FKBP14, FKBP4P1, FKBP5, FKTN, FLG,
FLG2, FLG-AS1, FLI1, FLJ14712, FLJ37644, FLJ41603, FLJ43752, FLT1, FLT3,
FLT3LG, FLT4, FLVCR2, FMN1, FMN2, FMO2, FMO5, FMOD, FNDC3A,
FNDC3B, FNDC5, FNDC9, FNIP1, FNIP2, FNTAL1, FNTAP2, FOLR2, FOSL1,
FOSL1P1, FOSL2, FOXC2, FOXD2-AS1, FOXE1, FOXF1, FOXF2, FOXJ2,
FOXJ3, FOXL1, FOXN2, FOXN4, FOXO1, FOXO3, FOXQ1, FOXS1, FPGT,
FPGT-TNNI3K, FPR1, FPR3, FREM3, FRG1, FRG1B, FRK, FRMD7, FRRS1,
FRS2, FSBP, FSD2, FSHR, FTH1P10, FTH1P11, FTH1P2, FTH1P23, FTH1P3,
FTH1P4, FTLP14, FTO-IT1, FUCA1, FUCA2, FUNDC2, FUNDC2P2, FUT3,
FUT5, FUT7, FVT1, FXC1, FXR1, FXYD2, FXYD5, FXYD6P2, FYB, FYCO1,
FYTTD1, FYTTD1P1, FZD4, FZD9, G3BP1, G3BP2, G6PC2, GAB3,
GABARAPL1, GABARAPL3, GABRA1, GABRA2, GABRR1, GABRR2, GAD1,
GAD2, GADD45A, GADD45B, GADD45G, GAL3ST2, GALK2, GALNT10,
GALNT14, GALNT3, GALNT7, GALNT8, GALNTL2, GALNTL5, GAN,
GAPDHP14, GAPDHP2, GAPDHP20, GAPDHP23, GAPDHP33, GAPDHP40,
GAPDHP47, GAPDHP49, GAPDHP55, GAPDHP58, GAPDHP60, GAPDHP61,
GAPDHP63, GAPDHP69, GAPDHP71, GAPDHP72, GAR1, GARS, GAS2, GAS5,
GBA3, GBAS, GBE1, GBP1, GBP1P1, GBP2, GBP4, GBP5, GCC1, GCFC2,
GCGR, GCLM, GCM1, GCNT1P1, GCNT3, GCNT4, GCNT7, GCOM1, GCOM2,
GCSAML, GDA, GDAP2, GDI2, GDI2P1, GDI2P2, GDNF, GDPGP1, GEM,
GEMIN2, GEMIN8P3, GFI1B, GFM1, GFPT1, GFPT2, GGCT, GGNBP1, GGT5,
GGT6, GH1, GH2, GHc-362H12.3, GHDC, GHITM, GHRHR, GHRLOS2, GHSR,
GIMAP4, GIMAP5, GIMAP6, GIMAP8, GIN1, GINS3, GJA5, GJA8, GJB3, GJD2,
GK3P, GKAP1, GK-AS1, GK-IT1, GLB1L3, GLCE, GLE1, GLI1, GLIPR1,
GLIPR1L2, GLIS3, GLO1, GLP1R, GLRA1, GLRA3, GLRB, GLRX2, GLRX3,
GLRXP3, GLT25D2, GLT8D1, GLT8D2, GLTP, GLTPP1, GMCL1, GMCL1P1,
GMFB, GMNN, GMPR, GMPS, GNA13, GNA15, GNAS, GNAS-AS1, GNB4,
GNE, GNG11, GNG12, GNG2, GNG4, GNGT1, GNGT2, GNL2, GNL3, GNL3LP1,
GNLY, GNPDA1, GNPDA2, GNRHR, GNS, GOLGA2, GOLGA2P4, GOLGA4,
GOLGA5, GOLGA6A, GOLGA7B, GOLGA8C, GOLGA8E, GOLGA8H, GOLGB1,
GOLPH3L, GOLSYN, GOLT1A, GOLT1B, GOPC, GORASP2, GOSR2, GOT1,
GOT2P2, GP1BA, GP2, GP5, GP6, GPAM, GPBP1, GPD1, GPD1L, GPD2, GPHN,
GPM6A, GPN3, GPNMB, GPR110, GPR115, GPR116, GPR144, GPR150, GPR158,
GPR158-AS1, GPR17, GPR177, GPR183, GPR19, GPR27, GPR3, GPR31, GPR34,
GPR37L1, GPR4, GPR52, GPR56, GPR6, GPR61, GPR65, GPR75, GPR84, GPR87,
GPR88, GPR89A, GPR97, GPRASP1, GPRC5D, GPRC6A, GPSM2, GPX1P2,
GPX2, GPX3, GRAMD1B, GRAMD1C, GRAMD3, GRAP, GRAPL, GREM1,
GREM2, GRHL3, GRIA2, GRIA4, GRIK1-AS1, GRIN2A, GRIP2, GRK7, GRM4,
GRM7-AS3, GRP, GRPEL2, GRPEL2P2, GS1-174L6.4, GS1-179L18.1, GS1-
184P14.2, GS1-211B7.1, GS1-251I9.2, GS1-251I9.3, GS1-251I9.4, GS1-304P7.1,
GS1-309P15.4, GS1-44D20.1, GS1-465N13.1, GS1-542M4.3, GS1-600G8.5,
GSDMA, GSN, GSN-AS1, GSPT1, GSTA2, GSTO1, GSTP1P1, GTDC1, GTF2A1,
GTF2A2, GTF2B, GTF2E2, GTF2F2, GTF2H2C, GTF2H5, GTF2IRD1P1, GTF3C3,
GTPBP10, GUCA2A, GUCY1A3, GUCY1B3, GUCY2C, GUK1, GUSBP2,
GUSBP4, GUSBP9, GVINP1, GXYLT1, GXYLT2, GYG1, GYG1P1, GYG1P3,
GYLTL1B, GYPB, GYPE, GYS2, GZMB, GZMM, H2AFB1, H2AFZ, H2AFZP3,
H2AFZP6, H3F3AP5, H3F3BP1, HABP4, HADH, HADHAP1, HAPLN1, HAPLN4,
HAR1A, HAR1B, HAS1, HAT1, HAUS1P3, HAUS3, HAUS4, HAUS6, HAUS6P1,
HAUS7, HAVCR2, HBEGF, HBS1L, HBXIP, HCFC1-AS1, HCFC2, HCFC2P1,
HCG4P3, HCG4P5, HCK, HCLS1, HCRT, HCST, HDAC11-AS1, HDAC9, HDDC3,
HDGFP1, HDHD1P1, HEATR1, HEATR4, HEBP2, HECA, HECTD1, HELQ,
HERC2P4, HERC2P5, HERC3, HERC4, HERC5, HERC6, HERPUD1, HESX1,
HEXA-AS1, HEXB, HEY1, HFE, HGD, HGF, HHAT, HHATL, HHATL-AS1,
HHIPL2, HHLA2, HIAT1, HIATL1, HIATL2, HIBADH, HIBCH, HIF1A,
HIGD1AP10, HIGD1AP11, HIGD1AP16, HILPDA, HIN1L, HINT1P1, HIPK1,
HIPK3, HIST1H1A, HIST1H1T, HIST1H2AI, HIST1H2AK, HIST1H2BE,
HIST1H2BI, HIST1H2BN, HIST1H3H, HIST1H4A, HIST1H4B, HIST1H4PS1,
HIST2H2BA, HIST2H2BB, HIST2H4B, HIVEP2, HLA-A, HLA-A29.1, HLA-B,
HLA-C, HLA-F, HLA-F-AS1, HLA-H, HLF, HLTF, HLX, HLXB9, HM13-IT1,
HMGB1, HMGB1P14, HMGB1P27, HMGB1P3, HMGB1P31, HMGB1P44,
HMGB1P9, HMGB2, HMGB2P1, HMGB3P1, HMGB3P17, HMGB3P5, HMGB3P6,
HMGB3P7, HMGN1P24, HMGN1P30, HMGN1P7, HMGN1P8, HMGN2P28,
HMGN2P41, HMGN2P6, HMGN2P8, HMGN4, HMGN5, HMGXB4, HMOX1,
HNRNPA1P14, HNRNPA1P15, HNRNPA1P26, HNRNPA1P28, HNRNPA1P3,
HNRNPA1P4, HNRNPA1P6, HNRNPA1P7, HNRNPA3P1, HNRNPA3P2,
HNRNPC, HNRNPCL1, HNRNPH2, HNRNPKP1, HNRNPKP2, HNRNPKP5,
HNRNPU-AS1, HNRPCP, HOMER2, HOMEZ, HOOK1, HOPX, HORMAD2,
HOXA7, HOXB4, HOXB5, HOXB6, HP, HPCAL4, HPGD, HPRT1, HPS5, HR,
HRASLS3, HRG, HRH1, HRNR, HS3ST2, HS6ST2, hsa-mir-142, hsa-mir-146a, hsa-
mir-1539, hsa-mir-2117, hsa-mir-3149, hsa-mir-3180-4, hsa-mir-4763, hsa-mir-6080,
hsa-mir-6723, HSBP1L1, HSD11B1, HSD17B12, HSD17B13, HSD17B4, HSD17B6,
HSD17B7P2, HSD17B8, HSD3BP2, HSD3BP5, HSDL2, HSF4, HSF5, HSFX1,
HSH2D, HSP90AA1, HSP90AA5P, HSP90AA6P, HSP90AB2P, HSP90AB3P,
HSP90AB6P, HSP90B1, HSP90B2P, HSP90B3P, HSPA13, HSPA1A, HSPA1B,
HSPA2, HSPA4L, HSPA5, HSPA6, HSPA7, HSPA9, HSPA9P1, HSPB1P1, HSPB3,
HSPB6, HSPB9, HSPD1, HSPD1P1, HSPD1P10, HSPD1P11, HSPD1P3, HSPD1P5,
HSPD1P6, HSPE1, HSPH1, HTATIP2, HTR1D, HTR1F, HTR2B, HTR3D, HTR3E,
HTR6, HTR7, HTRA3, HTRA4, HTT-AS1, HULC, HUS1B, HYAL4, IAH1, IAPP,
IARS, IARS2, IBSP, IBTK, ICAM1, ICAM2, ICAM4, ICAM5, ICK, ICOSLG,
ID2B, IDE, IDNK, IDO1, IDS, IDSP1, IER3, IER3IP1, IER5, IFI16, IFI30, IFI35,
IFI44, IFI44L, IFIH1, IFIT1, IFIT2, IFIT3, IFIT5, IFLTD1, IFNA5, IFNAR2,
IFNGR1, IFNW1, IFT20, IFT74, IFT88, IGBP1-AS2, IGBP1P1, IGBP1P4, IGF1R,
IGF2-AS, IGFBP4, IGFBP7, IGFL2, IGIP, IGKV1-16, IGKV1-17, IGKV1D-17,
IGLL3P, IGLV5-52, IGLV9-49, IGLVIV-59, IGSF11, IGSF11-AS1, IGSF5,
IKBKAP-IT1, IKBKGP1, IKZF2, IKZF5, IL10, IL10RB, IL11, IL12RB1, IL12RB2,
IL13RA1, IL13RA2, IL17B, IL17RB, IL17RE, IL18RAP, IL19, IL1A, IL1B, IL1R1,
IL1R2, IL1RL1, IL1RL2, IL1RN, IL20, IL20RA, IL20RB, IL21R, IL21R-AS1,
IL22RA1, IL23A, IL24, IL29, IL2RA, IL2RB, IL2RG, IL31, IL33, IL36A, IL36G,
IL5, IL5RA, IL6, IL6R, IL6ST, IL7R, IL8, ILDR1, ILF3-AS1, IMMP1L, IMMP2L,
IMMTP1, IMPA1P, IMPAD1, IMPDH1P11, IMPDH1P6, IMPG1, IMPG2, ING2,
INGX, INHBA, INHBA-AS1, INHBC, INPP4B, INS, INSC, INSIG1, INS-IGF2,
INSL3, INSL5, INSM2, INSRR, INTS4L2, INTS6, INTS6-AS1, INTS6P1, IPCEF1,
IPMK, IPO7, IPO7P1, IPO7P2, IPO8, IPO8P1, IQCB1, IQCB2P, IQCF3, IQCH,
IQCH-AS1, IQGAP1, IQUB, IRAK2, IRAK4, IRF1, IRF4, IRF5, IRF8, IRS2,
ISCA1, ISCA1P1, ISCA1P4, ISCA1P6, ISCU, ISG20, ISLR, ISM1, ISM1-AS1,
ISOC1, ITCH, ITGA10, ITGA2, ITGAD, ITGAM, ITGAV, ITGAX, ITGB2, ITGB3,
ITGB3BP, ITIH3, ITIH4, ITIH5, ITK, ITM2B, ITPK1-AS1, ITPR2, ITPR3,
ITPRIPL2, IZUMO2, J01415.10, J01415.13, J01415.14, J01415.2, J01415.23,
J01415.24, J01415.25, J01415.4, J01415.5, J01415.6, J01415.9, JAGN1, JAK1,
JAK2, JAKMIP3, JAZF1, JHDM1D, JKAMP, JMJD1C, JMY, JPH1, JRKL, JUNB,
KAAG1, KAT2B, KAT7, KATNA1, KB-1047C11.2, KB-1090H4.2, KB-1183D5.11,
KB-1208A12.3, KB-1460A1.5, KB-1562D12.3, KB-1592A4.14, KB-1615E4.2, KB-
1639H6.4, KB-1732A1.1, KB-1907C4.2, KB-1980E6.3, KB-431C1.3, KB-431C1.4,
KBTBD10, KBTBD2, KBTBD3, KBTBD5, KCMF1, KCNA1, KCNA2, KCNA5,
KCNAB1, KCNC1, KCNC4-AS1, KCND3-IT1, KCNE1, KCNE4, KCNF1, KCNG3,
KCNH1, KCNH1-IT1, KCNH2, KCNJ10, KCNJ13, KCNJ14, KCNJ15, KCNJ8,
KCNJ9, KCNK1, KCNK12, KCNK3, KCNK4, KCNK6, KCNK9, KCNMA1,
KCNMB2, KCNMB2-IT1, KCNN4, KCNQ1-AS1, KCTD13, KCTD14, KCTD6,
KCTD9, KCTD9P1, KCTD9P2, KDELC2, KDELR2, KDELR3, KDM1B, KDM4D,
KDR, KDSR, KHDC1L, KIAA0020, KIAA0146, KIAA0196-AS1, KIAA0226L,
KIAA0247, KIAA0319, KIAA0367, KIAA0368, KIAA0408, KIAA0430,
KIAA0494, KIAA0672, KIAA0947, KIAA1033, KIAA1107, KIAA1128,
KIAA1147, KIAA1274, KIAA1279, KIAA1383, KIAA1407, KIAA1429,
KIAA1430, KIAA1524, KIAA1551, KIAA1586, KIAA1598, KIAA1614,
KIAA1644, KIAA1704, KIAA1731, KIAA1737, KIAA2018, KIAA2022,
KIAA2026, KIF13A, KIF21A, KIF25, KIF26B, KIF27, KIF5B, KIRREL3,
KIRREL3-AS2, KLF1, KLF10, KLF11, KLF14, KLF3P1, KLF7-IT1, KLF8, KLF9,
KLHDC1, KLHDC5, KLHDC7B, KLHDC8A, KLHL1, KLHL12, KLHL15,
KLHL20, KLHL24, KLHL28, KLHL30, KLHL32, KLHL33, KLHL38, KLHL6,
KLHL7-AS1, KLHL8, KLK1, KLK11, KLK3, KLKB1, KLRC1, KNG1, KPNA1,
KPNA4, KRBOX1, KRBOX1-AS1, KRCC1, KRIT1, KRR1, KRT10, KRT12,
KRT13, KRT16P1, KRT16P2, KRT17, KRT18P10, KRT18P16, KRT18P17,
KRT18P29, KRT18P37, KRT18P4, KRT18P8, KRT222, KRT28, KRT32, KRT40,
KRT78, KRT80, KRT8P10, KRT8P22, KRT8P28, KRT8P30, KRT8P31, KRT8P37,
KRT8P41, KRT8P46, KRT8P7, KRT8P9, KRTAP10-1, KRTAP10-10, KRTAP10-2,
KRTAP10-7, KRTAP12-1, KRTAP12-3, KRTAP3-2, KRTAP3-3, KRTAP4-3,
KRTAP4-4, KRTAP5-4, KRTAP5-8, KTN1, KY, KYNU, L29074.3, L34079.1,
LA16c-306A4.1, LA16c-306E5.2, LA16c-306E5.3, LA16c-321D4.1, LA16c-
321D4.2, LA16c-325D7.2, LA16c-390E6.3, LA16c-390H2.4, LA16c-431H6.6,
LACC1, LACTB2, LAIR1, LAMA3, LAMB4, LAMC2, LAMP1, LAMP2, LAMP3,
LAMTOR3, LAMTOR3P2, LAP3P2, LAPTM4A, LAPTM5, LARP1B, LARP1P1,
LARP4, LARP4P, LARP7, LARS, LAT, LAX1, LBP, LCA5, LCA5L, LCE5A,
LCMT2, LCN10, LCN2, LCNL1, LCP2, LCT, LDHAL6FP, LDHBP1, LDHBP2,
LDHC, LDLRAP1, LEAP2, LECT1, LEMD1-AS1, LEMD3, LEPROTL1, LETMD1,
LGALS3, LGALS7B, LGALS8, LGALS9, LGI1, LGI2, LGR5, LGSN, LHFP, LIAS,
LIF, LILRA2, LILRA3, LILRB1, LILRB2, LILRB4, LIMCH1, LIMD1, LIMD1-
AS1, LIMS1, LIN54, LINC00152, LINC00159, LINC00161, LINC00184,
LINC00207, LINC00222, LINC00229, LINC00237, LINC00240, LINC00263,
LINC00266-1, LINC00271, LINC00284, LINC00303, LINC00312, LINC00334,
LINC00336, LINC00339, LINC00356, LINC00359, LINC00361, LINC00365,
LINC00381, LINC00391, LINC00398, LINC00426, LINC00441, LINC00449,
LINC00464, LINC00467, LINC00472, LINC00473, LINC00475, LINC00478,
LINC00485, LINC00494, LINC00511, LINC00516, LINC00521, LINC00525,
LINC00571, LINC00594, LINC00598, LINC00608, LINC00612, LINC00622,
LINC00642, LINC00643, LINC00654, LINC00657, LINC00659, LINC00662,
LINC00663, LINGO4, LIPH, LIPT1, LIX1L, LL0XNC01-116E7.2, LL0XNC01-
131B10.2, LL0XNC01-237H1.3, LL0XNC01-250H12.2, LL0XNC01-250H12.3,
LLPH, LMAN1, LMBRD1, LMBRD2, LMCD1, LMNA, LMO1, LMTK2, LNP1,
LNPEP, LNX1-AS2, LOC100128731, LOC100129673, LOC100129697,
LOC100129913, LOC100130291, LOC100130633, LOC100132060,
LOC100132117, LOC100132299, LOC100132444, LOC100133273,
LOC100133328, LOC100133600, LOC100133866, LOC100134018,
LOC100134188, LOC100144604, LOC133993, LOC144481, LOC145853,
LOC146517, LOC147727, LOC154761, LOC203547, LOC283481, LOC284230,
LOC284988, LOC285176, LOC285412, LOC339804, LOC387647, LOC388339,
LOC388681, LOC389493, LOC390530, LOC392501, LOC399744, LOC399959,
LOC400013, LOC401218, LOC401233, LOC440335, LOC440487, LOC440575,
LOC440737, LOC441246, LOC641825, LOC642755, LOC643031, LOC643310,
LOC643320, LOC643452, LOC643740, LOC644128, LOC644132, LOC645174,
LOC645196, LOC645317, LOC645586, LOC645609, LOC646110, LOC646135,
LOC646630, LOC647104, LOC647150, LOC647251, LOC647307, LOC649456,
LOC650200, LOC650215, LOC650254, LOC650832, LOC652668, LOC652881,
LOC653071, LOC653513, LOC653566, LOC653853, LOC654161, LOC728178,
LOC728431, LOC728576, LOC728937, LOC729086, LOC729314, LOC729362,
LOC729768, LOC730994, LOC731365, LONRF3, LOR, LOXHD1, LPAR4,
LPAR6, LPGAT1, LPIN1, LPL, LPP, LRFN2, LRFN5, LRIF1, LRIG1, LRIT3,
LRP11, LRP2BP, LRP4, LRP4-AS1, LRP8, LRR1, LRRC15, LRRC16A, LRRC2,
LRRC25, LRRC34, LRRC37A, LRRC37A15P, LRRC37A6P, LRRC37B,
LRRC37BP1, LRRC39, LRRC3C, LRRC4, LRRC52, LRRC8B, LRRC8C, LRRC8E,
LRRCC1, LRRD1, LRRFIP2, LRRIQ3, LRRIQ4, LRRK2, LRRTM3, LRRTM4,
LSAMP, LSAMP-AS1, LSG1, LSM3, LSM3P5, LSM5, LSP1, LSP1P2, LTBR, LTF,
LTV1, LUC7L2, LURAP1L, LVRN, LXN, LY6K, LY75, LY9, LY96, LYAR, LYN,
LYNX1, LYRM2, LYRM5, LYRM7, LYSMD2, LYST-IT1, LYVE1, LZIC,
MAB21L3, MAEL, MAFA, MAFIPL, MAGEA11, MAGEE2, MAGOH, MAGOH2,
MAGOHB, MAGT1, MAK16, MAL2, MALAT1, MAMDC2, MAN1A1, MAN1A2,
MAN1C1, MANEA, MANSC1, MANSC4, MAP1LC3A, MAP1LC3B,
MAP1LC3B2, MAP2, MAP2K1, MAP2K1P1, MAP2K3, MAP3K13, MAP3K14,
MAP3K14-AS1, MAP3K2, MAP3K5, MAP3K6, MAP3K7, MAP3K8, MAP6D1,
MAP7, MAP7D2, MAPK10, MAPK15, MAPK1IP1L, MAPK9, MAPKAPK2,
MAPRE1P1, MAPT, MAPT-AS1, MARCO, MARK1, MARK2P8, MARVELD1,
MAST4-AS1, MAT1A, MAT2A, MAT2B, MB, MB21D1, MBD2, MBD3L5,
MBD4, MBIP, MBL1P, MBLAC2, MBNL1, MBNL2, MBNL3, MBOAT4, MC1R,
MCEE, MCF2L2, MCFD2P1, MCHR1, MCMBP, MCMDC2, MCOLN2, MCPH1,
MCTP2, MCTS1, MDFIC, MDH1, MDH2, MDM2, ME1, ME2, ME3, MEAF6P1,
MED14, MED21, MED4, MED7, MED9, MEF2A, MEFV, MEG3, MEG8, MEG9,
MEGF9, MEIG1, MEIOB, MEIS1-AS1, MEIS1-AS2, MEIS2, MEIS3P1, MEPE,
MESP1, MESP2, MESTP3, METAP2, Metazoa_SRP, METRNL, METT10D,
METTL1, METTL14, METTL15, METTL15P1, METTL16, METTL20,
METTL21A, METTL21B, METTL21C, METTL23, METTL5, METTL7A, MFAP1,
MFAP1P1, MFAP3L, MFHAS1, MFSD1, MFSD2A, MGARP, MGAT1, MGAT2,
MGAT4A, MGAT5, MGC16291, MGC18216, MGC50722, MGEA5, MGP, MIA2,
MIA3, MID1, MIER3, MINA, MINOS1P1, MINOS1P3, MINPP1, MIOS, MIOX,
MIPEP, MIPEPP3, MIR106B, MIR1185-1, MIR1185-2, MIR1249, MIR1250,
MIR1254-1, MIR1255B1, MIR125B2, MIR1290, MIR129-2, MIR1302-11, MIR141,
MIR1470, MIR151B, MIR153-1, MIR1538, MIR155HG, MIR181C, MIR181D,
MIR185, MIR192, MIR194-2, MIR1974, MIR199B, MIR200A, MIR200B,
MIR200C, MIR22HG, MIR23A, MIR23B, MIR24-1, MIR26A2, MIR27A, MIR27B,
MIR299, MIR29B2, MIR29C, MIR3116-2, MIR3117, MIR3124, MIR3132,
MIR3174, MIR3180-1, MIR3183, MIR3184, MIR3188, MIR3189, MIR3192,
MIR3202-2, MIR320B2, MIR331, MIR342, MIR34A, MIR3605, MIR3613, MIR367,
MIR3671, MIR3680-1, MIR3680-2, MIR3682, MIR369, MIR3692, MIR370,
MIR374A, MIR376A1, MIR377, MIR378D2, MIR3916, MIR3918, MIR3926-2,
MIR3929, MIR3935, MIR3944, MIR3945, MIR409, MIR410, MIR412, MIR4258,
MIR4263, MIR4284, MIR429, MIR4319, MIR4426, MIR4458, MIR4461, MIR4484,
MIR4489, MIR4492, MIR4512, MIR4515, MIR4523, MIR4526, MIR4534, MIR455,
MIR4635, MIR4638, MIR4642, MIR4653, MIR4656, MIR4665, MIR4669,
MIR4672, MIR4682, MIR4685, MIR4689, MIR4701, MIR4712, MIR4714,
MIR4728, MIR4729, MIR4740, MIR4754, MIR4761, MIR4786, MIR4787, MIR493,
MIR499A, MIR5001, MIR5188, MIR5191, MIR5196, MIR541, MIR548AN,
MIR548D2, MIR548U, MIR5587, MIR559, MIR568, MIR5695, MIR5698,
MIR5699, MIR572, MIR574, MIR575, MIR589, MIR597, MIR609, MIR639,
MIR642A, MIR654, MIR655, MIR656, MIR659, MIR665, MIR670, MIR708, MIR7-
3, MIR758, MIR769, MIR770, MIR99B, MIRLET7DHG, MIS12, MIS18BP1,
MITD1, MK167IP, MKLN1, MKRN2, MKRN2-AS1, MKRN4P, MKX, MLC1,
MLIP, MLIP-AS1, MLKL, MLLT10P1, MLNR, MLPH, MMAA, MMADHC,
MMP1, MMP3, MMP7, MMRN1, MNAT1, MNS1, MNX1, MOB1A, MOBKL1B,
MOBP, MOCOS, MOCS2, MON2, MORC3, MORF4, MORF4L1P1, MORF4L2,
MORN2, MOSC2, MOSPD2, MOV10L1, MPC1, MPC1L, MPEG1, MPHOSPH10,
MPHOSPH8, MPLKIP, MPP1, MPP5, MPP6, MPP7, MPRIP-AS1, MPV17,
MPV17L, MR1, MRAP2, MRC1, MRE11A, MRFAP1L1, MRGPRE, MRO,
MRPL1, MRPL18, MRPL19, MRPL24, MRPL3, MRPL32, MRPL33, MRPL39,
MRPL3P1, MRPL42, MRPL46, MRPL48, MRPL50, MRPS10, MRPS15P2,
MRPS23, MRPS29P2, MRPS30, MRPS31, MRPS31P3, MRPS31P4, MRPS33,
MRPS36, MRPS5P3, MS4A10, MS4A14, MS4A4A, MS4A6A, MS4A7,
MSANTD4, MSI2, MSL2, MSMO1, MSR1, MST4, MSTN, MSX1, MT1A, MT1B,
MT1DP, MT1E, MT1F, MT1G, MT1H, MT1JP, MT1L, MT1M, MT1P1, MT1P3,
MT1X, MT1XP1, MT2A, MT4, MT-ATP6, MTBP, MT-CO1, MT-CO2, MT-CYB,
MTDH, MTE, MTERF, MTERFD1, MTF1, MTHFD1, MTHFD1P1, MTIF2, MTIF3,
MTMR2, MTMR6, MTMR8, MTND1P23, MT-ND2, MTND2P28, MT-ND3,
MTND3P10, MT-ND4, MT-ND4L, MTND4P10, MTND4P11, MTND4P14,
MTND4P15, MTND4P24, MTND4P26, MT-ND5, MTND5P19, MTND5P28, MT-
ND6, MTO1, MTPN, MTRNR2L3, MTRNR2L4, MTRNR2L8, MTRNR2L9,
MTRR, MTUS1, MTUS2-AS1, MTX3, MUC12, MUC13, MUC17, MUSTN1, MVP,
MX1, MXD1, MXI1, MXRA7, MYADML, MYBL1, MYCBP2, MYCT1, MYH11,
MYH15, MYH6, MYH7, MYL3, MYLIP, MYLK3, MYLPF, MYNN, MYO1D,
MYO1F, MYO3A, MYO3B, MYO5A, MYO5BP1, MYOCD, MYOF, MYOM1,
MYOT, MYPN, MZT1, N4BP2L1, N4BP2L2, NAA15, NAA20, NAA25, NAA30,
NAA35, NAA50, NAALAD2, NAALADL2, NAB1, NACA2, NACA3P, NACAP1,
NADKD1, NALCN, NALCN-AS1, NAMPT, NAMPTL, NANOS2, NANP, NANS,
NAP1L1P1, NAP1L1P2, NAP1L1P3, NAP1L2, NAP1L4P3, NAP1L5, NAPEPLD,
NAPG, NARS, NAT16, NAT8, NAV2, NAV2-AS1, NAV2-AS2, NAV2-AS3,
NBEAP1, NBL1, NBN, NBPF11, NBPF14, NBPF24, NBPF7, NBR1, NCAPG,
NCAPG2, NCBP1, NCBP2, NCBP2L, NCEH1, NCF1, NCF1C, NCF2, NCF4,
NCK1, NCKAP1, NCKAP1L, NCMAP, NCOA2, NCOA3, NCOA4, NCR1,
NCR3LG1, NCRNA00200, NDEL1, NDFIP1, NDFIP2, NDRG1, NDUFA4,
NDUFA5, NDUFAB1, NDUFAF1, NDUFAF2, NDUFAF4, NDUFAF5, NDUFB3,
NDUFC2, NDUFS4, NDUFV2, NEAT1, NECAB1, NECAP1P1, NEDD4, NEFH,
NEFHP1, NEIL2, NEK1, NEK10, NEK4, NELF, NELL2, NEO1, NET1, NEUROD2,
NEXN, NEXN-AS1, NFAM1, NFE2L2, NFIA, NFIC, NFIL3, NFIX, NFKBIA,
NFS1, NFU1, NFX1, NGEF, NGF, NGLY1, NHEJ1, NHLH2, NHLRC1, NHLRC3,
NID1, NIF3L1, NIN, NIPAL1, NIPAL4, NIPBL, NIPSNAP3B, NKAP, NKAPL,
NKIRAS1, NKRF, NKX2-2, NKX2-2-AS1, NKX2-3, NKX3-1, NKX3-2, NKX6-1,
NLF2, NLGN1, NLK, NLRC4, NLRC5, NLRP2, NLRP9, NMD3, NMD3P1,
NME2P1, NME5, NME7, NMI, NMNAT2, NMRK1, NMUR1, NMUR2, NNMT,
NOC3L, NOL10, NOL3, NOL4, NOL7, NOL8, NOLC1, NOP58, NOSTRIN,
NOTCH2NL, NOTO, NOV, NOX5, NOXRED1, NPAS2, NPAS4, NPAT, NPBWR1,
NPC1, NPL, NPM1P18, NPM1P21, NPM1P6, NPPC, NPS, NPTN, NPTX2, NPVF,
NPY, NPY6R, NQO1, NQO2, NR0B1, NR1D2, NR1H2, NR1I2, NR2C2AP, NR2E3,
NR3C1, NR3C2, NR4A2, NR4A3, NR5A2, NRCAM, NRD1, NRG1, NRG1-IT1,
NRG2, NRG3-AS1, NRG4, NRIP1, NRIP3, NRP2, NRSN1, NSA2, NSF, NSFP1,
NSL1, NSMAF, NSMCE1, NSRP1, NSUN2, NSUN3, NSUN6, NT5C1B, NT5CP1,
NTPCR, NTRK1, NUAK2, NUB1, NUCB2, NUCKS1, NUDCD2, NUDT12,
NUDT19, NUDT21, NUDT22, NUDT3, NUDT4, NUDT4P1, NUDT6, NUDT7,
NUDT9, NUFIP1, NUFIP1P, NUFIP2, NUGGC, NUP107, NUP155, NUP35,
NUP62CL, NUP98, NUPL1, NUPR1, NUPR1L, NUS1, NUS1P1, NWD1, NXF2B,
NXF5, NXNL1, NXPE3, NXT1, NXT2, NYAP2, O3FAR1, OAF, OAS2, OAS3,
OASL, OAT, OBFC1, OCIAD1, OCIAD1-AS1, OCLM, OCM2, ODC1, ODF4,
OFD1P17, OGDHL, OGFOD1, OGFOD1P1, OGN, OGT, OIP5, OIP5-AS1, OLFM1,
OLFM4, OLIG1, OLIG2, OLIG3, OLR1, OMA1, OMD, OMG, OMP, OPA1,
OPALIN, OPRD1, OPTC, OR10AC1P, OR10AD1, OR10H1, OR10H5, OR13A1,
OR1F12, OR1L3, OR1L8, OR1Q1, OR2A13P, OR2L13, OR3A3, OR3B1P,
OR4A46P, OR4K13, OR4N2, OR4X2, OR52B3P, OR5B2, OR5K2, OR6D1P,
OR6E1P, OR7D2, OR7E154P, OR7E91P, OR7L1P, OR8S1, OR8T1P, OR9A1P,
OR9A3P, OR9M1P, ORC2, ORC3, ORC3L, ORC4, ORC5, ORMDL1, orphan,
OSBP, OSBPL3, OSBPL6, OSBPL8, OSCAR, OSGEPL1, OSGIN2, OSM, OSMR,
OSTC, OSTCP1, OSTCP2, OSTCP4, OSTCP5, OSTCP6, OSTF1, OSTF1P1,
OSTM1, OTOGL, OTUD1, OTUD3, OTUD4, OTUD6B, OTUD7B, OVOL2,
OVOL3, OXCT2, OXCT2P1, OXGR1, OXNAD1, OXR1, OXSM, OXTR, P2RX2,
P2RX5, P2RX6, P2RX7, P2RY1, P2RY13, P2RY5, P4HA1, P4HA3, PA2G4P2,
PA2G4P4, PABPC1P1, PABPC1P10, PABPC1P11, PACRGL, PACSIN2,
PAFAH1B2, PAICSP4, PAIP2, PAK1IP1, PAK3, PALM2, PALM2-AKAP2,
PALMD, PAM, PAN2, PAN3-AS1, PAPD5, PAPOLA, PAPPA2, PAPSS2, PAQR3,
PARD3B, PARM1, PARP10, PARP12, PARP14, PARP15, PARP1P1, PARP8,
PARP9, PART1, PARVA, PARVB, PARVG, PATE1, PATE4, PATL1, PATL2,
PAX5, PAX6, PAX9, PBLD, PCA3, PCDH17, PCDH20, PCDH7, PCDHA3,
PCDHAC1, PCDHB1, PCDHB11, PCDHB13, PCDHB18, PCDHB3, PCDHGA1,
PCDHGA11, PCDHGA12, PCDHGA2, PCDHGA3, PCDHGA4, PCDHGA5,
PCDHGA6, PCDHGA8, PCDHGA9, PCDHGB1, PCDHGB2, PCDHGB3,
PCDHGC3, PCGF1, PCGF5, PCK1, PCLO, PCM1, PCMTD1P3, PCNA, PCNP,
PCNPP5, PCSK1, PCSK2, PCYOX1, PDCD1, PDCD10, PDCD1LG2, PDCD4,
PDCD6IP, PDCL, PDE10A, PDE12, PDE1B, PDE3A, PDE4D, PDE4DIP, PDE7B,
PDE8A, PDE8B, PDGFRL, PDIA3, PDIA3P, PDIA4, PDIA6, PDK4, PDLIM1,
PDLIM5, PDP2, PDPK2, PDS5A, PDX1, PDX1-AS1, PDZD8, PDZD9, PEA15,
PEAR1, PEBP1, PEG3, PELO, PEMT, PER3, PERP, PET117, PEX1, PEX11A,
PEX12, PEX13, PEX2, PEX3, PEX5L-AS1, PEX7, PFKFB2, PFN1P2, PFN1P7,
PFN2, PFTK1, PGAM1P11, PGAM1P7, PGAM4P1, PGBD3, PGBD4P3, PGBD4P7,
PGBD5, PGDP1, PGM2, PGM3, PGM5P1, PGPEP1L, PGR, PGRMC2, PHACTR2,
PHBP1, PHBP15, PHBP4, PHEX, PHEX-AS1, PHF1, PHF10, PHF11, PHF15,
PHF17, PHF6, PHGR1, PHKA1, PHKA1P1, PHLDA3, PHLPP2, PHOSPHO2,
PHTF2, PHYHIP, PHYHIPL, PI15, PI3, PIAS2, PIBF1, PICALM, PIGA, PIGB,
PIGC, PIGF, PIGH, PIGK, PIGN, PIGP, PIGR, PIGV, PIGY, PIK3C2A, PIK3C2G,
PIK3CA, PIK3CB, PIK3R6, PINX1, PION, PIR, PIRT, PITHD1, PITPNC1, PIWIL2,
PJA2, PKDREJ, PKHD1, PKIB, PKN2, PKP2, PKP4, PKP4P1, PLA1A, PLA2G12A,
PLA2G12AP1, PLA2G1B, PLA2G2C, PLA2G2E, PLA2G4A, PLA2G4C, PLA2G7,
PLAA, PLAC9, PLAGL1, PLAT, PLAU, PLAUR, PLB1, PLBD1, PLCB1, PLCB4,
PLCE1, PLCH2, PLCL2, PLCL2-AS1, PLCXD3, PLCZ1, PLD1, PLD6, PLEK,
PLEK2, PLEKHA1, PLEKHA2, PLEKHA3, PLEKHA8P1, PLEKHB2, PLEKHF2,
PLEKHH2, PLEKHM2, PLEKHN1, PLEKHS1, PLGLB1, PLGRKT, PLIN5,
PLOD2, PLP2, PLS1, PLSCR1, PLSCR2, PLVAP, PM20D1, PMCH, PMM2, PMS1,
PNLIP, PNLIPRP1, PNLIPRP2, PNMA2, PNO1, PNP, PNPLA2, PNPLA5,
PNPLA8, PNPO, PNRC1, PNRC2, POC1B, POC5, POF1B, POGK, POLD3, POLE3,
POLE4, POLG2, POLH, POLK, POLQ, POLR1D, POLR2B, POLR2C, POLR2J2,
POLR2K, POLR3B, POLR3G, POLR3GL, POMC, POMP, PON3, POP5, POP7,
POPDC3, POT1, POTEG, POU2AF1, POU4F1-AS1, PP2D1, PPA1, PPA2,
PPAPDC1B, PPBP, PPCDC, PPCS, PPIAP13, PPIAP14, PPIAP2, PPIAP20,
PPIAP3, PPIEL, PPIG, PPIL4, PPIL6, PPIP5K1, PPM1A, PPM1E, PPM1H, PPM1K,
PPME1, PPP1CB, PPP1R11, PPP1R14C, PPP1R15B, PPP1R1A, PPP1R1C, PPP1R2,
PPP1R27, PPP1R2P3, PPP1R2P6, PPP1R36, PPP1R3C, PPP1R3D, PPP2CA,
PPP2CB, PPP2R1B, PPP2R2C, PPP2R5A, PPP2R5E, PPP3CC, PPP3R1, PPP6C,
PPT1, PQLC3, PRAMEF19, PRB4, PRDM1, PRDM4, PRDX1, PRDX3P1, PRDX4,
PRELID1P2, PRELID1P4, PRELP, PREPL, PREX1, PRG2, PRG4, PRICKLE2-AS3,
PRICKLE3, PRIM2, PRIMA1, PRINS, PRKAB2, PRKACB, PRKAG2-AS1,
PRKCA, PRKCE, PRKCH, PRKRIR, PRKRIRP1, PRKRIRP6, PRKRIRP7,
PRKRIRP8, PRKRIRP9, PRKX, PRL, PRMT10, PRNP, PROCR, PROK2, PROP1,
PRORSD1P, PROSC, PROX1-IT1, PRPF18, PRPF38B, PRPF4, PRPF40A, PRPF6,
PRPH, PRPH2, PRPS1L1, PRPS2, PRR13, PRR16, PRRC1, PRRC2A, PRRT1,
PRRT3, PRRX2, PRSS1, PRSS23, PRSS3, PRSS3P1, PRSS3P2, PRSS41, PRSS48,
PRSS51, PRSS55, PRSS58, PRUNE, PRUNE2, PSAT1P3, PSCA, PSG4, PSG5,
PSG9, PSMA1, PSMA2P1, PSMA2P3, PSMA3, PSMA4, PSMA6, PSMA6P2,
PSMB1, PSMB10, PSMB8, PSMC1P1, PSMC1P10, PSMC1P5, PSMC1P9, PSMC2,
PSMC3, PSMC3IP, PSMC6, PSMD1, PSMD10, PSMD10P2, PSMD12, PSMD14,
PSMD6, PSME2P2, PSMG1, PSMG2, PSPC1-OT1, PSPHP1, PTAFR, PTAR1,
PTBP3, PTCD2, PTCHD3, PTCHD4, PTDSS1, PTEN, PTENP1, PTER, PTGDS,
PTGER4P2, PTGES3, PTGES3P1, PTGES3P2, PTGES3P3, PTGES3P4, PTGFR,
PTGIR, PTGR1, PTGS1, PTGS2, PTH2R, PTHLH, PTK2, PTMAP3, PTP4A1P6,
PTP4A2P1, PTPDC1, PTPN11, PTPN13, PTPN20A, PTPN20B, PTPN22, PTPN2P1,
PTPN3, PTPN4, PTPN7, PTPRC, PTPRE, PTPRH, PTPRJ, PTRH2, PTX3, PURA,
PURB, PUS10, PUS3, PUS7L, PVRIG, PVRL3, PVRL3-AS1, PVT1, PWWP2AP1,
PWWP2B, PXDC1, PXK, PYDC1, PYGL, PYGM, PYGO1, PYROXD1, PYURF,
PYY2, QPCT, QRFP, QRSL1P1, QTRTD1, R3HCC1L, RAB10, RAB11FIP1,
RAB11FIP5, RAB12, RAB14, RAB18, RAB1A, RAB21, RAB22A, RAB23,
RAB24, RAB27A, RAB2A, RAB30, RAB31, RAB33B, RAB39B, RAB3GAP1,
RAB3GAP2, RAB3IP, RAB40A, RAB40AL, RAB42, RAB5A, RAB6A, RAB7L1,
RAB9A, RAB9B, RABEP1, RABGAP1L, RABGEF1, RABGGTB, RABL3,
RAD17, RAD18, RAD50, RAD51AP2, RAD51C, RAD54B, RAET1E, RAET1G,
RAET1K, RAG1, RAI1-AS1, RALGAPA1P, RALGAPB, RAMP3, RANBP2,
RANBP3L, RANBP6, RANBP9, RANGAP1, RAP1A, RAP1B, RAP1GDS1,
RAPH1, RARRES1, RARRES3, RARS, RASA2, RASAL3, RASD1, RASD2,
RASGEF1A, RASGRF1, RASGRF2, RASGRP1, RASGRP3, RASL12, RASSF5,
RASSF6, RASSF8, RAX, RAX2, RB1, RBBP4P1, RBBP4P2, RBBP5, RBBP8,
RBFOX1, RBKS, RBL2, RBM11, RBM12B, RBM12B-AS2, RBM15, RBM18,
RBM20, RBM26-AS1, RBM27, RBM43, RBM45, RBM47, RBMX2P3, RBP4,
RBP7, RBPJL, RBPMSLP, RC3H2, RCAN2, RCBTB2, RCC2P4, RCC2P7, RCHY1,
RCSD1, RDH10, RDH14, RDX, RECQL, REEP5, REG1A, REG1B, REG1P,
REG3A, REG3G, RELB, REN, REP1, REPS2, RERGL, REXO2, RFC1, RFC3,
RFC4, RFC5, RFESD, RFESDP1, RFFL, RFKP1, RFPL3, RFPL3-AS1, RFTN1,
RFX6, RFX8, RGCC, RGL1, RGN, RGPD1, RGPD5, RGPD8, RGS1, RGS10,
RGS16, RGS17, RGS17P1, RGS20, RGS22, RGS5, RGS7BP, RGS9, RGS9BP,
RHBDD1, RHCG, RHD, RHEB, RHEBL1, RHOBTB1, RHOBTB3, RHOJ, RHOQ,
RHOQP1, RHOQP2, RHOQP3, RHOT1, RHOT1P1, RHOT1P3, RIBC2, RIC8B,
RICH2, RIIAD1, RILP, RIMBP3B, RIMBP3C, RIMKLBP1, RIN1, RIN2, RIN3,
RINL, RINT1, RIOK1, RIOK2, RIPK2, RIPK3, RIPPLY1, RIPPLY2, RLF, RLIM,
RLIMP1, RLIMP2, RLN1, RLN2, RLN3, RMND1, RMND5A, RN7SL1,
RNA5SP108, RNA5SP118, RNA5SP122, RNA5SP132, RNA5SP162, RNA5SP18,
RNA5SP187, RNA5SP203, RNA5SP217, RNA5SP219, RNA5SP261, RNA5SP262,
RNA5SP263, RNA5SP265, RNA5SP33, RNA5SP370, RNA5SP383, RNA5SP429,
RNA5SP450, RNA5SP462, RNA5SP494, RNA5SP496, RNA5SP53, RNase_MRP,
RNASE1, RNASE2, RNASE6, RNASEH1P2, RNASEH2B, RNASEH2CP1,
RNASEL, RNF103, RNF114, RNF13, RNF130, RNF133, RNF135, RNF138P1,
RNF14, RNF141, RNF145, RNF148, RNF168, RNF169, RNF180, RNF183,
RNF186, RNF19A, RNF19B, RNF2, RNF216-IT1, RNF219, RNF223, RNF32,
RNF6, RNFT1, RNGTT, RNLS, RNU12-2P, RNU1-5, RNU2-5P, RNU2-6P, RNU2-
79, RNU4-1, RNU4-8P, RNU5A-1, RNU6-10, RNU6-2, RNU6-33, RNU6-42,
RNU6-45, RNU6-8, RNU6V, RNU7-3P, RNU7-47P, RNU7-71P, RNY1, RNY1P6,
RNY3, RNY4P26, RNY4P4, RNY4P6, ROBO2, ROCK1, ROCK2, ROPN1B,
ROPN1L, RORA, RORC, RP1-102E24.8, RP1-102E24.9, RP1-102K2.6, RP11-
1006G14.1, RP11-1006G14.2, RP11-1008C21.1, RP11-100A13.1, RP11-100G15.10,
RP11-100G15.3, RP11-100L22.2, RP11-1016B18.1, RP11-1017G21.3, RP11-
1018J11.1, RP11-101E13.1, RP11-101E7.2, RP11-101P17.11, RP11-101P17.6,
RP11-1026M7.2, RP11-1029J19.5, RP11-102F4.2, RP11-102F4.3, RP11-102H24.1,
RP11-102L12.2, RP11-102M11.1, RP11-1033A18.1, RP11-104L21.2, RP11-
104O19.2, RP11-1055B8.3, RP11-105C20.1, RP11-1060G2.1, RP11-1060J15.4,
RP11-106M3.1, RP11-106M7.1, RP11-1070A24.1, RP11-1074O12.1, RP11-
1079K10.2, RP11-107I14.2, RP11-107M16.2, RP11-1084A12.1, RP11-1084A12.2,
RP11-1084J3.1, RP11-108B14.4, RP11-108M9.4, RP11-108M9.5, RP11-108O10.2,
RP11-1090M7.1, RP11-1090M7.2, RP11-1094M14.7, RP11-1096G20.5, RP11-
109A6.4, RP11-109D9.3, RP11-109D9.4, RP11-109E24.2, RP11-109G23.3, RP11-
10J21.3, RP11-10J21.4, RP11-10K16.1, RP11-10L12.4, RP11-10L7.1, RP11-
10N16.3, RP11-10N23.2, RP11-10O22.2, RP11-1101H11.1, RP11-1102P16.1, RP11-
1103G16.1, RP11-1105G2.3, RP11-1105G2.4, RP11-110H1.2, RP11-110H10.2,
RP11-110I1.5, RP11-110J1.2, RP11-1112C15.1, RP11-1112J20.1, RP11-1112J20.2,
RP11-1113L8.6, RP11-1114I9.1, RP11-111A21.1, RP11-111A22.1, RP11-111E14.1,
RP11-111F16.2, RP11-111K18.2, RP11-1124B17.1, RP11-112H10.4, RP11-
1134I14.3, RP11-1134I14.8, RP11-1136G11.6, RP11-113D6.10, RP11-113D6.6,
RP11-113K21.2, RP11-1140I5.1, RP11-114B7.6, RP11-114F10.2, RP11-114F10.3,
RP11-114F3.2, RP11-114F3.5, RP11-114H24.4, RP11-114H24.6, RP11-114L10.2,
RP11-114M5.1, RP11-115C10.1, RP11-115D19.1, RP11-115H15.1, RP11-115L11.1,
RP11-115N12.1, RP11-1166P10.9, RP11-1167A19.2, RP11-116O18.1, RP11-
117F22.1, RP11-117L5.4, RP11-118D22.3, RP11-118E18.4, RP11-118M9.3, RP11-
119B16.2, RP11-119D9.1, RP11-119F7.2, RP11-119N19.1, RP11-120C12.3, RP11-
120M18.2, RP11-1212A22.2, RP11-121L10.3, RP11-121P10.1, RP11-122A3.2,
RP11-122C9.1, RP11-122F14.3, RP11-123B3.6, RP11-123C21.1, RP11-123J14.2,
RP11-123M6.2, RP11-124A7.2, RP11-124D2.3, RP11-124D2.6, RP11-125H8.1,
RP11-125I23.3, RP11-125K10.4, RP11-1260E13.4, RP11-126O1.4, RP11-127B16.1,
RP11-127L20.6, RP11-1280I22.1, RP11-128A17.1, RP11-128A6.2, RP11-128A6.3,
RP11-128M1.1, RP11-1299A16.3, RP11-129B22.2, RP11-129B22.6, RP11-129B9.1,
RP11-129K20.2, RP11-12A1.1, RP11-12C17.2, RP11-12D24.6, RP11-12M5.4,
RP11-12M9.4, RP11-131H24.4, RP11-131K5.1, RP11-1334A24.6, RP11-133K1.6,
RP11-133L14.4, RP11-133O22.6, RP11-1348G14.1, RP11-1348G14.4, RP11-
1348G14.5, RP11-134E15.2, RP11-134G8.7, RP11-134G8.8, RP11-134L4.1, RP11-
134P9.1, RP11-135A24.2, RP11-135F9.1, RP11-135J2.3, RP11-137H2.4, RP11-
137L10.5, RP11-137N23.1, RP11-138I1.3, RP11-139I14.2, RP11-139J15.2, RP11-
13G14.4, RP11-13J8.1, RP11-13L2.2, RP11-13N13.5, RP11-13N13.6, RP11-
140A10.3, RP11-1415C14.4, RP11-141J13.4, RP11-142G7.2, RP11-142J21.2, RP11-
142L4.3, RP11-142M10.2, RP11-142O6.1, RP11-143E21.7, RP11-143M1.3, RP11-
144A16.1, RP11-144G7.2, RP11-144L1.2, RP11-144L1.8, RP11-144N1.1, RP11-
145M4.3, RP11-145M9.3, RP11-146E13.3, RP11-146F11.4, RP11-147I3.1, RP11-
147O5.1, RP11-149I2.4, RP11-149I9.2, RP11-14I17.1, RP11-150L8.3, RP11-
151D14.1, RP11-151G12.2, RP11-151H2.3, RP11-152F13.1, RP11-152F13.2, RP11-
152F13.4, RP11-152F13.5, RP11-152H18.4, RP11-152K4.2, RP11-152N13.5, RP11-
153F1.1, RP11-154B12.3, RP11-154P18.1, RP11-154P18.2, RP11-155G14.5, RP11-
155G15.2, RP11-156E6.1, RP11-156K13.2, RP11-156L14.1, RP11-156N15.1, RP11-
157G21.2, RP11-157L3.4, RP11-158I9.5, RP11-158I9.7, RP11-158M2.2, RP11-
158M2.4, RP11-158N24.1, RP11-159A18.1, RP11-159C21.4, RP11-159F24.3, RP11-
159H10.3, RP11-159H22.1, RP11-159H3.1, RP11-159J3.1, RP11-15A1.3, RP11-
15E18.1, RP11-15F12.1, RP11-15I11.3, RP11-160C18.2, RP11-160E2.6, RP11-
160H12.2, RP11-160H22.3, RP11-160N1.10, RP11-160O5.1, RP11-161E22.2, RP11-
161H23.11, RP11-161H23.5, RP11-161H23.9, RP11-162D9.3, RP11-162G10.4,
RP11-162G10.5, RP11-162G9.1, RP11-162J8.3, RP11-163E9.1, RP11-163E9.2,
RP11-163F15.1, RP11-164C1.2, RP11-164N3.3, RP11-165F24.2, RP11-165F24.5,
RP11-165H4.2, RP11-165N19.2, RP11-166A12.1, RP11-166B2.5, RP11-166B2.7,
RP11-166B2.8, RP11-166D19.1, RP11-166N17.3, RP11-166O4.1, RP11-166P13.4,
RP11-167H9.3, RP11-167H9.4, RP11-167H9.6, RP11-167N4.5, RP11-167P20.1,
RP11-167P22.3, RP11-168G16.1, RP11-168L22.2, RP11-169D4.1, RP11-169E6.1,
RP11-169K16.4, RP11-169K16.6, RP11-169K16.7, RP11-169L17.5, RP11-
169O17.5, RP11-16C1.1, RP11-16C18.3, RP11-16E18.3, RP11-16F15.1, RP11-
16K12.1, RP11-16L14.2, RP11-16M8.2, RP11-16N11.2, RP11-16P6.1, RP11-
170L3.4, RP11-170L3.8, RP11-170M17.1, RP11-171I2.1, RP11-172E9.2, RP11-
172F4.2, RP11-172F4.5, RP11-173A6.2, RP11-173B14.5, RP11-173D9.5, RP11-
173E2.1, RP11-173M1.4, RP11-174G6.1, RP11-174O3.1, RP11-175B9.3, RP11-
175O19.4, RP11-175P19.2, RP11-177H13.2, RP11-178A10.1, RP11-178C3.2, RP11-
178C3.3, RP11-178D12.1, RP11-178F10.2, RP11-178H8.3, RP11-179A7.2, RP11-
179G5.1, RP11-179G5.4, RP11-179H18.7, RP11-179H18.8, RP11-17A1.3, RP11-
17A4.1, RP1-117B12.4, RP11-17E2.2, RP11-17J14.2, RP11-17L5.2, RP11-17L5.4,
RP11-17M16.2, RP1-117O3.2, RP11-180O5.2, RP11-181C21.4, RP11-181C3.1,
RP11-181E22.1, RP11-181G12.4, RP11-181K3.4, RP11-182B22.2, RP11-182I10.4,
RP11-182J23.1, RP11-182L21.4, RP11-183C12.1, RP11-184E9.1, RP11-184E9.2,
RP11-184I16.3, RP11-184I16.4, RP11-184M15.1, RP11-184M15.2, RP11-185N2.1,
RP11-186F10.2, RP11-187A9.3, RP11-187C18.4, RP11-187C18.6, RP11-187E13.1,
RP11-187E13.2, RP11-18A15.1, RP11-18A3.4, RP11-18B3.3, RP11-18H7.1, RP1-
118J21.25, RP1-118J21.5, RP11-18J9.3, RP11-18M17.1, RP11-190A12.7, RP11-
190C22.1, RP11-190J1.3, RP11-190P13.2, RP11-191N8.2, RP11-192H23.5, RP11-
193H22.2, RP11-193H5.1, RP11-195B3.1, RP11-195C7.3, RP11-195E11.2, RP11-
196G18.23, RP11-196H14.2, RP11-196I18.4, RP11-197M22.2, RP11-197N18.2,
RP11-197N18.7, RP11-197P3.4, RP11-197P3.5, RP11-198M11.2, RP11-198M15.1,
RP11-198M6.5, RP11-19J3.5, RP11-1B20.1, RP11-1C8.6, RP11-1E1.1, RP11-
1E11.1, RP11-1E3.1, RP11-1H15.2, RP11-1L9.1, RP11-1M18.1, RP11-201P10.3,
RP11-203B9.4, RP11-203F10.1, RP11-203F10.5, RP11-203F10.6, RP11-203J24.8,
RP11-203M5.2, RP11-203M5.6, RP11-203M5.8, RP11-204K16.2, RP11-204M4.2,
RP11-204M4.3, RP11-205M20.7, RP11-206F17.2, RP11-206L10.11, RP11-
206L10.13, RP11-206L10.9, RP11-208G20.2, RP11-209D14.4, RP11-20B24.3,
RP11-20B24.6, RP11-20D14.3, RP11-20D14.4, RP11-20E24.1, RP11-20F24.5,
RP11-20I20.1, RP11-20I20.2, RP11-20I23.3, RP11-20J15.3, RP11-20O24.4, RP11-
210M15.1, RP11-210N13.1, RP11-211A18.1, RP11-211G3.2, RP11-211G3.3, RP11-
211N11.5, RP11-211N8.2, RP11-211N8.3, RP11-212D3.3, RP11-213G2.2, RP11-
213G2.3, RP11-213H15.1, RP11-213H15.3, RP11-214K3.5, RP11-215A21.2, RP11-
215G15.4, RP11-215N21.1, RP11-215P8.4, RP11-217B1.2, RP11-217B7.2, RP11-
217B7.3, RP11-217H19.1, RP11-218C23.1, RP11-218E20.2, RP11-218E20.3, RP11-
218E20.5, RP11-218M11.6, RP11-219B17.1, RP11-219B17.2, RP11-219B4.5, RP11-
219B4.6, RP11-219G10.2, RP11-21B23.1, RP11-21B23.2, RP11-21L19.1, RP11-
21L23.3, RP11-220C2.1, RP11-220D10.1, RP11-220I1.1, RP11-220I1.4, RP11-
220I1.5, RP11-221N13.2, RP11-221N13.3, RP11-222A11.1, RP11-223A3.1, RP11-
227B21.2, RP11-227D13.1, RP11-227D13.2, RP11-227F19.5, RP11-227G15.9,
RP11-227H15.4, RP11-227H4.5, RP11-229A12.2, RP11-229M1.2, RP11-229P13.20,
RP11-229P13.25, RP11-22B10.3, RP11-22B23.2, RP11-22C11.2, RP1-122K4.2,
RP1-122K4.3, RP11-22P4.1, RP11-22P4.2, RP11-231E4.4, RP11-231P20.2, RP11-
232D9.1, RP11-234B24.2, RP11-234B24.4, RP11-234K24.3, RP11-234N17.1, RP11-
235C23.5, RP11-235D19.2, RP11-235E17.2, RP11-235E17.3, RP11-235E17.5,
RP11-236P24.1, RP11-23B15.1, RP11-23B7.3, RP11-23E19.2, RP11-23F23.2,
RP11-23J9.3, RP11-23J9.7, RP11-23P13.6, RP11-240D10.4, RP11-241I20.3, RP11-
242C19.2, RP11-242D8.2, RP11-242O24.3, RP11-242O24.5, RP11-243A14.1, RP11-
243J18.2, RP11-244K5.1, RP11-244N20.7, RP11-244N9.6, RP11-245C17.2, RP11-
246K15.1, RP11-247I13.3, RP11-248C1.2, RP11-248J23.5, RP11-249L21.2, RP11-
24F11.2, RP11-24M17.4, RP11-250B16.1, RP11-252A24.3, RP11-252A24.5, RP11-
252I14.1, RP11-252M18.3, RP11-252P19.1, RP11-252P19.2, RP11-252P19.3, RP11-
253A20.1, RP11-253I19.3, RP11-254B13.3, RP11-254I22.1, RP11-254I22.3, RP11-
255B23.1, RP11-255G21.1, RP11-255J3.2, RP11-255N24.4, RP11-256K7.1, RP11-
257I8.1, RP11-257P3.3, RP11-258C19.4, RP11-258F22.1, RP11-259F16.3, RP11-
259G18.1, RP11-259O18.4, RP11-259P15.3, RP11-259P15.4, RP11-25C19.3, RP11-
25J23.3, RP11-260E18.1, RP11-261C10.6, RP11-262D11.2, RP11-262H14.4, RP11-
262H14.7, RP11-262M14.2, RP11-263E19.1, RP11-263K4.1, RP11-264B14.2, RP11-
264F23.1, RP11-264F23.4, RP11-264M12.2, RP11-265N6.1, RP11-266E14.1, RP11-
266K22.2, RP11-266L9.6, RP11-267M23.6, RP11-267N12.3, RP11-268G12.1,
RP11-268G12.3, RP11-269F20.1, RP11-269G24.3, RP11-270C12.3, RP11-
270M14.4, RP11-270M14.5, RP11-271C24.2, RP11-271K11.1, RP11-271K11.2,
RP11-271M24.2, RP11-272P10.2, RP11-273B20.3, RP11-273G15.1, RP11-274H2.2,
RP11-274H2.3, RP11-274H2.5, RP11-275F13.3, RP11-275H4.1, RP11-275I14.4,
RP11-275I4.1, RP11-275O4.3, RP11-276H1.2, RP11-276H1.3, RP11-276H19.1,
RP11-276H7.2, RP11-276H7.3, RP11-277A4.4, RP11-277I20.3, RP11-277J6.2,
RP11-277L2.3, RP11-278A23.2, RP11-278C7.1, RP11-278I4.2, RP11-279O9.4,
RP1-127B20.4, RP11-27G24.1, RP11-27I1.4, RP11-27K13.3, RP1-127L4.7, RP11-
27M24.1, RP11-27M24.3, RP11-280F2.2, RP11-280G19.2, RP11-280K24.4, RP11-
280O1.2, RP11-281J9.1, RP11-281O15.1, RP11-281O15.3, RP11-281P23.1, RP11-
282I1.1, RP11-283C24.1, RP11-283G6.3, RP11-283G6.4, RP11-283G6.5, RP11-
284B18.3, RP11-284M14.1, RP11-284N8.3, RP11-285G1.8, RP11-286E11.1, RP11-
286E11.2, RP11-286N3.2, RP11-286O18.1, RP11-287D1.1, RP11-287D1.3, RP11-
287F9.2, RP11-288E14.2, RP11-288G11.3, RP11-289H16.1, RP11-289K10.1, RP11-
28G8.1, RP11-290D2.3, RP11-290F20.3, RP11-290L1.4, RP11-292B8.1, RP11-
292F9.1, RP11-292F9.2, RP11-292O17.1, RP11-293A21.1, RP11-293B20.1, RP11-
293G6_A.3, RP11-293M10.5, RP11-294K24.4, RP11-295G20.2, RP11-295G24.5,
RP11-295H24.3, RP11-295P9.2, RP11-296A18.3, RP11-296A18.6, RP11-296A18.7,
RP11-296O14.2, RP11-296P7.4, RP11-297C4.3, RP11-297L17.6, RP11-297L6.2,
RP11-297M9.2, RP11-298I3.1, RP11-298J20.1, RP11-298J23.5, RP11-299G20.3,
RP11-299H22.1, RP11-299H22.3, RP11-299H22.4, RP11-299H22.5, RP11-299J3.6,
RP11-299M14.2, RP11-29H23.6, RP11-29M5.1, RP11-2C24.5, RP11-2E11.5, RP11-
2F9.3, RP1-12G14.7, RP11-2I17.1, RP11-2K6.2, RP11-2N1.2, RP11-2N5.1, RP11-
300J18.1, RP11-300J18.3, RP11-301M17.3, RP11-301N24.3, RP11-302F12.1, RP11-
302F12.10, RP11-302K17.3, RP11-302K17.7, RP11-303E16.3, RP11-303E16.5,
RP11-303O9.2, RP11-304F15.5, RP11-304L19.2, RP11-304L19.5, RP11-304M6.4,
RP11-304N14.2, RP11-305E17.4, RP11-305E17.6, RP11-305L7.3, RP11-305L7.6,
RP11-305M3.3, RP11-306G20.1, RP11-306I1.1, RP11-306I1.2, RP11-307B6.3,
RP11-307C12.11, RP11-307C19.1, RP11-307E17.2, RP11-307L3.2, RP11-307P22.1,
RP11-308N19.5, RP11-309E23.2, RP11-309N17.4, RP11-30J20.1, RP11-30K9.1,
RP11-30K9.2, RP11-30K9.5, RP11-30L15.4, RP11-30P6.1, RP11-30P6.6, RP11-
310E22.2, RP11-310E22.4, RP11-310H4.1, RP11-310H4.3, RP11-311D14.1, RP11-
311P8.1, RP11-312O7.2, RP11-312P12.2, RP11-313C15.1, RP11-313F23.4, RP11-
313F23.5, RP11-313I2.7, RP11-313L9.1, RP11-313P18.1, RP11-314A15.2, RP11-
314A20.2, RP11-314J18.6, RP11-314N13.3, RP11-315D16.2, RP11-315D16.3,
RP11-315O6.1, RP11-316K19.3, RP11-316M21.6, RP11-317B7.2, RP11-317G6.1,
RP11-317J10.2, RP11-317N12.1, RP11-317N8.4, RP11-317N8.5, RP11-317P15.4,
RP11-318A15.2, RP11-318G8.2, RP11-318G8.3, RP11-318L16.6, RP11-318M2.2,
RP11-319G6.1, RP11-319G6.3, RP11-319J24.3, RP11-31E13.2, RP1-131F15.2,
RP11-31H5.1, RP11-320A16.1, RP11-320H14.1, RP11-320M2.1, RP11-320N21.1,
RP11-320N7.3, RP11-320P7.1, RP11-320P7.2, RP11-321E8.5, RP11-321F8.4, RP11-
322D14.2, RP11-322F10.2, RP11-322F10.3, RP11-323F5.1, RP11-323F5.2, RP11-
323I15.5, RP11-323J4.1, RP11-323N10.1, RP11-324C10.1, RP11-324H6.5, RP11-
324I22.2, RP11-324O2.4, RP11-325E14.2, RP11-325E5.1, RP11-325F22.2, RP11-
325P15.2, RP11-325P15.3, RP11-326A19.5, RP11-326C3.12, RP11-326L17.1,
RP11-326L24.4, RP11-327F22.1, RP11-327F22.2, RP11-328C8.2, RP11-328J14.2,
RP11-328K22.1, RP11-329J18.2, RP11-32B5.1, RP11-32B5.7, RP11-32I2.1, RP11-
32M23.4, RP11-330C7.3, RP11-330M2.3, RP11-331F4.1, RP11-331F4.4, RP11-
331F9.4, RP11-332E3.2, RP11-332G1.1, RP11-332H17.1, RP11-332J15.2, RP11-
332O19.3, RP11-333I13.1, RP11-333J10.3, RP11-334L9.1, RP11-335O4.3, RP11-
336A10.5, RP11-337A23.6, RP11-337C18.4, RP11-337C18.8, RP11-338C15.3,
RP11-338C15.5, RP11-338L22.2, RP11-339B21.8, RP11-33I11.2, RP11-33N11.1,
RP11-340E6.2, RP11-340I6.6, RP11-340I6.7, RP11-340I6.8, RP11-341A11.2, RP11-
341G5.1, RP11-341G5.2, RP11-341N2.1, RP11-342M1.6, RP11-343H19.1, RP11-
343H5.4, RP11-343H5.6, RP11-343P16.1, RP11-344A5.1, RP11-344H11.5, RP11-
344N10.2, RP11-344N10.3, RP11-345J13.1, RP11-345L23.1, RP11-347C12.10,
RP11-347C18.3, RP11-347D21.1, RP11-347D21.3, RP11-348A11.4, RP11-348F1.2,
RP11-348H3.4, RP11-348I14.4, RP11-348P10.1, RP11-34C15.2, RP11-34F20.5,
RP11-34P1.2, RP11-34P13.10, RP11-34P13.7, RP11-350E12.4, RP11-350F4.2,
RP11-350G24.2, RP11-350G8.3, RP11-350J20.9, RP11-350N15.3, RP11-351E7.1,
RP11-351I21.6, RP11-351I24.1, RP11-351K16.4, RP11-351M8.1, RP11-352D13.6,
RP11-352M15.1, RP11-353B9.1, RP11-354M1.3, RP11-354M20.3, RP11-354P11.3,
RP11-354P11.8, RP11-355D13.3, RP11-355I22.2, RP11-355I22.5, RP11-355I22.6,
RP11-355I22.7, RP11-355K23B.1, RP11-356C4.3, RP11-356I2.2, RP11-356J5.5,
RP11-356M20.2, RP11-356O23.1, RP11-358B23.5, RP11-358L16.2, RP11-
358M11.1, RP11-358M11.4, RP11-358M14.2, RP11-359B20.1, RP11-359J14.2,
RP11-359K18.3, RP11-359M6.2, RP11-35G22.1, RP11-35J23.5, RP11-35N6.6,
RP11-35O15.1, RP11-360F5.1, RP11-360F5.3, RP11-360G10.2, RP11-361D15.1,
RP11-361F15.2, RP11-361I14.2, RP11-362A9.3, RP11-362L22.1, RP11-363G2.4,
RP11-363H12.1, RP11-363I22.3, RP11-363L24.2, RP11-363L24.3, RP11-364M6.1,
RP11-364P2.2, RP11-364P22.2, RP11-365D9.1, RP11-365H23.1, RP11-365O10.1,
RP11-365O16.3, RP11-366L5.1, RP11-366M4.1, RP11-366M4.11, RP11-366M4.12,
RP11-366O17.2, RP11-367B6.2, RP11-367E12.4, RP11-367F23.1, RP11-367G18.1,
RP11-367H5.7, RP11-367J11.4, RP11-367J7.4, RP11-368N21.2, RP11-368P15.3,
RP11-370I10.6, RP11-370I10.7, RP11-371E8.4, RP11-373A9.1, RP11-373J16.1,
RP11-373L24.1, RP11-373N22.3, RP11-374A4.1, RP11-374M1.6, RP11-374M1.8,
RP11-374M1.9, RP11-377D9.1, RP11-378I13.1, RP11-378J18.3, RP11-378J18.6,
RP11-379B8.1, RP11-379F4.1, RP11-379F4.4, RP11-379H8.1, RP11-379P15.1,
RP1-137D17.1, RP11-37L2.1, RP11-380F14.2, RP11-380G5.2, RP11-380G5.3,
RP11-381K20.2, RP11-382B18.2, RP11-382D8.5, RP11-382J12.1, RP11-383C5.3,
RP11-383C5.4, RP11-383C5.5, RP11-383D22.2, RP11-383J24.1, RP11-383J24.2,
RP11-384B12.2, RP11-384B12.3, RP11-384C4.2, RP11-384C4.3, RP11-384K6.1,
RP11-384M15.3, RP11-385D13.1, RP11-385J1.2, RP11-386C23.1, RP11-
386G11.10, RP11-386I14.3, RP11-386I23.1, RP11-386M24.1, RP11-386M24.3,
RP11-387D10.3, RP11-387H17.4, RP11-388C12.1, RP11-388C12.2, RP11-
388C12.5, RP11-388K12.1, RP11-388M20.9, RP11-389C8.2, RP11-389K14.3,
RP11-389O22.1, RP1-138B7.4, RP11-38G5.2, RP11-38L15.3, RP11-38L15.6, RP11-
38P22.2, RP11-390B4.3, RP11-390F4.10, RP11-390F4.3, RP11-390F4.8, RP11-
390F4.9, RP11-390G14.1, RP11-390K5.1, RP11-391L3.5, RP11-391M20.1, RP11-
392A22.2, RP11-392E22.5, RP11-392O17.1, RP11-392O18.1, RP11-392P7.1, RP11-
392P7.6, RP11-394B2.6, RP11-394D2.1, RP11-394O4.3, RP11-394O4.4, RP11-
395B7.4, RP11-395B7.7, RP11-395G23.3, RP11-395I14.2, RP11-395L14.17, RP11-
396C23.2, RP11-397C12.1, RP11-397E7.2, RP11-397E7.4, RP11-397F23.2, RP11-
397G5.2, RP11-397P13.6, RP11-398E10.1, RP11-398F12.1, RP11-398J5.1, RP11-
399D6.2, RP11-399E6.1, RP11-399E6.4, RP11-399F4.4, RP11-399K21.10, RP11-
399O19.6, RP11-399O19.8, RP11-39C10.1, RP11-39K24.13, RP11-39K24.2, RP1-
13D10.2, RP11-3D23.1, RP11-3G21.1, RP11-3J1.1, RP11-3J10.4, RP11-3J10.7,
RP11-3L8.3, RP11-3N2.1, RP11-3N2.13, RP11-3N2.5, RP11-3P17.3, RP11-400L8.2,
RP11-402G3.5, RP11-402L1.4, RP11-403A21.1, RP11-403A21.3, RP11-403B2.7,
RP11-403I13.9, RP11-403P14.1, RP11-403P17.2, RP11-404E16.1, RP11-404F10.2,
RP11-404G16.2, RP11-404P21.2, RP11-405A12.1, RP11-405F3.4, RP11-405F3.5,
RP11-405L18.1, RP11-406H4.1, RP11-407B7.1, RP11-407N17.3, RP11-407N17.5,
RP11-408H1.3, RP11-409C19.2, RP11-40F6.2, RP1-140K8.2, RP11-410C4.1, RP11-
410C4.4, RP11-410E4.1, RP11-411B10.4, RP11-411K7.1, RP11-412B14.2, RP11-
412D9.4, RP11-413B19.2, RP11-414H23.3, RP11-415C15.3, RP11-415D17.3, RP11-
415I12.3, RP11-415I12.5, RP11-415I12.6, RP11-415K20.1, RP11-416A17.6, RP11-
416K24.2, RP11-416N4.1, RP11-416N4.4, RP11-417B4.3, RP11-417L14.1, RP11-
417N10.3, RP11-417O11.6, RP11-418I22.2, RP11-418J17.1, RP11-419L20.2, RP11-
420A23.1, RP11-420B22.1, RP11-420G6.4, RP11-420K5.1, RP11-420O16.2, RP11-
421F16.3, RP11-421L21.2, RP11-421L21.3, RP11-421N8.1, RP11-422J8.1, RP11-
422P24.9, RP11-423H2.1, RP11-423H2.2, RP11-423H2.3, RP11-423O2.1, RP11-
423O2.2, RP11-423O2.5, RP11-424G14.1, RP11-424M24.5, RP11-425D10.10,
RP11-425I13.1, RP11-425I13.3, RP11-426J5.2, RP11-427D1.1, RP11-428C19.5,
RP11-428F8.2, RP11-428G5.7, RP11-429A20.3, RP11-429A20.4, RP11-429B14.1,
RP11-429D19.1, RP11-429G19.2, RP11-429J17.4, RP11-42A4.1, RP11-42I10.1,
RP11-42O15.2, RP11-430B1.1, RP11-430L17.1, RP11-431K24.1, RP11-431K24.3,
RP11-431M3.1, RP11-431N15.2, RP11-432F4.2, RP11-432I13.1, RP11-432I5.1,
RP11-432I5.6, RP11-432J24.2, RP11-432J24.3, RP11-432J24.6, RP11-432M8.9,
RP11-432N13.4, RP11-433O3.1, RP11-433P17.2, RP11-433P17.3, RP11-434H6.2,
RP11-434I12.2, RP11-434I12.3, RP11-435F13.2, RP11-435O5.2, RP11-436A20.4,
RP11-436G20.1, RP11-436H11.3, RP11-437F6.1, RP11-438D8.2, RP11-438N16.2,
RP11-438N5.2, RP11-439E19.9, RP11-439H8.4, RP11-439K3.3, RP11-439L12.1,
RP11-439L18.1, RP11-439L18.2, RP11-439L18.3, RP11-440G5.2, RP11-440I14.2,
RP11-440K22.1, RP11-440L14.3, RP11-441O15.3, RP11-442A13.1, RP11-
442A17.2, RP11-442N24_B.1, RP11-443A13.2, RP11-443D10.3, RP11-443G13.3,
RP11-443J23.1, RP11-443N24.1, RP11-444I9.2, RP11-444J21.2, RP11-445F6.2,
RP11-445L13_B.3, RP11-445P17.4, RP11-445P17.8, RP11-446E9.1, RP11-
446F17.3, RP11-446P9.1, RP11-447B18.1, RP11-447H19.3, RP11-448G4.2, RP11-
449D8.1, RP11-449H11.1, RP11-449L13.2, RP11-449M6.1, RP11-44D15.3, RP11-
44F14.11, RP11-44F14.2, RP11-44F14.8, RP11-44F21.3, RP11-44I10.3, RP11-
44M6.1, RP11-450D21.1, RP11-451H23.1, RP11-451K18.3, RP11-451O18.1, RP11-
452D12.1, RP11-452F19.3, RP11-452G18.1, RP11-452J21.2, RP11-452J6.2, RP11-
452K12.7, RP11-452L6.7, RP11-453E17.3, RP11-453N18.1, RP11-454E7.1, RP11-
454H13.6, RP11-454K7.1, RP11-454L1.2, RP11-454P21.1, RP11-455B2.7, RP11-
455G16.1, RP11-456H18.2, RP11-456I15.2, RP11-456J20.1, RP11-457M11.2, RP11-
458D21.2, RP11-458I7.1, RP11-459D22.1, RP11-459E5.1, RP11-459F3.3, RP11-
459F3.6, RP11-45M22.3, RP11-45M22.4, RP11-45M22.5, RP11-460C6.1, RP11-
460I13.6, RP11-460N11.2, RP11-460N11.3, RP11-461C13.1, RP11-461O7.1, RP11-
462B18.2, RP11-462D18.2, RP11-462G12.5, RP11-462G2.1, RP11-462G8.2, RP11-
463J10.3, RP11-463J17.1, RP11-463O12.3, RP11-463O9.5, RP11-464C19.2, RP11-
464F9.1, RP11-464O2.2, RP11-465B22.3, RP11-466A19.1, RP11-466A19.3, RP11-
466A19.8, RP11-466F5.8, RP11-466G12.2, RP11-466P24.2, RP11-467D18.2, RP11-
467H10.1, RP11-467I20.2, RP11-467I20.5, RP11-467L13.4, RP11-467L19.8, RP11-
467M13.1, RP11-467N20.5, RP11-468E2.2, RP11-468O2.1, RP11-469J4.3, RP11-
46A10.2, RP11-46A10.4, RP11-46A10.5, RP11-46A10.6, RP11-46C24.3, RP11-
46H11.12, RP11-46H11.3, RP1-146I3.1, RP11-46I8.3, RP11-46O21.2, RP11-
470E16.1, RP11-471J12.1, RP11-471L13.3, RP11-472B18.2, RP11-472I20.1, RP11-
472I20.2, RP11-472N13.2, RP11-473I1.10, RP11-473I1.6, RP11-473I1.9, RP11-
473J6.1, RP11-473M20.16, RP11-473N11.2, RP11-473O4.3, RP11-474B12.1, RP11-
474D1.2, RP11-474I11.7, RP11-474I16.8, RP11-474P12.3, RP11-475D8.1, RP11-
475I24.3, RP11-475I24.4, RP11-475O23.2, RP11-475O6.1, RP11-476C8.2, RP11-
477G18.2, RP11-478C1.7, RP11-478C6.4, RP11-478C6.5, RP11-478C6.6, RP11-
478H16.1, RP11-479A21.1, RP11-479G22.3, RP11-479O16.1, RP11-479O17.2,
RP11-479O9.2, RP11-47F1.1, RP11-47I22.1, RP11-47I22.2, RP11-47I22.3, RP11-
47K11.2, RP11-47P18.2, RP11-480D4.3, RP11-480G7.2, RP11-480I12.7, RP11-
481A12.5, RP11-481C4.1, RP11-481J2.2, RP11-482D24.2, RP11-483E7.1, RP11-
483F11.7, RP11-483P21.2, RP11-483P21.6, RP11-484D2.2, RP11-484D2.3, RP11-
484D2.5, RP11-484I6.6, RP11-484N16.1, RP11-484P15.1, RP11-484P15.2, RP11-
485M7.1, RP11-485O10.3, RP11-486B10.4, RP11-486F17.1, RP11-488C13.1, RP11-
488L18.1, RP11-488L18.3, RP11-488L18.6, RP11-488L4.1, RP11-488P3.1, RP11-
489G11.1, RP11-489G11.3, RP11-48B24.2, RP11-48B3.2, RP11-48D4.2, RP11-
48G14.2, RP1-148H17.1, RP11-490D19.6, RP11-490D19.8, RP11-490F3.1, RP11-
490H24.5, RP11-490K7.4, RP11-490N5.2, RP11-492E3.51, RP11-492I2.1, RP11-
492M23.2, RP11-493G17.4, RP11-494I9.1, RP11-494K3.2, RP11-495K9.3, RP11-
495K9.6, RP11-495K9.7, RP11-495P10.5, RP11-495P10.8, RP11-496B10.3, RP11-
496D1.2, RP11-496H1.1, RP11-497D6.2, RP11-497D6.3, RP11-498A13.1, RP11-
498B4.7, RP11-498D10.3, RP11-498D10.6, RP11-499E18.1, RP11-499O7.7, RP1-
149A16.16, RP1-149A16.3, RP11-49I11.1, RP11-49I4.3, RP11-49K24.4, RP11-
49O14.2, RP11-4C20.3, RP11-4C20.4, RP11-4F5.2, RP11-4M23.2, RP11-500G10.5,
RP11-500G10.6, RP11-500M8.4, RP11-503E24.2, RP11-503N18.1, RP11-503N18.2,
RP11-503N18.3, RP11-504G3.4, RP11-504P24.3, RP11-505K9.1, RP11-505K9.3,
RP11-505P4.6, RP11-505P4.7, RP11-506B4.3, RP11-506D12.5, RP11-507B12.2,
RP11-507E23.1, RP11-507F17.1, RP11-507J18.1, RP11-507J18.2, RP11-507K13.6,
RP11-507K2.3, RP11-508M1.3, RP11-508M1.5, RP11-508N12.2, RP11-508N22.4,
RP11-508N22.9, RP11-509E16.1, RP11-509J21.4, RP11-50B3.4, RP11-50C13.1,
RP11-50D9.3, RP11-510I21.1, RP11-510I6.2, RP11-510M2.2, RP11-511B23.2,
RP11-511H9.4, RP11-511P7.2, RP11-512G13.1, RP11-512N21.3, RP11-513D5.5,
RP11-513G11.2, RP11-513I15.6, RP11-513M16.2, RP11-513P18.2, RP11-514O12.4,
RP11-515O17.2, RP11-516A11.1, RP11-516C1.1, RP11-517A5.7, RP11-517B11.2,
RP11-517B11.7, RP11-517I3.1, RP11-517O13.3, RP11-517P14.7, RP11-518D3.3,
RP11-519G16.1, RP1-151B14.9, RP11-51B23.3, RP11-51J9.4, RP11-51L5.5, RP11-
51M24.1, RP11-51O6.1, RP11-521A24.1, RP11-521C10.1, RP11-521C20.4, RP11-
521D12.1, RP11-521D12.5, RP11-521L9.1, RP11-521L9.2, RP11-521O16.2, RP11-
522B15.3, RP11-522H2.3, RP11-522L3.6, RP11-523O18.5, RP11-524F11.1, RP11-
524O1.4, RP11-525A16.1, RP11-525A16.4, RP11-525G13.2, RP11-526I2.3, RP11-
526I8.2, RP11-526L22.1, RP11-526P5.2, RP11-527F13.1, RP11-527L4.5, RP11-
527N22.1, RP11-528A10.1, RP11-528A10.2, RP11-528N21.1, RP11-529H20.3,
RP11-529J17.2, RP1-152L7.1, RP11-530C5.1, RP11-530N7.2, RP11-530N7.3,
RP11-531A24.3, RP11-533E19.5, RP11-533F5.2, RP11-534G20.3, RP11-534I8.1,
RP11-534L20.5, RP11-535M15.1, RP11-535M15.2, RP11-536C10.12, RP11-
536C5.2, RP11-536I6.1, RP11-537A6.9, RP11-537P22.1, RP11-539E17.4, RP11-
539G18.2, RP11-53B2.1, RP11-53B2.3, RP11-53B2.4, RP11-53B2.5, RP11-53I6.4,
RP11-53I6.6, RP11-53L24.1, RP11-53M11.2, RP11-540A21.2, RP11-540O11.4,
RP11-543P15.1, RP11-544A12.5, RP11-544A12.8, RP11-544I20.2, RP11-545A16.3,
RP11-545A16.4, RP11-545E17.3, RP11-545G3.1, RP11-545I5.3, RP11-545M17.3,
RP11-546B15.1, RP11-546B15.2, RP11-546B8.3, RP11-546M4.1, RP11-546O6.4,
RP11-547D23.1, RP11-548B3.3, RP11-549B18.1, RP11-549B18.2, RP11-549B18.3,
RP11-54B9.2, RP11-54D18.3, RP11-54F2.1, RP11-54H19.8, RP11-54I5.1, RP11-
54O15.3, RP11-54O7.10, RP11-54O7.2, RP11-550A5.2, RP11-550F7.1, RP11-
550I24.2, RP11-551G24.2, RP11-551L14.1, RP11-552D8.1, RP11-552E10.1, RP11-
552E20.4, RP11-552F3.9, RP11-552O4.2, RP11-553D4.2, RP11-553L6.5, RP11-
553P9.1, RP11-554D14.5, RP11-554D14.6, RP11-554I8.1, RP11-555H23.1, RP11-
555K12.1, RP11-555M1.3, RP11-556I14.1, RP11-556I14.2, RP11-556O9.2, RP11-
556O9.3, RP11-557C18.3, RP11-557J10.4, RP11-55K22.2, RP11-55K22.5, RP11-
560G2.1, RP11-561B11.3, RP11-561B11.4, RP11-561O23.8, RP11-561P12.2, RP11-
562A8.4, RP11-562A8.5, RP11-562M8.4, RP11-563E2.2, RP11-563H8.2, RP11-
564C4.5, RP11-564C4.6, RP11-564D11.3, RP11-564P9.1, RP11-565J7.3, RP11-
566K11.4, RP11-566K19.8, RP11-567C2.1, RP11-567G24.3, RP11-567M16.2,
RP11-567M16.3, RP11-567M21.3, RP11-567N4.2, RP11-567O16.1, RP11-568A7.2,
RP11-568A7.3, RP11-568J23.1, RP11-568K15.1, RP11-569G13.2, RP11-569H14.1,
RP11-56B16.1, RP11-56B16.5, RP11-56F10.3, RP11-570D4.2, RP11-570P14.1,
RP11-571M6.13, RP11-572C15.3, RP11-572M11.1, RP11-573D15.1, RP11-
573G6.6, RP11-574F21.3, RP11-574K11.18, RP11-575A19.2, RP11-575B7.3, RP11-
575H3.1, RP11-575L16.2, RP11-576N17.5, RP11-577H5.1, RP11-578F5.1, RP11-
579O24.1, RP11-57A1.1, RP11-57C13.3, RP11-57C13.5, RP11-580I1.1, RP11-
580I1.2, RP11-580I1.3, RP11-582E3.4, RP11-583F2.2, RP11-583F2.5, RP11-
584P21.2, RP11-585F1.6, RP11-585P4.5, RP11-587D21.1, RP11-588G21.2, RP11-
588H23.3, RP11-588K22.2, RP11-589F5.3, RP11-58A11.2, RP11-58E21.3, RP11-
58O9.1, RP11-592N21.1, RP11-592O2.1, RP11-596D21.1, RP11-597A11.4, RP11-
597D13.7, RP11-597D13.9, RP11-597M12.1, RP11-599B13.3, RP11-599B13.7,
RP11-599J14.2, RP1-159A19.4, RP11-59E19.1, RP11-59H1.3, RP11-59J5.1, RP11-
59K5.1, RP1-159M24.1, RP1-15D7.1, RP11-5K23.3, RP11-5N23.2, RP11-5N23.3,
RP11-5P18.1, RP11-600F24.1, RP11-600F24.2, RP11-600G3.1, RP11-601I15.1,
RP11-602M11.3, RP11-603B24.2, RP11-603K19.1, RP11-603K19.2, RP11-606P2.1,
RP11-60A24.3, RP11-60C6.5, RP11-60I3.4, RP11-611O2.5, RP11-612B6.2, RP11-
613D13.4, RP11-613D13.5, RP11-613D13.7, RP11-613D13.8, RP11-613F22.5,
RP11-613F22.8, RP11-613G13.1, RP11-613M10.9, RP11-613M5.1, RP11-613M5.2,
RP11-613M5.3, RP11-614F17.2, RP11-614O9.1, RP11-616K22.1, RP11-616L12.4,
RP11-616M22.1, RP11-616M22.2, RP11-617J18.1, RP11-618G20.2, RP11-
619F23.2, RP11-619I22.1, RP11-61A14.1, RP11-61A14.2, RP11-61A14.3, RP11-
61I13.3, RP11-61L19.1, RP11-61L23.2, RP11-61N20.3, RP11-620J15.1, RP11-
620J15.2, RP11-621H8.2, RP11-622O11.1, RP11-622O11.5, RP11-624D11.2, RP11-
624D20.1, RP11-624G17.1, RP11-626E13.1, RP11-626G11.1, RP11-626G11.3,
RP11-627K11.1, RP11-627K11.3, RP11-628K18.1, RP11-629B11.4, RP11-62C3.8,
RP11-62H7.2, RP11-62I21.1, RP11-62L18.3, RP11-630I5.1, RP11-631M6.2, RP11-
632F7.3, RP11-632K20.2, RP11-635I10.1, RP11-637A17.1, RP11-637A17.2, RP11-
637I8.2, RP11-638I2.10, RP11-638I2.8, RP11-639F1.2, RP11-63A1.1, RP11-
63P12.6, RP11-63P12.7, RP11-640N20.8, RP11-640N20.9, RP11-641A6.5, RP11-
641C17.2, RP11-641D5.1, RP11-643G16.4, RP11-643M14.1, RP11-643M14.3,
RP11-644C3.1, RP11-644K8.1, RP11-645N11.2, RP11-645N11.3, RP11-646D13.1,
RP11-648L3.2, RP11-648M2.2, RP11-648O15.1, RP11-649E7.7, RP11-64C12.6,
RP11-64D24.4, RP1-164F3.8, RP11-64I24.2, RP11-64J19.1, RP11-64J4.2, RP11-
64K12.9, RP11-64K7.1, RP11-650L12.2, RP11-650P15.1, RP11-651L5.2, RP11-
651P23.4, RP11-653G8.2, RP11-653J6.1, RP11-654A16.1, RP11-654C22.2, RP11-
654D12.1, RP11-654G14.1, RP11-655H13.2, RP11-659E9.4, RP11-65E22.2, RP11-
65J21.3, RP11-65J3.1, RP11-65J3.14, RP11-65J3.6, RP11-65L3.1, RP11-660L16.2,
RP11-661C3.2, RP11-661G16.1, RP11-661G16.2, RP11-661I21.2, RP11-661O13.1,
RP11-662B19.1, RP11-662G23.1, RP11-662I13.2, RP11-662J14.2, RP11-662M24.1,
RP11-663P9.1, RP11-663P9.2, RP11-664D1.1, RP11-664I21.5, RP11-664I21.6,
RP11-666A1.3, RP11-666A1.5, RP11-666N19.1, RP11-667K14.3, RP11-667K14.5,
RP11-667K14.8, RP11-667M19.2, RP11-668G8.1, RP11-669E14.4, RP11-669N7.2,
RP11-66A2.2, RP11-66B24.2, RP11-66B24.4, RP11-66H6.3, RP11-66N11.5, RP11-
670E13.5, RP11-671J11.6, RP11-671M22.4, RP11-672L10.1, RP11-672L10.2,
RP11-672L10.3, RP11-673C5.1, RP11-673E1.3, RP11-673F18.1, RP11-674N23.1,
RP11-675F6.3, RP11-676J12.4, RP11-676J12.6, RP11-677M14.6, RP11-678B3.2,
RP11-679C8.2, RP11-67C2.2, RP1-167F1.2, RP11-67F24.1, RP1-167G20.1, RP11-
680C21.1, RP11-680E19.1, RP11-680F20.10, RP11-680F20.11, RP11-680F20.5,
RP11-680F20.9, RP11-680F8.3, RP11-680H20.2, RP11-684O24.1, RP11-685B13.2,
RP11-685B14.1, RP11-685N10.1, RP11-686D22.7, RP11-686G23.1, RP11-686G8.2,
RP11-687F6.4, RP11-687M24.3, RP11-688I9.2, RP11-689B22.2, RP1-168P16.1,
RP1-168P16.2, RP11-690I21.2, RP11-691H4.3, RP11-691H4.4, RP11-691L4.2,
RP11-692N5.1, RP11-692N5.2, RP11-695J4.2, RP11-696F12.1, RP11-696N14.1,
RP11-697E2.4, RP11-697M17.2, RP11-697N18.1, RP11-697N18.2, RP11-699L21.1,
RP11-69C17.1, RP11-69E11.4, RP11-69G7.1, RP11-69H7.2, RP11-69H7.3, RP11-
69L16.4, RP11-69M1.4, RP11-6B4.1, RP11-6D1.6, RP11-6E9.4, RP11-6F2.4, RP11-
6F2.5, RP11-6F2.7, RP11-6L6.2, RP11-6N17.2, RP11-6N17.3, RP11-6O2.3, RP11-
700F16.2, RP11-700F16.3, RP11-700H13.1, RP11-700H6.1, RP11-700H6.2, RP11-
700H6.4, RP11-700P18.1, RP11-700P18.2, RP11-701B16.2, RP11-701P16.1, RP11-
701P16.5, RP11-702L15.2, RP11-702L15.3, RP11-703H8.6, RP11-703H8.9, RP11-
704J17.5, RP11-705B14.1, RP11-705O1.8, RP11-706O15.5, RP11-706O15.7, RP11-
707G14.6, RP11-709A23.1, RP1-170D19.3, RP11-70F11.7, RP11-70J12.1, RP11-
70L8.1, RP11-710M11.1, RP11-711D18.2, RP11-713B9.1, RP11-713P17.4, RP11-
715F3.2, RP11-718G2.4, RP11-718G2.5, RP11-71C5.2, RP11-71G7.1, RP11-
71H17.1, RP11-720L2.3, RP11-723O4.8, RP11-723P16.2, RP11-726G1.1, RP11-
727A23.10, RP11-727A23.7, RP11-728B21.2, RP11-728F11.1, RP11-728F11.3,
RP11-728G15.1, RP11-728K20.1, RP11-728K20.2, RP11-729I10.2, RP11-730A19.5,
RP11-730A19.6, RP11-730A19.1, RP11-730K11.1, RP11-731D1.1, RP11-731I19.1,
RP11-732M18.3, RP11-734C14.1, RP11-734C14.2, RP11-734E19.1, RP11-734J24.1,
RP11-736K20.1, RP11-736K20.5, RP11-736K20.6, RP11-737O24.2, RP11-
737O24.3, RP11-737O24.4, RP11-738B7.1, RP11-738O11.13, RP11-739L10.1,
RP11-739N20.2, RP11-73B2.6, RP11-73E17.3, RP11-73K9.2, RP11-73M18.2,
RP11-73M7.6, RP11-73M7.9, RP11-742B18.1, RP11-744D14.1, RP11-744I24.2,
RP11-745A24.1, RP11-745L13.1, RP11-745L13.2, RP11-746E8.1, RP11-747H12.4,
RP11-748H22.1, RP11-748L13.2, RP11-748L13.3, RP11-74C1.2, RP11-74C1.4,
RP11-74E22.3, RP11-74E24.2, RP11-74K11.2, RP11-750B16.1, RP11-752D24.2,
RP11-752G15.8, RP11-752G15.9, RP11-753A21.1, RP11-753D20.3, RP11-
755B10.3, RP11-756H6.1, RP11-756P10.3, RP11-757C15.2, RP11-757C15.3, RP11-
757G14.1, RP11-757O6.4, RP11-758I14.3, RP11-758M4.1, RP11-758N13.1, RP11-
759L5.2, RP11-75C9.2, RP11-760H22.2, RP11-761I4.3, RP11-761N21.2, RP11-
762H13.1, RP11-762H8.2, RP11-762H8.3, RP11-762I7.4, RP11-763B22.3, RP11-
763B22.4, RP11-764K9.4, RP11-766F14.2, RP11-766N7.3, RP11-766N7.4, RP11-
767N6.2, RP11-767N6.7, RP11-768G7.1, RP11-769O8.2, RP11-770G2.2, RP11-
770G2.4, RP11-771K4.1, RP11-772E11.1, RP11-773H22.2, RP11-775A3.1, RP11-
775C24.3, RP11-775D22.2, RP11-775D22.3, RP11-777B9.3, RP11-778D9.9, RP11-
778J15.1, RP11-778J16.2, RP11-779O18.1, RP11-77G22.3, RP1-177G6.2, RP11-
77H9.6, RP11-77H9.8, RP11-77I22.2, RP11-77I22.3, RP11-77K12.3, RP11-781A6.1,
RP11-783L4.1, RP11-787D18.1, RP11-789C1.1, RP11-789C1.2, RP11-789C17.1,
RP11-789C17.3, RP11-789C17.5, RP11-78A19.3, RP11-78C3.1, RP11-78H18.2,
RP11-791G16.2, RP11-791J7.2, RP11-792D21.2, RP11-793G16.3, RP11-793H13.3,
RP11-793H13.4, RP11-793I11.1, RP11-794A8.1, RP11-794G24.1, RP11-795J1.1,
RP11-796G6.1, RP11-796G6.2, RP11-797H7.5, RP11-798G7.6, RP11-798K23.5,
RP11-798K3.2, RP11-799A12.1, RP11-799B12.2, RP11-799O21.1, RP11-79E3.1,
RP11-79L9.2, RP1-179N16.3, RP11-79N23.1, RP11-79P5.2, RP11-7D5.2, RP11-
7F17.1, RP11-7F17.3, RP11-7F17.5, RP11-7G23.4, RP11-7G23.6, RP11-7G23.8,
RP11-7G23.9, RP11-7I15.3, RP11-7I15.4, RP11-7K24.3, RP1-17K7.2, RP11-
7O14.1, RP11-800A18.4, RP11-800K23.2, RP11-803B1.2, RP11-804A23.4, RP11-
804L24.1, RP11-804M7.1, RP11-804N13.1, RP11-806H10.4, RP11-806K15.1,
RP11-806L2.6, RP11-806O11.1, RP11-807H7.2, RP11-809C18.1, RP11-809C18.3,
RP11-809M12.1, RP11-809N8.2, RP11-80B9.4, RP11-80F22.14, RP11-80F22.2,
RP11-80F22.5, RP11-80F22.6, RP11-80F22.7, RP11-80H5.4, RP11-80H5.7, RP11-
80H8.4, RP11-80I15.1, RP11-80I15.4, RP11-810P12.5, RP11-811I15.1, RP11-
813P10.2, RP11-814P5.1, RP11-815N9.2, RP11-819C21.1, RP11-819M15.1, RP11-
81H14.2, RP11-81H14.3, RP11-81H14.4, RP1-181J22.1, RP11-822E23.2, RP11-
824M15.3, RP11-826N14.2, RP1-182D15.2, RP11-82O18.1, RP11-82O19.2, RP11-
830F9.7, RP11-832N8.1, RP11-83B20.1, RP11-83J16.3, RP11-83N9.5, RP11-
841C19.3, RP11-843A23.1, RP11-844P9.1, RP11-845C23.2, RP11-848P1.2, RP11-
848P1.3, RP11-848P1.7, RP11-849F2.7, RP11-849N15.4, RP11-84A1.1, RP11-
84A14.4, RP11-84H6.1, RP11-851M3.1, RP11-854K16.2, RP11-857B24.5, RP11-
85A1.3, RP11-85G18.6, RP11-85G20.1, RP11-85G20.2, RP11-85K15.2, RP11-
860B13.3, RP11-861E21.2, RP11-861E21.3, RP11-862G15.1, RP11-863K10.4,
RP11-863K10.7, RP11-864J10.2, RP11-864J10.4, RP11-866E20.3, RP11-867G2.2,
RP11-867G2.5, RP11-867G23.10, RP11-867G23.12, RP11-867O8.5, RP11-
873E20.1, RP11-875O11.2, RP11-876N24.1, RP11-87N24.3, RP11-881L2.1, RP11-
881M11.1, RP11-881M11.2, RP11-883A18.3, RP11-884K10.5, RP11-885L14.1,
RP11-886H22.1, RP11-887P2.3, RP11-887P2.5, RP11-887P2.6, RP11-888D10.3,
RP11-889L3.1, RP11-88L24.4, RP11-890B15.2, RP11-893F2.15, RP11-893F2.9,
RP11-894P9.2, RP11-895K13.2, RP11-89B16.2, RP11-89C21.2, RP11-89C21.3,
RP11-89H19.1, RP11-89K21.1, RP11-89N17.1, RP11-8L2.1, RP11-8P11.3, RP11-
8P13.1, RP11-902B17.1, RP11-903H12.2, RP11-907B8.1, RP11-909M7.3, RP11-
90E9.1, RP11-90H3.1, RP11-90O23.1, RP11-90P13.1, RP11-90P5.1, RP11-91A18.1,
RP11-91H12.2, RP11-91I20.2, RP11-91K11.2, RP11-923I11.3, RP11-927P21.1,
RP11-927P21.2, RP11-927P21.6, RP11-92K15.1, RP11-932O9.1, RP11-932O9.7,
RP11-933H2.4, RP11-93B14.5, RP11-93K7.1, RP11-941H19.2, RP11-946L20.2,
RP11-94B19.5, RP11-94B19.6, RP11-94B19.7, RP11-94C24.6, RP11-950C14.7,
RP11-950C14.8, RP11-953B20.2, RP11-956A19.1, RP11-95P2.3, RP11-968A15.8,
RP11-96B5.2, RP11-96B5.4, RP11-96C23.9, RP11-96H19.1, RP11-96J19.1, RP11-
96K19.2, RP11-973H7.1, RP11-973N13.4, RP11-977B10.2, RP11-977G19.14, RP11-
97N19.2, RP11-97N5.2, RP11-981G7.1, RP11-981G7.2, RP11-982M15.6, RP11-
982M15.8, RP11-983P16.2, RP11-986E7.6, RP11-989F5.1, RP11-98L4.1, RP11-
993B23.3, RP11-99E15.1, RP11-99E15.2, RP11-9E13.2, RP1-20B11.2, RP1-20N2.6,
RP1-211D12.3, RP1-212P9.3, RP1-21O18.3, RP1-228P16.1, RP1-228P16.4, RP1-
228P16.7, RP1-232L22_B.1, RP1-232L24.3, RP1-239B22.5, RP1-239K6.1, RP1-
253P7.1, RP1-265C24.5, RP1-273G13.1, RP1-273G13.2, RP1-274L7.1, RP1-
276E15.1, RP1-278E11.3, RP1-281H8.3, RP1-283K11.2, RP1-292B18.4, RP1-
309F20.2, RP1-309F20.3, RP13-128O4.3, RP13-137A17.4, RP13-137A17.5, RP13-
143G15.3, RP13-143G15.4, RP1-315G1.1, RP13-16H11.1, RP13-210D15.4, RP13-
225O21.3, RP13-228J13.1, RP13-228J13.6, RP13-228J13.8, RP13-254B10.1, RP13-
258O15.1, RP13-262C2.3, RP13-331H15.1, RP13-347D8.1, RP13-436F16.1, RP13-
444K19.1, RP13-452N2.1, RP13-463N16.6, RP13-46H24.1, RP13-476E20.1, RP13-
487C10.1, RP13-488H8.1, RP1-34L19.1, RP13-503K1.4, RP13-516M14.1, RP13-
516M14.2, RP13-539F13.3, RP13-577H12.2, RP13-580F15.2, RP13-582O9.5, RP13-
585F24.1, RP13-608F4.1, RP13-638C3.6, RP13-644M16.5, RP13-650J16.1, RP13-
672B3.5, RP13-753N3.1, RP13-766D20.1, RP13-77O11.2, RP1-37N7.1, RP13-
870H17.3, RP13-88F20.1, RP13-895J2.3, RP13-923O23.6, RP13-98N21.3, RP13-
996F3.4, RP1-39G22.4, RP1-39G22.7, RP1-3J17.3, RP1-45I14.1, RP1-46F2.2, RP1-
46F2.3, RP1-56L9.7, RP1-60O19.1, RP1-67K17.3, RP1-67K17.4, RP1-68D18.4,
RP1-71H24.1, RP1-71H24.4, RP1-76B20.11, RP1-77H15.1, RP1-78O14.1, RP1-
7G5.6, RP1-80B9.2, RP1-80N2.3, RP1-85F18.5, RP1-8B1.4, RP1-90J20.11, RP1-
90J20.7, RP1-90J20.8, RP1-90J4.3, RP1-90L6.3, RP1-91G5.3, RP1-92O14.3, RP1-
93H18.1, RP1-95L4.4, RP1-97D16.1, RP1-99E18.2, RP3-322G13.7, RP3-322I12.2,
RP3-323K23.3, RP3-324O17.4, RP3-327A19.5, RP3-329A5.1, RP3-332B22.1, RP3-
340B19.2, RP3-340I3.1, RP3-340N1.2, RP3-341D10.1, RP3-343K2.4, RP3-344J20.2,
RP3-347M6.2, RP3-377H17.2, RP3-377O6.1, RP3-388E23.2, RP3-388N13.3, RP3-
393E18.1, RP3-393E18.2, RP3-395M20.2, RP3-395M20.3, RP3-395M20.8, RP3-
395M20.9, RP3-400B16.1, RP3-400N23.6, RP3-404K8.2, RP3-406P24.1, RP3-
408N23.4, RP3-410C9.1, RP3-412A9.15, RP3-414A15.2, RP3-417G15.1, RP3-
437I16.1, RP3-443C4.2, RP3-449M8.6, RP3-452M16.1, RP3-460G2.2, RP3-
461F17.1, RP3-462C17.1, RP3-462D8.2, RP3-467L1.4, RP3-471C18.1, RP3-
477M7.5, RP3-483K16.3, RP3-486I3.5, RP3-486I3.7, RP3-486L4.3, RP3-509I19.6,
RP3-511B24.4, RP3-512E2.2, RP3-521E19.2, RP3-521E19.3, RP3-522P13.2, RP3-
525L6.2, RP4-535B20.1, RP4-536B24.2, RP4-536B24.3, RP4-541C22.4, RP4-
545C24.5, RP4-549F15.1, RP4-550H1.4, RP4-553F4.6, RP4-562J12.1, RP4-
564F22.5, RP4-568F9.6, RP4-569M23.2, RP4-580O19.2, RP4-592A1.2, RP4-
593M8.1, RP4-594I10.2, RP4-594I10.3, RP4-595K12.2, RP4-597J3.1, RP4-
599G15.3, RP4-604A21.1, RP4-604K5.1, RP4-607I7.1, RP4-610C12.3, RP4-
610C12.4, RP4-612B15.3, RP4-612B18.1, RP4-612B18.3, RP4-614C15.2, RP4-
614C15.3, RP4-625H18.2, RP4-630A11.3, RP4-633I8.1, RP4-633I8.4, RP4-
635A23.4, RP4-635E8.1, RP4-641G12.4, RP4-651E10.4, RP4-656G21.1, RP4-
659J6.2, RP4-665J23.1, RP4-665N4.5, RP4-666F24.3, RP4-669H2.1, RP4-671G15.3,
RP4-682C21.5, RP4-684O24.5, RP4-697K14.3, RP4-705D16.3, RP4-706A16.3,
RP4-713B5.2, RP4-714D9.5, RP4-717I23.2, RP4-717I23.3, RP4-725G10.4, RP4-
736I12.1, RP4-737E23.2, RP4-738P15.1, RP4-753M9.1, RP4-755D9.1, RP4-
756H11.1, RP4-764D2.1, RP4-765A10.1, RP4-765C7.2, RP4-765H13.1, RP4-
777O23.2, RP4-778K6.3, RP4-779E11.3, RP4-781K5.8, RP4-782G3.1, RP4-
782L23.1, RP4-784A16.1, RP4-784A16.3, RP4-785G19.2, RP4-785G19.5, RP4-
791C19.1, RP4-791M13.4, RP4-791M13.5, RP4-794H19.1, RP4-794H19.2, RP4-
797C5.2, RP4-798A10.4, RP4-798C17.6, RP4-799P18.3, RP4-799P18.4, RP4-
800M22.1, RP4-803A2.1, RP4-803A2.2, RP4-803J11.2, RP4-805N21.1, RP4-
811H24.9, RP4-814D15.1, RP4-814D15.2, RP4-816N1.7, RP5-1002M8.4, RP5-
1007M22.2, RP5-1018A4.3, RP5-1021I20.1, RP5-1021I20.2, RP5-1022P6.3, RP5-
1022P6.7, RP5-1024G6.5, RP5-1024G6.7, RP5-1024N4.1, RP5-1028L10.1, RP5-
1029K10.2, RP5-1031J8.1, RP5-1033H22.2, RP5-1043L13.1, RP5-1044H5.1, RP5-
1044H5.2, RP5-1047A19.6, RP5-1050D4.5, RP5-1051J4.4, RP5-1053E7.1, RP5-
1053E7.3, RP5-1055C14.6, RP5-1056L3.3, RP5-1063M23.2, RP5-1067M6.3, RP5-
1068H6.3, RP5-1073O3.7, RP5-1077I2.3, RP5-1086D14.1, RP5-1091N2.2, RP5-
1104E15.6, RP5-1109J22.1, RP5-1115A15.1, RP5-1119A7.14, RP5-1121A15.3,
RP5-1121A15.4, RP5-1121E10.2, RP5-1125M8.5, RP5-1142J19.1, RP5-1142J19.2,
RP5-1148A21.3, RP5-1157M23.2, RP5-1160K1.1, RP5-1160K1.3, RP5-1160K1.8,
RP5-1170D6.1, RP5-1173A5.1, RP5-1178H5.2, RP5-1185H19.2, RP5-1185K9.1,
RP5-1198O20.4, RP5-817C23.1, RP5-823G15.5, RP5-836J3.1, RP5-837J1.2, RP5-
837M10.1, RP5-837M10.4, RP5-837O21.2, RP5-855F14.1, RP5-857K21.10, RP5-
857K21.14, RP5-857K21.3, RP5-857K21.4, RP5-862P8.2, RP5-867C24.1, RP5-
874C20.3, RP5-875H18.4, RP5-882O7.4, RP5-882O7.5, RP5-884G6.2, RP5-
886K2.1, RP5-886K2.3, RP5-894A10.2, RP5-905N1.2, RP5-907D15.3, RP5-
919F19.5, RP5-924G13.2, RP5-931E15.2, RP5-931K24.3, RP5-934G17.2, RP5-
934G17.6, RP5-937E21.8, RP5-940J5.3, RP5-940J5.8, RP5-943J3.1, RP5-951N9.1,
RP5-956O18.2, RP5-961K14.1, RP5-963E22.4, RP5-968D22.1, RP5-978I12.1, RP5-
981L23.1, RP5-983H21.3, RP5-983L19.1, RP5-983L19.2, RP5-991C6.4, RP5-
991G20.2, RP5-991O23.1, RP5-995J12.2, RP5-997D16.2, RP5-998N21.4, RP6-
109B7.2, RP6-109B7.3, RP6-109B7.4, RP6-11O7.2, RP6-186E3.1, RP6-191P20.4,
RP6-201G10.2, RP6-227L5.2, RP6-65G23.1, RPA1, RPA2, RPA3, RPA4, RPAP2,
RPAP3, RPE, RPF1, RPF2, RPF2P1, RPH3AL, RPIA, RPL10AP2, RPL11P5,
RPL12L3, RPL12P9, RPL13, RPL13AP17, RPL13AP6, RPL13AP7, RPL13P12,
RPL15, RPL15P2, RPL15P20, RPL17P1, RPL17P26, RPL18AP2, RPL18P10,
RPL18P11, RPL19, RPL19P21, RPL21, RPL21P28, RPL21P3, RPL22, RPL22P11,
RPL23, RPL23AP53, RPL23P2, RPL24, RPL26, RPL26P30, RPL26P37, RPL27AP,
RPL29P11, RPL29P24, RPL31P2, RPL31P58, RPL31P63, RPL31P7, RPL34,
RPL34P23, RPL34P31, RPL35AP26, RPL35AP30, RPL36AL, RPL36AP43,
RPL36AP45, RPL36P2, RPL37AP8, RPL37P2, RPL39, RPL39P3, RPL39P40,
RPL3L, RPL3P12, RPL5, RPL5P29, RPL7, RPL7AP14, RPL7AP26, RPL7AP34,
RPL7AP65, RPL7AP8, RPL7L1P8, RPL7L1P9, RPL7P57, RPL8P2, RPL9P5,
RPL9P9, RPN1, RPP25, RPP30, RPS10P16, RPS10P18, RPS10P2, RPS10P7,
RPS11P6, RPS13, RPS15AP40, RPS15P9, RPS17, RPS17P2, RPS18P9, RPS19BP1,
RPS20P2, RPS20P35, RPS23P8, RPS24, RPS24P17, RPS26P47, RPS27A,
RPS27AP1, RPS27AP2, RPS27L, RPS27P27, RPS2P41, RPS2P44, RPS2P45,
RPS2P48, RPS3A, RPS3AP26, RPS3AP34, RPS3AP35, RPS3AP39, RPS3AP44,
RPS3AP47, RPS3AP5, RPS3AP6, RPS3P1, RPS3P7, RPS4XP16, RPS4XP17,
RPS4XP19, RPS4XP22, RPS4Y1, RPS6, RPS6KC1, RPS6P16, RPS6P21, RPS7P2,
RPS8P3, RPSAP4, RPSAP47, RPSAP56, RQCD1, RRAD, RRAGC, RRAGD,
RRAS2, RRH, RRM1, RRM1-AS1, RRM2B, RRN3, RRP12, RSBN1, RSL24D1,
RSL24D1P1, RSL24D1P11, RSL24D1P3, RSPH10B2, RSPH6A, RSPO1, RSPO2,
RSU1, RTBDN, RTN3P1, RTN4, RTN4IP1, RTP4, RUFY4, RUNDC3B, RUNX1,
RUNX2, RUNX3, RUVBL1-AS1, RWDD1, RWDD4, RWDD4P2, RXFP3, RXRB,
RXRG, RYBP, RYR2, S100A11, S100A12, S100A2, S100A3, S100A5, S100A8,
S100A9, S100B, S1PR1, SACM1L, SACS-AS1, SAMD11, SAMD3, SAMD4B,
SAMD8, SAMD9, SAMD9L, SAMSN1, SAMSN1-AS1, SAP30, SAR1A, SAR1B,
SASH3, SASS6, SAT1, SATB1, SAV1, SAYSD1, SBDS, SBDSP1, SBNO2, SBSN,
SC5DL, SCAF11, SCAMP1, SCARB2, SCARF1, SCARNA11, SCARNA13,
SCARNA20, SCARNA21, SCARNA6, SCARNA7, SCARNA8, SCARNA9, SCD,
SCD5, SCDP1, SCEL-AS1, SCFD1, SCFD2, SCG2, SCG3, SCG5, SCGB1A1,
SCGB1D2, SCGB2A1, SCGB2B2, SCGB3A1, SCGN, SCIN, SCLT1, SCML1,
SCN1B, SCN4A, SCNN1B, SCO1, SCOC, SCRN3, SCRT2, SCXA, SDAD1,
SDAD1P1, SDAD1P2, SDAD1P4, SDCBP, SDCCAG3P1, SDCCAG3P2, SDE2,
SDF2L1, SDHB, SDHD, SDIM1, SDR16C5, SDR42E1, SDS, SDSL, SEC11C,
SEC14L3, SEC16B, SEC22A, SEC22B, SEC23A, SEC23B, SEC23IP, SEC24A,
SEC24D, SEC31A, SEC61G, SEC62, SEC62-AS1, SEC63, SEC63P1, SECISBP2L,
SECTM1, SEL1L, SEL1L2, SEL1L3, SELE, SELK, SELM, SELPLG, SEMA4A,
SEMA4B, SEMA5A, SEMA6D, SEMG1, SENP3-EIF4A1, SENP8, SEPHS1P4,
SEPHS1P6, SEPT10P1, SEPT2P1, SEPT7L, SEPT7P6, SEPT7P7, SEPT7P8,
SEPW1, SERF2, SERINC1, SERINC3, SERP1, SERPINA3, SERPINA4,
SERPINA5, SERPINB1, SERPINB2, SERPINB5, SERPINB6, SERPINB8,
SERPINC1, SERPINE1, SERTAD1, SERTAD4, SERTAD4-AS1, SESN1, SETD3,
SETD7, SETP18, SETP2, SETP20, SETP22, SETX, SF3A3P2, SF3B5, SFMBT1,
SFR1, SFRP4, SFT2D2, SFTPA1, SFTPC, SFXN5, SGCA, SGCE, SGIP1, SGK1,
SGK110, SGK3, SGMS1, SGMS2, SGOL1, SGOL1-AS1, SGPP1, SGPP2, SGSM1,
SH2B3, SH2D2A, SH2D5, SH2D6, SH3BGR, SH3GL2, SH3GLB1, SH3YL1,
SHCBP1, SHFM1, SHFM1P1, SHOC2, SHQ1, SHROOM1, SIAH2, SIAH3, SIDT1,
SIGLEC1, SIGLEC10, SIGLEC11, SIGLEC14, SIGLEC16, SIGLEC5, SIGLEC8,
SIGLEC9, SIK1, SIK2, SIKE1, SIPA1L2, SIRPB1, SIRT1, SIX1, SIX3, SIX3-AS1,
SIX4, SIX6, SKA2, SKA2L, SKIV2L2, SKP1, SKP1P1, SKP2, SLA, SLA2,
SLAIN2, SLAMF1, SLAMF7, SLAMF8, SLAMF9, SLBP, SLC10A4, SLC10A5,
SLC10A7, SLC11A1, SLC12A8, SLC14A2, SLC16A4, SLC16A6, SLC16A9,
SLC17A6, SLC18A2, SLC19A2, SLC1A1, SLC1A2, SLC1A4, SLC22A15,
SLC22A2, SLC22A24, SLC22A9, SLC25A17, SLC25A1P5, SLC25A24,
SLC25A30, SLC25A32, SLC25A34, SLC25A3P2, SLC25A40, SLC25A45,
SLC25A46, SLC25A52, SLC25A53, SLC25A5P1, SLC25A5P7, SLC25A6P4,
SLC25A6P5, SLC26A2, SLC26A3, SLC26A5, SLC27A2, SLC27A6, SLC28A2,
SLC28A3, SLC2A10, SLC2A12, SLC2A13, SLC2A2, SLC2A5, SLC2A9,
SLC30A2, SLC30A5, SLC30A8, SLC31A1, SLC31A1P1, SLC31A2, SLC33A1,
SLC34A2, SLC35A5, SLC35D3, SLC35E1P1, SLC35G1, SLC35G2, SLC35G6,
SLC36A4, SLC38A4, SLC39A12, SLC39A14, SLC39A4, SLC39A6, SLC39A8,
SLC3A1, SLC3A2, SLC40A1, SLC41A1, SLC44A1, SLC45A2, SLC45A3,
SLC46A3, SLC47A2, SLC4A10, SLC4A1AP, SLC4A4, SLC4A7, SLC52A3,
SLC5A1, SLC5A3, SLC5A4, SLC5A5, SLC6A13, SLC6A14, SLC6A17, SLC6A20,
SLC6A6, SLC7A1, SLC7A2, SLC7A5, SLC7A5P1, SLC8A1, SLC8A1-AS1,
SLC9A7P1, SLC9A9, SLC9A9-AS2, SLC9B2, SLC9C1, SLC9C2, SLCO1A2,
SLCO5A1, SLFN11, SLFN12, SLFN5, SLIRP, SLK, SLMAP, SLMO2, SLPI, SLU7,
SLX1B, SMAD1, SMARCA5, SMARCE1P6, SMC3, SMC5, SMCHD1, SMEK2,
SMG1P1, SMIM-2-IT1, SMNDC1, SMPDL3A, SNAI2, SNAP23, SNAP25, SNAP25-
AS1, SNAP29, SNAP91, SNAPC1, SNAPC5, SNCA, SNCB, SNHG10, SNHG12,
SNHG9, SNIP1, SNORA11, SNORA12, SNORA15, SNORA16, SNORA16B,
SNORA22, SNORA24, SNORA25, SNORA26, SNORA27, SNORA28, SNORA2B,
SNORA31, SNORA32, SNORA33, SNORA4, SNORA46, SNORA47, SNORA51,
SNORA55, SNORA57, SNORA58, SNORA5A, SNORA5C, SNORA60, SNORA65,
SNORA66, SNORA67, SNORA68, SNORA70, SNORA71A, SNORA71B,
SNORA71C, SNORA72, SNORA73, SNORA73B, SNORA75, SNORA7B,
SNORA81, SNORA84, SNORD100, SNORD11, SNORD111, SNORD112,
SNORD113, SNORD113-1, SNORD113-3, SNORD113-4, SNORD114-1,
SNORD114-10, SNORD114-12, SNORD114-13, SNORD114-17, SNORD114-18,
SNORD114-21, SNORD114-25, SNORD114-26, SNORD114-27, SNORD114-28,
SNORD114-3, SNORD114-4, SNORD114-5, SNORD114-6, SNORD114-7,
SNORD115-12, SNORD115-21, SNORD115-44, SNORD115-45, SNORD116-12,
SNORD116-13, SNORD116-16, SNORD116-29, SNORD116-4, SNORD116-8,
SNORD12, SNORD127, SNORD12C, SNORD14A, SNORD14E, SNORD19,
SNORD19B, SNORD1B, SNORD36C, SNORD37, SNORD3A, SNORD3B-1,
SNORD3B-2, SNORD43, SNORD51, SNORD53_SNORD92, SNORD56, SNORD6,
SNORD63, SNORD66, SNORD69, SNORD88, SNORD88A, snoU13, SNRNP27,
SNRPE, SNRPEP2, SNRPEP4, SNRPF, SNRPFP1, SNRPGP10, SNRPGP14,
SNRPGP2, SNRPGP4, SNTG1, SNW1, SNX10, SNX13, SNX14, SNX16,
SNX18P13, SNX18P3, SNX18P9, SNX19P2, SNX2, SNX20, SNX25, SNX25P1,
SNX29, SNX29P2, SNX3, SNX5, SNX5P1, SNX8, SOAT1, SOBP, SOCS1, SOCS2,
SOCS2-AS1, SOCS3, SOCS4, SOCS5, SOD1, SOD2, SOD3, SOGA3, SOHLH2,
SORL1, SORT1, SOS2, SOWAHA, SOWAHB, SOX18, SOX2-OT, SOX30, SOX6,
SP1, SP100, SP110, SP140, SP140L, SPA17, SPAG1, SPAG6, SPARCL1, SPATA1,
SPATA16, SPATA18, SPC24, SPC25, SPCS1, SPCS2, SPCS3, SPDYA, SPDYE6,
SPDYE8P, SPEF1, SPEM1, SPESP1, SPHK1, SPI1, SPICE1, SPINK13, SPINK2,
SPINK9, SPIRE1, SPOPL, SPP1, SPPL2A, SPR, SPRR1A, SPRR2A, SPRYD3,
SPRYD7, SPSB1, SPTB, SPTLC1, SPTLC1P1, SPTLC1P4, SPTLC1P5, SPTSSA,
SPTY2D1, SQRDL, SRBD1, SRD5A1, SRD5A1P1, SRGAP2, SRGAP2-AS1,
SRGAP2B, SRGAP3-AS1, SRGAP3-AS2, SRGAP3-AS4, SRGN, SRP54, SRP68P1,
SRP68P2, SRP72, SRPR, SRPRB, SRXN1, SSB, SSBP1, SSBP2, SSFA2, SSPN,
SSR1, SSR3, SSSCA1, SSX2IP, SSXP10, ST13, ST13P13, ST13P14, ST14, ST20,
ST3GAL5, ST3GAL6, ST3GAL6-AS1, ST7-AS2, ST7L, STAG2, STAMBPL1,
STAP1, STAP2, STARD13-AS2, STARD3NL, STAT3, STAT4, STAT5A, STAT5B,
STAU2, STAU2-AS1, STBD1, STEAP2, STEAP3, STEAP3-AS1, STEAP4, STIM2,
STK3, STK38L, STK4, STK4-AS1, STOM, STPG2, STRAP, STRCP1, STT3A,
STX11, STX12, STX17, STX19, STX1A, STXBP1, STXBP3, STXBP5, STXRP5-
AS1, STXBP6, STYXL1, SUB1P1, SUB1P3, SUB1P4, SUCLA2-AS1, SUCLA2P3,
SUCLG1, SUCLG2, SUCLG2P2, SUCO, SUDS3P1, SUGT1, SULT1A1, SULT1A2,
SULT1A3, SULT6B1, SUMO1, SUMO2P3, SUMO2P4, SUMO3, SUMO4, SUN3,
SURF2, SURF4, SUSD1, SUZ12, SVIP, SVOPL, SWAP70, SYBU, SYCE1, SYCE3,
SYCN, SYCP2, SYCP3, SYF2, SYF2P2, SYK, SYNCRIP, SYNE2, SYNGR4,
SYNM, SYNPO, SYPL1, SYT13, SYT14, SYT16, SYT4, SYT8, SYTL2, SYTL3,
TAB2, TABS-AS1, TAB3-AS2, TACC1, TACSTD2, TADA1, TAF1, TAF12,
TAF13, TAF1A, TAF1B, TAF1D, TAF4B, TAF5, TAF7, TAF7L, TAF9, TAF9B,
TAF9BP1, TAF9P3, TAGAP, TAGLN2P1, TAL2, TANC1, TANC2, TAOK3,
TAP1, TAPBP, TAPBPL, TARDBP, TARS, TAS1R1, TAS2R20, TAS2R3,
TAS2R31, TAS2R38, TAS2R4, TAS2R64P, TAT, TATDN2P2, TATDN3,
TAX1BP1, TBC1D12, TBC1D15, TBC1D22A, TBC1D23, TBC1D29, TBC1D2B,
TBC1D30, TBC1D3B, TBC1D3H, TBC1D3P1-DHX40P1, TBC1D3P6, TBC1D4,
TBC1D8, TBC1D8B, TBC1D9, TBCAP3, TBCCD1, TBK1, TBL1XR1, TBL1XR1-
AS1, TBP, TBPL1, TBR1, TBX19, TBX21, TBXA2R, TC2N, TCEA1, TCEA1P2,
TCEA1P4, TCEAL6, TCEANC, TCEB3B, TCERG1L, TCF15, TCF21, TCF4,
TCHH, TCL1B, TCL6, TCN1, TCP1, TCP10L, TCP11, TCP11L2, TCP1P1, TCTE3,
TCTEX1D1, TDGF1, TDGF1P4, TDGF1P6, TDP2, TDPX2, TDRD12, TDRD5,
TDRD6, TDRD9, TDRG1, TEAD4, TEC, TEK, TEKT1, TEP1, TERF1P2, TESC,
TEX10, TEX14, TEX19, TEX21P, TEX26-AS1, TEX29, TEX30, TEX35, TEX9,
TFAM, TFB2M, TFCP2, TFCP2L1, TFEB, TFEC, TFP1, TFP1, TFRC, TG, TGDS,
TGFA, TGFBR3, TGFBR3L, TGM2, TGM5, TGOLN2, THADA, THAP10, THAP5,
THAP9, THAP9-AS1, THBD, THEM2, THEM6, THEMIS2, THNSL1, THNSL2,
THOC7, THUMPD2, THUMPD3, TICAM2, TIFA, TIGD1, TIGD1L2, TIGD2,
TIGD7, TIGIT, TIMM17A, TIMM23, TIMM8A, TIMMDC1, TIMP2, TIMP4,
TINF2, TIPARP, TIPIN, TIPRL, TLK1, TLK1P1, TLK2P1, TLL1, TLL2, TLR1,
TLR2, TLR3, TLR4, TLR6, TLR9, TM2D1, TM2D3, TM4SF1, TM4SF18, TM7SF3,
TM9SF2, TM9SF3, TMA16P2, TMBIM6, TMC2, TMC5, TMCO1, TMCO2,
TMCO4, TMCO5B, TMED10, TMED10P, TMED10P1, TMED11P, TMED2,
TMED5, TMED6, TMED7, TMED7-TICAM2, TMEM100, TMEM106A,
TMEM106A-AS1, TMEM106C, TMEM108, TMEM109, TMEM110, TMEM111,
TMEM116, TMEM117, TMEM119, TMEM123, TMEM126A, TMEM126B,
TMEM133, TMEM135, TMEM14B, TMEM14C, TMEM150C, TMEM154,
TMEM156, TMEM158, TMEM161B, TMEM161B-AS1, TMEM163, TMEM165,
TMEM167A, TMEM167B, TMEM168, TMEM17, TMEM173, TMEM194A,
TMEM2, TMEM200A, TMEM208, TMEM209, TMEM211, TMEM212, TMEM219,
TMEM236, TMEM241, TMEM246, TMEM26, TMEM27, TMEM30A, TMEM30B,
TMEM33, TMEM38B, TMEM39A, TMEM40, TMEM41B, TMEM43, TMEM44-
AS1, TMEM48, TMEM5, TMEM54, TMEM57, TMEM59, TMEM60, TMEM61,
TMEM64, TMEM66, TMEM68, TMEM69, TMEM70, TMEM87A, TMEM8C,
TMEM97P1, TMEM99, TMEM9B, TMF1, TMIE, TMLHE, TMOD1, TMOD2,
TMOD4, TMPRSS11B, TMPRSS11F, TMPRSS12, TMPRSS15, TMPRSS2,
TMPRSS4, TMSB10P1, TMSB4XP2, TMSB4XP4, TMSB4Y, TMX1, TMX2,
TMX4, TncRNA, TNFAIP3, TNFAIP8, TNFAIP8L2, TNFRSF10C, TNFRSF11A,
TNFRSF11B, TNFRSF12A, TNFRSF14, TNFRSF17, TNFRSF1B, TNFRSF21,
TNFRSF4, TNFRSF8, TNFRSF9, TNFSF10, TNFSF11, TNFSF13, TNFSF13B,
TNFSF14, TNFSF9, TNIK, TNKS2, TNN, TNNI2, TNRC18P1, TNS1, TNS3, TNS4,
TOB2P1, TOMM22P4, TOMM22P5, TOMM7, TOMM70A, TOP1P1, TOPORS,
TOR1AIP1, TP53RK, TPD52, TPH1, TPH2, TPI1P3, TPM3P8, TPP1, TPP2, TPPP3,
TPRKB, TPRX1, TPT1, TPTE2P5, TPTE2P6, TPTEP1, TRAC, TRAF3IP2,
TRAF3IP3, TRAF5, TRAK2, TRAM1, TRAM1L1, TRAPPC11, TRAPPC4,
TRAPPC6B, TRAV27, TRAV30, TRDC, TREM1, TREM2, TREML1, TREML3P,
TREX2, TRIAP1, TRIB1, TRIM23, TRIM25, TRIM29, TRIM34, TRIM37, TRIM38,
TRIM4, TRIM47, TRIM48, TRIM54, TRIM60P17, TRIM63, TRIM7, TRIM72,
TRIM9, TRIP10, TRIP11, TRIP12, TRIQK, TRMT10A, TRMT10C, TRMT11,
TRMT12, TRMT1L, TRMT6, TRMT61B, TRNAI2, TRNT1, TROVE2, TRPC5OS,
TRPM3, TRPM6, TRPM7, TRPM8, TRPS1, TRPV3, TRUB1, TSACC, TSC22D1,
TSC22D3, TSEN15, TSFM, TSGA13, TSGA14, TSHB, TSHZ1, TSHZ2, TSHZ3,
TSLP, TSN, TSPAN1, TSPAN12, TSPAN13, TSPAN2, TSPAN5, TSPAN7,
TSPAN8, TSPEAR, TSPYL1, TSPYL2, TSPYL5, TSPYL6, TSSK1A, TSSK1B,
TSTD2, TTBK2, TTC1, TTC13, TTC15, TTC17, TTC22, TTC27, TTC30A,
TTC39B, TTC3P1, TTC4P1, TTC7B, TTF1, TTF2, TTI2, TTLL11-IT1, TTLL7-IT1,
TTPA, TUBA1B, TUBA1C, TUBA3GP, TUBD1, TUBE1, TUFT1, TULP3P1,
TWF1, TWIST1, TWISTNB, TWSG1, TXK, TXLNB, TXLNG, TXN, TXNDC11,
TXNDC15, TXNDC2, TXNDC5, TXNDC9, TXNL1, TXNRD1, TYMS, TYRO3P,
TYROBP, TYW1B, TYW3, U1, U2, U3, U4, U47924.19, U47924.27, U52111.12,
U6, U6atac, U7, U73169.1, U8, U82695.10, UAP1, UBA2, UBA5, UBA7, UBAC2-
AS1, UBASH3A, UBE2A, UBE2B, UBE2CP2, UBE2D3, UBE2D3P1, UBE2D3P2,
UBE2D4, UBE2F, UBE2HP1, UBE2J1, UBE2L4, UBE2L5P, UBE2QL1, UBE2U,
UBE2V1P1, UBE2V2, UBE3A, UBE3C, UBL3, UBL4B, UBLCP1, UBP1,
UBQLN1, UBQLN2, UBR1, UBR3, UBR7, UBTFL6, UBXN2B, UBXN4, UBXN6,
UBXN8, UCHL1, UCHL3, UCHL5, UCP1, UFL1, UFM1, UFSP1, UFSP2, UGDH,
UGDH-AS1, UGGT2, UGP2, UGT2B15, UGT8, UHMK1, UHRF1BP1,
UHRF1BP1L, ULBP1, ULBP2, ULK4, ULK4P2, UMODL1, UNC50, UNC5CL,
UPF2, UPF3AP3, UPP2, UPRT, UQCRB, UQCRBP1, UQCRBP3, URI1, USE1,
USMG5, USMG5P1, USO1, USP1, USP12, USP12PY, USP16, USP2, USP25,
USP31, USP32P1, USP32P2, USP38, USP43, USP44, USP46, USP46-AS1, USP48,
USP49, USP50, USP7, USP8, USP9X, USPL1, UTF1, UTP14C, UTP15, UTP18,
UTP3, UTS2, VAMP1, VAMP2, VAMP4, VAMP5, VAMP8, VAPA, VAPB,
VAT1L, Vault, VAV1, VAV3, VBP1, VCAM1, VCPIP1, VDAC1, VDAC1P1,
VDAC1P2, VDAC1P6, VDAC3, VDR, VEGFA, VGF, VHLL, VIMP, VIP, VLDLR,
VMA21, VMP1, VN1R104P, VN1R110P, VN1R14P, VN1R5, VN2R19P, VOPP1,
VPREB3, VPS13C, VPS37A, VPS37C, VPS4A, VPS54, VRK2, VSIG10, VSX1,
VTI1B, VTI1BP1, VTI1BP2, VTRNA1-1, VTRNA1-2, VERNA1-3, VWA3A,
VWA3B, VWC2L, VWDE, VWF, WAC, WAPAL, WARS, WASF2, WASF4P,
WASL, WBP1LP2, WBP2NL, WBP4, WBSCR22, WBSCR27, WDFY2, WDFY3-
AS2, WDFY4, WDPCP, WDR12, WDR16, WDR3, WDR36, WDR38, WDR4,
WDR41, WDR43, WDR44, WDR47, WDR49, WDR52-AS1, WDR53, WDR5B,
WDR61, WDR67, WDR69, WDR76, WDR82P1, WDR89, WDYHV1, WEE2,
WFDC10B, WFDC3, WFIKKN2, WHAMM, WHAMMP2, WHAMMP3, WI2-
1896O14.1, WI2-2373I1.1, WISP3, WLS, WNT10A, WNT16, WNT4, WNT7B,
WNT9A, WSB2, WSCD2, WTAPP1, WWC1, WWC2-AS1, WWC3-AS1, WWP1,
WWTR1-AS1, WWTR1-IT1, XAF1, XBP1, XBP1P1, XCR1, XDH, XG, XIAP-AS1,
XIRP1, XIST, XKR9, XPA, XPO1, XPO4, XPOT, XPOTP1, XPR1, XRRA1,
XXbac-B444P24.10, XXbac-B476C20.10, XXbac-B476C20.11, XXbac-
B476C20.14, XXbac-B476C20.17, XXbac-BPG55C20.7, XX-C2158C12.2, XX-
C2158C6.1, XX-C2158C6.3, XX-FW80269A6.1, XX-FW83128A1.2, XXyac-
YX155B6.2, XXyac-YX65C7_A.2, XXYLT1-AS2, XYLB, Y_RNA, YARS,
YBX1P1, YBX1P2, YBX1P3, YIPF4, YIPF5, YIPF6, YOD1, YRDCP2, YSK4,
YTHDC2, YWHAH, YWHAQ, YWHAQP6, YWHAZP5, YY2, Z69666.2,
Z73979.1, Z83001.1, Z84721.4, Z95704.3, Z98881.1, ZAN, ZAR1, ZBP1, ZBTB1,
ZBTB10, ZBTB20, ZBTB20-AS1, ZBTB20-AS3, ZBTB20-AS4, ZBTB24, ZBTB25,
ZBTB38, ZBTB4, ZBTB40-IT1, ZBTB6, ZBTB7A, ZBTB8A, ZBTB8OSP1,
ZC3H12C, ZC3H15, ZC3H6, ZC3HAV1, ZCCHC14, ZCCHC2, ZCCHC7,
ZCCHC9, ZCRB1, ZCWPW2, ZDHHC19, ZDHHC2, ZDHHC20-IT1, ZDHHC20P4,
ZEB1, ZEB1-AS1, ZEB2, ZFAND1, ZFAND5, ZFC3H1, ZFP1, ZFP106, ZFP14,
ZFP2, ZFP3, ZFP62, ZFP82, ZFP91, ZFYVE28, ZFYVE9, ZFYVE9P1, ZHX1,
ZIM3, ZMAT1, ZMAT3, ZMAT5, ZMPSTE24, ZMYM5, ZMYM6, ZMYND10,
ZMYND11, ZMYND12, ZNF10, ZNF12, ZNF136, ZNF138, ZNF14, ZNF146,
ZNF148, ZNF165, ZNF17, ZNF175, ZNF181, ZNF182, ZNF189, ZNE19, ZNF197-
AS1, ZNF22, ZNF221, ZNF225, ZNF230, ZNF234, ZNF239, ZNF24, ZNF248,
ZNF25, ZNF252, ZNF252P, ZNF252P-AS1, ZNF256, ZNF257, ZNF263, ZNF271,
ZNF275, ZNF280B, ZNF280D, ZNF281, ZNF283, ZNF285, ZNF295, ZNF295-AS1,
ZNF300P1, ZNF302, ZNF322, ZNF322P1, ZNF323, ZNF331, ZNF33A, ZNF33AP1,
ZNF343, ZNF347, ZNF350, ZNF354A, ZNF365, ZNF37A, ZNF385D, ZNF395,
ZNF397, ZNF41, ZNF415, ZNF417, ZNF420, ZNF429, ZNF434, ZNF439, ZNF441,
ZNF460, ZNF461, ZNF471, ZNF483, ZNF484, ZNF485, ZNF493, ZNF502,
ZNF503-AS1, ZNF506, ZNF518B, ZNF519, ZNF525, ZNF540, ZNF546, ZNF547,
ZNF556, ZNF558, ZNF559, ZNF563, ZNF564, ZNF567, ZNF569, ZNF57, ZNF570,
ZNF571, ZNF572, ZNF583, ZNF585A, ZNF585B, ZNF594, ZNF595, ZNF596,
ZNF600, ZNF616, ZNF619, ZNF625, ZNF630, ZNF638-IT1, ZNF639, ZNF643,
ZNF652, ZNF658, ZNF658B, ZNF662, ZNF664, ZNF665, ZNF670, ZNF676,
ZNF684, ZNF697, ZNF702P, ZNF705A, ZNF706, ZNF717, ZNF721, ZNF732,
ZNF736, ZNF750, ZNF763, ZNF766, ZNF770, ZNF774, ZNF782, ZNF79, ZNF791,
ZNF799, ZNF80, ZNF800, ZNF806, ZNF816, ZNF823, ZNF826P, ZNF829,
ZNF831, ZNF835, ZNF839P1, ZNF84, ZNF841, ZNF844, ZNF845, ZNF850,
ZNF859P, ZNF860, ZNF876P, ZNF878, ZNF890P, ZNF91, ZNF98, ZNFX1,
ZNHIT6, ZNRF2, ZNRF3-AS1, ZNRF3-IT1, ZP4, ZPLD1, ZRSR1, ZSCAN10,
ZSCAN12P1, ZSWIM5P2, ZUFSP, ZW10, ZXDA, ZXDB, ZYG11B, ZZEF1, ZZZ3

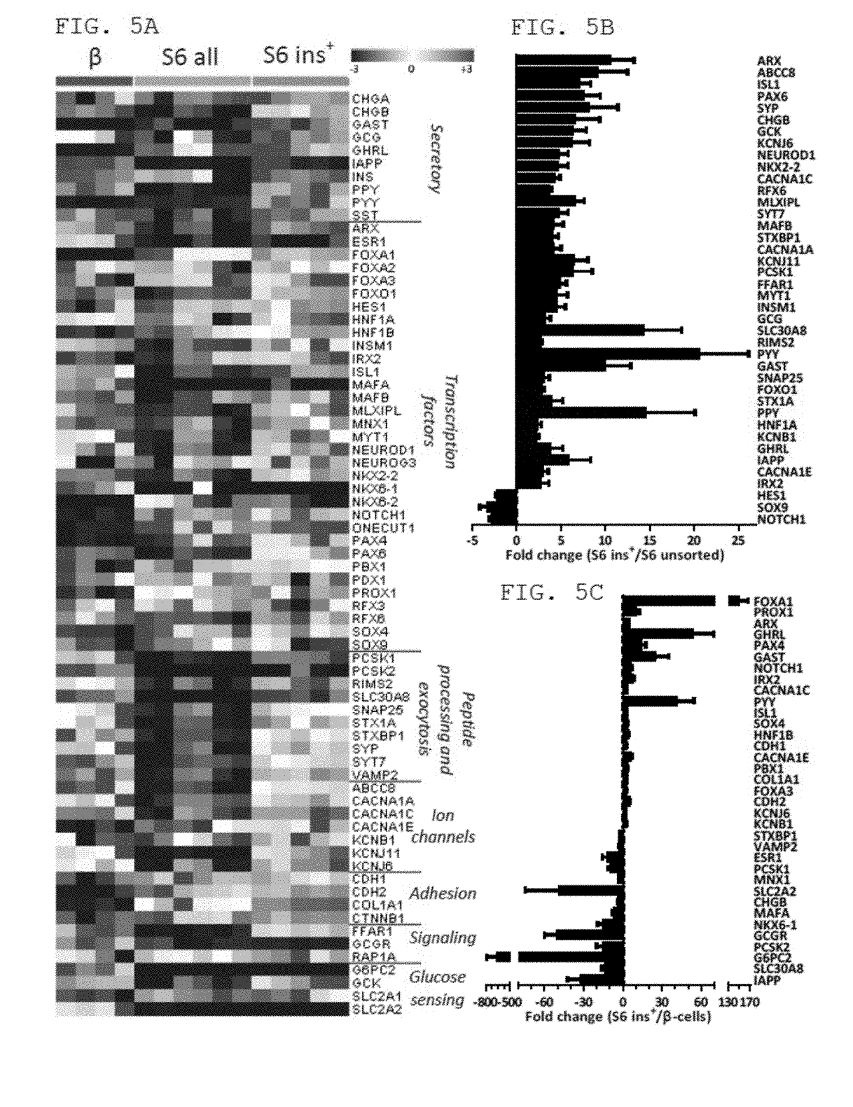

It is to be understood that the levels of expression of one or more of the genes listed in Table 2 are depicted in FIG. 5C as a fold change in expression of in vitro-differentiated insulin-positive β-like cells compared to mature β-cells.

It should also be appreciated that any gene listed in Table 2 can be used as a marker for detecting mature β-cells by measuring the level of expression of the gene in a cell, culture, cell line, tissue, or population of cells (e.g., suspected of being β-cells), wherein if the level of expression of the gene in the cell, culture, cell line, tissue, or population of cells is elevated (for example, as depicted in FIG. 5C), the cell, culture, cell line, tissue, or population of cells comprises mature β-cells.

Those skilled in the art will also appreciate that any two or more of the genes listed in Table 2 can be used in combinations of up to N genes (where N is a positive integer greater than or equal to 2) as markers for detecting mature β-cells by measuring the levels of expression of the combination of genes in a cell, culture, cell line, tissue, or population of cells (e.g., suspected of being β-cells), wherein if the levels of expression of the combination of genes in the cell, culture, cell line, tissue, or population of cells is elevated (for example, as depicted in FIG. 5C), the cell, culture, cell line, tissue, or population of cells comprises mature β-cells.

In some aspects, a marker of β-cell functional immaturity includes one, any combination or sub-combination, or all genes which are have a higher expression level in fetal β-cells or in vitro-differentiated insulin-positive β-like cells compared to mature β-cells.

Exemplary genes which are differentially expressed in mature β-cells compared to in vitro-differentiated insulin-positive β-like cells are listed in Table 2C.

TABLE 2C
Genes differentially expressed between mature β-cells compared
to in vitro-differentiated insulin-positive β-like cells

A1CF, ABHD5, ABHD9, ACACB, ACBD7, ACCN2, ACCN4, ACOT7, ACOX2,
ADAMTS9, ADAMTSL2, ADAP2, ADH1A, ADORA2A, ADSL, AEBP1, AFP,
AGPAT9, AGR2, AMBP, ANGPTL2, ANKRD43, ANKS1B, ANKS4B, ANO6,
ANO9, ANTXR2, AOX1, APC2, APCDD1L, APOA1, APOB, APOC1, APOC3,
APOE, APOH, AQP10, ARHGAP24, ARHGAP4, ARHGEF3, ARID3B, ARID5B,
ARL3, ARL9, ARX, ASAP2, ASAP3, ASB9, ASGR1, ATCAY, ATP6V1E2,
AUTS2, B2M, B3GAT1, BAG3, BAMB1, BARD1, BASP1, BCAR3, BMP5, BPIL2,
BTBD17, BTG3, C10orf10, C10orf140, C12orf27, C13orf15, C15orf57, C15orf59,
C16orf59, C1orf127, C1orf133, C1orf158, C1QTNF5, C21orf119, C21orf63,
C2CD4B, C2orf32, C2orf70, C3orf21, C3orf32, C3orf57, C5, C5orf13, C5orf38,
C5orf4, C5orf53, C6orf115, C6orf117, C6orf89, C7orf23, C7orf52, CBA, C8orf44,
C8orf47, C9orf123, C9orf66, C9orf9, C9orf95, CABLES1, CABP7, CADM4,
CALB2, CAPN13, CARD10, CASR, CBX7, CCBP2, CCDC109B, CCDC110,
CCDC74B, CCND3, CCPG1, CD14, CD44, CD5, CD55, CDC20, CDH10, CDH12,
CDH22, CDH4, CDK6, CDKN1A, CDKN3, CDO1, CDS1, CEACAM1, CEBPA,
CEBPD, CENPV, CENTA1, CFLAR, CGN, CGNL1, CHGB, CHKA, CHPF,
CHST1, CHST13, C1B1, CLDN1, CLDN11, CLDN6, CLRN3, CNN3, CNRIP1,
CNTNAP2, COL4A5, COX7A1, CPT1C, CPVL, CPXM1, CRABP1, CRH,
CRYBA2, CSRP2, CTGF, CTNNBIP1, CTSF, CTXN1, CYB5R2, CYBA, CYP2J2,
CYP2U1, CYR61, CYTH2, CYYR1, DACH2, DACT3, DBC1, DBN1, DCHS1,
DCX, DDC, DDEF2, DENND2D, DGCR5, DHCR24, DKK3, DKK4, DLG2, DLK1,
DLL3, DNAJC12, DNAJC15, DNM3, DOCK10, DOCK6, DOCK8, DPEP1, DPP4,
DRAM1, DSC2, DSCR6, DUSP6, DYNC1I1, EDG4, EFEMP2, EFHD1, EFNB2,
EGR1, EHBP1L1, EIF2B3, ELA3A, ELAVL3, ELF4, ELFN2, ELMO1, ELOVL2,
EMID1, ENC1, ENPP2, ENPP5, ENTPD3, EPAS1, EPDR1, ERBB3, ERO1LB,
EXOC7, F10, FABP7, FAM102A, FAM105A, FAM107A, FAM110B, FAM125B,
FAM159B, FAM176B, FAM181B, FAM18A, FAM46B, FAM50B, FAT3, FEV,
FGB, FGFR3, FGFR4, FHOD3, FICD, FKBP10, FKBP1B, FKBP5, FLJ14712,
FLJ23834, FLJ25404, FLJ31568, FLJ41603, FLJ43752, FLNC, FNDC3B, FOXA1,
FRMD4A, FSCN1, FSTL5, FXYD2, FXYD5, FXYD6, FZD2, G6PC2, GAL3ST1,
GALK1, GALNT14, GALNTL1, GAS2, GAS2L3, GATA4, GBP2, GC, GCGR,
GDPD5, GEM, GFRA3, GGH, GHDC, GHR, GHRL, GJA1, GLDC, GLIS3,
GLT25D2, GLT8D2, GLTPD2, GLYCTK, GNG3, GOLSYN, GPBAR1, GPC2,
GPC3, GPC4, GPER, GPM6A, GPR112, GPR137C, GPR37, GPR64, GPX7, GSN,
GSTM1, GSTM2, GUCA2B, GUCY1A3, H3F3B, HABP2, HABP4, HADH,
HAPLN4, HBQ1, HECA, HECW2, HES6, HHATL, HHEX, HIC2, HIST1H2BK,
HIST1H4K, HIST2H2AA3, HIST2H2AA4, HIST2H2AC, HLA-A29.1, HLA-B,
HLA-F, HLXB9, HMGCS1, HMGCS2, HMP19, HN1, HNF4G, HOPX, HS3ST4,
HSF4, HSPA1A, HSPA2, HSPA4L, HSPA6, IAH1, IAPP, ICAM2, IER3, IER5,
IFI35, IFIT1, IGDCC3, IGF2BP2, IGF2BP3, IGFBP2, IGFBP4, IGFBP5, IGSF9,
IL11RA, IL13RA1, IL17RB, IL20RA, IMPA2, IQGAP2, IRX2, ISG20, ISX, ITIH4,
ITM2A, ITPR3, KANK1, KCNG3, KCNK1, KCNK3, KCNT2, KCTD12,
KIAA0247, KIAA0319, KIAA0363, KIAA0367, KIAA0408, KIAA0514,
KIAA0672, KIAA1644, KIAA1683, KIAA1688, KIF12, KLF9, KLHDC8A,
KLHDC8B, KLKB1, KRT19, LAMB1, LAMB2, LARGE, LASS1, LDHA,
LDLRAP1, LIMCH1, LIN28B, LINGO1, LINGO4, LLGL1, LMO4,
LOC100129913, LOC100130154, LOC100132117, LOC100133999,
LOC100134134, LOC100134265, LOC100144604, LOC133993, LOC154761,
LOC388494, LOC389332, LOC389493, LOC390705, LOC399744, LOC399959,
LOC401720, LOC441066, LOC643740, LOC643911, LOC644390, LOC644670,
LOC645233, LOC645566, LOC647251, LOC647307, LOC647886, LOC649841,
LOC650200, LOC728431, LOC729137, LOC729314, LOC729970, LOXL3, LPAR1,
LPAR2, LPHN2, LPPR5, LRCH2, LRRC56, LRRN2, LY6H, LY96, LZTS1,
M6PRBP1, MAFA, MAMDC2, MAMLD1, MAN1C1, MAP3K6, MAPK12,
MAPK15, 3-Mar, MARCKSL1, MATN2, ME3, MEG3, MEP1A, MET, METTL7A,
MFAP4, MFGE8, MFNG, MGAT3, MGAT4A, MGAT4C, MGC16291, MGC16384,
MGC18216, MGC39900, MIAT, MMP7, MNX1, MR1, MRAP2, MSL3L1, MT1X,
MTE, MUC13, MVP, MX1, MYL5, MYLIP, MYO10, MYO16, MYO1D, MYOM1,
NAP1L5, NBPF8, NDRG2, NDUFAE2, NETO2, NEURL1B, NFIA, NFIX,
NIPSNAP1, NKD2, NKX6-1, NLF2, NLGN4X, NMNAT2, NOS1AP, NPEPL1,
NPHS1, NPTX2, NPY, NQO2, NR0B1, NR2F1, NR3C2, NSMCE1, NYNRIN,
OLFM1, ONECUT2, OSBP2, OVGP1, OXCT2, OXGR1, PABPC1L, PAFAH1B3,
PALM2, PARM1, PAX4, PAX9, PBX2, PCDHB2, PCSK1, PCSK2, PDE8B, PDK4,
PDLIM7, PDX1, PDZD3, PDZD8, PDZK1, PDZK1P1, PEG3, PENK, PFKFB2,
PGM5, PHGDH, PHLDA3, PHYHIPL, PIR, PLA2G1B, PLA2G4C, PLCH2, PLCL2,
PLCXD3, PLEKHA9, PLEKHB1, PLS3, PLTP, PLXDC2, PLXNA1, PLXNB1,
PNCK, PNLIPRP2, PNMA3, PNMT, PON3, PPAP2B, PPM1E, PPP2R2C,
PPP2R3B, PRAGMIN, PRAMEF19, PRICKLE1, PRKCH, PRKD1, PRNP, PROC,
PRODH2, PROM1, PROX1, PRPH, PRR15L, PRSS1, PRSS23, PRSS8, PRUNE2,
PSMB8, PTGR1, PTGS2, PTPN3, PTPRD, PTPRT, PURA, PXDN, QPRT,
RAB11FIP5, RAB31, RAB34, RAPH1, RASD1, RASGRP1, RBBP8, RBP4,
RBPMS2, RCOR2, RDH12, RENBP, RGL1, RGMA, RGS9, RHBDF1, RHOC,
RHOU, RICH2, RIN2, RNASEL, RND3, RNF144, RNF144A, RNF165, ROR2,
RORC, RPRM, RPS19BP1, RYR1, S100A10, S100A16, S100A4, SCARA3, SCD5,
SCGB2A1, SCIN, SDF2L1, SELENBP1, SELM, SEMA3A, SEMA6C, 9-Sep,
SERP1, SERPINA1, SERPINA10, SERPINF1, SERPINF2, SERTAD4, SFRP1,
SGMS2, SH3GL2, SHC3, SHISA2, SIRPA, SIX4, SLC12A5, SLC16A9, SLC17A6,
SLC17A8, SLC17A9, SLC1A4, SLC25A34, SLC27A3, SLC30A8, SLC35D3,
SLC38A1, SLC39A5, SLC44A3, SLC45A3, SLCO2A1, SMYD3, SNCAIP, SNHG9,
SNORA12, SNORD114-3, SNX26, SOCS2, SORBS1, SORL1, SOX11, SPATA18,
SPATS2L, SPIRE1, SPON2, SRRM4, SRXN1, SSPO, ST6GAL1, ST6GALNAC5,
STC2, STEAP2, STMN1, STMN4, STOM, STRA6, SURF4, SVIL, SYK, SYNGR4,
SYT13, TAP1, TCF3, TGEBR2, TGEBR3, TGIF2, TGOLN2, TH, THBS3, TICAM2,
TIMP2, TLE6, TM7SF2, TMEM109, TMEM163, TMEM190, TMEM27, TMEM56,
TMEM61, TMEM86B, TMEM98, TMSB15A, TNFRSF21, TNFRSF25, TOX3,
TP53I3, TP53INP1, TPST2, TRIM24, TRIM4, TRIM46, TRIOBP, TRIP6, TRO,
TSHZ3, TSPAN14, TSPAN6, TSPYL5, TST, TTLL10, UCP2, UNC5A, UNC5CL,
VANGL2, VASN, VAV3, VCAN, VGF, VIL1, VIM, VSTM2, VSTM2L, VTN,
VWCE, WDR86, WEE1, WNT4, WSCD2, XBP1, XKR4, ZBTB20, ZDHHC8P,
ZFP3, ZFPM1, ZFR2, ZNF217, ZNF331, ZNF385D, ZNF439, ZNF532, ZNF618

It should be appreciated that any gene listed in Table 2C can be used as a marker for distinguishing between mature β-cells and immature β-cells by measuring the level of expression of the gene in a cell, culture, cell line, tissue, or population of cells (e.g., suspected of being β-cells), wherein if the level of expression of the gene in the cell, culture, cell line, tissue, or population of cells is similar to the level of expression in mature β-cells then the cell, culture, cell line, tissue, or population of cells comprises mature β-cells, and wherein if the level of expression of the gene in the cell, culture, cell line, tissue, or population of cells is similar to the level of expression in in vitro-differentiated β-cells, then the cell, culture, cell line, tissue, or population of cells comprises immature β-cells.

Those skilled in the art will also appreciate that any two or more of the genes listed in Table 2C can be used in combinations of up to N genes (where N is a positive integer greater than or equal to 2) as markers for distinguishing mature β-cells from immature β-cells by measuring the levels of expression of the combination of genes in a cell, culture, cell line, tissue, or population of cells (e.g., suspected of being β-cells), wherein if the levels of expression of the combination of genes in the cell, culture, cell line, tissue, or population of cells are similar to the levels of expression of the same combination of genes in mature β-cells then the cell, culture, cell line, tissue, or population of cells comprises mature β-cells, and wherein if the levels of expression of the combination of genes in the cell, culture, cell line, tissue, or population of cells are similar to the levels of expression of the same combination of genes in in vitro-differentiated insulin-positive β-like cells then the cell, culture, cell line, tissue, or population of cells comprises immature β-cells

In some aspects, a marker of β-cell functional immaturity includes one, any combination or sub-combination, or all genes which are have a higher expression level in fetal β-cells or in vitro-differentiated insulin-positive β-like cells compared to mature β-cells.

In some embodiments of this and other aspects of the invention, genes which have a higher expression level in fetal β-cells compared to mature β-cells are listed in Table 3 (Table 3 includes both Tables 3A and 3B)

TABLE 3A
Genes having higher expression levels in fetal
β-cells compared to mature β-cells

SOX4, LZTR1, CSRNP3, HHEX, PROX1, RFX1, SOX8, ZNF423,
LHX4, SOX11, ISX, NFIB, PAX4, LMO4, NOTCH1, ASCL2, TCF3,
RCOR2, EGR1, FOS, MYCN, EVI1, LZTS1, GHRL, PYY, HNF1A,
INSM1, IRX2, ISL1, MYT1, NEUROG3, ACSS1, GCK, HCN3,
KCNJ4, COL1A1, FFAR1

TABLE 3B
Genes having higher expression levels in fetal β-cells compared to mature β-cells

A1CF, AACS, AADAT, ABAT, ABCC10, ABCC5, ABHD9, ABP1,
ACACA, ACAP3, ACBD7, ACCN1, ACCS, ACP2, ACVR1C, ADPRHL2,
ADRA2A, ADSL, AGAP6, AGAP8, AGBL4, AGPAT4, AGRN, AHCY, AKT1,
ALDH7A1, ALMS1, ALPP, AMACR, AMT, ANKDD1A, ANKRD30B,
ANKRD36B, ANKRD50, ANKS1B, ANO6, ANTXR2, ANXA4, AOF2, AP1M2,
AP2A2, APEX1, ARC, ARF5, ARGLU1, ARHGEF19, ARHGEF4, ARID3A,
ARID3B, ARIH2, ARL3, ARRDC1, ARVCF, ASAP3, ASB9, ASCL2, ASGR1,
ASS1, ATG16L2, ATG2A, ATP2B4, ATP6V1B1, ATXN2L, ATXN7L2, AUTS2,
AXIN2, B4GALNT4, B4GALT5, BACE1, BAMBI, BASP1, BAT2L, BAZ2B,
BCL11A, BCS1L, BEND5, BIK, BLZF1, BMS1P5, BOP1, BPHL, BRD3, BRF1,
BRSK2, BTBD17, BZRAP1, C10orf114, C10orf140, C10orf33, C10orf35, C11orf41,
C11orf61, C12orf27, C12orf51, C12orf76, C14orf102, C14orf139, C14orf159,
C14orf79, C14orf85, C15orf63, C16orf59, C16orf62, C16orf7, C16orf93, C17orf76,
C18orf45, C1orf53, C1orf59, C1orf93, C1QTNF4, C1QTNF6, C20orf12, C21orf29,
C21orf45, C21orf54, C21orf56, C22orf29, C2orf54, C2orf68, C3orf21, C3orf50,
C3orf59, C5orf13, C6orf124, C6orf134, C6orf192, C6orf26, C6orf59, C7orf38,
C8ORFK32, C9orf117, C9orf167, C9orf45, C9orf66, C9orf7, CABIN1, CABP7,
CACNG4, CAD, CADM4, CALB2, CALCOCO1, CALHM2, CALY, CAMKV,
CAPSL, CARD10, CARD8, CARD9, CASP2, CATSPER2, CBLB, CBLN2, CBX2,
CCDC125, CCDC14, CCDC22, CCDC8, CCDC88B, CCDC88C, CCNF, CCNL1,
CDAN1, CDC25B, CDH12, CDH3, CDK10, CDK5RAP3, CDK9, CDO1, CECR6,
CELSR2, CELSR3, CENPV, CEP135, CEP164, CEP27, CEP78, CES2, CES8,
CFLAR, CGNL1, CHD4, CHD7, CHD8, CHFR, CHGA, CHST12, CIDEB, CKAP2,
CKB, CLASP1, CLIC6, CLIP3, CLPS, CLRN3, CNN3, CNNM3, CNOT6,
CNTNAP2, CNTNAP4, COL16A1, COL18A1, COL1A2, COL3A1, COL4A5,
COL5A1, COL5A2, COL6A1, COL8A2, COPG2IT1, COPS7B, CORO1B, COX19,
CPT1C, CRABP1, CRMP1, CROCC, CRYBA2, CSF2RA, CSNK1E, CSNK1G2,
CSRNP3, CTNNBIP1, CTPS, CTSK, CTXN1, CX3CL1, CXXC4, CYB5A,
CYP2C8, CYP2J2, CYP2W1, CYTH2, DACT3, DAGLB, DARC, DBC1, DBN1,
DCAF7, DCDC5, DCHS1, DCLRE1C, DCX, DDAH2, DDB1, DDC, DDX27,
DECR2, DENND2A, DENR, DKEZp761P0423, DKK3, DLG5, DLK1, DMAP1,
DMWD, DNAH1, DNALI1, DNHD2, DOCK6, DOCK8, DOK4, DOLPP1, DPEP1,
DPYSL3, DRD1IP, DSCR6, DTX1, DUSP1, DUSP18, DUSP9, DUT, E2F5, EBF1,
EBF4, ECEL1, EDG4, EFCAB4A, EFEMP2, EFNA4, EFNB1, EFNB2, EFS, EGR1,
EHD1, EIF2AK4, EIF2C1, ELAVL3, ELFN2, ELMO2, EMID1, EML1, ENHO,
EP400, EPC1, EPHA4, EPHB2, EPOR, ERGIC1, ERMAP, ETV3, EVI1, EVL,
EVPL, EXD3, EXOC7, EXOSC10, EYA2, F10, FABP5, FABP5L2, FAM110B,
FAM116B, FAM117B, FAM163A, FAM175A, FAM176B, FAM195B, FAM32A,
FAM39E, FAM40A, FAM73A, FAM83E, FAM92B, FANCE, FARP1, FBXL10,
FBXW9, FCGRT, FER1L5, FFAR1, FFAR3, FGD1, FGD3, FGFR3, FGFR4, FIT1,
FKBP1P1, FKRP, FLJ10246, FLJ25363, FLJ30092, FLJ31568, FLJ35258,
FLJ36131, FLJ39827, FLJ41649, FLJ42627, FLJ44124, FLJ46309, FLJ46906,
FLJ90757, FLRT2, FMNL2, FNBP1L, FOS, FOXJ1, FOXO4, FOXP4, FRAS1,
FRAT2, FSTL3, FTCD, FUT4, FXYD2, FXYD3, FXYD6, FYN, FZD3, FZD9,
GABBR1, GAL3ST1, GALM, GALNS, GALNT3, GALNTL1, GAS2L3, GAS8,
GATM, GATS, GGH, GGT7, GJB1, GJC2, GLB1L2, GLIPR2, GLYCTK, GNL3L,
GNMT, GOLGA8A, GPBAR1, GPC1, GPC2, GPER, GPR114, GPR125, GPR137C,
GPR42, GPR98, GPT, GPT2, GPX7, GRK4, GRK5, GRM4, GSTM1, GSTM2,
GTF21P1, GUSBL1, GUSBL2, H19, H2AFY2, HCFC1, HCN3, HDAC6, HEXDC,
HHATL, HHEX, HIATL2, HIC2, HIF3A, HIST1H4J, HIST1H4K, HN1, HNF1A,
HNRNPA3, HNRNPH1, HNRNPR, HNRNPU, HNRPA1L-2, HNRPA1P4,
HNRPH1, HPCA, HSBP1, HSD17B7, HSPC047, HSPC268, HTT, HYMAI, IDH2,
IFFO2, IFT140, IGDCC3, IGDCC4, IGF2BP2, IGF2BP3, IGFALS, IGFBP5, IGSF9,
IGSF9B, IL11RA, IMPA2, INPP5E, INSM1, INTS3, IP6K2, ISL1, ISX, ITM2A,
ITPA, ITPRIP, JARID2, JMJD2B, KATNB1, KCND1, KCNH3, KCNH6, KCNH8,
KCNJ4, KCNQ1OT1, KCNQ2, KCNT2, KDM5B, KIAA0114, KIAA0182,
KIAA0427, KIAA0492, KIAA0773, KIAA0889, KIAA0892, KIAA0895L,
KIAA0922, KIAA1217, KIAA1543, KIAA1545, KIAA1614, KIAA1688,
KIAA1843, KIF12, KIF3C, KIFC2, KLHDC8B, KLHL28, KNDC1, KRT19, KRT80,
KTELC1, LAMA1, LAMB1, LAMC1, LANCL2, LARGE, LARP4B, LARS2,
LDLRAD3, LEPREL1, LFNG, LHX4, LIME1, LIMS2, LIN52, LINGO1, LINGO2,
LLGL1, LLGL2, LMNB2, LMO3, LMO4, LOC100127975, LOC100128062,
LOC100128098, LOC100128126, LOC100128288, LOC100128374,
LOC100128510, LOC100128729, LOC100128974, LOC100129022,
LOC100129211, LOC100129387, LOC100129441, LOC100129502,
LOC100129580, LOC100129608, LOC100129905, LOC100129975,
LOC100130053, LOC100130276, LOC100130353, LOC100130516,
LOC100130835, LOC100131017, LOC100131541, LOC100131718,
LOC100131786, LOC100132323, LOC100132347, LOC100132564,
LOC100132585, LOC100132727, LOC100132740, LOC100132810,
LOC100132901, LOC100133144, LOC100133516, LOC100133840,
LOC100133999, LOC100134053, LOC100134134, LOC100134144,
LOC100134159, LOC100134241, LOC100134265, LOC100134361,
LOC100134584, LOC100134821, LOC100170939, LOC123688, LOC145837,
LOC149134, LOC153561, LOC158301, LOC201725, LOC202781, LOC284167,
LOC284297, LOC284422, LOC284757, LOC338758, LOC341230, LOC387934,
LOC389168, LOC389765, LOC401098, LOC402112, LOC440157, LOC440345,
LOC440704, LOC441268, LOC442041, LOC442582, LOC552889, LOC642031,
LOC642741, LOC642852, LOC642909, LOC642956, LOC644935, LOC644979,
LOC645233, LOC645452, LOC645566, LOC645648, LOC646044, LOC646897,
LOC646996, LOC647251, LOC647691, LOC648024, LOC648057, LOC648059,
LOC648526, LOC648852, LOC652002, LOC652377, LOC652900, LOC653157,
LOC653210, LOC653352, LOC653629, LOC653829, LOC654103, LOC727877,
LOC728105, LOC728153, LOC728411, LOC728448, LOC728452, LOC728457,
LOC728485, LOC728499, LOC728519, LOC728565, LOC728608, LOC728640,
LOC728643, LOC728661, LOC728779, LOC729120, LOC729351, LOC729559,
LOC729978, LOC730173, LOC730993, LOC730995, LOC732360, LOC791120,
LOC90113, LOC91461, LOC92497, LOXL1, LOXL3, LPAR2, LPHN1, LPHN2,
LPPR5, LRAP, LRCH2, LRCH3, LRP1, LRPPRC, LRRC3, LRRC37B2, LRRC56,
LRRC8A, LRRN2, LUC7L, LZTR1, LZTS1, MAFB, MAGT1, MAMDC4, MAOA,
MAP3K12, MAP3K4, MAP4K2, MAPKAPK3, MARCKS, MARCKSL1, MARK3,
MARVELD3, MAST1, MATN1, MBD4, MBD6, MCF2L, MED12, MED24,
MED25, MEG3, MEG8, MEGF8, MEIS1, MEIS2, MEMO1, MEX3A, MEX3D,
MFAP4, MFGE8, MFNG, MGAT3, MGC16121, MGC16384, MGC16703,
MGC3032, MGC39900, MGC50722, MGC52000, MGC61598, MICAL1, MIR2116,
MIR886, MKNK1, MLL, MLXIPL, MMP15, MMP23A, MMP23B, MOV10, MPL,
MSI1, MSL3L1, MST1, MSTP9, MTF2, MTG1, MTHFD1L, MTHFR, MTMR4,
MUM1, MXD4, MYB, MYBPC2, MYCN, MYH10, MYL7, MYO10, MYO5C,
MYO7A, MYOM3, MYT1, MYT1L, N4BP1, N4BP2, N6AMT1, NARF, NASP,
NAT11, NBPF8, NDN, NDST1, NDUFS2, NECAB3, NEK8, NETO2, NEURL,
NEURL1B, NEURL4, NEUROD1, NFIB, NGFRAP1, NINL, NIPSNAP1, NISCH,
NKTR, NLRP2, NNAT, NOTCH1, NPC1L1, NPEPL1, NPHS1, NPIP, NT5DC2,
NTNG2, NUBPL, NUMBL, NUP62, NXN, NYNRIN, ODZ3, OSBP2, OTUB1,
OVGP1, P2RY11, PABPC1L, PABPN1, PACSIN3, PAFAH1B3, PALM, PANK1,
PAPSS1, PARD6G, PAX4, PBX2, PBX4, PCDH24, PCDHB19P, PCGF2, PCNT,
PCYOX1L, PDCD4, PDE5A, PDGFD, PDGFRB, PDZD3, PEAR1, PEG3, PGAM2,
PGD, PH-4, PHF16, PHLDB1, PIK3C2B, PIK3IP1, PIP5K2B, PIWIL4, PKDCC,
PKN2, PLA2G4F, PLAGL1, PLAGL2, PLCG1, PLCH2, PLCXD1, PLDN,
PLEKHA5, PLEKHA9, PLEKHB1, PLEKHF1, PLEKHG3, PLEKHH1, PLIN5,
PLS3, PLSCR3, PLXNA1, PLXNA3, PLXNB1, PNMA3, PNMT, PODN, POLRMT,
PPAPDC3, PPFIA4, PPFIBP1, PPOX, PPP1R13B, PPP1R14C, PPP1R16A,
PPP1R9A, PPP4C, PRAGMIN, PRICKLE1, PRKCABP, PRKCDBP, PRKCG,
PRKCQ, PRKCZ, PRKD1, PRO0628, PRO1853, PRODH, PRODH2, PROM1,
ProSAPiP1, PROX1, PRR15L, PRR3, PRR5, PTBP2, PTCH1, PTK7, PTPRE,
PTPRF, PTRH1, PXDN, PYCR1, QDPR, QPRT, QRFPR, R3HDM1, RAB11FIP3,
RAB11FIP4, RAB25, RAB26, RAB36, RABL2B, RAGE, RANBP1, RAPGEF4,
RAPGEF5, RASAL1, RASIP1, RASL11B, RASSF4, RAX2, RAXL1, RBM15B,
RBM5, RBM6, RBM9, RBP1, RCC2, RCE1, RCN3, RCOR2, REC8, RELN, RFX1,
RFX7, RGAG4, RGL3, RGMA, RHBDL2, RHOB, RIMBP2, RNF112, RNF165,
RNF183, RNF31, RNF43, RNF44, RNU1A3, RNY1, RNY3, ROBO2, ROGDI,
ROR2, RXRA, SAMD14, SAPS3, SARM1, SARS2, SBK1, SCARB1, SCIN,
SCN9A, SCTR, SDC1, SDK2, SDR9C7, SEMA3A, SEMA4C, SEMA6A, SEMA6C,
2-Sep, 9-Sep, SERINC2, SERPINF2, SETDB1, SFRS14, SFRS8, SGPL1, SH3BP5L,
SHROOM3, SLC12A9, SLC16A9, SLC22A16, SLC25A14, SLC25A27, SLC26A4,
SLC27A3, SLC29A1, SLC2A8, SLC35E1, SLC38A1, SLC39A10, SLC44A3,
SLC4A8, SLC5A8, SLIT2, SMA4, SMARCC1, SMARCD2, SMCR5, SMO,
SNAPC4, SNORA28, SNORA6, SNORA67, SNORA80, SNORD36C, SNORD56,
SNRNP70, SNX26, SOX11, SOX12, SOX4, SOX8, SPINK1, SPIRE2, SPNS2,
SPOCK2, SPON1, SRPX, SSBP3, SSTR2, ST6GAL1, ST6GALNAC5, STAG3L2,
STAG3L3, STK36, STMN1, STMN2, STRA6, SULF1, SV2B, SVIL, SYNJ2BP,
TACC3, TAF15, TARBP1, TBC1D14, TBC1D24, TBC1D3I, TBC1D4, TBCB,
TCEAL7, TCF1, TCF20, TCF3, TCF4, TCN2, TDG, TDRD1, TECPR1, TEKT2,
TESC, TET1, TGFB3, TH, THBS3, THOC1, TIA1, TIAF1, TIMM22, TJP2, TLE2,
TLE4, TLK2, TM6SF1, TMEFF2, TMEM106A, TMEM132A, TMEM132B,
TMEM134, TMEM137, TMEM169, TMEM190, TMEM196, TMEM206, TMEM5,
TMEM53, TMEM91, TMEM98, TMPRSS6, TMSB10, TMSB15A, TNFAIP8L1,
TNFSF15, TOP1P2, TP53INP1, TRA2A, TRIM46, TRO, TRPM2, TRPM5, TRPM8,
TSC2, TSC22D3, TSNAXIP1, TSPAN14, TSPAN17, TSPAN32, TSPAN33,
TSPAN5, TSPAN6, TTC38, TTC5, TTLL3, TUBB1, TUBB2B, TUBB3, TUBB4,
TYRO3, UBE2O, UBE2Q1, UCP2, UGT2B7, UNC13B, UNC5A, UPF3A, USH1C,
USP49, VANGL2, VASH1, VASH2, VIL1, VPS37D, VPS8, VWCE, WDR48,
WDR6, WDR74, WDR86, WIPI2, WNK2, WNT5B, WSB1, YPEL1, YRDC,
ZBED4, ZBTB46, ZBTB47, ZBTB48, ZCCHC11, ZDHHC14, ZDHHC8P, ZFP90,
ZFPM1, ZFYVE21, ZMIZ2, ZNF133, ZNF135, ZNF193, ZNF217, ZNF248,
ZNF281, ZNF296, ZNF300, ZNF362, ZNF423, ZNF431, ZNF503, ZNF512B,
ZNF518A, ZNF532, ZNF551, ZNF608, ZNF609, ZNF618, ZNF649, ZNF652,
ZNF667, ZNF69, ZNF692, ZNF696, ZNF711, ZNF773, ZNF785, ZNF786, ZNF827,
ZNF85, ZSWIM7

It is to be understood that the levels of expression of one or more of the genes listed in Table 3 are depicted in FIG. 6C as a fold change in expression of mature β-cells compared to fetal β-cells.

It should also be appreciated that any gene listed in Table 3 can be used as a marker for detecting immature β-cells or fetal β-cells by measuring the level of expression of the gene in a cell, culture, cell line, tissue, or population of cells (e.g., suspected of being β-cells), wherein if the level of expression of the gene in the cell, culture, cell line, tissue, or population of cells is elevated (for example, as depicted in FIG. 6C), the cell, culture, cell line, tissue, or population of cells comprises immature β-cells or fetal β-cells.

Those skilled in the art will also appreciate that any two or more of the genes listed in Table 3 can be used in combinations of up to N genes (where N is a positive integer greater than or equal to 2) as markers for detecting immature β-cells or fetal β-cells by measuring the levels of expression of the combination of genes in a cell, culture, cell line, tissue, or population of cells (e.g., suspected of being β-cells), wherein if the levels of expression of the combination of genes in the cell, culture, cell line, tissue, or population of cells is elevated (for example, as depicted in FIG. 6C), the cell, culture, cell line, tissue, or population of cells comprises mature β-cells.

In some embodiments of this and other aspects of the invention, genes having higher expression levels in in vitro-differentiated insulin-positive β-like cells compared to mature β-cells are listed in Table 4 (Table 4 includes both Table 4A and Table 4B).

TABLE 4A
Genes having higher expression levels in vitro-differentiated insulin-
positive β-like cells compared to mature β-cells

ZNF217, ISX, PBX2, HNF4G, ELF4, CEBPA, RCOR2, MSL3L1,
EGR1, TCF3, LZTS1, TGIF2, HES6, AEBP1, LMO4, SOX11,
IRX2, FEV, NR2F1, CHGA, GAST, GCG, GHRL, NTS, PYY,
ARX, FOXA1, FOXA3, GATA4, IRX2, ISL1, NOTCH1, ONECUT2,
PAX4, PROX1, SOX4, ACOX2, RIMS3, SLC2A1, SLC2A3,
CACNA1E, HCN3, KCNB1, KCNJ2, DLL3, DPP4, HHEX, FOXA1

TABLE 4B
Genes having higher expression levels in vitro-differentiated insulin-
positive β-like cells compared to mature β-cells

7SK, A1CF, A2M-AS1, A2ML1-AS2, A2MP1, A3GALT2P, A4GALT, AAA1,
AACS, AACSP1, AAMP, AASS, AB015752.4, ABC7-42389800N19.1, ABCA11P,
ABCA12, ABCA4, ABCA7, ABCB10, ABCC1, ABCC10, ABCC4, ABCC6,
ABCC6P1, ABCC6P2, ABCD1, ABCD4, ABCG4, ABCG5, ABHD11, ABHD11-
AS2, ABHD12B, ABHD14A, ABHD14A-ACY1, ABHD15, ABHD9, ABL1,
ABLIM2, ABLIM3, ABO, ABT1P1, ABTB1, ABTB2, AC000032.2, AC000078.5,
AC000120.7, AC002066.1, AC002116.7, AC002310.17, AC002310.7, AC002365.1,
AC002398.5, AC002472.11, AC002472.13, AC002477.1, AC002480.3, AC002480.5,
AC002539.1, AC003090.1, AC003102.3, AC003664.1, AC003988.1, AC004053.1,
AC004057.1, AC004152.5, AC004158.2, AC004158.3, AC004160.4, AC004166.7,
AC004221.2, AC004231.2, AC004381.6, AC004386.4, AC004448.5, AC004449.6,
AC004475.1, AC004538.3, AC004540.5, AC004543.2, AC004593.2, AC004623.2,
AC004799.3, AC004837.4, AC004840.9, AC004862.6, AC004893.10, AC004893.11,
AC004895.4, AC004906.3, AC004947.2, AC004957.5, AC004967.11, AC004967.7,
AC004980.10, AC004980.7, AC004980.9, AC004985.12, AC004987.8, AC004987.9,
AC005000.1, AC005003.1, AC005009.1, AC005009.2, AC005013.5, AC005019.3,
AC005029.1, AC005034.3, AC005042.4, AC005071.1, AC005086.4, AC005154.5,
AC005154.8, AC005204.2, AC005215.1, AC005256.1, AC005262.3, AC005300.5,
AC005306.3, AC005324.6, AC005324.8-001, AC005326.2, AC005329.1,
AC005329.7, AC005330.2, AC005391.3, AC005488.11, AC005513.1, AC005519.4,
AC005534.6, AC005540.3, AC005562.2, AC005592.1, AC005592.3, AC005609.1,
AC005625.1, AC005682.5, AC005682.6, AC005702.3, AC005702.4, AC005754.1,
AC005757.6, AC005775.2, AC005841.1, AC005943.4, AC005943.5, AC005944.2,
AC005971.3, AC006000.5, AC006003.3, AC006014.8, AC006023.8, AC006028.10,
AC006042.7, AC006111.1, AC006116.1, AC006116.20, AC006132.1, AC006156.1,
AC006160.5, AC006370.2, AC006372.1, AC006372.4, AC006372.5, AC006445.7,
AC006465.4, AC006509.6, AC006547.14, AC006547.8, AC006946.15,
AC007000.10, AC007000.12, AC007256.5, AC007285.7, AC007308.6, AC007383.3,
AC007387.2, AC007391.2, AC007395.4, AC007401.1, AC007403.3, AC007464.1,
AC007551.2, AC007557.1, AC007557.3, AC007563.3, AC007563.4, AC007563.5,
AC007875.2, AC007881.4, AC007969.4, AC008063.2, AC008073.7, AC008073.9,
AC008088.4, AC008103.5, AC008278.3, AC008440.10, AC008674.1, AC008676.1,
AC008687.1, AC008697.1, AC008746.3, AC008993.2, AC008993.5, AC009065.1,
AC009110.1, AC009120.5, AC009133.12, AC009133.15, AC009133.20,
AC009227.2, AC009264.1, AC009336.24, AC009480.3, AC009492.1, AC009505.2,
AC009542.2, AC009945.4, AC009955.8, AC009963.5, AC009965.2, AC010095.7,
AC010127.3, AC010132.11, AC010148.1, AC010325.1, AC010336.1, AC010492.2,
AC010492.4, AC010507.5, AC010525.6, AC010536.2, AC010615.1, AC010620.1,
AC010649.1, AC010677.5, AC010729.1, AC010731.2, AC010746.4, AC010761.10,
AC010761.13, AC010761.9, AC010894.3, AC010894.4, AC010904.1, AC010976.2,
AC010982.2, AC010997.1, AC011290.4, AC011306.2, AC011343.1, AC011385.2,
AC011443.1, AC011450.1, AC011475.1, AC011484.1, AC011491.1, AC011497.1,
AC011551.1, AC011558.5, AC011718.1, AC011742.3, AC011899.9, AC012123.1,
AC012358.4, AC012358.7, AC012363.7, AC012368.1, AC012445.1, AC012485.2,
AC012499.1, AC012506.4, AC012668.2, AC013283.1, AC013400.2, AC013402.2,
AC013439.4, AC013460.1, AC013472.3, AC013472.4, AC015691.13, AC015815.2,
AC015815.6, AC015818.11, AC015818.6, AC015842.1, AC015849.2, AC015884.1,
AC016405.1, AC016670.1, AC016683.5, AC016700.5, AC016708.2, AC016712.2,
AC016716.2, AC016722.1, AC016722.2, AC016723.4, AC016725.4, AC016757.3,
AC016773.1, AC016894.1, AC017116.8, AC018359.1, AC018638.1, AC018648.5,
AC018693.6, AC018705.5, AC018737.6, AC018738.2, AC018799.1, AC018804.1,
AC018804.6, AC018865.5, AC018865.8, AC018865.9, AC018892.9, AC019068.2,
AC019070.1, AC019100.3, AC019118.2, AC019185.4, AC019205.1, AC020550.7,
AC020743.3, AC020907.3, AC020915.1, AC021860.1, AC022007.4, AC022007.5,
AC022182.1, AC022182.2, AC022210.2, AC022400.1, AC022498.1, AC022532.1,
AC022819.2, AC023480.1, AC023490.1, AC023490.2, AC023590.1, AC024162.2,
AC024475.1, AC024580.1, AC024896.1, AC024937.2, AC024937.4, AC024937.6,
AC024940.1, AC025165.1, AC025165.8, AC025171.1, AC026956.1, AC027119.1,
AC027307.1, AC027601.1, AC027612.1, AC037445.1, AC048382.4, AC051649.6,
AC055764.1, AC055811.5, AC060834.2, AC060834.3, AC061961.2, AC061975.1,
AC061992.2, AC062016.1, AC063976.7, AC064836.3, AC064852.5, AC066593.1,
AC068014.1, AC068020.1, AC068129.2, AC068134.10, AC068137.1, AC068499.6,
AC068522.4, AC068535.3, AC068580.6, AC068587.2, AC068641.1, AC068718.1,
AC068831.10, AC068831.3, AC068831.6, AC069277.2, AC069278.4, AC069294.1,
AC069513.4, AC072052.7, AC073052.1, AC073130.3, AC073133.2, AC073236.3,
AC073283.4, AC073342.12, AC073415.2, AC073850.6, AC073869.2, AC073869.20,
AC073957.15, AC074019.2, AC074212.5, AC074212.6, AC074289.1, AC074363.1,
AC074389.6, AC078842.3, AC078883.3, AC078941.1, AC078942.1, AC079233.1,
AC079354.5, AC079586.1, AC079610.2, AC079753.1, AC079776.1, AC079776.3,
AC079780.3, AC079781.5, AC079781.8, AC079790.2, AC079807.2, AC080091.1,
AC080125.1, AC083855.4, AC083862.6, AC083949.1, AC084018.1, AC084117.3,
AC084125.4, AC087289.1, AC087650.1, AC090103.1, AC090519.5, AC090559.1,
AC090587.5, AC090602.2, AC090627.1, AC091038.1, AC091153.4, AC091633.3,
AC091878.1, AC092106.1, AC092159.2, AC092171.1, AC092171.4, AC092291.1,
AC092329.1, AC092338.5, AC092535.1, AC092535.3, AC092619.1, AC092620.3,
AC092638.2, AC092641.2, AC092675.3, AC092798.1, AC092839.1, AC092839.4,
AC092902.1, AC093106.5, AC093107.7, AC093159.1, AC093375.1, AC093415.3,
AC093415.5, AC093609.1, AC093616.4, AC093627.10, AC093627.6, AC093668.3,
AC093690.1, AC093724.2, AC093734.11, AC093734.13, AC093818.1, AC093838.4,
AC093838.7, AC096574.5, AC096669.2, AC097374.2, AC097468.4, AC097499.2,
AC097635.4, AC097721.1, AC098474.1, AC098784.2, AC098826.5, AC099489.1,
AC099668.5, AC100830.3, AC100830.5, AC100852.2, AC103828.1, AC103965.1,
AC104113.3, AC104131.1, AC104532.2, AC104532.4, AC104655.3, AC104809.3,
AC105052.1, AC105339.1, AC106782.18, AC106782.20, AC106801.1, AC106869.2,
AC107057.2, AC108025.2, AC108039.2, AC108142.1, AC108479.2, AC109333.10,
AC109642.1, AC109828.1, AC110079.1, AC110373.1, AC110619.1, AC110619.2,
AC110781.3, AC110781.5, AC110926.4, AC112211.2, AC112211.3, AC112229.1,
AC114273.1, AC114730.11, AC114730.8, AC115618.1, AC116609.2, AC116614.1,
AC117372.1, AC118138.2, AC123023.1, AC124057.5, AC125232.1, AC125238.2,
AC125634.1, AC126365.4, AC127496.1, AC130888.1, AC131180.3, AC131180.4,
AC131971.3, AC132186.1, AC132216.1, AC133633.1, AC133919.6, AC135048.13,
AC136704.1, AC137723.5, AC137934.1, AC138035.2, AC138623.1, AC138649.1,
AC139100.3, AC139530.1, AC139887.4, AC140061.2, AC140061.8, AC140542.2,
AC141586.5, AC141928.1, AC142528.1, AC145124.1, AC147651.2, AC159540.2,
AC226118.1, ACAA1, ACAP3, ACBD4, ACBD7, ACCN2, ACCN4, ACE, ACER1,
ACHE, ACIN1, ACOT11, ACOT7, ACOX2, ACOX3, ACP2, ACPT, ACR, ACSF2,
ACSS3, ACTG1P1, ACTL6B, ACTL7B, ACTN3, ACTN4P2, ACTR1A, ACTR1B,
ACTR3P2, ACVR1C, ACVR2A, ACVR2B, ACVR2B-AS1, AD000090.2,
AD000864.1, AD000864.5, ADAD2, ADAM11, ADAM12, ADAM19, ADAM2,
ADAM23, ADAM33, ADAM7, ADAM8, ADAMTS10, ADAMTS12, ADAMTS13,
ADAMTS15, ADAMTS16, ADAMTS17, ADAMTS18, ADAMTS19, ADAMTS2,
ADAMTS20, ADAMTS6, ADAMTS7, ADAMTS8, ADAMTS9, ADAMTS9-AS1,
ADAMTS9-AS2, ADAMTSL1, ADAMTSL3, ADAMTSL4, ADAMTSL5, ADAP1,
ADARB2, ADCK1, ADCY2, ADCY3, ADCY5, ADCY6, ADCY8, ADCYAP1R1,
ADD1, ADD2, ADH1A, ADH1B, ADH5P3, ADH6, ADORA2A, ADRA2B,
ADRA2C, ADRB1, ADRBK1, ADSL, AE000658.27, AE000659.23, AEBP1, AES,
AF013593.1, AF038458.5, AF064858.6, AF064863.1, AF121898.1, AF131217.1,
AF146191.4, AF165138.7, AF196970.3, AF235103.1, AF238380.3, AF238380.5,
AFAP1, AFAP1L1, AFAP1L2, AFF2, AFF3, AFG3L2P1, AFMID, AFP, AGAP11,
AGAP1-IT1, AGAP2, AGAP7, AGBL4-IT1, AGFG2, AGPAT1, AGPAT4, AGR2,
AGRN, AGT, AGTR1, AGXT2L2, AHCY, AHDC1, AHNAK, AHSG, AIF1L,
AIFM3, AIM1L, AIM2, AIP, AIPL1, AJ003147.9, AJ006998.2, AJAP1, AJUBA,
AK1, AK3P3, AK3P5, AK5, AKAP12, AKAP16BP, AKAP8L, AKNA, AKR1B1P1,
AKR1B1P2, AKR1B1P7, AKR1C1, AKR1C2, AKR1C4, AKR1E2, AKT1S1,
AKT2, AL008721.1, AL020996.1, AL022341.1, AL022344.7, AL022476.2,
AL031005.1, AL033532.1, AL035610.2, AL049542.1, AL049610.1, AL049840.1,
AL078621.4, AL078621.5, AL117340.1, AL121578.7, AL121952.1, AL132768.1,
AL132772.1, AL133458.1, AL133492.3, AL137145.1, AL137145.2, AL158069.1,
AL158835.1, AL161626.1, AL161652.1, AL161772.1, AL161908.1, AL161915.1,
AL162151.3, AL162458.1, AL163953.3, AL353671.4, AL353698.1, AL353791.1,
AL353898.1, AL353898.3, AL355149.1, AL355388.1, AL356475.1, AL356776.1,
AL357515.1, AL358813.1, AL359851.1, AL365502.1, AL391421.1, AL445199.1,
AL512355.4, AL590233.1, AL590763.5, AL591025.1, AL591069.1, AL591845.1,
AL592494.4, AL592528.1, AL645728.1, AL645728.2, AL672183.2, ALDH16A1,
ALDH1A1, ALDH1L1-AS2, ALDH2, ALDH3A1, ALDH3B2, ALDH4A1,
ALDH7A1, ALDH7A1P1, ALG12, ALG1L, ALKBH7, ALMS1-IT1, ALMS1P,
ALOX15, ALPK2, ALPL, ALPP, ALX3, AMBP, AMBRA1, AMDHD1, AMDHD2,
AMELY, AMH, AMHR2, AMICA1, AMIGO2, AMMECR1L, AMN, AMOT,
AMOTL1, AMOTL2, AMPH, AMT, ANAPC11, ANG, ANGPT1, ANGPT2, ANK1,
ANK2, ANK3, ANKDD1A, ANKFN1, ANKLE1, ANKRD1, ANKRD13B,
ANKRD13D, ANKRD18B, ANKRD18CP, ANKRD20A10P, ANKRD20A11P,
ANKRD24, ANKRD30BL, ANKRD34B, ANKRD35, ANKRD36B, ANKRD44,
ANKRD50, ANKRD53, ANKRD61, ANKRD63, ANKS1A, ANKS1B, ANKS3,
ANKS4B, ANKS6, ANLN, ANO1, ANO1-AS1, ANO2, ANO3, ANO4, ANO6,
ANO7L1, ANO8, ANO9, ANP32AP1, ANP32BP3, ANTXR2, ANXA1, ANXA10,
ANXA13, ANXA2R, ANXA4, AOAH, AOF2, AOX1, AP000251.2, AP000266.7,
AP000269.1, AP000275.65, AP000320.6, AP000322.53, AP000344.3, AP000344.4,
AP000345.1, AP000345.2, AP000345.4, AP000346.2, AP000347.4, AP000350.4,
AP000354.4, AP000355.2, AP000356.2, AP000357.4, AP000361.2, AP000462.3,
AP000640.10, AP000640.8, AP000662.4, AP000692.9, AP000721.4, AP000765.1,
AP000797.3, AP000807.1, AP000857.1, AP000867.1, AP000997.1, AP001055.6,
AP001056.1, AP001065.15, AP001092.5, AP001187.1, AP001347.6, AP001439.2,
AP001468.1, AP001468.58, AP001469.5, AP001469.9, AP001615.9, AP001623.1,
AP001626.2, AP001628.6, AP001877.1, AP002380.1, AP002387.1, AP002954.3,
AP003068.12, AP003068.23, AP003068.9, AP003419.15, AP003419.16,
AP003774.4, AP004290.1, AP005482.1, AP006216.10, AP006216.11, AP006216.12,
AP006285.1, AP006285.6, AP1G2, AP1M1, AP1M2, AP1S1, AP2A2, AP2S1,
AP3B2, AP3S1, APBA2, APBB1, APC2, APCDD1, APEH, APEX1, APLP1,
APOA1, APOA1BP, APOA2, APOA4, APOB, APOBEC3A, APOBEC3D,
APOBEC3F, APOC1, APOC2, APOC3, APOE, APOH, APOM, APOPT1, APRT,
AQP10, AQP11, AQP5, AQP6, AQP7, AQP7P2, AQP7P3, AQP8, ARAP2, ARC,
ARD1A, ARF1P2, ARFGAP2, ARG1, ARGLU1, ARHGAP10, ARHGAP15,
ARHGAP17, ARHGAP22, ARHGAP23, ARHGAP28, ARHGAP33, ARHGAP35,
ARHGAP36, ARHGAP39, ARHGAP4, ARHGAP40, ARHGAP42P2, ARHGAP6,
ARHGAP8, ARHGAP9, ARHGDIG, ARHGEF1, ARHGEF10, ARHGEF16,
ARHGEF17, ARHGEF19, ARHGEF19-AS1, ARHGEF2, ARHGEF25, ARHGEF39,
ARHGEF4, ARHGEF40, ARHGEF7-AS1, ARID1A, ARID3A, ARID3B, ARID3C,
ARIH2, ARL10, ARL2, ARL2BPP10, ARL3, ARL5AP4, ARMC12, ARMC3,
ARMC5, ARMCX6, ARNT2, ARNTL2, ARPC4-TTLL3, ARPP21, ARRB1,
ARRDC1, ARSA, ARSFP1, ARS1, ART5, ARTN, ARVCF, ARX, AS3MT, ASAP2,
ASAP3, ASCL1, ASGR1, ASGR2, ASIC1, ASIC2, ASIC3, ASIC4, ASIP, ASL,
ASPSCR1, ASS1, ASS1P1, ASS1P10, ASS1P11, ASS1P4, ASS1P5, ASS1P7,
ASS1P9, ASTN1, ASTN2, ATCAY, ATG16L2, ATG2A, ATG4B, ATG9A, ATG9B,
ATHL1, ATN1, ATOH8, ATP11C, ATP12A, ATP13A1, ATP13A2, ATP13A5,
ATP13A5-AS1, ATP1A2, ATP1A3, ATP1A4, ATP1B2, ATP2A1, ATP2B1,
ATP2B2, ATP2B3, ATP2C2, ATP4B, ATP5A1P3, ATP5C1P1, ATP5G1P4,
ATP5G1P8, ATP5G2, ATP5G2P1, ATP5G2P3, ATP5LP2, ATP5SL, ATP6V0C,
ATP7B, ATP8A2, ATP8B1, ATP8B2, ATP8B3, ATPAF1, ATPAF1-AS1, ATRIP,
ATXN2L, ATXN7L1, ATXN7L2, ATXN7L3, AURKB, AURKC, AUTS2, AVP,
AVPR1B, AVPR2, AXIN1, AXIN2, AXL, AZI1, AZU1, B3GALT1, B3GALT6,
B3GAT1, B3GAT3, B3GNT1, B3GNTL1, B4GALNT4, B4GALT5, BAALC,
BAHCC1, BAI1, BAI2, BAI3, BAIAP2-AS1, BAIAP2L1, BAIAP2L2, BAMBI,
BAP1, BARD1, BASP1, BAX, BBC3, BCAM, BCR3, BCAS1, BCAS3, BCAT2,
BCHE, BCKDHA, BCKDHB, BCL11B, BCL2, BCL2L12, BCL7A, BCORL1,
BCRP1, BCRP3, BCYRN1, BDH2, BDKRB1, BDNF, BEND4, BEND5, BGLAP,
BGN, BHLHE22, BHMT, BICC1, BIK, BIN1, BIRC6, BIRC7, BLK, BLM, BLZF1,
BMF, BMP1, BMP2KL, BMP3, BMP4, BMP6, BMP7, BMS1P1, BMS1P4, BNC1,
BNC2, BOC, BOK-AS1, BOLA2, BOLA2B, BPHL, BPIFA1, BPIFB2, BPIFB4,
BRAT1, BRD1, BRD3, BRF1, BRI3, BRI3P1, BRIP1, BRS3, BRSK2, BRWD1-
AS1, BSCL2, BSG, BSN, BTBD11, BTBD17, BTBD18, BTBD2, BTBD8, BTD,
BTF3P13, BTF3P14, BTF3P2, BTG2, BTN2A3P, BTNL3, BUB1, BUB1B, BVES,
BVES-AS1, BX255923.1, BX322557.10, BX470102.3, BX842568.1, BX936347.1,
BZRAP1, C10orf105, C10orf111, C10orf112, C10orf114, C10orf116, C10orf131,
C10orf140, C10orf35, C10orf54, C10orf6, C10orf82, C10orf91, C11orf16, C11orf21,
C11orf35, C11orf40, C11orf41, C11orf45, C11orf49, C11orf54, C11orf61, C11orf72,
C11orf84, C11orf87, C11orf9, C12orf27, C12orf39, C12orf42, C12orf50, C12orf70,
C12orf71, C12orf74, C12orf76, C14orf102, C14orf166B, C14orf169, C14orf176,
C14orf180, C14orf2, C14orf23, C14orf39, C14orf80, C15orf27, C15orf38-AP3S2,
C15orf62, C16orf13, C16orf54, C16orf59, C16orf68, C16orf7, C16orf71, C16orf79,
C16orf85, C16orf86, C16orf87, C16orf89, C16orf92, C16orf93, C16orf95, C16orf96,
C17orf103, C17orf108, C17orf109, C17orf110, C17orf47, C17orf50, C17orf53,
C17orf61, C17orf62, C17orf70, C17orf75, C17orf96, C17orf98, C19orf12, C19orf24,
C19orf25, C19orf33, C19orf35, C19orf48, C19orf51, C19orf54, C19orf6, C19orf60,
C19orf71, C19orf73, C19orf76, C19orf80, C19orf81, C1GALT1, C1orf100,
C1orf106, C1orf111, C1orf122, C1orf123, C1orf126, C1orf141, C1orf148, C1orf158,
C1orf159, C1orf165, C1orf167, C1orf168, C1orf173, C1orf188, C1orf191, C1orf21,
C1orf210, C1orf220, C1orf222, C1orf226, C1orf228, C1orf229, C1orf233, C1orf53,
C1orf85, C1orf86, C1orf93, C1orf95, C1QL1, C1QL2, C1QL4, C1QTNF1,
C1QTNF4, C1QTNF5, C1QTNF6, C1QTNF8, C1QTNF9, C1R, C1RL, C1RL-AS1,
C20orf112, C20orf151, C20orf152, C20orf160, C20orf166, C20orf166-AS1,
C20orf194, C20orf195, C20orf201, C20orf27, C20orf46, C20orf56, C20orf96,
C21orf2, C21orf45, C21orf56, C21orf58, C21orf62, C21orf90, C22orf31, C22orf36,
C22orf43, C22orf45, C2CD4C, C2CD4D, C2orf40, C2orf54, C2orf62, C2orf63,
C2orf66, C2orf68, C2orf72, C2orf74, C2orf79, C2orf80, C2orf88, C3orf10, C3orf21,
C3orf22, C3orf32, C3orf49, C3orf50, C3orf55, C3orf56, C3orf57, C3orf59, C3orf65,
C3orf80, C4orf48, C4orf50, C4orf6, C5, C5orf13, C5orf38, C5orf4, C5orf47,
C5orf52, C5orf65, C6orf108, C6orf118, C6orf123, C6orf124, C6orf134, C6orf141,
C6orf163, C6orf165, C6orf183, C6orf185, C6orf192, C6orf195, C6orf59, C6orf85,
C7orf26, C7orf34, C7orf50, C7orf61, C8A, C8B, C8G, C8orf16, C8orf22, C8orf31,
C8orf34, C8orf44, C8orf48, C8orf49, C8orf56, C8orf73, C8orf82, C8orf86,
C9orf114, C9orf123, C9orf129, C9orf16, C9orf169, C9orf170, C9orf172, C9orf173,
C9orf174, C9orf45, C9orf47, C9orf50, C9orf66, C9orf69, C9orf7, C9orf89, C9orf96,
CA10, CA2, CA4, CA9, CABIN1, CABLES2, CACFD1, CACHD1, CACNA1A,
CACNA1B, CACNA1C, CACNA1C-AS1, CACNA1C-AS2, CACNA1C-AS4,
CACNA1C-IT3, CACNA1E, CACNA1F, CACNA1H, CACNA1I, CACNA1S,
CACNA2D2, CACNA2D3, CACNA2D3-AS1, CACNB1, CACNB3, CACNB4,
CACNG1, CACNG2, CACNG4, CACNG6, CACNG7, CACNG8, CAD, CADM3,
CADM4, CADPS2, CALB1, CALB2, CALCA, CALCB, CALCOCO1, CALCR,
CALHM1, CALHM2, CALHM3, CALM3, CALML6, CALR3, CALY, CAMK1,
CAMK1D, CAMK2A, CAMK4, CAMKV, CAMSAP3, CAP2, CAPG, CAPN1,
CAPN10, CAPN14, CAPN5, CAPN6, CAPN9, CAPNS1, CAPS, CAPSL, CARD10,
CARD14, CARD8, CARHSP1, CARM1, CASC5, CASKIN1, CASKIN2, CASP16,
CASP3, CASZ1, CATSPERG, CAV1, CAV2, CBFA2T2, CBFA2T3, CBLB, CBLC,
CBLN1, CBLN2, CBLN3, CBLN4, CBR3, CBR3-AS1, CBS, CBWD7, CBX1,
CBX1P1, CBX2, CBX3P4, CBX3P5, CBX5, CBX6, CBX8, CC2D1A, CCBL1,
CCBP2, CCDC101, CCDC102A, CCDC106, CCDC107, CCDC116, CCDC128,
CCDC135, CCDC136, CCDC13-AS1, CCDC14, CCDC142, CCDC144NL,
CCDC148, CCDC150, CCDC151, CCDC153, CCDC154, CCDC157, CCDC160,
CCDC162P, CCDC169, CCDC171, CCDC22, CCDC23, CCDC24, CCDC27,
CCDC28B, CCDC3, CCDC33, CCDC38, CCDC40, CCDC42, CCDC57, CCDC58,
CCDC60, CCDC62, CCDC63, CCDC64, CCDC65, CCDC68, CCDC69, CCDC71,
CCDC71L, CCDC74A, CCDC74B, CCDC74B-AS1, CCDC78, CCDC8, CCDC80,
CCDC85A, CCDC88B, CCDC88C, CCDC92, CCDC96, CCDC99, CCHCR1, CCK,
CCKAR, CCL1, CCL14, CCM2, CCNA1, CCNB1, CCNB2, CCNF, CCNJL,
CCNJP2, CCNT2P1, CCR9, CCT5, CCT6B, CD109, CD164L2, CD2, CD247,
CD248, CD276, CD300A, CD320, CD36, CD37, CD3G, CD7, CD79A, CD8B,
CD99, CDAN1, CDC20, CDC20P1, CDC25A, CDC25B, CDC25C, CDC42BPB,
CDC42EP1, CDC42EP2, CDC42-IT1, CDC45, CDCA3, CDCA7, CDCA7L,
CDCA8, CDCP2, CDH10, CDH11, CDH12, CDH12P4, CDH13, CDH15, CDH16,
CDH17, CDH2, CDH20, CDH23, CDH24, CDH3, CDH4, CDH7, CDH8, CDH9,
CDHR2, CDHR3, CDHR5, CDIPT, CDK10, CDK16, CDK18, CDK3, CDK5,
CDK9, CDKAL1, CDKN2AIPNLP2, CDKN2D, CDKN3, CDO1, CDR1, CDR2L,
CDRT15, CDS1, CDT1, CDX2, CDY4P, CDYL2, CEACAM1, CEACAM16,
CEACAM19, CEACAMP2, CEBPA, CEBPA-AS1, CECR2, CECR6, CECR7,
CELF3, CELF5, CELSR1, CELSR2, CELSR3, CEMP1, CEND1, CENPA, CENPE,
CENPF, CENPI, CENPJ, CENPK, CENPM, CENPO, CENPT, CENPV, CENPVP1,
CENPVP2, CENPVP3, CENTA1, CEP135, CEP164, CEP170P1, CEP250, CEP55,
CEP78, CER1, CERCAM, CERS1, CERS4, CES5AP1, CFD, CFHR5, CFL1,
CFL1P1, CFL1P2, CFL1P6, CFP, CGA, CGN, CGNL1, CGREF1, CHAC1, CHAT,
CHCHD5, CHCHD6, CHD5, CHD8, CHFR, CHGA, CHID1, CHKA, CHMP1A,
CHMP6, CHN2, CHORDC2P, CHRD, CHRFAM7A, CHRM2, CHRM3-AS2,
CHRM5, CHRNA2, CHRNA4, CHRNA7, CHRNB4, CHRND, CHRNE, CHST1,
CHST12, CHST13, CHST14, CHST3, CHST5, CHST6, CHST7, CHST9-AS1,
CHSY3, CHTF18, CIAPIN1, CIAPIN1P, CIB2, CIC, CICP13, CILP, CILP2, CIP29,
CISD3, CIT, CITED4, CIZ1, CKAP2, CKAP2L, CKB, CKM, CKMT2, CKS1BP6,
CKS2, CLASP1, CLASRP, CLCF1, CLCN1, CLCN2, CLCN5, CLCN6, CLCN7,
CLCNKA, CLCNKB, CLCP2, CLDN1, CLDN11, CLDN15, CLDN18, CLDN19,
CLDN20, CLDN6, CLDND2, CLEC11A, CLEC16A, CLEC2D, CLEC3B, CLIC1,
CLIC1P1, CLIC3, CLIC4, CLIC5, CLIP2, CLIP3, CLN3, CLPP, CLRN1-AS1,
CLRN3, CLSPN, CLSTN2, CLSTN3, CLTC-IT1, CLU, CLVS2, CMTM1, CMTM2,
CMTM3, CMYA5, CNDP1, CNFN, CNGA1, CNGA4, CNGB1, CNGB3, CNIH2,
CNIH3, CNN1, CNN2, CNN3, CNOT6, CNPY1, CNPY3, CNPY4, CNR1, CNTD2,
CNTF, CNTFR, CNTLN, CNTN2, CNTN3, CNTNAP2, CNTNAP3, CNTNAP3B,
CNTNAP4, CNTNAP5, COBRA1, COCH, COL11A1, COL12A1, COL14A1,
COL18A1, COL19A1, COL1A1, COL1A2, COL21A1, COL22A1, COL23A1,
COL25A1, COL2A1, COL3A1, COL4A1, COL4A2, COL4A2-AS1, COL4A2-AS2,
COL4A5, COL4A6, COL5A1, COL5A2, COL6A1, COL6A2, COL6A4P2, COL6A6,
COL7A1, COL8A2, COL9A1, COL9A2, COL9A3, COLQ, COMMD4, COMT,
COPS5P, COQ2, COQ4, CORIN, CORO1A, CORO1B, CORO2A, CORO2B,
COTL1, COX10, COX11, COX4I2, COX5BP6, COX6B2, COX8C, CPAMD8,
CPB2, CPB2-AS1, CPM, CPN1, CPNE1, CPNE2, CPNE5, CPNE7, CPNE9, CPS1,
CPSF1, CPSF1P1, CPSF4, CPT1B, CPT1C, CPVL, CPXM1, CPZ, CR1,
CR769776.4, CRABP1, CRAT, CRB1, CRB2, CREB3L3, CRELD1, CRH, CRHBP,
CRHR1, CRHR2, CRIP3, CRISPLD1, CRISPLD2, CRMP1, CRNDE, CROCC,
CRTAC1, CRTAM, CRTC1, CRYBA2, CRYBA4, CRYBB1, CRYBB3, CRYGD,
CRYGN, CRYM, CRYM-AS1, CRYZP1, CSAD, CSDC2, CSF1, CSF2RA, CSF3,
CSF3R, CSK, CSMD1, CSMD2, CSMD3, CSNK1A1P1, CSNK1E, CSNK1G2,
CSPG4, CSPG4P5, CSPG5, CSRNP3, CSRP2, CST1, CST3, CST9LP1, CTA-
134P22.2, CTA-150C2.13, CTA-221G9.10, CTA-221G9.11, CTA-221G9.7, CTA-
250D10.19, CTA-250D10.23, CTA-254O6.1, CTA-256D12.12, CTA-282F2.3, CTA-
30512.1, CTA-331F8.1, CTA-373H7.7, CTA-384D8.20, CTA-384D8.31, CTA-
407F11.6, CTA-445C9.15, CTA-796E4.4, CTA-85E5.10, CTA-929C8.6, CTA-
941F9.9, CTA-963H5.5, CTA-984G1.5, CTAGE15P, CTB-113P19.1, CTB-
113P19.4, CTB-118P15.2, CTB-127C13.1, CTB-129P6.4, CTB-134H23.1, CTB-
161M19.2, CTB-171A8.1, CTB-174D11.1, CTB-174D11.2, CTB-175E5.7, CTB-
178M22.2, CTB-17P3.4, CTB-181F24.1, CTB-186G2.1, CTB-186H2.3, CTB-
25B13.12, CTB-25B13.6, CTB-25B13.9, CTB-31O20.8, CTB-33O18.3, CTB-
35F21.3, CTB-37A13.1, CTB-41I6.2, CTB-43E15.4, CTB-47B11.3, CTB-49A3.2,
CTB-49A3.4, CTB-50L17.10, CTB-50L17.5, CTB-55O6.12, CTB-58E17.5, CTB-
58E17.9, CTB-66B24.1, CTB-78F1.1, CTB-79E8.2, CTB-85P21.2, CTB-88F18.3,
CTBP2P1, CTC-209H22.2, CTC-210G5.1, CTC-215O4.4, CTC-218H9.1, CTC-
232P5.1, CTC-232P5.3, CTC-232P5.4, CTC-235G5.2, CTC-235G5.3, CTC-250I14.6,
CTC-250P20.1-001, CTC-260E6.8, CTC-260F20.7, CTC-265N9.1, CTC-268N12.2,
CTC-268N12.3, CTC-278L1.1, CTC-281F24.1, CTC-297N7.1, CTC-297N7.7, CTC-
304I17.5, CTC-325H20.2, CTC-325J23.3, CTC-325L16.1, CTC-327F10.4, CTC-
329D1.2, CTC-338M12.7, CTC-338M12.9, CTC-340A15.2, CTC-360G5.1, CTC-
367F4.1, CTC-367J11.1, CTC-378H22.2, CTC-379B2.4, CTC-398G3.1, CTC-
425O23.2, CTC-429L19.3, CTC-451P13.1, CTC-454I21.4, CTC-455F18.3, CTC-
458I2.2, CTC-462L7.1, CTC-463N11.3, CTC-467M3.1, CTC-470C15.1, CTC-
478M6.1, CTC-479C5.10, CTC-479C5.11, CTC-484M2.1, CTC-497E21.3, CTC-
497E21.4, CTC-507E12.1, CTC-510F12.2, CTC-510F12.4, CTC-518P12.6, CTC-
523E23.1, CTC-535M15.2, CTC-548K16.5, CTC-558O2.1, CTC-558O2.2, CTC-
575I10.1, CTC-756D1.1, CTC-756D1.2, CTC-786C10.1, CTD-2001E22.2, CTD-
2008L17.1, CTD-2008L17.2, CTD-2011F17.2, CTD-2012K14.1, CTD-2012K14.2,
CTD-2012K14.3, CTD-2012K14.6, CTD-2012M11.3, CTD-2020K17.1, CTD-
2020K17.3, CTD-2021A8.2, CTD-2026D20.2, CTD-2027G2.1, CTD-2033A16.2,
CTD-2033D15.1, CTD-2034I4.2, CTD-2035E11.3, CTD-2044J15.1, CTD-
2047H16.4, CTD-2049J23.2, CTD-2054N24.1, CTD-2054N24.2, CTD-2073O6.1,
CTD-2083E4.4, CTD-2085J24.4, CTD-2089N3.1, CTD-2089N3.2, CTD-2089N3.3,
CTD-2090I13.2, CTD-2095E4.2, CTD-2102P23.1, CTD-2104P17.2, CTD-2116F7.1,
CTD-2132N18.2, CTD-2134A5.3, CTD-2134A5.4, CTD-2145A24.4, CTD-2147F2.1,
CTD-2152M20.2, CTD-2173J9.3, CTD-2173L22.4, CTD-2184D3.1, CTD-
2189E23.1, CTD-2192J16.11, CTD-2194D22.3, CTD-2194D22.4, CTD-2196E14.3,
CTD-2196E14.4, CTD-2196E14.5, CTD-2199O4.3, CTD-2201E18.3, CTD-
2201G16.1, CTD-2201G3.1, CTD-2201I18.1, CTD-2206G10.1, CTD-2206N4.4,
CTD-2223O18.1, CTD-2224J9.7, CTD-2227C6.2, CTD-2228K2.7, CTD-2230M5.1,
CTD-2231H16.1, CTD-2235C13.1, CTD-2235C13.2, CTD-2258A20.5, CTD-
2262B20.1, CTD-2265O21.7, CTD-2269E23.2, CTD-2270L9.4, CTD-2281E23.1,
CTD-2290C23.1, CTD-2290P7.1, CTD-2293H3.1, CTD-2297D10.2, CTD-
2298J14.2, CTD-2301A4.1, CTD-2311M21.2, CTD-2320G14.2, CTD-2320O4.2,
CTD-2330K9.3, CTD-2335O3.3, CTD-2336H13.1, CTD-2339F6.1, CTD-2339L15.3,
CTD-2342J14.6, CTD-2358C21.4, CTD-2373J6.1, CTD-2376I20.1, CTD-2383M3.1,
CTD-2514K5.4, CTD-2515A14.1, CTD-2515H24.4, CTD-2516F10.2, CTD-
2517M14.5, CTD-2517M22.14, CTD-2517M22.16, CTD-2517M22.17, CTD-
2517O10.5, CTD-2518G19.3, CTD-2523D13.2, CTD-2524L6.2, CTD-2528L19.4,
CTD-2530H12.4, CTD-2530H12.7, CTD-2530H12.8, CTD-2532D12.4, CTD-
2536I1.1, CTD-2538A21.1, CTD-2540B15.11, CTD-2540B15.7, CTD-2540B15.8,
CTD-2540B15.9, CTD-2541J13.2, CTD-2542C24.1, CTD-2542L18.1, CTD-
2547G23.2, CTD-2547H18.1, CTD-2552B11.2, CTD-2555K7.2, CTD-2561B21.3,
CTD-2561B21.7, CTD-2562J15.4, CTD-2562J15.6, CTD-2562J17.7, CTD-
2562J17.9, CTD-2576D5.4, CTD-2576F9.2, CTD-2583A14.1, CTD-2587H24.5,
CTD-2588C8.6, CTD-2589H19.4, CTD-2589M5.5, CTD-2600O9.1, CTD-2611K5.6,
CTD-2616J11.4, CTD-2621I17.6, CTD-2623N2.5, CTD-2632K10.1, CTD-2639E6.4,
CTD-2647L4.1, CTD-2647L4.4, CTD-2651B20.1, CTD-2651C21.3, CTD-2655K5.1,
CTD-2659N19.10, CTD-2659N19.2, CTD-2659N19.9, CTD-3006G17.2, CTD-
3025N20.2, CTD-3032H12.1, CTD-3051D23.1, CTD-3060P21.1, CTD-3064H18.1,
CTD-3065J16.6, CTD-3065J16.9, CTD-3074O7.5, CTD-3080P12.3, CTD-3088G3.6,
CTD-3096M3.2, CTD-3105H18.7, CTD-3105H18.9, CTD-3113P16.5, CTD-
3113P16.7, CTD-3118D7.1, CTD-3126B10.1, CTD-3148I10.1, CTD-3179P9.2,
CTD-3203P2.1, CTD-3233P19.1, CTDP1, CTDSP1, CTF1, CTGF, CTHRC1,
CTNNAL1, CTNNBIP1, CTNND2, CTNS, CTPS2, CTRL, CTSD, CTSE, CTSL1P2,
CTSL1P3, CTTN, CTXN1, CUBN, CUEDC2, CUL1, CUL7, CUL9, CUX2,
CX3CL1, CXADR, CXCL1, CXCL11, CXCL12, CXCL14, CXCL6, CXCR7,
CXorf30, CXorf31, CXorf58, CXorf68, CXorf69, CXXC4, CYB5R2, CYB5R3,
CYBA, CYGB, CYHR1, CYLN2, CYMP, CYP11A1, CYP24A1, CYP26B1,
CYP27C1, CYP2C19, CYP2C9, CYP2D6, CYP2D7P1, CYP2E1, CYP2F1P,
CYP2S1, CYP2W1, CYP3A4, CYP46A1, CYP4A22-AS1, CYP4B1, CYP4F27P,
CYP4F29P, CYP4F30P, CYP4F31P, CYP4F35P, CYP4X1, CYP51P3, CYP7B1,
CYP8B1, CYR61, CYSLTR2, CYTH1, CYTH2, CYTH3, CYTH4, CYTL1,
DAAM2, DAB1, DAB2, DAB2IP, DACT1, DACT2, DACT3, DACT3-AS1,
DAGLA, DAGLB, DALRD3, DAND5, DAO, DAPK3, DARC, DBC1, DBF4B,
DBH, DBH-AS1, DBIP1, DBN1, DBNDD1, DBNL, DBX2, DCAF12L1,
DCAF12L2, DCAF15, DCAF4, DCBLD2, DCC, DCHS1, DCLK1, DCN, DCPS,
DCTN1, DCUN1D2-AS2, DCX, DCXR, DDA1, DDAH2, DDB1, DDC, DDEF2,
DDIT4L, DDN, DDX11, DDX11L1, DDX18P5, DDX18P6, DDX39A, DDX3YP1,
DDX49, DDX51, DEAF1, DECR2, DEF6, DEFB109P3, DEFB115, DEGS2,
DENND1A, DENND1C, DENND2A, DENND3, DENND5B-AS1, DEPDC1,
DEPDC1B, DEPDC4, DEPDC7, DFNA5, DGAT1, DGAT2, DGAT2L6, DGCR6L,
DGKH, DGKI, DGKK, DGKQ, DHCR24, DHCR7, DHDH, DHFR, DHODH,
DHPS, DHRS1, DHRS13, DHRS3, DHRSX, DHX34, DIAPH2-AS1, DIAPH3,
DIO3, DIO3OS, DIRAS3, DIRC3, DISP2, DKEZp761P0423, DKK1, DKK2, DKK3,
DKKL1, DKKL1P1, DLEC1, DLEU7, DLG4, DLG5, DLGAP1, DLGAP2,
DLGAP3, DLGAP5, DLK1, DLL1, DLL3, DLL4, DLX2, DLX3, DMAP1, DMBT1,
DMD, DMD-AS1, DMD-AS2, DMKN, DMPK, DMRT2, DMRTC1, DMRTC1B,
DMWD, DNAAF3, DNAH1, DNAH10, DNAH10OS, DNAH11, DNAI2, DNAJB13,
DNAJB5, DNAJC11, DNAJC17, DNAJC27-AS1, DNAJC4, DNAL4, DNALI1,
DNASE1, DNASE1L1, DNASE1L3, DNM1P46, DNMBP, DNMBP-AS1,
DNMT3A, DOC2A, DOC2B, DOC2GP, DOCK2, DOCK5, DOCK6, DOCK7,
DOCK8, DOK1, DOK4, DOK5, DOK6, DOLK, DOPEY2, DPEP1, DPF1, DPF3,
DPP10, DPP3, DPP4, DPPA4, DPY19L2P1, DPYD, DPYSL3, DPYSL4, DPYSL5,
DQX1, DRAP1, DRAXIN, DRD1, DRD1IP, DRD2, DRD4, DRD5, DRP2, DSC2,
DSC3, DSCAM, DSCAM-IT1, DSCAML1, DSCR6, DSCR9, DSEL, DSG2, DSG4,
DTL, DTX1, DTX3, DTX4, DUOX1, DUSP1, DUSP10, DUSP12, DUSP15,
DUSP18, DUSP22, DUSP6, DUSP8, DUSP9, DUT, DUTP2, DUTP6, DVL2,
DYNLRB2, DYRK1B, DYSF, DZANK1, E2F1, E2F2, E2F5, E2F7, E2F8, E4F1,
EBF1, EBF2, EBF3, EBF4, EBP, ECE1, ECH1, EDAR, EDC4, EDG4, EEF1A1P12,
EEF1A1P3, EEF1A1P31, EEF1B2P1, EEF1DP1, EEF2, EFCAB1, EFCAB4A,
EFCAB4B, EFCAB8, EFEMP1, EFEMP2, EFHD2, EFNA1, EFNA2, EFNA3,
EFNA4, EFNB1, EFNB2, EFNB3, EGFEM1P, EGFL6, EGFL7, EGLN2, EGLN3,
EGR1, EHD2, EHMT1, EIF2AK4, EIF2S2, EIF2S2P2, EIF3C, EIF4A1, EIF4A1P6,
EIF4A1P7, EIF4BP2, EIF4EBP3, EIF5AP4, ELAVL2, ELAVL3, ELF4, ELF5,
ELFN1, ELFN2, ELMO1-AS1, ELMOD1, ELOVL2, ELOVL2-AS1, ELP6, EMC10,
EME2, EMID1, EMID2, EMILIN1, EMILIN2, EMILIN3, EML2, EMX2, EN2,
ENC1, ENDOG, ENGASE, ENHO, ENO4, ENPEP, ENPP1, ENPP6, ENTHD2,
ENTPD6, ENTPD8, EP300, EPB42, EPB49, EPHA1, EPHA10, EPHA3, EPHA4,
EPHA6, EPHA7, EPHB1, EPHB2, EPHB3, EPHB4, EPHB6, EPHX3, EPN1, EPN3,
EPOR, EPPK1, EPS15L1, EPS8, EPS8L1, EPS8L2, EPS8L3, ERBB2, ERBB3,
ERBB4, ERC2, ERCC2, ERF, ERGIC3, ERI3, ERI3-IT1, ERN1, ERN2, ERRFI1-
IT1, ERVMER34-1, ESPL1, ESPN, ESPNL, ESRP2, ESRRAP1, ESRRB, ESYT3,
ETNK2, ETV1, ETV3L, ETV4, ETV6, EVC, EVC2, EVI2A, EVL, EXD3, EXO1,
EXOC3L1, EXOC3L4, EXOC7, EXOSC10, EXOSC5, EXT1, EXTL1, EXTL2,
EYA1, EYA2, EYA4, EZH2, EZH2P1, F10, F10-AS1, F2, F2R, F2RL2, F5, F7,
FABP3, FABP5, FABP5P1, FABP5P2, FABP5P7, FABP6, FABP7, FABP7P1,
FADS2, FADS6, FAH, FAHD2B, FAHD2CP, FAIM2, FAM100B, FAM101A,
FAM101B, FAM107B, FAM108A1, FAM108A5P, FAM108A6P, FAM109A,
FAM110B, FAM110D, FAM111B, FAM113A, FAM115B, FAM116B, FAM117B,
FAM122C, FAM123A, FAM125A, FAM125B, FAM126A, FAM127A, FAM127C,
FAM129C, FAM131A, FAM131B, FAM131C, FAM132A, FAM136A, FAM138B,
FAM154B, FAM155B, FAM156B, FAM157A, FAM157C, FAM159A, FAM163A,
FAM166B, FAM168B, FAM171A2, FAM175A, FAM176B, FAM179A, FAM180B,
FAM181B, FAM184A, FAM185A, FAM185BP, FAM186B, FAM188A,
FAM189A1, FAM189A2, FAM189B, FAM18A, FAM193A, FAM194A, FAM195B,
FAM196A, FAM196B, FAM198A, FAM198B, FAM19A1, FAM19A5, FAM201B,
FAM203A, FAM203B, FAM20A, FAM211B, FAM212B, FAM213B, FAM215B,
FAM21B, FAM220CP, FAM221B, FAM227A, FAM228A, FAM22A, FAM22E,
FAM22F, FAM22G, FAM27D1, FAM27E2, FAM32A, FAM3D, FAM40A,
FAM43B, FAM46B, FAM57A, FAM57B, FAM58BP, FAM58DP, FAM59B,
FAM5B, FAM5C, FAM60BP, FAM60CP, FAM64A, FAM65A, FAM65B,
FAM65C, FAM66B, FAM66C, FAM66D, FAM69B, FAM70B, FAM71E1,
FAM72B, FAM76B, FAM78A, FAM83A, FAM83B, FAM83D, FAM83F, FAM83H,
FAM84A, FAM86B1, FAM86B2, FAM86C2P, FAM86FP, FAM86GP, FAM86HP,
FAM86JP, FAM86KP, FAM89A, FAM89B, FAM90A1, FAM92B, FAM99B,
FANCA, FANCE, FANCG, FANCI, FAR1-IT1, FARP1, FARP1-AS1, FARP2,
FASN, FAT2, FAT3, FATE1, FBLIM1, FBLL1, FBLN1, FBLN2, FBLN5, FBLN7,
FBN1, FBN2, FBN3, FBP2, FBRS, FBRSL1, FBXL10, FBXL12, FBXL19,
FBXL19-AS1, FBXL20, FBXL21, FBXL22, FBXL7, FBXO10, FBXO15, FBXO2,
FBXO24, FBXO40, FBXO44, FBXO5, FBXW4, FBXW9, FCGR1A, FCGR2B,
FCGRT, FCHO1, FCHSD1, FCHSD2, FCRLB, FDFT1, FDX1L, FDX1P1, FDXR,
FER1L4, FER1L6, FER1L6-AS2, FERMT1, FES, FEV, FEZF1, FGB, FGD1, FGD3,
FGD5, FGD6, FGF1, FGH11, FGF12, FGF12-AS2, FGF12-AS3, FGF13, FGF13-
AS1, FGF14, FGF17, FGF18, FGF5, FGFR1, FGFR2, FGFR3, FGFR3P6, FGFR4,
FGFRL1, FHAD1, FHIT, FHL1, FHL2, FHL3, FHOD1, FHOD3, FIBCD1, FIBP,
FIG.N, FIS1, FITM1, FJX1, FKBP10, FKBP1B, FKBP8, FKBP9, FKBP9L, FKRP,
FLJ13305, FLJ23834, FLJ25404, FLJ31568, FLJ34690, FLJ37228, FLJ44048,
FLJ44124, FLJ44342, FLJ46309, FLNA, FLNC, FLOT2, FLRT1, FLRT2, FLRT3,
FLYWCH1, FLYWCH2, FMNL1, FMNL2, FMO1, FN1, FNDC1, FNDC4, FNDC8,
FOLR1, FOS, FOSB, FOXA1, FOXA3, FOXB1, FOXD3, FOXD4L1, FOXD4L6,
FOXH1, FOXI2, FOXI3, FOXJ1, FOXM1, FOXO3B, FOXO4, FOXP1-AS1,
FOXP3, FOXP4, FOXRED1, FPGS, FRAT1, FRAT2, FREM1, FREM2, FRMD4A,
FRMD4B, FRMD5, FRMD6, FRMD8P1, FRMPD2, FRMPD3, FSCN1, FSCN2,
FSCN3, FSD1, FSIP2, FST, FSTL1, FSTL3, FSTL5, FTCD, FTCD-AS1, FTH1P15,
FTH1P5, FTL, FTLP12, FTLP3, FTOP1, FTX, FUOM, FUT1, FUT6, FUT9, FUZ,
FXYD3, FXYD6, FXYD7, FZD2, FZD3, FZD7, FZR1, G6PC, G6PC3, GAA, GAB1,
GAB2, GABARAP, GABBR2, GABRA3, GABRA5, GABRB1, GABRB2, GABRE,
GABRG1, GABRG3, GABRP, GABRQ, GAGE10, GAL, GAL3ST1, GAL3ST3,
GAL3ST4, GALE, GALK1, GALM, GALNS, GALNT1, GALNT13, GALNT5,
GALNT6, GALNT9, GALNTL1, GALNTL4, GALNTL6, GALR1, GALR3, GALT,
GAPDHP21, GAPDHP32, GAPDHP37, GAPDHP42, GAPDHP44, GAPDHP52,
GAPDHP59, GAPDHP62, GAPDHP66, GAPDHP68, GAPDHS, GARNL3, GAS1,
GAS2L3, GAS6, GAS6-AS1, GAS7, GAS8, GAST, GATA1, GATA3, GATA4,
GATA5, GATA6, GATM, GATS, GATSL2, GATSL3, GBA2, GBAP1, GBP6,
GBX1, GC, GCAT, GCDH, GCG, GCHFR, GCKR, GCLC, GCN1L1, GCNT1,
GCNT2, GCNT6, GCSHP4, GDAP1L1, GDF10, GDF11, GDF5, GDF5OS, GDF6,
GDPD2, GDPD3, GDPD4, GDPD5, GEMIN7, GEMIN8P4, GET4, GFER, GFI1,
GFOD2, GFRA1, GFRA2, GFRA3, GGA1, GGH, GGT1, GGT3P, GGT7, GHR,
GHRH, GHRL, GHRLOS, GIF, GIGYF1, GIMAP7, GINS1, GINS4, GIP, GIPC1,
GIPC2, GIPR, GIT1, GJA1, GJA3, GJA9, GJB1, GJB6, GJC1, GJC2, GJC3, GJD3,
GJD4, GLB1L, GLB1L2, GLDC, GLDCP, GLDN, GLI2, GLI3, GLI4, GLOD5,
GLP2R, GLRA4, GLRX, GLS, GLT1D1, GLTPD1, GLTPD2, GLTSCR1,
GLUD1P6, GLUL, GLULP3, GLULP4, GLYATL1, GLYATL1P1, GLYATL1P2,
GLYATL2, GLYCTK, GLYCTK-AS1, GMFG, GMIP, GMNC, GNA14, GNAI2,
GNAO1, GNAQP1, GNAT3, GNB1L, GNB2, GNG13, GNG3, GNG8, GNL3L,
GNMT, GNRH2, GOLGA6L1, GOLGA6L10, GOLGA6L5, GOLGA6L7P,
GOLGA8A, GOLGA8F, GOLGA8J, GOLIM4, GOT2P3, GPA33, GPAT2,
GPATCH3, GPBAR1, GPC1, GPC2, GPC3, GPC4, GPC5, GPC5-AS1, GPC6,
GPC6-AS2, GPER, GPHA2, GPM6B, GPR108, GPR111, GPR112, GPR113,
GPR114, GPR119, GPR123, GPR126, GPR128, GPR133, GPR137, GPR137C,
GPR141, GPR142, GPR143, GPR143P, GPR146, GPR148, GPR153, GPR156,
GPR160, GPR161, GPR162, GPR173, GPR179, GPR18, GPR182, GPR26, GPR37,
GPR45, GPR62, GPR64, GPR68, GPR78, GPR82, GPR83, GPR85, GPR98,
GPRC5A, GPRC5B, GPRC5C, GPRIN2, GPS1, GPSM1, GPT, GPT2, GPX1, GPX7,
GPX8, GRAMD1A, GRAMD2, GRAP2, GRB14, GRB7, GRHL2, GRHPR, GR1A1,
GRID1, GRID2, GRID2IP, GRIK1, GRIK3, GRIK4, GRIN2C, GRIN2D, GRIN3A,
GRIN3B, GRINA, GRK1, GRK4, GRK5, GRM2, GRM3, GRM5, GRM5-AS1,
GRM7, GRM8, GRN, GRTP1, GRTP1-AS1, GS1-124K5.2, GS1-124K5.3, GS1-
124K5.7, GS1-124K5.8, GS1-124K5.9, GS1-256O22.3, GS1-388B5.3, GS1-421I3.4,
GS1-5L10.1, GSDMB, GSG1, GSG1L, GSG2, GSPT2, GSTA4, GSTK1, GSTM1,
GSTM2, GSTM5, GSTP1, GSTT1, GSTT2, GSTT2B, GSTZ1, GTDC2, GTF2F1,
GTF2H2B, GTF2IP1, GTF3C5, GTSE1, GUCA1A, GUCA2B, GULOP, GULP1,
GUSBP5, GUSBP8, GYG2, GYG2P1, GYPC, GYS1, H19, H1FNT, H1FX, H1FX-
AS1, H2AFJ, H2AFY2, H2AFZP1, H2BFS, H3F3A, H3F3AP1, H3F3B, H3F3C,
H6PD, HAAO, HABP2, HAGH, HAGHL, HAL, HAMP, HAND1, HAO2, HAP1,
HAPLN3, HAPLN4, HARS, HAUS1P1, HAVCR1, HBE1, HBQ1, HCFC1, HCN1,
HCN3, HCN4, HDAC1, HDAC10, HDAC11, HDAC2, HDAC6, HDAC7, HDC,
HDHD1A, HEATR7B1, HEATR8, HECTD4, HECW1, HECW2, HELB, HELLS,
HEPACAM, HEPACAM2, HEPH, HEPHL1, HERC2P8, HES1, HES3, HES4,
HES5, HES6, HES7, HEXDC, HEXIM2, HEY2, HEYL, HFM1, HGFAC, HHEX,
HHIP, HHIP-AS1, HHIPL1, HIC1, HIC2, HIF3A, HIGD1A, HIGD1AP1, HIGD2A,
HIGD2B, HINT2, HIP1, HIP1R, HIPK4, HIST1H1C, HIST1H1E, HIST1H2AC,
HIST1H2AE, HIST1H2AG, HIST1H2BG, HIST1H2BJ, HIST1H2BK, HIST1H2BO,
HIST1H3A, HIST1H3D, HIST1H3E, HIST1H4I, HIST1H4J, HIST1H4K,
HIST2H2AA3, HIST2H2AA4, HIST2H2AB, HIST2H2AC, HIST2H2BE,
HIST2H3PS2, HIST2H4A, HIST3H2A, HIST3H2BB, HIST3H3, HK1, HK2,
HK2P1, HK3, HKDC1, HLA-B, HLA-DQA1, HLA-DQB1, HLA-DQB1-AS1,
HLTF-AS1, HM13-AS1, HMBS, HMG20B, HMGA1, HMGA1P1, HMGA1P2,
HMGA1P3, HMGA1P4, HMGA2, HMGB1P10, HMGB1P24, HMGB3P22,
HMGB3P32, HMGB3P8, HMGCR, HMGCS1, HMGCS2, HMGN1P26,
HMGN1P35, HMGN1P4, HMGN2, HMGN2P10, HMGN2P17, HMGN2P31,
HMGN2P42, HMGN3P1, HMHA1, HMP19, HMSD, HN1, HNF1A, HNF1A-AS1,
HNF1B, HNF4A, HNF4G, HNRNPA1P22, HNRNPA2B1, HNRNPA3P3, HNRNPU,
HOMER3, HOTAIRM1, HOXA1, HOXA2, HOXA3, HOXA4, HOXA-AS2, HOXA-
AS3, HOXB2, HOXB3, HOXB-AS1, HOXB-AS3, HOXC4, HOXD1, HOXD3,
HOXD-AS1, HPCAL1, HPD, APDL, HPN, HPS1, HPSE, HRASLS5, HRC, HRH2,
HRH3, HRK, HS1BP3, HS3ST3A1, HS3ST3B1, HS3ST4, HS3ST5, HS3ST6,
HS6ST1, HS6ST1P1, HS6ST3, hsa-mir-3187, hsa-mir-3195, HSBP1, HSD11B1L,
HSD11B2, HSD17B14, HSD17B2, HSD3B1, HSD3B7, HSF2BP, HSPA12B,
HSPB2, HSPB8, HSPBP1, HSPD1P21, HSPG2, HTR1A, HTR1B, HTR1E, HTRA1,
HTT, HUNK, HYAL1, HYAL2, HYAL3, HYI-AS1, ID3, IDH1, IDH1-AS1, IDH2,
IDH3B, IDH3G, IDI1, IDI2-AS1, IDUA, IER2, IER5L, IFI6, IFITM1, IFITM10,
IFITM3, IFITM5, IFNWP19, IFT140, IFT27, IGBP1P3, IGDCC3, IGDCC4, IGF1,
IGF2, IGF2BP1, IGF2BP2, IGF2BP3, IGFALS, IGFBP1, IGFBP2, IGFBP5,
IGFBPL1, IGFL1, IGFL4, IGFLR1, IGLON5, IGLV7-43, IGLV7-46, IGSF10,
IGSF21, IGSF22, IGSF23, IGSF3, IGSF8, IGSF9, IHH, IKBKE, IL11RA, IL12A,
IL12B, IL13, IL15, IL16, IL17C, IL17RD, IL1RAP, IL1RAPL1, IL23R, IL27RA,
IL28RA, IL31RA, IL32, IL34, IL4R, IL6STP1, ILF3, ILVBL, IMPA2, IMPDH1P4,
INF2, ING4, INHA, INMT, INO80, INPP5E, INPP5J, INPPL1, INSR, INTS1,
INTS3, INTS9, IPO9, IPW, IQCA1, IQCC, IQCE, IQCJ-SCHIP1, IQGAP2,
IQGAP3, IQSEC3, IRF3, IRX1, IRX2, IRX3, IRX4, IRX5, ISG15, ISL1, ISLR2,
ISOC2, ISPD, ISX, ISYNA1, ITGA11, ITGA2B, ITGA6, ITGA7, ITGA8, ITGAL,
ITGB4, ITGB5, ITGB6, ITGB7, ITGB8, ITGBL1, ITIH1, ITIH2, ITLN2, ITM2A,
ITM2BP1, ITM2C, ITPA, ITPK1, ITPR1, ITPRIP, ITPRIPL1, IVD, IVNS1ABP,
IYD, IZUMO4, J01415.7, JAK3, JAM2, JARID2, JMJD4, JMJD7-PLA2G4B,
JMJD8, JPH2, JPH3, JPH4, JRK, JUN, JUP, KAL1, KALP, KALRN, KANK1,
KANK2, KANK3, KANK4, KANSL1-AS1, KARSP2, KAT2A, KATNAL2,
KATNB1, KAZALD1, KAZN, KB-1205A7.1, KB-1269D1.8, KB-1471A8.1, KB-
1507C5.3, KB-1507C5.4, KB-1562D12.1, KB-1562D12.2, KB-1683C8.1, KB-
1896H10.1, KB-318B8.7, KBTBD12, KCNA4, KCNAB2, KCNB1, KCNC2,
KCNC3, KCND1, KCND2, KCNE1L, KCNE2, KCNE3, KCNG1, KCNG2, KCNH3,
KCNH5, KCNH6, KCNH8, KCNIP2, KCNIP3, KCNIP4, KCNJ12, KCNJ2, KCNJ4,
KCNJ5, KCNJ6, KCNJ6-IT1, KCNK10, KCNK15, KCNMB1, KCNMB4, KCNN1,
KCNN3, KCNQ1, KCNQ1DN, KCNQ1OT1, KCNQ2, KCNQ4, KCNQ5, KCNS1,
KCNS2, KCNS3, KCNT1, KCNT2, KCNV1, KCNV2, KCTD11, KCTD12,
KCTD15, KCTD16, KCTD17, KCTD5, KDELC1P1, KDM2B, KDM4A-AS1,
KDM4B, KDM5B, KDM5B-AS1, KDM8, KEL, KHDRBS2, KHK, KHSRP,
KHSRPP1, KIAA0101, KIAA0182, KIAA0195, KIAA0232, KIAA0363, KIAA0391,
KIAA0427, KIAA0514, KIAA0528, KIAA0664L3, KIAA0753, KIAA0754,
KIAA0895L, KIAA0913, KIAA0930, KIAA1045, KIAA1161, KIAA1211,
KIAA1239, KIAA1324L, KIAA1522, KIAA1543, KIAA1545, KIAA1549,
KIAA1549L, KIAA1602, KIAA1671, KIAA1683, KIAA1688, KIAA1755,
KIAA1875, KIAA1984, KIAA1984-AS1, KIF11, KIF12, KIF13B, KIF14, KIF15,
KIF17, KIF18A, KIF18B, KIF19, KIF1C, KIF20A, KIF21B, KIF23, KIF26A,
KIF3C, KIF6, KIF7, KIFC2, KIR3DX1, KIRREL, KIRREL2, KIRREL3-AS3,
KISS1R, KIT, KITLG, KLB, KLC1, KLC3, KLC4, KLF15, KLF16, KLF2, KLF4,
KLF5, KLF6, KLHDC3, KLHDC7A, KLHDC8B, KLHL10, KLHL13, KLHL14,
KLHL17, KLHL22, KLHL25, KLHL2P1, KLHL4, KLK10, KLK13, KLK15, KLK6,
KLRC2, KLRC4, KLRC4-KLRK1, KLRG1, KLRK1, KNCN, KNDC1, KPNA3-IT1,
KPRP, KPTN, KRBA1, KRBA2, KREMEN2, KRR1P1, KRT15, KRT16P3,
KRT17P2, KRT18P12, KRT18P15, KRT18P20, KRT18P28, KRT18P32,
KRT18P34, KRT18P5, KRT19, KRT19P1, KRT23, KRT4, KRT42P, KRT5, KRT7,
KRT73, KRT81, KRT8P15, KRT8P26, KRT8P32, KRT8P36, KRT8P45, KRT8P8,
KRT8P9, KRTAP5-1, KRTAP5-10, KRTAP5-2, KRTAP5-5, KRTAP5-9, KRTDAP,
KTI12, L1CAM, L1TD1, L3MBTL1, LA16c-306A4.2, LA16c-312E8.2, LA16c-
313D11.10, LA16c-313D11.11, LA16c-313D11.9, LA16c-329F2.1, LA16c-
349E10.1, LA16c-360H6.3, LA16c-361A3.3, LA16c-366D3.1, LA16c-380A1.1,
LA16c-381G6.1, LA16c-385E7.1, LA16c-390E6.5, LA16c-395F10.1, LA16c-
395F10.2, LA16c-60G3.7, LAG3, LAMA1, LAMB1, LAMB2, LAMC1, LAMP5,
LAMTOR2, LAMTOR3P1, LANCL2, LANCL3, LARGE, LARP4B, LASP1,
LASS1, LASS4, LATS2, LAYN, LBX2, LBX2-AS1, LCAT, LCK, LCN12, LCN15,
LCP1, LDB1, LDB2, LDHA, LDHAL6A, LDHAL6EP, LDHAP2, LDHAP3,
LDHAP5, LDHB, LDHD, LDLRAD1, LDLRAD2, LECT2, LEFTY2, LEKR1,
LEMD2, LENEP, LEO1, LEPRE1, LEPREL1, LEPREL2, LEPREL4, LFNG,
LGALS12, LGALS14, LGALS2, LGI4, LGR6, LHCGR, LHFP, LHFPL1, LHFPL3,
LHPP, LHX1, LHX3, LHX4, LHX5, LHX6, LHX9, LIG1, LILRA6, LILRB3,
LILRB5, LIMD2, LIMK1, LIMK2, LIMS2, LIN28A, LIN28B, LIN7A, LIN9,
LINC00029, LINC00051, LINC00086, LINC00087, LINC00158, LINC00173,
LINC00174, LINC00176, LINC00202, LINC00205, LINC00208, LINC00210,
LINC00261, LINC00265, LINC00304, LINC00311, LINC00313, LINC00319,
LINC00333, LINC00337, LINC00338, LINC00340, LINC00341, LINC00348,
LINC00379, LINC00403, LINC00404, LINC00427, LINC00458, LINC00470,
LINC00479, LINC00482, LINC00487, LINC00491, LINC00501, LINC00514,
LINC00515, LINC00526, LINC00539, LINC00568, LINC00577, LINC00589,
LINC00595, LINC00599, LINC00605, LINC00607, LINC00614, LINC00616,
LINC00618, LINC00621, LINC00634, LINC00636, LINC00638, LINC00648,
LINC00649, LINC00650, LINC00652, LINC00669, LINC00672, LINGO1,
LINGO3, LIPC, LIPE, LIPG, LIPJ, LIPT2, LL0XNC01-116E7.1, LL0XNC01-
221F2.2, LL0XNC01-237H1.2, LL22NC03-2H8.4, LL22NC03-86G7.1, LLGL1,
LLGL2, LMBR1L, LMF1, LMF2, LMNB1, LMNB2, LMO3, LMO4, LMOD2,
LMTK3, LMX1B, LOC100128326, LOC100129076, LOC100129086,
LOC100129211, LOC100129905, LOC100129975, LOC100130154,
LOC100130276, LOC100130886, LOC100130914, LOC100131859,
LOC100132112, LOC100132491, LOC100132740, LOC100132942,
LOC100133477, LOC100133516, LOC100133609, LOC100133697,
LOC100133772, LOC100133923, LOC100133999, LOC100134053,
LOC100134134, LOC100134144, LOC100134265, LOC100134291,
LOC100134530, LOC100134634, LOC134997, LOC149134, LOC158301,
LOC202781, LOC220686, LOC285074, LOC338758, LOC339290, LOC341457,
LOC387934, LOC388494, LOC389332, LOC389599, LOC389634, LOC389765,
LOC390705, LOC401098, LOC401357, LOC401720, LOC439953, LOC440063,
LOC440926, LOC441066, LOC441268, LOC641950, LOC642299, LOC642852,
LOC643313, LOC643389, LOC643911, LOC644390, LOC644596, LOC644670,
LOC644914, LOC644919, LOC645233, LOC645566, LOC646044, LOC646301,
LOC647346, LOC647886, LOC648526, LOC648852, LOC649841, LOC649999,
LOC650157, LOC653210, LOC653505, LOC653829, LOC654244, LOC728153,
LOC728440, LOC728457, LOC728492, LOC728661, LOC728728, LOC729021,
LOC729120, LOC729137, LOC729660, LOC729816, LOC729970, LOC730235,
LOC730284, LOC730286, LOC730740, LOC730993, LOC732360, LOC81691,
LOC92659, LOC93556, LOXL1, LOXL2, LOXL3, LOXL4, LPA, LPAL2, LPAR1,
LPAR2, LPAR3, LPCAT2, LPCAT4, LPHN1, LPHN2, LPHN3, LPIN3, LPPR2,
LPPR5, LRAT, LRCH2, LRCH4, LRFN1, LRFN3, LRFN4, LRGUK, LRIG3,
LRMP, LRP1, LRP1B, LRP2, LRP3, LRP5, LRPAP1, LRRC14, LRRC14B,
LRRC16B, LRRC17, LRRC20, LRRC23, LRRC24, LRRC26, LRRC27, LRRC28,
LRRC29, LRRC31, LRRC32, LRRC33, LRRC37A3, LRRC37B2, LRRC41,
LRRC43, LRRC45, LRRC4B, LRRC4C, LRRC53, LRRC55, LRRC56, LRRC61,
LRRC63, LRRC69, LRRC7, LRRC71, LRRC8A, LRRN1, LRRN2, LRRN4,
LRRN4CL, LRSAM1, LRTM1, LRTM2, LSM3P2, LSM3P3, LSMD1, LSR, LSS,
LTBP1, LTC4S, LTK, LUM, LUZP2, LY6E, LY6H, LYPD4, LYPD6, LYPD6B,
LYPLA2, LYZ, LZTR1, LZTS1, LZTS2, M6PRBP1, MAB21L1, MACROD1,
MACROD2-AS1, MAD1L1, MAD2L2, MADCAM1, MAFG-AS1, MAG,
MAGEA4, MAGEA8, MAGEB17, MAGEC3, MAGED1, MAGED2, MAGED4,
MAGED4B, MALL, MAMDC4, MAMLD1, MAMSTR, MAN1B1, MAN2B1,
MAN2C1, MANEAL, MAOA, MAOB, MAP2K2, MAP2K4, MAP2K4P1,
MAP2K6, MAP3K1, MAP3K10, MAP3K11, MAP3K12, MAP3K3, MAP3K4,
MAP4K1, MAPK11, MAPK12, MAPK15, MAPK3, MAPK4, MAPK6, MAPK7,
MAPK8IP1, MAPK8IP2, MAPK8IP3, MARCKS, MARCKSL1, MARK4, MASP1,
MAST1, MAST2, MATK, MATN1, MATN2, MATN3, MATN4, MAVS, MAZ,
MBD3, MBD6, MBOAT7, MCAM, MCAT, MCF2L, MCHR2, MCM10, MCM2,
MCM3APAS, MCM7, MCTP1, MDFI, MDGA2, MDK, MECR, MED12, MED12L,
MED14-AS1, MED16, MED22, MED24, MED25, MED27, MEF2C, MEGF10,
MEGF6, MEGF8, MEIS3, MEIS3P1, MEIS3P2, MELK, MEMO1, MEMO1P1,
MEN1, MEP1A, MERTK, MESTP4, MET, Metazoa_SRP, METT11D1, METTL22,
METTL7B, METTL9, MEX3A, MEX3B, MEX3D, MFAP2, MFAP4, MFAP5,
MFGE8, MFNG, MFSD10, MFSD2B, MFSD3, MFSD7, MGAT3, MGAT4B,
MGAT4C, MGAT5B, MGC16121, MGC16384, MGC20983, MGC39900, MGLL,
MGMT, MGRN1, MIAT, MICAL1, MICAL2, MICALL1, MID1IP1-AS1, MIEN1,
MIER2, MIF, MIIP, MINK1, MIP, MIR103A2, MIR1203, MIR1260B, MIR1268A,
MIR127, MIR1276, MIR135A1, MIR135A2, MIR137HG, MIR149, MIR181A2HG,
MIR1915, MIR1972-1, MIR205HG, MIR210HG, MIR221, MIR25, MIR2682,
MIR296, MIR3164, MIR3176, MIR3180-4, MIR3190, MIR31HG, MIR320E,
MIR33B, MIR3615, MIR3622A, MIR3646, MIR3648, MIR3677, MIR3685,
MIR3687, MIR378H, MIR3934, MIR3942, MIR421, MIR425, MIR4253, MIR4295,
MIR4308, MIR431, MIR433, MIR4442, MIR4453, MIR4454, MIR4477A, MIR4479,
MIR4502, MIR4504, MIR4644, MIR4650-1, MIR4686, MIR4697, MIR4730,
MIR4737, MIR4750, MIR4766, MIR4768, MIR4782, MIR4783, MIR4793, MIR483,
MIR486, MIR497HG, MIR503, MIR5094, MIR548I1, MIR5685, MIR573, MIR578,
MIR593, MIR600HG, MIR608, MIR618, MIR619, MIR621, MIR631, MIR640,
MIR643, MIR647, MIR661, MIR662, MIR762, MIR92B, MIR98, MIRLET7D,
MIRLET7F1, MIRLET7G, MIXL1, MKI67, MKL1, MKNK1, MKRN3, MKRN7P,
MLANA, MLL4, MLLT3, MLLT4-AS1, MLN, MLST8, MMD2, MME, MME-AS1,
MMEL1, MMP11, MMP14, MMP15, MMP16, MMP17, MMP2, MMP21,
MMP23A, MMP23B, MMP25, MMP26, MMRN2, MN1, MND1, MNT, MOB2,
MOB3A, MOCS1P1, MOGAT2, MOK, MORG1, MORN1, MORN3, MORN4,
MOSC1, MOV10, MOXD1, MOXD2P, MPL, MPND, MPPED1, MPPED2, MPST,
MPZ, MPZL2, MRAP, MRC2, MRGPRF, MRPL23-AS1, MRPL28, MRPL2P1,
MRPL35P2, MRPL41, MRPS26, MRPS34, MRPS36, MRPS6, MRS2P2, MRVI1,
MRVI1-AS1, MS4A8B, MSANTD1, MSC, MSH4, MSI1, MSL3, MSL3L1, MSLN,
MSLNL, MSMB, MSN, MSNP1, MSRA, MSRB3, MST1, MST1P2, MST1P9,
MST1R, MSX2, MSX2P1, MTA1, MTCH2, MTF2, MTG1, MTHFD1L, MTHFR,
MTL5, MTMR14, MTMR4, MTMR9LP, MTND1P28, MTND5P1, MTND6P21,
MTNR1B, MTRNR2L1, MTRNR2L10, MTRNR2L5, MTTP, MTX1P1, MUC1,
MUC16, MUC19, MUC20, MUC3A, MUC5AC, MUC5B, MURC, MUSK, MVD,
MVK, MX2, MXD4, MXRA5, MXRA8, MYADML2, MYBL2, MYBPC1,
MYBPC2, MYCBPAP, MYCN, MYH1, MYH10, MYH13, MYH16, MYH4,
MYH7B, MYL4, MYL5, MYL6B, MYL6P2, MYL6P3, MYL9, MYLK, MYLK4,
MYLK-AS1, MYO10, MYO15A, MYO15B, MYO16, MYO18A, MYO18B,
MYO1A, MYO1C, MYO1G, MYO1H, MYO5C, MYO7A, MYO7B, MYO9B,
MYOM2, MYOM3, MYOZ3, MZF1, MZT2A, MZT2B, N4BP2, NAA10, NAA40,
NAA60, NAAA, NABP1, NACAD, NADK, NAGK, NAGLU, NAGPA, NAGS,
NANOS3, NAP1L1, NAP1L4P1, NAP1L6, NAPRT1, NAPSA, NARF, NASP,
NAT14, NAT6, NAV3, NBEAL2, NBPF8, NCAM1, NCAPD2P1, NCAPH, NCBP2-
AS1, NCCRP1, NCF1B, NCK2, NCKAP5L, NCKIPSD, NCOA5, NCS1, NDC80,
NDNF, NDOR1, NDRG2, NDST1, NDST3, NDST4, NDUFA13, NDUFA3,
NDUFA7, NDUFA9P1, NDUFB11, NDUFS7, NDUFS8, NEB, NECAB2, NECAB3,
NEDD9, NEFL, NEIL1, NEIL3, NEK2, NEK6, NEK8, NELL1, NES, NETO1,
NETO2, NEU4, NEURL1B, NEURL2, NEUROD4, NEUROG1, NEUROG3,
NFATC1, NFATC4, NFE2L3, NFRKB, NFYC, NGB, NGFR, NGFRAP1, NHLH1,
NHLRC4, NHS, NHSL1, NHSL2, NICN1, NICN1-AS1, NINJ1, NINL, NIPSNAP1,
NISCH, NKAIN2, NKAIN3, NKAIN4, NKD2, NKIRAS2, NKPD1, NKX2-5,
NKX6-2, NKX6-3, NLGN2, NLGN3, NLGN4X, NLN, NLRP12, NLRP6, NLRP9P,
NLRX1, NMBR, NME2, NME3, NME4, NME8, NMRK2, NNAT, NODAL, NOG,
NOP10, NOP16, NOS1, NOS1AP, NOS2, NOS2P3, NOSIP, NOTCH1, NOTCH2,
NOTCH3, NOTUM, NOVA1-AS1, NOVA2, NOX1, NOX4, NOXA1, NOXO1,
NPAS1, NPAS3, NPB, NPC1L1, NPDC1, NPEPL1, NPFFR1, NPFFR2, NPHP4,
NPHS1, NPHS2, NPIPL1, NPIPL2, NPM1P19, NPPB, NPR3, NPRL3, NPSR1,
NPSR1-AS1, NPTX1, NPTXR, NPW, NPY1R, NPY2R, NPY5R, NR1H3, NR1H4,
NR2F1, NR2F2, NR2F6, NR4A1, NR5A1, NR6A1, NRAP, NRARP, NRBP2, NREP,
NRG3, NRGN, NRK, NRL, NRN1, NRSN2, NRXN2, NRXN3, NSDHL, NSFL1C,
NSG1, NSRP1P1, NSUN5P1, NSUN5P2, NT5C3L, NT5DC2, NT5DC3, NT5E,
NT5M, NTF3, NTF4, NTHL1, NTM, NTN3, NTN4, NTN5, NTNG1, NTNG2,
NTRK3, NTS, NUBP2, NUBPL, NUCB1, NUCB1-AS1, NUDT11, NUDT14,
NUDT16L1, NUDT8, NUP210P3, NUS1P2, NUTF2, NXF2, NXF3, NXN, NXPE2,
NXPH2, NXPH4, NYAP1, NYNRIN, OBSCN, OBSL1, OCA2, OCM, ODF3B,
ODF3L1, ODF3L2, ODZ2, ODZ3, OGG1, OIT3, OLAH, OLFM3, OLFML1,
OLFML2A, OLFML3, ONECUT1, ONECUT2, ONECUT3, OPA1-AS1, OPA3,
OPCML, OPLAH, OPN5, OPRK1, OPRL1, OR10D1P, OR10V2P, OR11H7,
OR11Q1P, OR13C5, OR13E1P, OR13J1, OR13K1P, OR2A1, OR2A20P, OR2A3P,
OR2AJ1, OR2L3, OR2L5, OR2W3, OR51E2, OR5BH1P, OR7E158P, OR7E161P,
ORAI1, ORC6, OSBP2, OSBPL10-AS1, OSBPL1A, OSBPL7, OSGEPL1-AS1,
OSR1, OSR2, OSTN, OTC, OTOF, OTOG, OTOP1, OTOP3, OTUB1, OTX1,
OTX2, OVGP1, OVOL1, OXER1, OXLD1, P2RX1, P2RX6P, P2RY14, P2RY4,
P2RY6, P4HTM, PABPC1L, PABPC1L2A, PABPC1L2B, PABPC4L, PABPN1,
PABPN1L, PACS2, PACSIN3, PADI2, PAEP, PAFAH1B3, PAG1, PAGE2, PAH,
PAK4, PAK7, PALD1, PALM, PALM3, PAMR1, PANK1, PANK4, PANX2,
PAOX, PAPPA, PAPPA-AS1, PAPSS1, PAQR4, PAQR6, PAQR7, PAQR9,
PARD6G, PARP3, PARP4P2, PARP6, PARPBP, PATZ1, PAX2, PAX3, PAX4,
PAX7, PAX9, PBK, PBOV1, PBX1, PBX2, PBX2P1, PBX4, PBXIP1, PC, PCAT1,
PCBP4, PCCA-AS1, PCDH10, PCDH11X, PCDH15, PCDH18, PCDH19, PCDH8,
PCDH9, PCDHA1, PCDHA10, PCDHA11, PCDHA12, PCDHA13, PCDHA2,
PCDHA4, PCDHA5, PCDHA6, PCDHA7, PCDHA8, PCDHA9, PCDHB19P,
PCDHB2, PCDHGA10, PCDHGB4, PCDHGB6, PCDHGB8P, PCDHGC4,
PCED1A, PCED1B, PCED1B-AS1, PCGF2, PCNPP, PCNT, PCOLCE, PCOLCE-
AS1, PCP2, PCSK1N, PCSK4, PCSK5, PCSK6, PCYT1B, PCYT2, PDC, PDDC1,
PDE11A, PDE1A, PDE1C, PDE2A, PDE3B, PDE4A, PDE4C, PDE6B, PDE6C,
PDE7A, PDE9A, PDGFB, PDGFC, PDGFD, PDGFRB, PDIA2, PDK2, PDLIM3,
PDLIM4, PDLIM7, PDRG1, PDSS1, PDXP, PDZD3, PDZD4, PDZK1, PDZK1P1,
PDZRN4, PEBP4, PECR, PELI3, PENK, PER1, PES1P2, PEX11G, PEX6, PFDN4,
PFKFB1, PFKFB4, PFKL, PFKP, PFN1P1, PFN1P3, PGA5, PGAM2, PGAM4,
PGAP3, PGC, PGD, PGF, PGLS, PGLYRP1, PGM5, PGM5-AS1, PGM5P2,
PGPEP1, PHACTR1, PHACTR3, PHB2, PHBP10, PHBP12, PHF1, PHF16, PHF23,
PHGDH, PHLDA1, PHLDB1, PHLDB3, PHOX2A, PHPT1, PHRF1, PHTF1,
PHYHD1, PI16, PI4KA, PI4KAP1, PI4KAP2, PIANP, PICK1, PID1, PIDD,
PIEZO1, PIEZO2, PIF1, PIGL, PIGQ, PIGT, PIK3C2B, PIK3IP1, PIK3R1, PILRA,
PIN1, PINK1, PIP4K2B, PIP5K1B, PIP5K1C, PIP5K1P1, PIP5KL1, PISD, PITX1,
PITX2, PITX3, PIWIL1, PKD1, PKD1L2, PKD1P1, PKD2L1, PKDCC, PKHD1L1,
PKLR, PKM, PKMP1, PKMP3, PKMP4, PKMYT1, PKN1, PKN3, PKNOX2, PKP1,
PLA2G10, PLA2G12B, PLA2G2A, PLA2G4B, PLA2G4E, PLA2G4F, PLAC2,
PLAC8L1, PLAGL2, PLBD2, PLCB2, PLCD1, PLCD3, PLCG1, PLCG2, PLCH1-
AS2, PLCL1, PLD2, PLD3, PLD4, PLD5, PLDN, PLEC, PLEKHA8, PLEKHA9,
PLEKHB1, PLEKHD1, PLEKHF1, PLEKHG1, PLEKHG2, PLEKHG3,
PLEKHG4B, PLEKHG5, PLEKHG6, PLEKHG7, PLEKHH1, PLEKHH3,
PLEKHJ1, PLEKHO1, PLEKHO2, PLG, PLGLA, PLGLB2, PLIN1, PLIN2, PLIN3,
PLIN4, PLK1, PLK5, PLLP, PLN, PLOD1, PLS3, PLSCR3, PLSCR5, PLTP,
PLXDC1, PLXDC2, PLXNA1, PLXNB1, PLXNB3, PMAIP1, PMCHL1, PMCHL2,
PMEL, PMF1, PMM1, PMPCA, PNCK, PNKD, PNKP, PNMA3, PNMA6A,
PNMA6C, PNMA6D, PNMT, PNOC, PNPLA3, PNPLA7, POC1B-GALNT4,
PODNL1, PODXL, PODXL2, POLA1, POLD1, POLD2, POLDIP3, POLE, POLL,
POLN, POLR2KP1, POM121, POM121B, POM121L9P, POMT1, POPDC2,
POSTN, POTEF, POTEJ, POTEKP, POU2F2, POU3F1, POU3F2, POU3F4,
POU4F1, POU5F1B, POU5F1P3, POU5F1P4, POU5F1P6, POU6F2, POU6F2-AS1,
POU6F2-AS2, PPAP2B, PPAPDC3, PPARG, PPATP1, PPBPP2, PPDPF, PPEF1,
PPEF2, PPFIA2, PPFIA3, PPFIA4, PPFIBP1, PPFIBP2, PPIA, PPIAL4A, PPIAP11,
PPIAP21, PPIC, PPIL1P1, PPIL2, PPM1AP1, PPM1F, PPM1M, PPM1N, PPOX,
PPP1CA, PPP1CC, PPP1R11P1, PPP1R14A, PPP1R16A, PPP1R16B, PPP1R17,
PPP1R26, PPP1R2P10, PPP1R32, PPP1R3F, PPP1R3G, PPP2R1A, PPP2R2B,
PPP2R3A, PPP2R3B, PPP2R4, PPP2R5B, PPP3CA, PPP3R2, PPP4C, PPP4R4,
PPP6R2, PPY, PQLC2, PRAGMIN, PRAM1, PRAP1, PRB3, PRCD, PRDM12,
PRDM13, PRDM14, PRDM16, PRDM6, PRDM8, PRDX2, PRDX2P1, PRELID1P1,
PRELID2, PREX2, PRH2, PRICKLE1, PRICKLE2, PRICKLE4, PRKACG,
PRKAG3, PRKAR1B, PRKCB, PRKCG, PRKCSH, PRKCZ, PRKD1, PRKD2,
PRKG1, PRKG2, PRKRIP1, PRKY, PRLHR, PRMT1, PRMT8, PRO1853, PROC,
PROCA1, PRODH, PRODH2, PROK1, PROM1, PROM2, ProSAPiP1, PROX1,
PROX1-AS1, PRPF31, PRR12, PRR15L, PRR19, PRR22, PRR25, PRR3, PRR4,
PRR5, PRR5-ARHGAP8, PRRG2, PRRG3, PRRG4, PRRT2, PRRT4, PRSS12,
PRSS16, PRSS27, PRSS29P, PRSS30P, PRSS33, PRSS35, PRSS37, PRSS44,
PRSS45, PRSS46, PRSS50, PRSS8, PRTFDC1, PRTG, PRTN3, PRX, PSAT1P1,
PSAT1P4, PSD, PSD2, PSG1, PSG3, PSKH1, PSMC1P7, PSPN, PSRC1, PSTPIP2,
PTBP2, PTCHD1, PTCHD2, PTDSS2, PTF1A, PTGER1, PTGER2, PTGER3,
PTGER4, PTGES, PTGIS, PTH1R, PTK6, PTK7, PTMS, PTN, PTOV1, PTPN14,
PTPN18, PTPN2P2, PTPN5, PTPN6, PTPRB, PTPRCAP, PTPRD, PTPRF, PTPRG,
PTPRN2, PTPRO, PTPRQ, PTPRR, PTPRS, PTPRT, PTPRVP, PTPRZ1, PTRHD1,
PTTG1, PTTG1IP, PTTG4P, PURG, PVR, PVRL2, PVRL4, PWRN1, PXDN,
PXMP2, PYCARD, PYCR1, PYGB, PYROXD2, PYY, QARS, QDPR, QPCTL,
QPRT, QRSL1P3, QTRT1, R3HDML, RAB11B, RAB11FIP1P1, RAB11FIP3,
RAB17, RAB19, RAB1B, RAB1C, RAB25, RAB26, RAB27B, RAB33A, RAB34,
RAB36, RAB37, RAB38, RAB3A, RAB3D, RAB3IL1, RAB40C, RAB43,
RAB43P1, RAB6B, RABEP2, RABGGTA, RABL2A, RABL2B, RABL6, RAC3,
RAD51, RAD54L2, RAD9A, RADIL, RAGE, RALGDS, RALY, RAMP1, RAMP2,
RAMP2-AS1, RANBP1, RANBP10, RANBP17, RANBP3, RANGRF, RANP4,
RANP8, RAP1AP, RAP1GAP, RAP1GAP2, RAPGEF1, RAPGEF3, RAPGEF4,
RAPGEF4-AS1, RAPGEFL1, RAPSN, RARG, RARRES2, RASA3, RASA4CP,
RASGRP2, RASGRP4, RASIP1, RASL10B, RASL11B, RASSF10, RASSF2,
RASSF7, RASSF9, RAVER1, RBBP4P4, RBBP4P5, RBBP9, RBM10, RBM14-
RBM4, RBM24, RBM33, RBM44, RBM46, RBM5, RBM6, RBMS1, RBMS1P1,
RBMS2P1, RBMS3, RBMX2P5, RBP1, RBP2, RBP3, RBP5, RBPMS2, RCADH5,
RCC1, RCC2, RCCD1, RCE1, RCN3, RCOR2, RD3, RDH12, RDH16, RDM1,
REC8, RECQL4, RECQL5, REEP1, REEP2, REEP6, REG4, RELN, REM2, RENBP,
RERE, RERG, RERG-IT1, RESP18, RET, REXO1, RFNG, RFPL1, RFPL2, RFTN2,
RFWD2P1, RFX2, RFX4, RFX5, RFXANK, RGAG4, RGL2, RGL3, RGMA,
RGPD2, RGPD6, RGS12, RGS14, RGS3, RGS6, RGS8, RHBDF1, RHBDL1,
RHBDL2, RHBG, RHOA-IT1, RHOB, RHOBTB2, RHOC, RHOF, RHOH,
RHOT1P2, RHOT2, RHOU, RHOV, RHOXF1, RHPN1, RHPN1-AS1, RHPN2,
RIMBP2, RIMS1, RIMS3, RIMS4, RIPK4, RIT2, RLBP1, RLTPR, RMI2, RMST,
RNA5SP115, RNA5SP174, RNA5SP195, RNA5SP210, RNA5SP260, RNA5SP284,
RNA5SP298, RNA5SP316, RNA5SP320, RNA5SP340, RNA5SP364, RNA5SP37,
RNA5SP39, RNA5SP395, RNA5SP434, RNA5SP440, RNA5SP442, RNA5SP48,
RNA5SP482, RNA5SP493, RNA5SP495, RNA5SP507, RNA5SP78, RNA5SP82,
RNase_MRP, RNASE10, RNASE4, RNASEH1P1, RNASEH2A, RNASEH2B-AS1,
RND1, RND2, RND3, RNF112, RNF113B, RNF122, RNF144, RNF144A,
RNF144A-AS1, RNF150, RNF157, RNF165, RNF17, RNF182, RNF207, RNF208,
RNF215, RNF220, RNF224, RNF24, RNF26, RNF2P1, RNF31, RNF43, RNF44,
RNF6P1, RNF7P1, RNU12, RNU4-2, RNU5D-1, RNU5E-8P, RNU5F-1, RNU5F-
4P, RNU6-30, RNU6-48, RNU6-50, RNU6-60, RNU7-16P, RNU7-40P, RNU7-49P,
RNU7-57P, RNY1P4, RNY4P20, ROBO3, ROGDI, ROMO1, ROR2, ROS1, RP1-
102G20.4, RP1-102H19.6, RP11-1000B6.2, RP11-1000B6.3, RP11-1000B6.5, RP11-
1007J8.1, RP11-1008C21.2, RP11-100G15.7, RP11-100N21.1, RP11-1012A1.4,
RP11-101C21.1, RP11-101E14.2, RP11-1023L17.2, RP11-1024P17.1, RP11-
102M11.2, RP11-1035H13.3, RP11-1038A11.1, RP11-1038A11.3, RP11-103G8.1,
RP11-103G8.2, RP11-103H7.5, RP11-103J17.2, RP11-104D21.3, RP11-104E19.1,
RP11-104H15.8, RP11-104H15.9, RP11-104N10.1, RP11-1055B8.4, RP11-
1055B8.8, RP11-1058N17.1, RP11-105C19.2, RP11-106A1.2, RP11-106A1.3, RP11-
106E15.1, RP11-1072C15.1, RP11-1072C15.4, RP11-107F6.3, RP11-1081M5.1,
RP11-1082L8.1, RP11-1084I9.2, RP11-1085N6.5, RP11-108E14.1, RP11-108K14.4,
RP11-108L7.4, RP11-108L7.7, RP11-108M12.3, RP11-1099M24.8, RP11-109D20.1,
RP11-109I13.2, RP11-109L13.1, RP11-109N23.4, RP11-109P14.9, RP11-10A14.5,
RP11-10J21.5, RP11-10L12.2, RP11-10N23.5, RP11-10O17.3, RP11-1100L3.8,
RP11-110G21.1, RP11-110L15.2, RP11-1112C15.2, RP11-1114A5.4, RP11-111F5.2,
RP11-111G23.1, RP11-111H3.1, RP11-111I12.1, RP11-112J1.3, RP11-112N23.1,
RP11-1136G11.8, RP11-113I24.1, RP11-1143G9.2, RP11-1143G9.4, RP11-
114G22.1, RP11-114H7.1, RP11-1151B14.4, RP11-115C9.2, RP11-115K3.1, RP11-
115K3.2, RP11-115P21.1, RP11-1167A19.6, RP11-116K4.1, RP11-118B22.4, RP11-
118E18.2, RP11-1191J2.2, RP11-119F19.4, RP11-119F7.3, RP11-119F7.4, RP11-
119F7.5, RP11-119H12.4, RP11-11M20.2, RP11-11N5.1, RP11-11N9.4, RP11-
120B7.1, RP11-120M18.5, RP11-1212A22.1, RP11-1212A22.4, RP11-121G22.3,
RP11-121L11.1, RP11-1220K2.2, RP11-1223D19.1, RP11-122C5.3, RP11-
122D10.1, RP11-122K13.12, RP11-122K13.7, RP11-123K19.1, RP11-123K19.2,
RP11-124L5.7, RP11-124N14.3, RP11-124N14.4, RP11-124N19.3, RP11-124N2.1,
RP11-124O11.2, RP11-1258F18.1, RP11-126K1.6, RP11-126L15.4, RP11-126O1.5,
RP11-1277A3.2, RP11-127I20.7, RP11-128P10.1, RP11-129B22.1, RP11-129I19.2,
RP11-129M6.1, RP11-12J10.3, RP11-12M5.3, RP11-130F10.1, RP11-1319K7.1,
RP11-131L23.1, RP11-131N11.4, RP11-132A1.3, RP11-132G10.2, RP11-132N15.1,
RP11-133K1.7, RP11-133N21.10, RP11-134K1.2, RP11-134P9.3, RP11-135A24.4,
RP11-135J2.4, RP11-1365D11.1, RP11-136K7.1, RP11-136K7.3, RP11-1376P16.1,
RP11-1376P16.2, RP11-1379J22.3, RP11-1379J22.5, RP11-138B4.1, RP11-138H8.6,
RP11-1391J7.1, RP11-1396O13.1, RP11-1396O13.2, RP11-139F4.1, RP11-13A1.1,
RP11-13K12.1, RP11-13L2.4, RP11-1415C14.3, RP11-142A5.1, RP11-142C4.5,
RP11-142C4.6, RP11-142G1.2, RP11-142I20.1, RP11-143K11.1, RP11-143K11.5,
RP11-143M1.2, RP11-144G6.9, RP11-144O23.20, RP11-146D12.2, RP11-146F11.1,
RP11-147L13.2, RP11-148G20.1, RP11-148K1.12, RP11-148L24.1, RP11-
148O21.3, RP11-148O21.4, RP11-149F8.9, RP11-149I23.3, RP11-14C22.4, RP11-
150O12.1, RP11-152F13.3, RP11-152F13.7, RP11-152L20.2, RP11-153K11.3,
RP11-153K16.1, RP11-154D17.1, RP11-154D3.1, RP11-154D6.1, RP11-154J22.1,
RP11-156F12.1, RP11-156G14.6, RP11-156P1.3, RP11-157J24.1, RP11-157L3.5,
RP11-158J3.2, RP11-158L12.2, RP11-158M2.5, RP11-159D12.5, RP11-159F24.5,
RP11-159H22.2, RP11-159K7.2, RP11-159M11.2, RP11-159N11.3, RP11-15B17.1,
RP11-15B17.2, RP11-15N24.4, RP11-160A10.2, RP11-161H23.8, RP11-161M6.2,
RP11-162J8.2, RP11-163N6.2, RP11-164C12.1, RP11-164H5.1, RP11-165F24.3,
RP11-165J3.6, RP11-165O3.2, RP11-165P7.1, RP11-166O4.5, RP11-167H9.5,
RP11-167N4.2, RP11-167N4.4, RP11-168G16.2, RP11-168K11.2, RP11-168K11.3,
RP11-168O16.1, RP11-168O16.2, RP11-169D4.2, RP11-16E12.1, RP11-16E12.2,
RP11-16E23.3, RP11-16F15.2, RP11-16L21.7, RP11-170N11.1, RP11-171G2.1,
RP11-173A16.1, RP11-173A16.2, RP11-173D14.3, RP11-173D9.3, RP11-173G21.1,
RP11-173M1.8, RP11-174M13.2, RP11-174O3.3, RP11-175D17.3, RP11-175I17.4,
RP11-175P13.2, RP11-176F3.7, RP11-176H8.1, RP11-177B4.1, RP11-177F11.1,
RP11-177N22.2, RP11-178D16.1, RP11-178L8.3, RP11-17A4.2, RP11-17G12.2,
RP11-17G12.3, RP11-17M24.2, RP11-180A12.4, RP11-180I22.2, RP11-180P8.3,
RP11-181B18.1, RP11-181G12.2, RP11-181K12.2, RP11-182I10.3, RP11-182J1.12,
RP11-182J1.16, RP11-182J1.5, RP11-182L21.2, RP11-183E9.3, RP11-183G22.1,
RP11-183G22.3, RP11-187C18.3, RP11-187O7.1, RP11-187O7.3, RP11-188C12.2,
RP11-188C12.3, RP11-18D7.2, RP11-18I14.7, RP11-190P13.1, RP11-191G24.1,
RP11-192C21.1, RP11-192H23.7, RP11-195E2.1, RP11-196G11.1, RP11-196H14.4,
RP11-198M6.2, RP11-199F11.2, RP11-19O2.2, RP11-1C8.5, RP11-1E6.1, RP11-
1H8.3, RP11-1L12.3, RP11-1O10.1, RP11-203I2.1, RP11-203M5.4, RP11-204K16.1,
RP11-204L24.2, RP11-206L10.1, RP11-206L10.3, RP11-206L10.8, RP11-206P5.2,
RP11-209A2.1, RP11-209D14.2, RP11-20B24.4, RP11-20B24.7, RP11-20D14.6,
RP11-20G13.1, RP11-20G13.2, RP11-20G13.3, RP1-120G22.11, RP11-20I23.1,
RP11-20I23.11, RP11-20I23.6, RP11-20I23.8, RP11-20L24.1, RP11-20P5.2, RP11-
210M15.2, RP11-211A18.2, RP11-212D19.5, RP11-212F11.1, RP11-212I21.4,
RP11-213G2.5, RP11-214C8.2, RP11-214D15.2, RP11-214O1.2, RP11-215D10.1,
RP11-215E13.1, RP11-215P8.1, RP11-215P8.3, RP11-216C10.1, RP11-216M21.1,
RP11-216N14.7, RP11-216N14.8, RP11-216N14.9, RP11-218C14.5, RP11-
218E20.6, RP11-219B4.3, RP11-219B4.7, RP11-219G17.4, RP11-21A7A.2, RP11-
21A7A.3, RP11-21A7A.4, RP11-21B21.4, RP1-121G13.3, RP11-21G15.1, RP11-
21L23.2, RP11-21L23.4, RP11-21M24.2, RP11-21M24.3, RP11-21N7.2, RP11-
223I10.1, RP11-224O19.2, RP11-224O19.4, RP11-225B17.1, RP11-225H22.4,
RP11-225H22.5, RP11-225N10.1, RP11-227G15.2, RP11-227G15.6, RP11-
227G15.8, RP11-227H15.5, RP11-227J5.3, RP11-229C3.2, RP11-229P13.2, RP11-
229P13.22, RP11-229P13.23, RP11-22A3.2, RP11-22B23.1, RP11-22M7.2, RP1-
122O8.7, RP1-122P22.2, RP11-22P6.2, RP11-230B22.1, RP11-231C14.3, RP11-
231C14.4, RP11-233G1.4, RP11-234B24.5, RP11-235E17.4, RP11-235E17.6, RP11-
238K6.1, RP11-239E10.2, RP11-239L20.5, RP11-23J18.1, RP11-23J9.5, RP11-
23P13.4, RP11-240L7.4, RP11-243M5.1, RP11-244C20.1, RP11-244F12.3, RP11-
244N9.4, RP11-244O19.1, RP11-245J9.4, RP11-247A12.1, RP11-247A12.2, RP11-
247I13.7, RP11-248B10.2, RP11-24M17.3, RP11-24M17.7, RP11-250B2.3, RP11-
250B2.5, RP11-251A15.4, RP11-251G23.2, RP11-252E2.1, RP11-252K23.1, RP11-
252K23.2, RP11-255A11.21, RP11-255G12.2, RP11-258B16.1, RP11-259G18.2,
RP11-259G18.3, RP11-259O2.1, RP11-259P1.1, RP11-25B7.1, RP11-25H12.1, RP1-
125I3.2, RP11-25K19.1, RP11-260M19.2, RP11-261N11.8, RP11-261P9.4, RP11-
262H14.1, RP11-262H14.10, RP11-262H14.11, RP11-262I2.2, RP11-263K19.4,
RP11-263K19.6, RP11-264B14.1, RP11-264B17.4, RP11-264B17.5, RP11-264L1.4,
RP11-264P13.2, RP11-265B8.4, RP11-265D17.2, RP11-266K4.1, RP11-266K4.9,
RP11-266L9.1, RP11-266L9.2, RP11-266L9.3, RP11-266L9.4, RP11-266L9.5,
RP11-266N13.2, RP11-266O8.1, RP11-267D19.2, RP11-267J23.4, RP11-267M23.4,
RP11-268I9.1, RP11-268I9.3, RP11-268P4.4, RP11-269C23.3, RP11-269C4.2,
RP11-269G24.4, RP11-26H16.1, RP11-26J3.1, RP11-26M5.2, RP11-271O3.1, RP11-
272D12.2, RP11-272J7.4, RP11-273B20.1, RP11-273G15.2, RP11-274B21.2, RP11-
274B21.3, RP11-274B21.4, RP11-274B21.5, RP11-274J15.2, RP11-274J7.3, RP11-
274K13.2, RP11-276A18.2, RP11-276H19.2, RP11-276M12.1, RP11-277B15.1,
RP11-278L15.2, RP11-278L15.6, RP11-279F6.1, RP11-279F6.2, RP11-279F6.3,
RP11-279N8.3, RP1-127H14.3, RP11-281P23.2, RP11-282K24.2, RP11-283I3.1,
RP11-283I3.2, RP11-284A20.2, RP11-285G1.2, RP11-286H14.4, RP11-286H14.6,
RP11-286H14.8, RP11-287A8.6, RP11-288G3.4, RP11-288L9.4, RP1-128O3.6,
RP11-290C10.1, RP11-290F5.2, RP11-290L1.3, RP11-290P14.2, RP11-291L22.3,
RP11-292D4.3, RP11-292F22.3, RP11-293D9.2, RP11-295G12.1, RP11-295M3.2,
RP11-295P9.3, RP11-295P9.6, RP11-296L22.4, RP11-296L22.8, RP11-297D21.2,
RP11-297D21.3, RP11-297L17.2, RP11-297P16.3, RP11-298J20.4, RP11-298P3.4,
RP11-299G20.2, RP11-29H23.4, RP11-2C24.4, RP11-2C24.6, RP11-2E17.1, RP1-
12G14.6, RP11-300E4.2, RP11-302B13.1, RP11-302I18.1, RP11-302J23.1, RP11-
302M6.2, RP11-303E16.7, RP11-303G3.6, RP11-304C16.3, RP11-304F15.7, RP11-
304L19.1, RP11-304L19.3, RP11-304L19.4, RP11-304L19.8, RP11-304L20.1, RP11-
304M2.2, RP11-305F18.1, RP11-306K13.1, RP11-307C19.2, RP11-307I14.3, RP11-
307P5.1, RP11-308B16.1, RP11-308D16.2, RP11-309I15.1, RP11-309L24.2, RP1-
130H16.16, RP11-30K9.4, RP11-310E22.5, RP11-310I24.1, RP11-310P5.2, RP11-
311F12.1, RP11-311P8.2, RP11-313A24.1, RP11-313D6.4, RP11-313P13.3, RP11-
313P18.2, RP11-314M24.1, RP11-314P12.2, RP11-315I14.3, RP11-315I20.3, RP11-
316E14.6, RP11-316M1.3, RP11-316M21.7, RP11-317M11.1, RP11-317N8.2, RP11-
317O24.2, RP11-317P15.5, RP11-318C24.1, RP11-318C24.2, RP11-318K12.3,
RP11-318M2.3, RP11-319G5.1, RP11-319G9.3, RP11-31F15.1, RP11-31F19.1, RP1-
131H7.2, RP11-31I10.2, RP11-31I22.4, RP11-320G24.1, RP11-320L11.2, RP11-
320M16.1, RP11-321E8.1-001, RP11-322M19.2, RP11-324F21.1, RP11-324H6.7,
RP11-325F22.5, RP11-325L7.1, RP11-325L7.2, RP11-325P15.1, RP11-326C3.4,
RP11-326E22.1, RP11-326F20.5, RP11-326K13.4, RP11-327L3.1, RP11-328C8.4,
RP11-328K4.1, RP11-328L11.1, RP11-328P23.2, RP11-329A14.1, RP11-329J18.4,
RP11-329L6.1, RP11-32K4.2, RP11-330L19.1, RP11-330L19.4, RP11-330M19.1,
RP11-330M2.4, RP11-331F9.3, RP11-332H18.3, RP11-332H18.4, RP11-332H18.5,
RP11-332K15.1, RP11-333A23.2, RP11-333E1.1, RP11-333E13.4, RP11-334A14.2,
RP11-334E6.12, RP11-334J6.6, RP11-336N8.2, RP11-337C18.7, RP11-338K17.8,
RP11-339A11.2, RP11-339A7.1, RP11-33B1.2, RP11-33B1.3, RP11-33B1.4, RP11-
340E6.1, RP11-341G5.3, RP11-342C23.4, RP11-342M1.2, RP11-342M3.5, RP11-
343B5.1, RP11-343C2.7, RP11-343J3.2, RP11-344A16.2, RP11-344B5.4, RP11-
344E13.1, RP11-344E13.3, RP11-344E13.4, RP11-344E21.1, RP11-345J4.6, RP11-
345K20.2, RP11-345M22.2, RP11-346C20.2, RP11-346E8.1, RP11-347C12.2, RP11-
347C12.3, RP11-347C18.4, RP11-347H15.4, RP11-348M3.2, RP11-349A8.3, RP11-
349G13.2, RP11-349J5.2, RP11-349N19.2, RP11-34A14.3, RP11-34E5.2, RP11-
34E5.4, RP11-34P13.11, RP11-34P13.12, RP11-34P13.13, RP11-34P13.14, RP11-
350D17.2, RP11-350E12.5, RP11-350G13.1, RP11-350G8.7, RP11-350J20.4, RP11-
351A11.1, RP11-351I21.2, RP11-351J23.1, RP11-353H3.1, RP11-353N14.1, RP11-
353N14.5, RP11-354A14.1, RP11-354E11.2, RP11-354K4.1, RP11-354P11.2, RP11-
354P11.4, RP11-356C4.5, RP11-356K23.1, RP11-356O9.2, RP11-357C3.3, RP11-
357K6.1, RP11-357N13.3, RP11-357N13.7, RP11-358B23.1, RP11-358D14.2, RP11-
358L16.4, RP11-358L22.1, RP11-358M11.2, RP11-359I18.1, RP11-359M6.3, RP11-
359N11.2, RP11-359P5.1, RP11-35N6.1, RP11-360D2.2, RP11-360L9.4, RP11-
361H10.2, RP11-361H10.3, RP11-361M10.3, RP11-362F19.1, RP11-362K2.2,
RP11-363E6.3, RP11-363J20.2, RP11-363N22.2, RP11-364C11.2, RP11-364L4.1,
RP11-365D23.4, RP11-365F18.1, RP11-365F18.3, RP11-365N19.2, RP11-365P13.3,
RP11-366I13.3, RP11-366K18.3, RP11-366L20.2, RP11-366M4.3, RP11-367J11.2,
RP11-367O10.1, RP11-368I23.1, RP11-368I23.2, RP11-368I7.2, RP11-368I7.4,
RP11-368J21.3, RP11-368J22.2, RP11-368M16.3, RP11-368N21.1, RP11-369E15.1,
RP11-369E15.4, RP1-136J15.5, RP11-36N20.2, RP11-370B11.1, RP11-370I10.4,
RP11-371A19.2, RP11-371I1.2, RP11-373A6.1, RP11-373L8.2, RP11-374M1.7,
RP11-374P20.4, RP11-375B1.3, RP11-375F2.2, RP11-376M2.2, RP11-377D9.2,
RP11-377D9.3, RP11-377K22.3, RP11-377N20.1, RP11-378A13.1, RP11-378E13.3,
RP11-379B18.1, RP11-379B18.4, RP11-379B18.5, RP11-379C10.4, RP11-379F12.4,
RP11-379K17.4, RP11-379K17.9, RP11-379K22.2, RP11-37C7.3, RP11-37E23.5,
RP11-380D23.2, RP11-380I10.2, RP11-380I10.4, RP11-380M21.2, RP11-381E24.1,
RP11-381P6.1, RP11-382A18.1, RP11-382A20.2, RP11-382A20.5, RP11-382A20.6,
RP11-382A20.7, RP11-382D8.3, RP11-382J24.2, RP11-382M14.1, RP11-383B4.4,
RP11-383F6.1, RP11-383G10.1, RP11-383H13.1, RP11-384K6.2, RP11-384K6.6,
RP11-384L8.1, RP11-384P7.6, RP11-385J23.1, RP11-386G11.3, RP11-386G21.1,
RP11-386M24.5, RP11-388B24.3, RP11-388G3.1, RP11-388M20.1, RP11-
388M20.2, RP11-388M20.6, RP11-388M20.7, RP11-388P9.2, RP11-389G6.3, RP11-
389J22.1, RP1-138B7.5, RP11-38J22.3, RP11-38J22.6, RP11-38O14.1, RP11-
390F4.6, RP11-390P2.4, RP11-392B6.1, RP11-392E22.3, RP11-393I2.2, RP11-
393N4.2, RP11-394J1.2, RP11-395B7.2, RP11-395C3.1, RP11-395G17.1, RP11-
395P16.1, RP11-395P17.3, RP11-397A15.4, RP11-397A16.1, RP11-397D12.7,
RP11-397E7.1, RP11-397G17.1, RP11-397P13.7, RP11-398K22.12, RP11-399D2.1,
RP11-39E3.5, RP11-39K24.4, RP11-39M21.1, RP11-39M21.2, RP11-3B12.1, RP11-
3B12.4, RP11-3B7.1, RP11-3D4.2, RP11-3D4.3, RP11-3K24.1, RP11-3L10.3, RP11-
3L21.2, RP11-3N13.2, RP11-400N9.1, RP11-401F2.3, RP11-401F24.4, RP11-
401P9.4, RP11-401P9.5, RP11-401P9.6, RP11-403I13.5, RP11-403I13.6, RP11-
403P17.4, RP11-404K5.2, RP11-404P21.3, RP11-405A12.2, RP11-405L18.2, RP11-
406A9.2, RP11-408A13.1, RP11-408B11.2, RP11-408H20.1, RP11-408H20.2, RP11-
408O19.3, RP11-409K20.8, RP1-140A9.1, RP11-40H20.1, RP11-410D17.2, RP11-
410L14.1, RP11-410N8.3, RP11-410N8.4, RP11-411B10.2, RP11-411N4.1, RP11-
413E1.4, RP11-413H22.2, RP11-414H17.5, RP11-414H17.8, RP11-414P19.1, RP11-
415C15.2, RP11-417B4.2, RP11-417F21.2, RP11-417L19.4, RP11-419C23.1, RP11-
41L14.1, RP11-420K8.2, RP11-420N3.2, RP11-421E14.2, RP11-423F24.3, RP11-
424C20.2, RP11-424E7.3, RP11-425D10.2, RP11-425I13.2, RP11-425M5.5, RP11-
426A6.7, RP11-426A6.8, RP11-426A6.9, RP11-426L16.3, RP11-429J17.2, RP11-
429J17.5, RP11-429J17.6, RP11-429J17.8, RP11-429P3.2, RP11-429P3.3, RP11-
42D20.1, RP1-142L7.5, RP11-430B1.2, RP11-430C7.2, RP11-430C7.4, RP11-
430H10.2, RP11-430H10.3, RP11-430H12.2, RP11-430I15.4, RP11-431M7.3, RP11-
432B6.3, RP11-432I5.8, RP11-432J9.5, RP11-432M8.17, RP11-433J8.1, RP11-
433J8.2, RP11-434B12.1, RP11-434C1.1, RP11-434C1.2, RP11-434O22.1, RP11-
435F13.1, RP11-436A20.3, RP11-436D10.3, RP11-436D23.1, RP11-436H11.1,
RP11-436I9.6, RP11-436M15.3, RP11-438D14.2, RP11-439A17.9, RP11-439C15.2,
RP11-439C15.4, RP11-439E19.10, RP11-439E19.3, RP11-439E19.8, RP11-43N5.1,
RP11-441F2.1, RP11-441F2.2, RP11-443B7.1, RP11-443D10.2, RP11-444D3.1,
RP11-444I9.3, RP11-444J21.1, RP11-445F12.1, RP11-445H22.3, RP11-445H22.4,
RP11-445L6.3, RP11-446J8.1, RP11-449I17.4, RP11-449P1.1, RP11-449P15.1,
RP11-44D19.1, RP11-44F21.5, RP11-44M6.3, RP11-44N21.1, RP11-451M19.3,
RP11-452D2.2, RP11-452K12.4, RP11-452L6.5, RP11-453E17.2, RP11-
453F18_B.1, RP11-454K7.3, RP11-454L9.2, RP11-455F5.2, RP11-455F5.5, RP11-
456O19.2, RP11-456O19.4, RP11-457M11.5, RP11-458D21.1, RP11-458F8.1,
RP11-458F8.2, RP11-459F6.3, RP11-459K23.2, RP11-45H22.1, RP11-45J1.1, RP1-
145M24.1, RP11-45P15.4, RP11-460I13.2, RP11-460N20.4, RP11-460N20.5, RP11-
461F16.4, RP11-461O14.1, RP11-462B18.3, RP11-462G12.1, RP11-462G12.2,
RP11-462G2.2, RP11-462L8.1, RP11-463C14.1, RP11-463D19.1, RP11-463H12.1,
RP11-463O9.1, RP11-464D20.6, RP11-464F9.19, RP11-465B22.5, RP11-465I4.2,
RP11-465L10.7, RP11-465N4.2, RP11-466A19.6, RP11-466C23.4, RP11-466F5.9,
RP11-466P24.7, RP11-467D6.1, RP11-467J12.1, RP11-467L20.10, RP11-467L20.7,
RP11-468E2.4, RP11-468E2.9, RP11-468H14.2, RP11-469A15.2, RP11-469N6.1,
RP11-46C20.1, RP11-46C24.5, RP11-46C24.8, RP11-46D6.1, RP11-46F15.2, RP11-
470L19.2, RP11-472M19.2, RP11-473M14.2, RP11-473M20.11, RP11-473M20.15,
RP11-473M20.5, RP11-473M20.7, RP11-473M20.8, RP11-473O4.5, RP11-474D1.3,
RP11-474D14.2, RP11-474L23.3, RP11-476C8.3, RP11-476E15.3, RP11-476M19.2,
RP11-477D19.2, RP11-478B9.1, RP11-478C1.8, RP11-478C19.2, RP11-478C6.1,
RP11-478H13.1, RP11-479P21.1, RP11-480A16.1, RP11-480I12.4, RP11-480I12.5,
RP11-480N24.4, RP11-481K16.2, RP11-482D24.3, RP11-482M8.1, RP11-483C6.1,
RP11-484D2.4, RP11-485G7.5, RP11-485G7.6, RP11-485M7.3, RP11-486A14.1,
RP11-486A14.2, RP11-486G15.1, RP11-486O13.2, RP11-486O13.4, RP11-
486P11.1, RP11-487E1.1, RP11-488C13.5, RP11-488C13.6, RP11-488I20.5, RP11-
48B3.3, RP11-490G2.2, RP11-490M8.1, RP11-491F9.1, RP11-491F9.5, RP11-
491F9.6, RP11-492A10.1, RP11-492E3.2, RP11-493E3.1, RP11-493K19.3, RP11-
493K23.4, RP11-495O11.1, RP11-495P10.3, RP11-496I9.1, RP11-497E19.1, RP11-
497E19.2, RP11-497G19.1, RP11-497H16.2, RP11-497H16.4, RP11-497H16.6,
RP11-497H16.7, RP11-497H16.8, RP11-498B4.5, RP11-498D10.4, RP11-498P14.5,
RP11-499F3.1, RP11-499F3.2, RP1-149A16.12, RP11-49C24.1, RP11-49C9.2,
RP11-49K24.6, RP1-149L1.1, RP11-49M9.2, RP11-49O14.3, RP11-4E23.2, RP11-
4F22.2, RP11-4K3_A.2, RP11-500G22.2, RP11-501C14.5, RP11-501C14.7, RP11-
503C24.6, RP11-503G7.2, RP11-504A18.1, RP11-504I13.3, RP11-504P24.4, RP11-
504P24.5, RP11-505E24.2, RP11-505E24.3, RP11-506H20.1, RP11-50B3.2, RP11-
50E11.3, RP1-150O5.3, RP11-510H23.1, RP11-510J16.3, RP11-510J16.5, RP11-
510M2.4, RP11-510N19.5, RP11-512H23.2, RP11-512M8.3, RP11-513D4.1, RP11-
513D5.2, RP11-513G11.4, RP11-514P8.2, RP11-516A11.3, RP11-517C16.2, RP11-
517C16.4, RP11-517P14.2, RP11-517P14.6, RP11-519G16.2, RP1-151B14.6, RP11-
51M18.1, RP11-520B13.4, RP11-520D19.2, RP11-521B24.5, RP11-521C20.3,
RP11-521I2.3, RP11-522B15.1, RP11-522N14.1, RP11-522N14.2, RP11-524F11.2,
RP11-524L6.2, RP11-525K10.3, RP11-526N18.1, RP11-528A4.2, RP11-529F4.1,
RP11-529H22.1, RP11-529K1.4, RP1-152L7.3, RP11-530A18.1, RP11-531A21.5,
RP11-531A24.5, RP11-533K9.3, RP11-535A19.1, RP11-535K18.2, RP11-538I12.3,
RP11-539I5.1, RP11-539L10.2, RP11-53B2.2, RP11-540O11.1, RP11-540O11.6,
RP11-541P9.1, RP11-541P9.3, RP11-542B15.1, RP11-542C16.1, RP11-542C16.2,
RP11-543C4.1, RP11-543C4.3, RP11-543D5.2, RP11-543H15.2, RP11-544L8_B.4,
RP11-544M22.3, RP11-545A16.1, RP11-545M17.1, RP11-545M17.2, RP11-
545N8.3, RP11-546B8.5, RP11-546K22.1, RP11-547I7.2, RP11-547L9.1, RP11-
548H18.2, RP11-548K12.12, RP11-54A4.2, RP11-54C4.1, RP11-54K16.2, RP11-
54O7.1, RP11-54O7.3, RP11-551M15.1, RP11-552F3.4, RP11-552J9.9, RP11-
552M11.2, RP11-552M11.4, RP11-554A11.5, RP11-554A11.9, RP11-554D14.4,
RP11-554D14.7, RP11-554D20.1, RP11-554F20.1, RP11-556H2.3, RP11-556O5.1,
RP11-556O5.3, RP11-556O5.4, RP11-556O5.5, RP11-557J10.3, RP11-561B11.6,
RP11-561C5.4, RP11-561N12.5, RP11-561N12.6, RP11-562A8.3, RP11-565H13.2,
RP11-565J7.1, RP11-565N2.1, RP11-566K19.5, RP11-567C20.2, RP11-567J20.1,
RP11-567M16.1, RP11-568N6.1, RP11-569G9.7, RP11-56B16.2, RP11-56G10.2,
RP11-571E6.1, RP11-571F15.3, RP11-571M6.1, RP11-571M6.8, RP11-572M11.3,
RP11-572M11.4, RP11-573I11.2, RP11-574H13.2, RP11-574K11.16, RP11-
574K11.20, RP11-575G13.2, RP11-575L7.2, RP11-576D8.4, RP11-578F21.10,
RP11-578F21.2, RP11-578F21.3, RP11-578F21.5, RP11-578F21.9, RP11-578O24.2,
RP11-57A19.2, RP11-57H12.3, RP11-57K17.1, RP11-582E3.2, RP11-582J16.3,
RP11-584P21.4, RP11-585F1.10, RP11-586D19.2, RP11-58A12.3, RP11-58G13.1,
RP11-58K22.2, RP11-592B15.3, RP11-593F23.1, RP11-594N15.2, RP11-594N15.3,
RP11-597A11.3, RP11-598E7.5, RP11-598F7.6, RP11-598O12.1, RP11-598P20.3,
RP11-59D5_B.2, RP11-59J16.1, RP11-5J2.2, RP11-5L12.1, RP11-5N11.2, RP11-
5N11.3, RP11-5P18.10, RP11-5P22.1, RP11-600F24.7, RP11-603J24.14, RP11-
603J24.6, RP11-60H5.1, RP11-60L3.1, RP11-611D20.2, RP11-611L7.1, RP11-
613F7.1, RP11-615I2.1, RP11-615I2.2, RP11-615I2.3, RP11-615I2.7, RP11-615J4.4,
RP11-616K22.3, RP11-616M22.3, RP11-617D20.1, RP11-617F23.1, RP11-
618N24.1, RP11-619A14.2, RP11-61A14.4, RP11-61E11.2, RP11-61J19.4, RP11-
621K7.1, RP11-621L6.2, RP11-624G17.3, RP11-624L12.1, RP11-624L4.1, RP11-
625L16.3, RP11-626G11.5, RP11-627G18.1, RP11-627G18.3, RP11-627G23.1,
RP11-628O18.1, RP11-629G13.1, RP11-629O4.1, RP11-62C3.6, RP11-62E14.2,
RP11-632F7.1, RP11-635L1.3, RP11-635N19.1, RP11-638I2.2, RP11-638I2.4,
RP11-638I2.6, RP1-163G9.1, RP1-163M9.6, RP11-640I15.1, RP11-640N20.6, RP11-
641A6.9, RP11-642P15.1, RP11-643G16.3, RP11-644F5.10, RP11-645C24.5, RP11-
646J21.3, RP11-649A18.4, RP11-649A18.5, RP11-649A18.7, RP11-64C12.2, RP11-
64K12.10, RP11-650K20.3, RP11-651P23.5, RP11-651P23.6, RP11-655M14.12,
RP11-655M14.13, RP11-656D10.6, RP11-656G20.1, RP11-657O9.1, RP11-65F13.2,
RP11-65I12.1, RP11-65M17.3, RP11-65N13.4, RP11-660I20.1, RP11-661A12.9,
RP11-661D19.1, RP11-662J14.1, RP11-662M24.2, RP11-665G4.1, RP11-666A20.3,
RP11-666A8.11, RP11-666A8.7, RP11-666A8.9, RP11-666I19.2, RP11-669B18.1,
RP11-669N7.3, RP11-66D17.5, RP11-66N24.3, RP11-66N24.4, RP11-670P16.3,
RP11-671E7.1, RP11-671M22.1, RP11-671M22.5, RP11-673P17.2, RP11-674E16.1,
RP11-675F6.4, RP11-676J12.8, RP11-676J15.1, RP11-677M14.2-001, RP11-
677M14.3, RP11-677M14.7, RP11-677O4.1, RP11-677O4.6, RP11-678G15.2, RP1-
167A14.2, RP11-67L3.5, RP11-67M1.1, RP11-680F20.4, RP11-680F8.1, RP11-
681L4.1, RP11-681L8.1, RP11-683L23.1, RP11-685I11.1, RP11-685M7.3, RP11-
685M7.5, RP11-686D22.8, RP11-686F15.2, RP11-687F6.1, RP11-689K5.3, RP11-
68I18.10, RP11-68I3.11, RP11-68I3.2, RP11-68I3.4, RP11-68I3.5, RP11-68L1.2,
RP1-168L15.5, RP11-68L18.1, RP11-690G19.3, RP11-691H4.1, RP11-692C23.1,
RP11-694D5.1, RP11-695J20.1, RP11-696L21.2, RP11-696P8.2, RP11-697E2.6,
RP11-697E22.2, RP11-697M17.1, RP11-69D4.3, RP11-69D4.5, RP11-69I8.3, RP11-
69M1.3, RP11-6J21.2, RP11-701H24.4, RP11-703I16.1, RP11-704M14.1, RP11-
705C15.2, RP11-705C15.3, RP11-705C15.4, RP11-705O24.1, RP11-706O15.1,
RP11-706O15.3, RP11-707G14.3, RP11-707G14.7, RP11-708L7.6, RP11-709A23.2,
RP11-70C1.3, RP11-70F11.2, RP11-711G10.1, RP11-711M9.1, RP11-712L6.7,
RP11-713C5.1, RP11-714L20.1, RP11-715J22.2, RP11-715J22.3, RP11-715J22.4,
RP11-715J22.6, RP11-717D12.1, RP11-718B12.2, RP11-718B12.3, RP11-718O11.1,
RP11-719K4.1, RP11-719K4.2, RP11-71E19.1, RP11-71H17.7, RP11-723C11.2,
RP11-723O4.2, RP11-723O4.9, RP11-728F11.4, RP1-172N19.3, RP11-731J8.1,
RP11-731J8.2, RP11-732A19.5, RP11-732A19.6, RP11-732A19.8, RP11-732A19.9,
RP11-734C14.10, RP11-736N17.4, RP11-739G5.1, RP11-744H18.1, RP11-744I24.3,
RP11-745A24.2, RP11-745C15.2, RP11-745O10.2, RP11-747H12.6, RP11-
748L13.1, RP11-74C13.4, RP11-74H8.1, RP11-74K19.1, RP11-753D20.1, RP11-
753H16.5, RP11-754B17.1, RP11-755B10.2, RP11-755F10.1, RP11-757C15.4,
RP11-757F18.3, RP11-757G1.5, RP11-758C21.1, RP11-75A9.1, RP11-75A9.3,
RP11-75C9.1, RP11-75N4.2, RP11-760D2.10, RP11-760N9.1, RP11-761E20.1,
RP11-762B21.5, RP11-763B22.9, RP11-764K9.1, RP11-764K9.2, RP11-764K9.3,
RP11-767I20.1, RP11-767L7.1, RP11-768F21.1, RP11-769O8.3, RP11-76C10.5,
RP11-76E16.2, RP11-76H14.2, RP11-76K13.3, RP11-770G2.5, RP11-771F20.1,
RP11-773H22.1, RP11-779O18.3, RP11-77H9.2, RP11-77H9.5, RP11-77P16.4,
RP11-780K2.1, RP11-781P6.1, RP11-783K16.13, RP11-783K16.5, RP11-785H5.1,
RP11-785H5.2, RP11-788H18.1, RP11-78F17.1, RP11-790G19.2, RP11-791G16.4,
RP11-793H13.9, RP11-795F19.1, RP11-795F19.5, RP11-796E2.4, RP11-797A18.3,
RP11-797E24.3, RP11-797M17.1, RP11-798G7.5, RP11-79C6.1, RP11-79H23.3,
RP11-800A3.7, RP11-802E16.3, RP11-803B1.5, RP11-806L2.2, RP11-807H7.1,
RP11-809N8.5, RP11-809O17.1, RP11-80A15.1, RP11-80B9.1, RP11-80H18.3,
RP11-815J21.2, RP11-815J4.7, RP11-817O13.2, RP11-817O13.8, RP11-81H3.2,
RP11-81K13.1, RP11-81K2.1, RP11-820I16.1, RP11-823P9.1, RP11-823P9.3, RP11-
826N14.4, RP11-829H16.2, RP11-829H16.3, RP11-82C23.2, RP11-82K18.2, RP11-
82L18.2, RP11-82L18.4, RP11-82L7.4, RP1-182O16.2, RP11-82O19.1, RP11-
831A10.1, RP11-834C11.11, RP11-834C11.3, RP11-834C11, 4, RP11-834C11.7,
RP11-839D17.2, RP11-839G9.1, RP11-83A24.1, RP11-83M16.3, RP11-83M16.4,
RP11-83M16.5, RP11-83M16.6, RP11-841C19.6, RP11-843B15.2, RP11-844P9.2,
RP11-845C23.3, RP11-846F4.12, RP11-848D3.4, RP11-848G14.2, RP11-848G14.5,
RP11-848P1.4, RP11-848P1.5, RP11-849F2.5, RP11-849H4.4, RP11-849I19.1,
RP11-84A14.5, RP1-184J9.2, RP11-84O12.2, RP11-84P7.2, RP11-854K16.1, RP11-
855A2.3, RP11-855A2.5, RP11-855O10.2, RP11-856F16.2, RP11-85B7.2, RP11-
862G15.2, RP11-862L9.3, RP11-863K10.2, RP11-863P13.4, RP11-867G2.8, RP11-
867G23.2, RP11-867G23.3, RP11-867G23.4, RP11-867G23.8, RP11-86H7.1, RP11-
86H7.7, RP11-871F6.2, RP11-875O11.1, RP11-876N24.2, RP1-187B23.1, RP11-
87C12.5, RP11-87G24.3, RP11-87H9.2, RP11-87H9.3, RP11-87N24.2, RP11-
883G14.2, RP11-883G14.3, RP11-885B4.1, RP11-889L3.4, RP11-88H10.2, RP11-
88H9.1, RP11-88H9.2, RP11-88I18.4, RP11-88I21.1, RP11-88K11.1, RP11-
88M19.3, RP11-893F2.5, RP11-903H12.3, RP11-907D1.2, RP11-909N17.3, RP11-
90B22.2, RP11-90B9.2, RP11-90D4.1, RP11-90D4.2, RP11-90H3.2, RP11-90K6.1,
RP11-90M11.1, RP11-91A18.4, RP11-91A18.5, RP11-91I20.3, RP11-91K8.2, RP11-
91K9.1, RP11-91N2.3, RP11-91P24.5, RP11-91P24.7, RP11-923I11.4, RP11-
923I11.5, RP11-923I11.6, RP11-923I11.7, RP11-930O11.1, RP11-930P14.1, RP11-
930P14.2, RP11-939C17.2, RP1-193H18.2, RP11-93I21.3, RP11-93K22.11, RP11-
93O14.2, RP11-944L7.2, RP11-944L7.4, RP11-945A13.1, RP11-945C19.1, RP11-
945C19.4, RP11-94C24.8, RP11-94M14.2, RP11-958N24.1, RP11-958N24.2, RP11-
95M15.1, RP11-95M15.2, RP11-95O2.1, RP11-95O2.5, RP11-95P13.1, RP11-
961A15.1, RP11-96C23.10, RP11-96C23.7, RP11-96C23.8, RP11-96D1.5, RP11-
96G10.1, RP11-96H17.2, RP11-96K19.4, RP11-96L7.2, RP11-972L6.2, RP11-
973N13.2, RP11-974F13.6, RP11-977G19.5, RP11-97O12.2, RP1-197O17.2, RP11-
981G7.3, RP11-981P6.1, RP11-986G18.1, RP11-989F5.3, RP11-98G7.1, RP11-
998D10.2, RP11-998D10.7, RP11-99A14.1, RP1-199J3.5, RP11-9M16.2, RP1-
200G19.1, RP1-203P18.1, RP1-206D15.5, RP1-228H13.1, RP1-234P15.4, RP1-
240B8.3, RP1-240K6.3, RP1-241P17.1, RP1-241P17.3, RP1-249I4.2, RP1-257A7.4,
RP1-261D10.1, RP1-265C24.8, RP1-266L20.4, RP1-266L20.9, RP1-269M15.3, RP1-
272J12.1, RP1-272L16.1, RP1-273P12.3, RP1-279N11.1, RP1-283E3.4, RP1-
288H2.2, RP1-288L1.4, RP1-288L1.5, RP1-292L20.1, RP1-295F6.2, RP1-302D9.3,
RP1-304B14.3, RP1-308E4.1, RP13-100B2.4, RP1-310O13.12, RP1-310O13.7,
RP13-131K19.2, RP13-140E4.1, RP13-15E13.1, RP13-15M17.1, RP13-192B19.2,
RP13-221M14.5, RP13-225O21.2, RP13-25N22.1, RP13-262C2.1, RP13-279N23.2,
RP1-32I10.10, RP13-36G14.3, RP13-371D18.2, RP13-379L11.2, RP13-395E19.2,
RP13-39P12.2, RP13-39P12.3, RP13-401N8.1, RP13-439H18.7, RP13-467H17.1,
RP13-494C23.1, RP1-34B20.4, RP13-516M14.4, RP13-608F4.4, RP13-638C3.2,
RP13-726E6.1, RP13-726E6.2, RP1-37C10.3, RP13-7D7.1, RP13-884E18.2, RP13-
890H12.2, RP13-895J2.7, RP13-93L13.2, RP13-977J11.2, RP13-98N21.2, RP13-
991F5.2, RP13-991F5.4, RP1-40E16.11, RP1-40E16.8, RP1-4G17.2, RP1-56J10.8,
RP1-59D14.1, RP1-59D14.6, RP1-5O6.6, RP1-60O19.2, RP1-63G5.5, RP1-63G5.7,
RP1-64K7.4, RP1-67A8.3, RP1-67M12.2, RP1-85F18.6, RP1-89D411, RP1-
90G24.10, RP1-90G24.6, RP1-90L6.2, RP1-97J1.2, RP3-324N14.2, RP3-329E20.2,
RP3-330M21.5, RP3-336K20_B.2, RP3-337H4.8, RP3-354N19.3, RP3-370M22.8,
RP3-377D14.1, RP3-382I10.3, RP3-399L15.3, RP3-404F18.5, RP3-405J10.4, RP3-
406P24.3, RP3-410B11.1, RP3-412A9.10, RP3-414A15.11, RP3-415N12.1, RP3-
423B22.5, RP3-428L16.1, RP3-434P1.6, RP3-439F8.1, RP3-441A12.1, RP3-
449O17.1, RP3-462E2.3, RP3-465N24.5, RP3-467K16.4, RP3-468B3.2, RP3-
470B24.5, RP3-473L9.3, RP3-476K8.3, RP3-476K8.4, RP3-477O4.5, RP3-
481A17.1, RP3-497J21.1, RP3-508I15.10, RP3-508I15.14, RP3-510D11.2, RP3-
510L9.1, RP3-510O8.4, RP3-511B24.5, RP3-512B11.3, RP3-514A23.2, RP3-
518E13.2, RP3-522P13.3, RP3-523C21.1, RP3-523K23.2, RP3-525N10.2, RP4-
530I15.6, RP4-533D7.4, RP4-533D7.5, RP4-534N18.2, RP4-536B24.4, RP4-
537K23.4, RP4-539M6.14, RP4-541C22.5, RP4-545K15.3, RP4-545K15.5, RP4-
545L17.7, RP4-549L20.2, RP4-555D20.2, RP4-555L14.4, RP4-560B9.1, RP4-
562J12.2, RP4-566D2.1, RP4-575N6.2, RP4-583P15.10, RP4-583P15.11, RP4-
584D14.6, RP4-584D14.7, RP4-591N18.2, RP4-593C16.3, RP4-601K24.1, RP4-
601P9.2, RP4-603I14.3, RP4-604G5.1, RP4-612J11.1, RP4-620F22.3, RP4-622L5.7,
RP4-633O19_A.1, RP4-635E18.7, RP4-639F20.1, RP4-639F20.3, RP4-647C14.2,
RP4-647C14.5, RP4-650F12.2, RP4-655J12.3, RP4-663N10.1, RP4-665J23.2, RP4-
669L17.10, RP4-669P10.16, RP4-673D20.3, RP4-673D20.4, RP4-676L2.1, RP4-
686C3.7, RP4-694B14.4, RP4-697K14.7, RP4-701O16.5, RP4-706L14.2, RP4-
710M16.2, RP4-724E16.2, RP4-728D4.2, RP4-730K3.3, RP4-734P14.4, RP4-
738P11.4, RP4-742J24.2, RP4-745E8.2, RP4-747G18.5, RP4-753P9.3, RP4-
756H11.3, RP4-758J18.10, RP4-758J18.7, RP4-758J18.8, RP4-758J18.9, RP4-
763G1.1, RP4-765C7.1, RP4-773A18.4, RP4-773N10.5, RP4-777D9.2, RP4-
781B1.2, RP4-781K5.4, RP4-782L23.2, RP4-784A16.2, RP4-791K14.2, RP4-
792G4.2, RP4-796I8.1, RP4-798A17.5, RP4-798C17.5, RP4-799P18.2, RP4-
799P18.5, RP4-800G7.2, RP4-811H24.6, RP5-1024C24.1, RP5-1025A1.2, RP5-
1039K5.13, RP5-1041C10.3, RP5-1052I5.1, RP5-1061H20.3, RP5-1061H20.4, RP5-
1063M23.1, RP5-1068B5.3, RP5-1071N3.1, RP5-1086D14.3, 105-1091E12.1, RP5-
1096D14.6, RP5-1099D15.1, RP5-1101C3.1, RP5-1103B4.3, RP5-1107A17.4, RP5-
1119A7.11, RP5-1119D9.4, RP5-1142A6.3, RP5-1142A6.9, RP5-1172N10.2, RP5-
1177E19.2, RP5-1184F4.5, RP5-1187M17.10, RP5-1189B24.4, RP5-827C21.1, RP5-
837I24.1, RP5-837I24.6, RP5-839B4.8, RP5-854E16.2, RP5-864K19.4, RP5-
875H3.2, RP5-875O13.1, RP5-881L22.4, RP5-881L22.5, RP5-882C2.2, RP5-
884C9.2, RP5-890O3.3, RP5-894D12.3, RP5-894H24.2, RP5-896L10.1, RP5-
901A4.1, RP5-902P8.10, RP5-902P8.12, RP5-906A24.2, RP5-914M6.1, RP5-
914P20.5, RP5-915N17.3, RP5-928E24.2, RP5-942I16.1, RP5-955M13.3, RP5-
955M13.4, RP5-968J1.1, RP5-968P14.2, RP5-977B1.10, RP5-977B1.11, RP5-
982E9.1, RP5-991G20.1, RP5-997D24.3, RP5-998N21.7, RP6-105D16.1, RP6-
159A1.2, RP6-170F5.2, RP6-239D12.1, RP6-24A23.3, RP6-24A23.7, RP6-33F8.1,
RP6-65G23.3, RP9P, RPGRIP1, RPL12P33, RPL12P8, RPL13AP20, RPL17P25,
RPL18AP7, RPL18P13, RPL19P12, RPL21P116, RPL21P119, RPL21P120,
RPL21P122, RPL21P132, RPL21P2, RPL21P4, RPL21P54, RPL23AP10,
RPL23AP80, RPL31P52, RPL32P3, RPL32P34, RPL34P27, RPL35AP31,
RPL35AP33, RPL36P4, RPL37P1, RPL39L, RPL3P3, RPL3P9, RPL5P30,
RPL6P25, RPL6P30, RPL7AP11, RPL7AP58, RPL7AP73, RPL7L1P11, RPL7P14,
RPL7P3, RPL7P47, RPL9P18, RPL9P2, RPL9P32, RPL9P8, RPLP0P2, RPRM,
RPRML, RPS12P28, RPS15AP10, RPS15AP16, RPS15AP18, RPS15AP5, RPS15P5,
RPS16P8, RPS19P3, RPS20P15, RPS20P33, RPS23P6, RPS26P11, RPS26P31,
RPS26P49, RPS26P55, RPS26P6, RPS27P12, RPS27P16, RPS27P25, RPS2P1,
RPS2P5, RPS2P7, RPS3AP49, RPS3P6, RPS5P3, RPS6KA2, RPS6KA2-IT1,
RPS6KB2, RPS6KL1, RPS7P14, RPS9P1, RPSAP14, RPSAP43, RPSAP52,
RPSAP8, RPSAP9, RPTOR, RPUSD1, RRAS, RRM2, RRM2P3, RRN3P3, RRP7B,
RSF1-IT1, RSL24D1P2, RSL24D1P5, RSPO3, RSPO4, RTEL1P1, RTKN, RTKN2,
RTL1, RTN2, RTN4R, RTN4RL1, RTN4RL2, RUNDC3A, RWDD4P1, RXFP4,
RYK, RYKP1, RYR1, S100A1, S100A10, S100A13, S100A14, S100A16, S100A4,
S100P, S100Z, S1PR2, S1PR3, S1PR4, SAC3D1, SAE1, SALL1, SALL3, SALL4P5,
SAMD10, SAMD14, SAMD15, SAMD4A, SAMD5, SAMHD1, SAMM50,
SAP30BP, SAPCD2, SARDH, SARM1, SARS2, SASH1, SBF1P1, SBK1, SBK2,
SBSPON, SC65, SCAF1, SCAMP4, SCAMP5, SCAP, SCARA3, SCARB1,
SCARF2, SCARNA11, SCARNA16, SCARNA18, SCARNA2, SCARNA24,
SCCPDH, SCEL, SCHIP1, SCML4, SCN11A, SCN1A, SCN2B, SCN3A, SCN4B,
SCN5A, SCN7A, SCN9A, SCNN1D, SCNN1G, SCRG1, SCRN2, SCT, SCUBE1,
SCUBE2, SCUBE3, SDC1, SDC3, SDC4, SDC4P, SDHAP3, SDHDP2, SDHDP6,
SDK1, SDK2, SDR9C7, SEC14L1, SEC14L4, SEC14L5, SEC14L6, SEC1P,
SEC31B, SEC61A2, SELENBP1, SELI, SELL, SELV, SEMA3A, SEMA3B,
SEMA3C, SEMA3D, SEMA3F, SEMA3G, SEMA4C, SEMA4D, SEMA4F,
SEMA4G, SEMA5B, SEMA6B, SEMA6C, SEMA7A, SENP7, SEPHS1P1, SEPN1,
SEPX1, SERHL2, SERINC2, SERINC5, SERPINA1, SERPINA10, SERPINA11,
SERPINB9, SERPIND1, SERPINF1, SERPINF2, SERPING1, SERPINH1,
SERPIN11, SERPINI2, SERTM1, SET, SETD1A, SETDB1, SETP17, SETP4,
SETP6, SEZ6, SEZ6L, SEZ6L2, SF3A2, SFRP1, SFRP2, SFRP5, SFRS6, SFTA3,
SFTPB, SFTPD, SGCD, SGCZ, SGK494, SGSH, SGTA, SH2B1, SH2B2, SH2D3A,
SH2D3C, SH2D7, SH3BP1, SH3BP2, SH3BP4, SH3BP5, SH3D21, SH3GL1P3,
SH3GLB2, SH3KBP1, SH3PXD2B, SH3RF1, SH3RF3, SH3RF3-AS1, SHANK1,
SHANK2, SHANK3, SHARPIN, SHBG, SHC1P1, SHC2, SHC3, SHC4, SHD, SHF,
SHH, SHISA2, SHISA4, SHISA6, SHISA7, SHISA9, SHKBP1, SHOX2, SHPK,
SHRM, SHROOM3, SHROOM4, SI, SIAH2-AS1, SIDT2, SIGIRR, SIGLEC15,
SIGLEC6, SIK3-IT1, SIN3B, SIPA1, SIRPA, SIRPAP1, SIRT2, SIRT3, SIRT4,
SIT1, SIX5, SKA1, SKA3, SKIDA1, SKOR1, SKOR2, SLC10A1, SLC12A1,
SLC12A2, SLC12A3, SLC12A4, SLC12A5, SLC12A7, SLC12A9, SLC13A3,
SLC13A5, SLC14A1, SLC15A1, SLC15A4, SLC16A1, SLC16A11, SLC16A13,
SLC16A3, SLC16A5, SLC16A7, SLC16A8, SLC17A1, SLC17A3, SLC17A7,
SLC17A8, SLC17A9, SLC18A1, SLC18A3, SLC18B1, SLC19A1, SLC19A3,
SLC1A3, SLC1A5, SLC1A6, SLC1A7, SLC22A11, SLC22A14, SLC22A16,
SLC22A18, SLC22A18AS, SLC22A20, SLC22A23, SLC22A3, SLC22A4,
SLC22A7, SLC22A8, SLC23A1, SLC24A3, SLC24A6, SLC25A1, SLC25A14P1,
SLC25A15, SLC25A18, SLC25A1P1, SLC25A20, SLC25A23, SLC25A29,
SLC25A33, SLC25A38, SLC25A41, SLC25A5, SLC25A5P2, SLC25A5P3,
SLC25A6P2, SLC26A1, SLC26A10, SLC26A11, SLC26A4, SLC26A4-AS1,
SLC26A6, SLC26A8, SLC27A1, SLC27A3, SLC29A1, SLC29A2, SLC29A3,
SLC29A4, SLC29A4P1, SLC2A1, SLC2A14, SLC2A1-AS1, SLC2A3, SLC2A4,
SLC2A4RG, SLC2A8, SLC30A10, SLC30A3, SLC32A1, SLC34A3, SLC35C2,
SLC35D2, SLC35E3, SLC35E4, SLC35F1, SLC35F4, SLC36A1, SLC37A4,
SLC38A1, SLC38A11, SLC38A3, SLC38A5, SLC38A7, SLC38A9, SLC39A13,
SLC39A2, SLC39A5, SLC41A3, SLC43A1, SLC43A3, SLC44A2, SLC44A3,
SLC44A5, SLC45A1, SLC46A2, SLC4A1, SLC4A11, SLC4A2, SLC4A3,
SLC52A1, SLC5A11, SLC5A12, SLC5A6, SLC5A7, SLC5A8, SLC5A9, SLC6A1,
SLC6A11, SLC6A12, SLC6A15, SLC6A16, SLC6A18, SLC6A19, SLC6A2,
SLC6A3, SLC6A4, SLC6A5, SLC6A8, SLC7A10, SLC7A11-AS1, SLC7A14,
SLC7A15P, SLC7A3, SLC7A4, SLC7A5P2, SLC7A7, SLC8A2, SLC9A2, SLC9A3,
SLC9A3P2, SLC9A3R1, SLC9A5, SLC9A7, SLCO1B1, SLCO2A1, SLCO4C1,
SLCO6A1, SLFN13, SLIT1, SLIT2, SLIT3, SLITRK1, SLITRK2, SLITRK3,
SLITRK4, SLITRK5, SLMO1, SLN, SMAD5-AS1, SMAD6, SMAD9-AS1,
SMAGP, SMARCA2, SMARCA4, SMARCA5-AS1, SMARCAL1, SMARCB1,
SMARCC1, SMARCE1P1, SMARCE1P2, SMC2, SMCR2, SMCR5, SMG5, SMG6,
SMIM1, SMN2, SMO, SMOC1, SMOC2, SMPD3, SMPD4, SMPDL3B, SMTN,
SMTNL2, SMUG1, SMYD2, SMYD3, SNAI3, SNAP47-AS1, SNAPC3, SNAPC4,
SNCAIP, SNCG, SNED1, SNHG11, SNN, SNORA11, SNORA12, SNORA15,
SNORA2, SNORA2A, SNORA30, SNORA31, SNORA34, SNORA42, SNORA46,
SNORA53, SNORA66, SNORA67, SNORA7, SNORA70, SNORA70G,
SNORA71B, SNORA75, SNORA77, SNORA79, SNORA80B, SNORA9,
SNORD101, SNORD123, SNORD12B, SNORD15A, SNORD23, SNORD46,
SNORD56, SNORD60, SNORD7, SNORD70, SNORD83A, SNORD91A,
SNORD96B, snoU13, SNRPD2P1, SNRPGP9, SNTA1, SNTG2, SNX15, SNX26,
SNX30, SNX32, SNX33, SNX8, SOAT2, SOCS2P2, SOGA1, SOHLH1, SORBS1,
SORBS3, SORCS1, SORCS2, SORCS3, SOS1-IT1, SOSTDC1, SOWAHD, SOX1,
SOX10, SOX11, SOX12, SOX15, SOX17, SOX2, SOX21, SOX21-AS1, SOX3,
SOX4, SOX8, SP3P, SP4, SP6, SP7, SPACA3, SPACA4, SPAG4, SPAG5, SPAG7,
SPANXA2-OT1, SPARC, SPATA20, SPATA2L, SPATA8, SPATC1, SPATS1,
SPATS2L, SPDEF, SPDYC, SPDYE2, SPDYE3, SPEG, SPG20OS, SPG7, SPHK2,
SPIB, SPIN2A, SPIN4-AS1, SPINK1, SPINK5, SPINT1, SPINT2, SPIRE2, SPN,
SPNS1, SPNS2, SPNS3, SPOCK1, SPOCK2, SPOCK3, SPON2, SPPL2B, SPRN,
SPSB3, SPSB4, SPTAN1, SPTBN4, SPTLC3, SPTSSB, SQLE, SRC, SRCAP,
SRCIN1, SRCRB4D, SRD5A2, SREBF2, SRGAP1, SRGAP3, SRL, SRP68P3,
SRP72P1, SRPX, SRRM1P3, SRRM2, SRRM2-AS1, SRRM3, SRRM4, SRRM5,
SRSF10P1, SSBP3, SSBP4, SSH3, SSPO, SST, SSTR1, SSTR2, SSTR5, ST13P2,
ST13P20, ST3GAL2, ST3GAL3, ST6GAL1, ST6GALNAC1, ST6GALNAC3,
ST6GALNAC4P1, ST6GALNAC5, ST8SIA1, ST8SIA2, ST8SIA4, ST8SIA5,
STAB2, STAC, STAG3, STAG3L1, STAG3L2, STAG3L3, STARD10, STARD10-
AS1, STARD3, STARD4-AS1, STARD5, STARD8, STARD9, STC1, STC2,
STEAP1, STK10, STK11IP, STK16, STK17B, STK25, STK32C, STK36, STMN1,
STMN2, STMN3, STMN4, STON1, STON2, STRA6, STRADB, STRN4, STX10,
STX16-NPEPL1, STX1B, STXBP2, STYK1, SUCNR1, SUFU, SUGP1, SUGT1P2,
SULT1B1, SULT1C2, SULT1C4, SULT1E1, SULT4A1, SUMO2P6, SUPT5H,
SUPT6H, SUSD2, SUV39H1, SUV420H2, SV2B, SV2C, SVEP1, SVIL, SYCE1L,
SYCE2, SYDE1, SYDE2, SYMPK, SYN2, SYN3, SYNCRIP, SYNDIG1L, SYNE1-
AS1, SYNE3, SYNE4, SYNGAP1, SYNGR1, SYNJ2BP, SYNPO2L, SYNPR,
SYNPR-AS1, SYPL2, SYT1, SYT10, SYT14L, SYT15, SYT17, SYT2, SYT3,
SYT6, SYTL5, SZT2-AS1, TAAR1, TAB1, TAC1, TAC3, TAC4, TACC2, TACC3,
TACR1, TACR3, TADA3, TAF13P2, TAF6, TAF6L, TAGLN, TAP2, TARBP1,
TARBP2, TARDBPP2, TAS2R12, TAS2R18, TAS2R42, TAS2R5, TAS2R67P,
TAX1BP3, TAZ, TBC1D10C, TBC1D13, TBC1D14, TBC1D16, TBC1D17,
TBC1D25, TBC1D26, TBC1D3, TBC1D3C, TBC1D3F, TBC1D3G, TBC1D3P2,
TBC1D7, TBCAP2, TBCB, TBKBP1, TBL1Y, TBX1, TBX10, TBX15, TBX2,
TBX20, TBX22, TBX3, TBX4, TBX6, TCAP, TCEA2, TCEAL7, TCEAL8,
TCEANC2, TCEB1P19, TCEB2, TCEB2P2, TCF2, TCF24, TCF25, TCF3, TCF7,
TCF7L1, TCIRG1, TCN2, TCP10, TCTE1, TCTN1, TCTN2, TDG, TDH, TDO2,
TEAD2, TEAD3, TECPR1, TECRL, TEKT2, TEKT3, TEKT4, TEKT5, TELO2,
TEPP, TERF1P3, TERT, TET1, TET3, TEX15, TEX22, TEX264, TFAMP1,
TFAP2A, TFAP2B, TFAP2E, TFB1M, TFDP2, TFF1, TFF2, TFF3, TFPI2, TFPT,
TFR2, TGFB1, TGFB1I1, TGFB2, TGFBI, TGFBR2, TGIF2, TGM1, TGM3, TH,
THA1P, THAP3, THAP7, THAP8, THBS1, THBS3, THBS4, THEG, THEM5,
THG1L, THOC1, THOC5, THOP1, THRAP3P1, THRSP, THSD1, THSD4,
THSD7A, THSD7B, THY1, TIA1, TIAM2, TICRR, TIGD5, TIMELESS, TIMM13,
TIMM17B, TIMM22, TIMM8AP1, TIMM8BP2, TIMP3, TINAGL1, TJP2, TJP3,
TK1, TLE1, TLE1P1, TLE2, TLE3, TLE6, TLR10, TLR5, TLX2, TLX3, TM4SF19,
TM4SF19-AS1, TM4SF20, TM4SF4, TM4SF5, TM6SF1, TM6SF2, TM7SF2,
TM9SF4, TMC3, TMC4, TMC6, TMC7, TMC8, TMED1, TMEFF1, TMEFF2,
TMEM102, TMEM105, TMEM114, TMEM120A, TMEM120B, TMEM129,
TMEM130, TMEM132A, TMEM132B, TMEM132D, TMEM132E, TMEM141,
TMEM143, TMEM145, TMEM147, TMEM150A, TMEM150B, TMEM151A,
TMEM151B, TMEM160, TMEM164, TMEM169, TMEM17, TMEM171,
TMEM175, TMEM184B, TMEM190, TMEM191A, TMEM191C, TMEM198B,
TMEM200C, TMEM201, TMEM202, TMEM204, TMEM206, TMEM213,
TMEM217, TMEM222, TMEM229A, TMEM240, TMEM249, TMEM25,
TMEM252, TMEM35, TMEM37, TMEM38A, TMEM41B, TMEM47, TMEM50A,
TMEM52, TMEM53, TMEM56, TMEM59L, TMEM63A, TMEM63B, TMEM72,
TMEM74, TMEM74B, TMEM79, TMEM8, TMEM80, TMEM86A, TMEM86B,
TMEM88B, TMEM89, TMEM8A, TMEM91, TMEM92, TMEM98, TMIGD2,
TMPRSS11CP, TMPRSS11GP, TMPRSS13, TMPRSS3, TMPRSS6, TMPRSS9,
TMSB10, TMSB15A, TMSB15B, TMSB4XP1, TMTC1, TMTC4, TNC, TNFAIP2,
TNFAIP8L1, TNFAIP8L3, TNFRSF10A, TNFRSF10D, TNFRSF18, TNFRSF25,
TNFSF15, TNKS1BP1, TNNC2, TNNI1, TNNI3, TNNT1, TNNT2, TNNT3, TNP1,
TNR, TNRC18, TNRC18P3, TNRC9, TOMM40L, TONSL, TOP1MT, TOP1P2,
TOP2A, TOP3B, TOR1B, TOR2A, TOR4A, TOX2, TOX3, TP53, TP53AIP1,
TP53I11, TP53I13, TP53I3, TP53INP1, TP63, TPBG, TPCN1, TPD52L1, TPGS1,
TPI1P1, TPM1, TPM2, TPM3P4, TPM3P6, TPRN, TPRXL, TPSAB1, TPSD1,
TPST2, TPTE2P1, TPX2, TRA2A, TRABD2A, TRABD2B, TRAF1, TRAF2,
TRAF4, TRAF7, TRAIP, TRANK1, TRAP1, TRAPPC12-AS1, TRAPPC5,
TRAPPC6A, TRBC2, TRBV26OR9-2, TRDN, TREH, TRH, TRHDE-AS1, TRHR,
TRIL, TRIM16, TRIM17, TRIM22, TRIM24, TRIM28, TRIM3, TRIM36, TRIM41,
TRIM45, TRIM46, TRIM5, TRIM50, TRIM55, TRIM58, TRIM6, TRIM62,
TRIM67, TRIM71, TRIM73, TRIM74, TRIOBP, TRIP13, TRIP6, TRMT2A,
TRMT2B-AS1, TRO, TROAP, TRPA1, TRPC4, TRPC4AP, TRPC5, TRPC7,
TRPC7-AS1, TRPM4, TRPM5, TRPT1, TRPV1, TRPV2, TRPV4, TRPV5, TRRAP,
TRUB2, TSC2, TSC22D4, TSEN54, TSGA10IP, TSHR, TSKS, TSKU, TSNARE1,
TSNAXIP1, TSPAN10, TSPAN11, TSPAN14, TSPAN15, TSPAN18, TSPAN32,
TSPAN33, TSPAN4, TSPAN6, TSPY26P, TSSC1, TSSC1-IT1, TSSC4, TSSK3,
TSSK4, TST, TTBK1, TTC12, TTC16, TTC24, TTC29, TTC31, TTC34, TTC36,
TTC38, TTC39DP, TTC40, TTC6, TTC9B, TTK, TTLL1, TTLL10, TTLL10-AS1,
TTLL12, TTLL3, TTLL6, TTLL9, TTN-AS1, TTR, TTYH1, TTYH3, TUBA3D,
TUBA3FP, TUBA4B, TUBB2B, TUBB3, TUBB4A, TUBB8, TUBBP2, TUBBP5,
TUBGCP2, TUBGCP6, TUFMP1, TUSC3, TUSC5, TXNDC12, TXNRD2, TYRO3,
TYSND1, U1, U2, U2AF1L4, U3, U4, U4atac, U52111.14, U52112.12, U6, U6atac,
U7, U73166.2, U73167.7, U8, U82695.9, UACA, UBA52P5, UBASH3B, UBBP1,
UBE2C, UBE2E1-AS1, UBE2L6, UBE2NL, UBE2O, UBE2Q1, UBE2R2, UBE2S,
UBE2SP1, UBE2SP2, UBE3AP2, UBQLN4P1, UBQLNL, UBXN7-AS1, UCKL1,
UCN2, UCP2, UCRC, UFC1, UGT2B11, UGT2B4, UGT2B7, UGT3A1, UGT3A2,
UHRF1, UHRF2P1, ULBP3, ULK1, ULK2, UNC119, UNC13B, UNC13C,
UNC45B, UNC5A, UNC5B, UNC5C, UNC93B1, UNC93B6, UNCX, UOX, UPB1,
UPF3A, UPK1A, UPK1A-AS1, UPK1B, UPK3A, UPK3B, UPP2-IT1, URM1,
UROC1, USF1, USF2, USH1C, USH2A, USHBP1, USP11, USP12-AS2, USP18,
USP19, USP20, USP22, USP24, USP27X, USP30-AS1, USP32P3, USP47, USP49,
USP5, USP9YP10, USP9YP5, UST, VAC14, VANGL1, VANGL2, VASH1, VASN,
VAT1, VAV2, VCAN, VCAN-AS1, VDAC1P4, VEGFB, VEGFC, VENTX,
VEPH1, VGLL1, VGLL2, VGLL3, VIL1, VIM, VIPR1, VIPR1-AS1, VIPR2, VIT,
VMAC, VN1R48P, VN2R17P, VNN2, VNN3, VPS11, VPS13A-AS1, VPS28,
VPS37D, VPS51, VPS53, VPS8, VRK1, VRTN, VSIG1, VSIG10L, VSIG2, VSTM2,
VSTM2A, VSTM2B, VSTM2L, VSTM4, VSTM5, VTCN1, VTN, VWA1, VWA2,
VWA5B1, VWA5B2, VWCE, WAS, WASF1, WASH2P, WASH7P, WBP1,
WBP1L, WBSCR16, WBSCR17, WBSCR28, WDR13, WDR18, WDR24, WDR27,
WDR34, WDR48, WDR54, WDR59, WDR6, WDR60, WDR62, WDR64, WDR72,
WDR81, WDR86, WDR86-AS1, WDR87, WDR90, WDTC1, WEE1, WFDC1,
WFDC2, WHSC1, WI2-81516E3.1, WI2-89031B12.1, WIPF3, WISP2, WIZ, WNK2,
WNK4, WNT1, WNT11, WNT5A, WNT5B, WNT6, WNT8A, WNT8B, WNT9B,
WSB1, WTIP, WWC2, WWTR1, XAGE2B, XCL1, XIRP2, XKR4, XKR5, XKR7,
XKR8, XKRX, XPNPEP1, XRCC1, XRCC2, XRCC3, XXbac-B135H6.15, XXbac-
B33L19.4, XXbac-B444P24.8, XXbac-B461K10.4, XXbac-B562F10.11, XXbac-
BPG181M17.6, XXbac-BPG55C20.3, XX-C283C717.1, XXyac-YM21GA2.4,
XXYLT1, XYLT1, Y_RNA, YAF2, YAP1, YAP1P1, YBX1P4, YBX1P6, YBX2,
YIPF7, YPEL1, YPEL3, YPEL4, yR211F11.2, YWHAZP4, YWHAZP6, Z83844.1,
Z93241.1, Z95704.5, Z98256.1, ZACN, ZAP70, ZBED1, ZBED4, ZBTB12B,
ZBTB16, ZBTB32, ZBTB37, ZBTB45, ZBTB46, ZBTB48, ZBTB7C, ZBTB8B,
ZC3H12D, ZCCHC11, ZCCHC12, ZCCHC24, ZCCHC3, ZDHHC1, ZDHHC11,
ZDHHC12, ZDHHC16, ZDHHC22, ZDHHC8, ZDHHC8P, ZDHHC8P1, ZEB2P1,
ZER1, ZFAND2B, ZFAT, ZFHX2, ZFHX4, ZFP42, ZFP90, ZFP91-CNTF, ZFP92,
ZFPM1, ZFPM2, ZFR2, ZFX, ZFX-AS1, ZFYVE1, ZFYVE19, ZFYVE21, ZG16B,
ZIC1, ZIC2, ZIC3, ZIC5, ZMAT4, ZMIZ2, ZMYM2-IT1, ZMYM3, ZMYM4-AS1,
ZMYND15, ZMYNDI9, ZNF133, ZNF157, ZNF192P1, ZNF192P2, ZNF205,
ZNF217, ZNF219, ZNF26, ZNF264, ZNF276, ZNF281, ZNF282, ZNF296, ZNF300,
ZNF316, ZNF319, ZNF335, ZNF346, ZNF358, ZNF362, ZNF385A, ZNF385B,
ZNF385C, ZNF408, ZNF414, ZNF423, ZNF428, ZNF431, ZNF444, ZNF445,
ZNF446, ZNF467, ZNF469, ZNF474, ZNF48, ZNF488, ZNF497, ZNF500,
ZNF512B, ZNF517, ZNF518A, ZNF521, ZNF532, ZNF534, ZNF536, ZNF541,
ZNF557, ZNF560, ZNF578, ZNF579, ZNF581, ZNF608, ZNF618, ZNF628,
ZNF629, ZNF646, ZNF652P1, ZNF653, ZNF66P, ZNF677, ZNF678, ZNF687,
ZNF688, ZNF689, ZNF69, ZNF692, ZNF695, ZNF696, ZNF703, ZNF704,
ZNF705E, ZNF707, ZNF71, ZNF710, ZNF711, ZNF713, ZNF714, ZNF724P,
ZNF726, ZNF730, ZNF738, ZNF74, ZNF740, ZNF764, ZNF768, ZNF771, ZNF775,
ZNF777, ZNF784, ZNF786, ZNF787, ZNF788, ZNF804A, ZNF807, ZNF812,
ZNF815P, ZNF821, ZNF837, ZNF84, ZNF843, ZNF847P, ZNF85, ZNF853,
ZNF865, ZNF883, ZNF90, ZNRF1, ZNRF4, ZP2, ZSCAN1, ZSCAN2, ZSCAN5B,
ZSCAN5C, ZSWIM4, ZSWIM7, ZWILCH, ZWINT

It is to be understood that the levels of expression of one or more of the genes listed in Table 4 are depicted in FIG. 5C as a fold change in expression of in vitro-differentiated insulin-positive β-like cells compared to mature β-cells.

It should also be appreciated that any gene listed in Table 4 can be used as a marker for detecting immature β-cells or in vitro-differentiated insulin-positive β-like cells by measuring the level of expression of the gene in a cell, culture, cell line, tissue, or population of cells (e.g., suspected of being β-cells), wherein if the level of expression of the gene in the cell, culture, cell line, tissue, or population of cells is elevated (for example, as depicted in FIG. 5C), the cell, culture, cell line, tissue, or population of cells comprises immature β-cells or in vitro-differentiated insulin-positive β-like cells.

Those skilled in the art will also appreciate that any two or more of the genes listed in Table 4 can be used in combinations of up to N genes (where N is a positive integer greater than or equal to 2) as markers for detecting immature β-cells or in vitro-differentiated insulin-positive β-like cells by measuring the levels of expression of the combination of genes in a cell, culture, cell line, tissue, or population of cells (e.g., suspected of being β-cells), wherein if the levels of expression of the combination of genes in the cell, culture, cell line, tissue, or population of cells is elevated (for example, as depicted in FIG. 5C), the cell, culture, cell line, tissue, or population of cells comprises immature β-cells or in vitro-differentiated insulin-positive β-like cells.

In some aspects, the present invention provides a method of identifying the functional maturity of β-cells. An exemplary method of identifying the functional maturity of β-cells comprises (a) obtaining a putative β-cell or a population of putative β-cells; and (b) detecting an expression level in the β-cell or the population of β-cells of one or more genes listed in Table 1, Table 2, Table 3 or Table 4, wherein: (i) an elevated level of expression of one or more genes listed in Table 1 or Table 2 in the β-cell or the population of β-cells indicates that the β-cell or the population of β-cells are functionally mature β-cells; and (ii) an elevated level of expression of one or more genes listed in Table 3 or Table 4 in the β-cell or the population of β-cells indicates that the β-cell or the population of β-cells are functionally immature β-cells.

In some aspects, the present invention provides a method of identifying mature β-cells. An exemplary method of identifying mature β-cells comprises (a) obtaining a putative β-cell or a population of putative β-cells; and (b) detecting an expression level in the β-cell or the population of β-cells of one or more genes listed in Table 1 or Table 2, wherein: (i) an elevated level of expression of one or more genes listed in Table 1 or Table 2 in the β-cell or the population of β-cells indicates that the β-cell or the population of β-cells are mature β-cells.

In some embodiments of this and other aspects of the invention, the presence of elevated levels expression comprises at least a 2 fold increase, a 3 fold increase, a 4 fold increase, a 5 fold increase, or N-fold increase (where N is a positive integer) in the levels of expression of the one or more genes listed in Table 1 or Table 2 in the β-cell or the population of β-cells compared to the levels of expression of the same one or more genes in immature β-cells.

In some embodiments of this and other aspects of the invention, the presence of elevated levels expression comprises at least a 2 fold increase, a 3 fold increase, a 4 fold increase, a 5 fold increase, or N-fold increase (where N is a positive integer) in the levels of expression of the one or more genes listed in Table 3 or Table 4 in the β-cell or the population of β-cells compared to the levels of expression of the same one or more genes in mature β-cells.

In some aspects, the present invention provides a method of identifying fetal β-cells. An exemplary method of identifying fetal β-cells comprises (a) obtaining a putative β-cell or a population of putative β-cells; and (b) detecting an expression level in the β-cell or the population of β-cells of one or more genes listed in Table 3, wherein: (i) an elevated level of expression of one or more genes listed in Table 3 in the β-cell or the population of β-cells indicates that the β-cell or the population of β-cells are fetal β-cells.

In some embodiments of this and other aspects of the invention, the presence of elevated levels expression comprises at least a 2 fold increase, a 3 fold increase, a 4 fold increase, a 5 fold increase, or N-fold increase (where N is a positive integer) in the levels of expression of the one or more genes listed in Table 3 in the β-cell or the population of β-cells compared to the levels of expression of the same one or more genes in mature β-cells.

In some aspects, the present invention provides a method of identifying in vitro-differentiated insulin-positive β-like cells. An exemplary method of identifying in vitro-differentiated insulin-positive β-like cells, comprises: (a) obtaining a putative β-cell or the population of putative β-cells; and (b) detecting an expression level in the β-cell or the population of β-cells of one or more genes listed in Table 4, wherein: (i) an elevated level of expression of one or more genes listed in Table 4 in the β-cell or the population of β-cells indicates that the β-cell or the population of β-cells are in vitro-differentiated insulin-positive β-like cells.

In some embodiments of this and other aspects of the invention, the presence of elevated levels expression comprises at least a 2 fold increase, a 3 fold increase, a 4 fold increase, a 5 fold increase, or N-fold increase (where N is a positive integer) in the levels of expression of the one or more genes listed in Table 4 in the β-cell or the population of β-cells compared to the levels of expression of the same one or more genes in mature β-cells.

In some embodiments of this and other aspects of the invention, the elevated levels expression of the one or more genes listed in Table 3 and Table 4 are depicted in FIG. 5C. Those skilled in the art will appreciate how to interpret the negative relative expression levels depicted in FIG. 5C as positive levels of elevated expression.

In some embodiments of this and other aspects of the invention, the elevated levels expression of the one or more genes listed in Tables 2 and 4 are depicted in FIG. 5C. Those skilled in the art will appreciate how to interpret the negative relative expression levels depicted in FIG. 5C as positive levels of elevated expression.

In some aspects, an exemplary method of distinguishing mature and immature β-cells comprises: (a) obtaining a putative β-cell or a population of putative β-cells; (b) measuring expression of a group of genes in the β-cell or the population of β-cells to produce an expression profile of the β-cell or the population of β-cells; (c) comparing the expression profile of the β-cell or the population of β-cells to any or all of: (i) a reference mature β-cell expression profile selected from the group consisting of a first group of genes having higher expression levels in mature β-cells compared to fetal β-cells, wherein the first group of genes is selected from the group consisting of STAT4, NPAS2, STAT3, NPAS2, STAT3, PBX3, NR3C2, DDIT3, SIX4, ETV5, SIX2, TP53, BCL6, MESP1, HOPX, BHLHB3, EPAS1, KCNK3, GPI, CHGB, ALDOA, MAFA, SYT7, IAPP, WNT4, PDK3, KCNK1, SLC2A2, ESR1, G6PC2, and a second group of genes having higher expression levels in mature β-cells compared to insulin-positive β-like cells, wherein the second group of genes is selected from the group consisting of XBP1, NFIA, PURA, PDX1, NR3C2, MNX1, GLIS3, EPAS1, HSF4, TSHZ3, MAFA, NKX6-1, HOPX, RORC, NFIX, PEG3, CEBPD, KLF9, STX1A, KCNMA1, PDX1, CHGB, MNX1, PCSK2, NKX6.1, GLIS3, KCNK12, KCNK3, GCGR, KCNK1, SLC30A8, PCSK1, MAFA, ESR1, SLC2A2, IAPP, G6PC2, STXBP1, KCNH2, KCNMB2, UCN3, and WNT4; and (ii) a reference immature fetal β-cell expression profile selected from the group consisting of a third group of genes having higher expression levels in fetal β-cells compared to mature β-cells, wherein said third group of genes is selected from the group consisting of LZTS1, EVI1, MYCN, FOS, EGR1, RCOR2, TCF3, ASCL2, NOTCH1, LMO4, PAX4, NFIB, ISX, SOX11, LHX4, ZNF423, SOX8, RFX1, PROX1, HHEX, CSRNP3, LZTR1, SOX4, NKX6.2, COL1A1, PAX4, KCNH6, RIMS3, PROX1, SOX4, ACSS1, GHRL, NOTCH1, KCNN3, GCK, PYY, HCN3, KCNJ4, and a fourth group of genes having higher expression levels in insulin-positive β-like cells compared to mature β-cells, wherein said fourth group of genes is selected from the group consisting of FOXA1, HHEX, NR2F1, FEV, IRX2, SOX11, PAX4, ONECUT2, LMO4, AEBP1, HES6, TGIF2, LZTS1, TCF3, GATA4, ARX, EGR1, RCOR2, CEBPA, ELF4, HNF4G, PBX2, ISX, ZNF217, NTS, GAST, RIMS3, CACNA1E, PYY, SCT, FOXA1, GATA4, KCNH6, ARX, DLL3, NOTCH1, IRX2, DPP4, PAX4, ACOX2, KCNB1, PROX1, GHRL, SLC2A1, ONECUT2, and SLC2A3; and (d) distinguishing mature and immature β-cells, wherein the β-cell or the population of β-cells are: (i) mature β-cells if the expression profile of the β-cell or the population of β-cells exhibits a pattern of expression similar to either reference mature β-cell expression profile; or (ii) immature β-cells if the expression profile of the β-cell or the population of β-cells exhibits a pattern of expression similar to either reference immature β-cell expression profile expression profile.

### Signaling Pathways Enriched in Mature and Immature β-Cells

The present invention contemplates distinguishing mature and immature β-cells by assessing enrichment of one or more signaling pathways enriched in mature and/or immature β-cells in a β-cell or population of β-cells.

Generally, detecting enrichment of a signaling pathway enriched in mature β-cells compared to fetal β-cells or in vitro-differentiated insulin-positive β-like cells in a β-cell or population of β-cells is indicative that the β-cell or population of β-cells is functionally mature, whereas detecting the absence of enrichment of the same signaling pathway in the β-cell or population of β-cells may be indicative that the β-cell or population of β-cells is functionally immature. Conversely, detecting enrichment of a signaling pathway enriched in fetal β-cells or in vitro-differentiated insulin-positive β-like cells compared to mature β-cells in a β-cell or population of β-cells is indicative that the β-cell or population of β-cells is functionally immature, whereas detecting the absence of enrichment of the same signaling pathway in the β-cell or population of β-cells may be indicative that the β-cell or population of β-cells is functionally mature.

The present invention contemplates assessing enrichment of a signaling pathway according to any technique available to the skilled artisan. In some embodiments of this and other aspects of the invention, detecting the presence or absence of enrichment of signaling pathways comprises conducting a Gene Set Enrichment Analysis (GSEA).

In some aspects, one or more markers of β-cell functional maturity include signaling pathways which are enriched in mature β-cells compared to fetal β-cells or in vitro-differentiated insulin-positive β-like cells.

In some aspects, one or more markers of β-cell functional immaturity include signaling pathways which are enriched in fetal β-cells or in vitro-differentiated insulin-positive β-like cells compared to mature β-cells.

In some aspects, an exemplary method of distinguishing mature and immature β-cells comprises: (a) obtaining a putative β-cell or a population of putative β-cells; (b) assessing enrichment of a signaling pathway to produce a signaling pathway enrichment plot of the β-cell or the population of β-cells, wherein the signaling pathway is selected from the group consisting of an unfolded protein response signaling pathway, an insulin synthesis and secretion signaling pathway, and a metal ion SLC transporters signaling pathway; and (c) distinguishing mature and immature β-cells, wherein the β-cell or the population of β-cells are: (i) mature β-cells if the signaling pathway enrichment plot of the β-cell or the population of β-cells indicates that at least one of the unfolded protein response signaling pathway, the insulin synthesis and secretion signaling pathway, and the metal ion SLC transporters signaling pathway are enriched in the β-cell or the population of β-cells; or (ii) immature β-cells if the signaling pathway enrichment plot of the β-cell or the population of β-cells indicates that none of the unfolded protein response signaling pathway, the insulin synthesis and secretion signaling pathway, and the metal ion SLC transporters signaling pathway are enriched in the β-cell or the population of β-cells.

### Biological Processes Enriched in Mature and Immature β-Cells

The present invention contemplates distinguishing mature and immature β-cells by assessing enrichment of one or more biological processes enriched in mature and/or immature β-cells in a β-cell or population of β-cells.

Generally, detecting enrichment of a biological process enriched in mature β-cells compared to fetal β-cells or in vitro-differentiated insulin-positive β-like cells in a β-cell or population of β-cells is indicative that the β-cell or population of β-cells is functionally mature, whereas detecting the absence of enrichment of the same biological process in the β-cell or population of β-cells may be indicative that the β-cell or population of β-cells is functionally immature. Conversely, detecting enrichment of a biological process enriched in fetal β-cells or in vitro-differentiated insulin-positive β-like cells compared to mature β-cells in a β-cell or population of β-cells is indicative that the β-cell or population of β-cells is functionally immature, whereas detecting the absence of enrichment of the same biological process in the β-cell or population of β-cells may be indicative that the β-cell or population of β-cells is functionally mature.

The present invention contemplates assessing enrichment of biological processes according to any technique available to the skilled artisan. In some embodiments of this and other aspects of the invention, assessing enrichment of biological processes comprises conducting a Gene Ontology (GO). In some embodiments, the Gene Ontology comprises DAVID's Gene Ontology.

In some aspects, one or more markers of β-cell functional maturity include biological processes which are enriched in mature β-cells compared to fetal β-cells or in vitro-differentiated insulin-positive β-like cells.

In some aspects, one or more markers of β-cell functional immaturity include biological processes which are enriched in fetal β-cells or in vitro-differentiated insulin-positive β-like cells compared to mature β-cells.

In some aspects, an exemplary method of distinguishing mature and immature β-cells, comprises: (a) obtaining a putative β-cell or a population of putative β-cells; (b) assessing enrichment of a biological process to determine if the biological process is enriched in the β-cell or the population of β-cells, wherein the biological process is selected from the group consisting of: (i) generation of precursor metabolites and energy, (ii) oxidation reduction, (iii) vesicle-mediated transport, (iv) electron transport chain, (v) monosaccharide metabolic process, (vi) cell morphogenesis, (vii) cellular component morphogenesis, (viii) cell projection organization, (ix) Wnt receptor signaling pathway, (x) cell projection morphogenesis, (xi) cytoskeleton organization, (xii) sterol biosynthetic process, (xiii) cholesterol biosynthetic process, (xiv) actin filament-based process, (xv) actin cytoskeleton organization, (xvi) sterol metabolic process, and (xvii) neuron projection development; (c) distinguishing mature and immature β-cells, wherein the β-cell or the population of β-cells are: (i) mature β-cells if at least one of the biological processes selected from the group consisting of (i), (ii), (iii), (iv), and (v) is enriched in the β-cell or the population of β-cells; or (ii) immature β-cells if at least one of the biological processes selected from the group consisting of (vi), (vii), (viii), (ix), (x), (xi), (xii), (xiii), (xiv), (xv), (xvi), and (xvii) is enriched in the β-cell or the population of β-cells.

Methods of Identifying Agents that Modulate β-Cell Maturity

The markers of the present invention can be measured in β-cells or populations of β-cells to assay for agents that modulate β-cell maturity (e.g., agents that induce β-cells to mature into functionally mature β-cells or agents that induce mature β-cells to become functionally immature β-cells). Identification of agents (or factors) that induce functional β-cell maturation can be used for the in vitro production of a virtually unlimited supply of functionally mature β-cells for administration to a human or animal in need of such functionally mature β-cells (e.g., an individual suffering from a disorder associated with immature β-cells, e.g., diabetes). Identification of agents (or factors) that induce mature β-cells to dedifferentiate into functionally immature β-cells can be used to understand mechanisms underlying disorders associated with immature β-cells, as well as to identify conditions in culture which might need to be inhibited to produce functionally mature β-cells in vitro.

Accordingly, in some aspects, the present invention provides methods of identifying a candidate agent that modulates the functional maturity of β-cells.

An exemplary method of identifying a candidate agent that modulates the functional maturity of β-cells, comprises: (a) contacting a β-cell or a population of β-cells with a test agent; (b) monitoring expression of a group of genes in the β-cell or the population of β-cells, in the presence of the test agent, to produce an expression profile of the β-cell or the population of β-cells; (c) comparing the expression profile of the β-cell or the population of β-cells to: (i) a reference mature β-cell expression profile selected from the group consisting of a first group of genes having higher expression levels in mature β-cells compared to fetal β-cells, wherein the first group of genes is selected from the group consisting of KCNK3, GPI, CHGB, ALDOA, MAFA, SYT7, IAPP, WNT4, PDK3, KCNK1, SLC2A2, ESR1, G6PC2, and a second group of genes having higher expression levels in mature β-cells compared to insulin-positive β-like cells, wherein the second group of genes is selected from the group consisting of STX1A, KCNMA1, PDX1, CHGB, MNX1, PCSK2, NKX6.1, GLIS3, KCNK12, KCNK3, GCGR, KCNK1, SLC30A8, PCSK1, MAFA, ESR1, SLC2A2, IAPP, G6PC2, STXBP1, KCNH2, KCNMB2, UCN3, and WNT4; (ii) a reference immature β-cell expression profile selected from the group consisting of a third group of genes having higher expression levels in fetal β-cells compared to mature β-cells, wherein said third group of genes is selected from the group consisting of NKX6.2, COL1A1, PAX4, KCNH6, RIMS3, PROX1, SOX4, ACSS1, GHRL, NOTCH1, KCNN3, GCK, PYY, HCN3, and KCNJ4, and a fourth group of genes having higher expression levels in insulin-positive β-like cells compared to mature β-cells, wherein said fourth group of genes is selected from the group consisting of NTS, GAST, RIMS3, CACNA1E, PYY, SCT, FOXA1, GATA4, KCNH6, ARX, DLL3, NOTCH1, IRX2, DPP4, PAX4, ACOX2, KCNB1, PROX1, GHRL, SLC2A1, ONECUT2, and SLC2A3; and (d) identifying the test agent as a candidate agent that modulates the functional maturity of β-cells, wherein: (i) the test agent is a candidate agent that induces β-cells to become functionally mature if the β-cell or the population of β-cells exhibits a pattern of expression similar to the either reference mature β-cell expression profile in the presence of the test agent; (ii) the test agent is a candidate agent that induces β-cells to become functionally immature if the β-cell or the population of β-cells exhibits a pattern of expression similar to either reference immature β-cell expression profile.

Another exemplary method of identifying a candidate agent that modulates the functional maturity of β-cells, comprises: (a) contacting a β-cell or a population of β-cells with a test agent; (b) assessing enrichment of a signaling pathway in the presence of the test agent to produce a signaling pathway enrichment plot of the β-cell or the population of β-cells, wherein the signaling pathway is selected from the group consisting of an unfolded protein response signaling pathway, an insulin synthesis and secretion signaling pathway, and a metal ion SLC transporters signaling pathway; (c) identifying the test agent as a candidate agent that modulates the functional maturity of β-cells, wherein: (i) the test agent is a candidate agent that induces β-cells to become functionally mature if the signaling pathway enrichment plot of the β-cell or the population of β-cells indicates that at least one of the unfolded protein response signaling pathway, the insulin synthesis and secretion signaling pathway, and the metal ion SLC transporters signaling pathway are enriched in the β-cell or the population of β-cells; or (ii) the test agent is a candidate agent that induces β-cells to become functionally immature if the signaling pathway enrichment plot of the β-cell or the population of β-cells indicates that none of the unfolded protein response signaling pathway, the insulin synthesis and secretion signaling pathway, and the metal ion SLC transporters signaling pathway are enriched in the β-cell or the population of β-cells.

Still another exemplary method of identifying a candidate agent that modulates the functional maturity of β-cells, comprises: (a) contacting a β-cell or a population of β-cells with a test agent; (b) assessing enrichment of a biological process in the presence of the test agent to determine if the biological process is enriched in the β-cell or the population of β-cells, wherein the biological process is selected from the group consisting of (i) generation of precursor metabolites and energy, (ii) oxidation reduction, (iii) vesicle-mediated transport, (iv) electron transport chain, (v) monosaccharide metabolic process, (vi) cell morphogenesis, (vii) cellular component morphogenesis, (viii) cell projection organization, (ix) Wnt receptor signaling pathway, (x) cell projection morphogenesis, (xi) cytoskeleton organization, (xii) sterol biosynthetic process, (xiii) cholesterol biosynthetic process, (xiv) actin filament-based process, (xv) actin cytoskeleton organization, (xvi) sterol metabolic process, and (xvii) neuron projection development; and (c) identifying the test agent as a candidate agent that modulates the functional maturity of β-cells, wherein: (i) the test agent is a candidate agent that induces β-cells to become functionally mature if at least one biological process selected from the group consisting of (i), (ii), (iii), (iv), and (v) is enriched in the β-cell or the population of β-cells; or (ii) the test agent is a candidate agent that induces β-cells to become functionally immature if at least one biological process selected from the group consisting of (vi), (vii), (viii), (ix), (x), (xi), (xii), (xiii), (xiv), (xv), (xvi), and (xvii) is enriched in the β-cell or the population of β-cells.

It should be appreciated that candidate β-cell maturity modulating agents identified according to the methods of the invention may be used in methods of treating disorders associated with immature β-cells. For example, an agent that induces immature β-cells to become functionally mature β-cells can be used to treat a disorder associated with immature β-cells. Accordingly, in some embodiments of this and other aspects of the invention, the candidate agent is a candidate agent that modulates a disorder associated with immature β-cells. In some embodiments of this and other aspects of the invention, the disorder is diabetes. In some embodiments of this and other aspects of the invention, the disorder is pre-diabetes.

Those skilled in the art will appreciate how to perform the identification methods (e.g., identifying agents for modulating β-cell maturity, identifying agents that modulate disorders associated with immature β-cells, etc.) of present invention using routine protocols available to the skilled artisan (e.g., high-throughput screening, combinatorial chemistry, in silico screening, etc.).

It should be appreciated that a wide variety of test agents can be used in the methods (e.g., a small molecule, polypeptide, peptide, nucleic acid, oligonucleotide, lipid, carbohydrate, or hybrid molecule).

In another aspect, the present invention provides methods of identifying the functional maturity of an individual's β-cells.

An exemplary method of identifying the functional maturity of an individual's β-cells, comprises: (a) obtaining a biological sample comprising β-cells from the individual; (b) measuring expression of a group of genes in the biological sample to produce an expression profile of the individual's β-cells; (c) comparing the expression profile of the individual's β-cells to: (i) a reference mature β-cell expression profile selected from the group consisting of a first group of genes having higher expression levels in mature β-cells compared to fetal β-cells, wherein the first group of genes is selected from the group consisting of KCNK3, GPI, CHGB, ALDOA, MAFA, SYT7, IAPP, WNT4, PDK3, KCNK1, SLC2A2, ESR1, G6PC2, and a second group of genes having higher expression levels in mature β-cells compared to insulin-positive β-like cells, wherein the second group of genes is selected from the group consisting of STX1A, KCNMA1, PDX1, CHGB, MNX1, PCSK2, NKX6.1, GLIS3, KCNK12, KCNK3, GCGR, KCNK1, SLC30A8, PCSK1, MAFA, ESR1, SLC2A2, IAPP, G6PC2, STXBP1, KCNH2, KCNMB2, UCN3, and WNT4; and (ii) a reference immature β-cell expression profile selected from the group consisting of a third group of genes having higher expression levels in fetal β-cells compared to mature β-cells, wherein said third group of genes is selected from the group consisting of NKX6.2, COL1A1, PAX4, KCNH6, RIMS3, PROX1, SOX4, ACSS1, GHRL, NOTCH1, KCNN3, GCK, PYY, HCN3, and KCNJ4, and a fourth group of genes having higher expression levels in insulin-positive β-like cells compared to mature β-cells, wherein said fourth group of genes is selected from the group consisting of NTS, GAST, RIMS3, CACNA1E, PYY, SCT, FOXA1, GATA4, KCNH6, ARX, DLL3, NOTCH1, IRX2, DPP4, PAX4, ACOX2, KCNB1, PROX1, GHRL, SLC2A1, ONECUT2, and SLC2A3; and (d) identifying the functional maturity of the individual's β-cells, wherein: (i) the individual's β-cells are functionally mature if the β-cell in the biological sample exhibit a pattern of expression similar to either reference mature β-cell expression profile; or (ii) the individual's β-cells are functionally immature if the β-cells in the biological sample exhibit a pattern of expression similar to either reference immature β-cell expression profile.

Another exemplary method of identifying the functional maturity of an individual's β-cells, comprises: (a) obtaining a biological sample comprising β-cells from the individual; (b) assessing enrichment of a signaling pathway to produce a signaling pathway enrichment plot of the β-cells in the biological sample, wherein the signaling pathway is selected from the group consisting of an unfolded protein response signaling pathway, an insulin synthesis and secretion signaling pathway, and a metal ion SLC transporters signaling pathway; (c) identifying the functional maturity of the individual's β-cells, wherein: (i) the individual's β-cells are functionally mature if the signaling pathway enrichment plot indicates that at least one of the unfolded protein response signaling pathway, the insulin synthesis and secretion signaling pathway, and the metal ion SLC transporters signaling pathway are enriched in the β-cell in the biological sample; or (ii) the individual's β-cells are functionally immature if the signaling pathway enrichment plot indicates that none of the unfolded protein response signaling pathway, the insulin synthesis and secretion signaling pathway, and the metal ion SLC transporters signaling pathway are enriched in the β-cells in the biological sample.

Still another exemplary method of identifying the functional maturity of an individual's β-cells, comprises: (a) obtaining a biological sample comprising β-cells from the individual; (b) assessing enrichment of a biological process in the biological sample, wherein the biological process is selected from the group consisting of (i) generation of precursor metabolites and energy, (ii) oxidation reduction, (iii) vesicle-mediated transport, (iv) electron transport chain, (v) monosaccharide metabolic process, (vi) cell morphogenesis, (vii) cellular component morphogenesis, (viii) cell projection organization, (ix) Wnt receptor signaling pathway, (x) cell projection morphogenesis, (xi) cytoskeleton organization, (xii) sterol biosynthetic process, (xiii) cholesterol biosynthetic process, (xiv) actin filament-based process, (xv) actin cytoskeleton organization, (xvi) sterol metabolic process, and (xvii) neuron projection development; and (c) identifying the functional maturity of the individual's β-cells, wherein: (i) the individual's β-cells are functionally mature if at least one biological process selected from the group consisting of (i), (ii), (iii), (iv), and (v) is enriched in the biological sample; or (ii) the individual's β-cells are functionally immature if at least one biological process selected from the group consisting of (vi), (vii), (viii), (ix), (x), (xi), (xii), (xiii), (xiv), (xv), (xvi), and (xvii) is enriched in the biological sample.

The present invention contemplates obtaining a biological sample comprising β-cells from the individual according to any technique available to the skilled artisan. The individual from which the biological sample is obtained may be a healthy individual, or an individual suffering from a disorder associated with functionally immature β-cells.

In some embodiments of this and other aspects of the invention, if the individual's β-cells are functionally immature, the individual is in need of functionally mature β-cells.

In some embodiments of this and other aspects of the invention, the individual is a human or animal.

In some embodiments of this and other aspects of the invention, the functional maturity of the individual's β-cells is identified before β-cells are administered to the individual. In some embodiments of this and other aspects of the invention, the functional maturity of the individual's β-cells is identified after β-cells have been administered to the individual.

In some embodiments of this and other aspects of the invention, the biological sample comprises pancreatic tissue. In some embodiments of this and other aspects of the invention, the biological sample comprises islets of Langerhans.

The markers of the present invention can be used for selecting functionally mature β-cells for administration to a human or animal subject in need of such functionally mature β-cells, as well as determining whether in vitro β-like cells administered to a human or animal subject are functionally mature in vivo.

In some embodiments of this and other aspects of the invention, administering the in vitro-differentiated β-like cells to the human or animal subject comprises transplanting the β-like cells to the human or animal subject (e.g., into a kidney capsule of the human or animal subject). Other suitable methods of administering the in vitro-differentiated β-like cells to the human or animal subject are apparent to the skilled artisan.

In some instances, it may be desirable to conduct a glucose tolerance test on the human or animal subject to detect levels of human fasting C-peptide in the human or animal subject. It should be appreciated that if the levels of fasting human C-peptide levels detected are above a background level after administration of β-like cells to the human or animal subject, the administered β-like cells are functionally mature in vivo. In this way, fasting human C-peptide levels can be used as an additional marker to confirm that the administered β-like cells have functionally matured in vivo (e.g., the functionally mature β-like cells are glucose-responsive β-cells).

In some embodiments of this and other aspects of the invention, the β-cell or population of β-cells is obtained from an in vitro source.

In some embodiments of this and other aspects of the invention, the in vitro source of β-cells is a culture of differentiating stem cells. As used herein, “stem cell” refers to a cell that has the ability to differentiate into a cell of any type. Examples of stem cells that can be used in the methods of the present invention include embryonic stem cells obtained by culturing a pre-implantation early embryo, embryonic stem cells obtained by culturing an early embryo prepared by somatic cell nuclear transfer, and induced pluripotent stem cells obtained by transferring appropriate transcription factors to a somatic cell to reprogram the cell. A variety of protocols for obtaining the stem cells for use in the methods of the present invention are available to the skilled artisan.

In some embodiments of this and other aspects of the invention, the stem cells are human embryonic stem cells (hESCs). In some embodiments of this and other aspects of the invention, the stem cells are induced pluripotent stem cells (iPSCs). In some embodiments of this and other aspects of the invention, the induced pluripotent stem cells are derived from reprogramming human somatic cells. The human somatic cells can be obtained from a healthy human or a human suffering from a disorder associated with immature or abnormal β-cells.

β-cells obtained from a culture of differentiating stem cells are referred to herein as “in vitro-differentiating β-cells” or “in vitro-differentiating β-cells.” As used herein, “in vitro-differentiated β-cell” refers to a β-cell or β-like cell obtained by differentiating a stem cell in vitro. An “in vitro-differentiating β-cell” refers to a cell (or cells) in the process of differentiating into a β-cell or β-like cell.

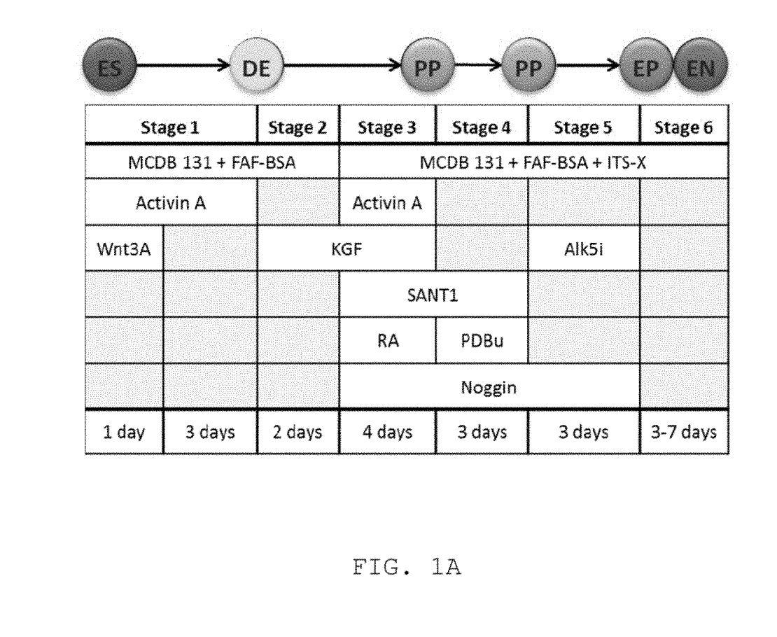

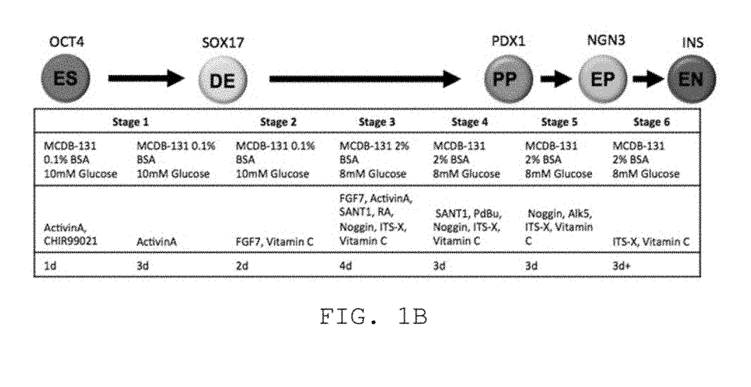

Generally, the in vitro-differentiated β-cells obtained in accordance with the methods of the present invention are derived by differentiating one or more stem cells into β-cells or β-like cells. The present invention contemplates any culturing protocol that is capable of differentiating stem cells into β-cells or β-like cells. FIGS. 1A and 1B illustrate exemplary culturing protocols. Additional examples of suitable protocols have been reviewed by Liew (Liew C G. Rev Diabet Stud 7(2), 82-92 (2010), incorporated herein by reference in its entirety.)

In some embodiments of this and other aspects of the invention, the in vitro source includes a cell bank (e.g., cryopreserved β-cells), a cell line, a cell culture (e.g., in vitro-differentiated β-cells), a cell population, and combinations thereof.

In some embodiments of this and other aspects of the invention, the in vitro source is an artificial tissue or organ (e.g., a pancreas, pancreatic islets, etc.).

In some embodiments of this and other aspects of the invention, the β-cell is obtained from an in vivo source.

In some embodiments of this and other aspects of the invention, the in vivo source is an individual that has received an administration of β-cells. In such embodiments, an individual can be administered functionally mature β-cells (e.g., via transplantation) and the markers and methods of the present invention can be used to confirm that the administered β-cells remain mature post-administration. Alternatively, an individual can be administered functionally immature β-cells (e.g., in vitro-differentiated insulin positive β-like cells) and the markers and methods of the present invention can be used to determine whether the functionally immature β-cells have matured in vivo.

In some embodiments of this and other aspects of the invention, the in vivo source is an individual suffering from a disorder associated with immature β-cells (e.g., diabetes or pre-diabetes).

In some embodiments of this and other aspects of the invention, the in vivo source is an individual suffering from a β-cell disorder selected from the group consisting of a disorder associated with immature β-cells, a disorder associated with destruction of β-cells, a disorder associated with dysfunctional β-cells, and a disorder associated with an insufficient number of β-cells.

In some embodiments of this and other aspects of the invention, the in vivo source is an individual suspected of being in need of functionally mature β-cells. In such embodiments, the methods of identifying the functional maturity of β-cells can be adapted for use in methods of identifying individuals in need of functionally mature β-cells. For example, a biological sample comprising β-cells can be obtained from the individual and the β-cells in the biological sample can be assessed for their maturity in accordance with the methods of the present invention.

In some embodiments of this and other aspects of the invention, the in vivo source is a tissue or organ obtained from a donor individual. In such embodiments, the markers or methods of the present invention can be used to determine whether the β-cells in the tissue or organ (e.g., pancreas, islets of Langerhans, etc.) are functionally mature before transplanting the tissue or organ into the recipient individual.

In some embodiments of this and other aspects of the invention, the individual is a human or animal individual.

In some embodiments of this and other aspects of the invention, the present invention contemplates sorting the immature and mature β-cells identified. Sorting immature and mature β-cells can be helpful for selecting mature β-cells that might be useful for administration to an individual in need of mature β-cells.

Suitable methods of sorting cells are apparent to the skilled artisan. In some embodiments of this and other aspects of the invention, sorting the functionally immature and mature β-cells identified in the population of β-cells is achieved by fluorescence-activated cell sorting (FACS). In some embodiments of this and other aspects of the invention, sorting the functionally immature and mature β-cells identified in the population of β-cells is achieved by intracellular-FACS (IC-FACS).

In some embodiments of this and other aspects of the invention, the FACS comprises staining at least one antibody specific for a putative β-cell surface marker selected from the group consisting of ABCA3, CD79B, FXYD2, KCNB2, NLGN1, PTPRU, SLC6A9, ABCC8, CD8A, GCGR, KCNF1, NPR2, ROBO1, SORL1, ABCG1, CDH2, GPR120, KCNG3, NRCAM, RTN4, SVOP, ACSL1, CDH22, GPR19, KCNH2, PCDHA1, SEMA5A, TGFBR3, ATP1B2, CHRNA5, GRIA2, KCNMA1, PCDHA3, SERP2, TRPM2, CACNA1H, CYB561, KCNH1, KCNQ2, PIGU, SLC17A6, TRPM5, CADM1, EFNB3, IGSF11, MADD, PLXNA2, SLC43A2, TSPAN13, CASR, FFAR1, IL17RB, NEO1, PRRG2, SLC6A6, and UNC5A.

It should be appreciated that FACS analysis can be performed in combination with the methods for detecting markers of the present invention to sort β-cells expressing certain markers and quantify the percentage and levels of expression of those markers, as well as to analyze global gene expression patterns.

In some embodiments of this and other aspects of the invention, the present invention contemplates quantifying the sorted functionally immature and mature β-cells identified in the population of β-cells.

In some embodiments of this and other aspects of the invention, the present invention contemplates preserving the sorted functionally mature β-cells (e.g., cryopreservation of the cells in appropriate reagents).

In some embodiments of this and other aspects of the invention, the present invention contemplates conducting a GSIS assay on the β-cell or population of β-cells to determine whether the β-cell or the population of β-cells exhibits an appropriate GSIS response.

In some embodiments of this and other aspects of the invention, the mature β-cell is a human β-cell.

In some embodiments of this and other aspects of the invention, the fetal β-cell is a human β-cell.

In some embodiments of this and other aspects of the invention, the insulin-positive β-like cell is derived from human cells selected from the group consisting of human embryonic stem cells, reprogrammed human somatic cells, and induced human pluripotent stem cells.

In some aspects, the present invention provides a method of delivering a molecule of interest to a β-cell or a population of β-cells. An exemplary method of delivering a molecule of interest to a β-cell or a population of β-cells, comprises: contacting the β-cell or the population of β-cells with a composition comprising the molecule of interest conjugated to an antibody that binds to a putative β-cell surface marker selected from the group consisting of ABCA3, CD79B, FXYD2, KCNB2, NLGN1, PTPRU, SLC6A9, ABCC8, CD8A, GCGR, KCNF1, NPR2, ROBO1, SORL1, ABCG1, CDH2, GPR120, KCNG3, NRCAM, RTN4, SVOP, ACSL1, CDH22, GPR19, KCNH2, PCDHA1, SEMA5A, TGFBR3, ATP1B2, CHRNA5, GRIA2, KCNMA1, PCDHA3, SERP2, TRPM2, CACNA1H, CYB561, KCNH1, KCNQ2, PIGU, SLC17A6, TRPM5, CADM1, EFNB3, IGSF11, MADD, PLXNA2, SLC43A2, TSPAN13, CASR, FFAR1, IL17RB, NEO1, PRRG2, SLC6A6, and UNC5A.

The present invention contemplates delivering any molecule of interest to a β-cell or a population of β-cells. In some embodiments, the molecule of interest is a compound selected from small organic or inorganic molecules; saccharines; oligosaccharides; polysaccharides; biological macromolecules, e.g., peptides, proteins, and peptide analogs and derivatives; peptidomimetics; nucleic acids and nucleic acid analogs and derivatives (including but not limited to siRNAs, shRNAs, antisense RNAs, a ribozymes, and apatamers); an extract made from biological materials such as bacteria, plants, fungi, or animal cells; animal tissues; naturally occurring or synthetic compositions; and any combinations thereof.

As used herein, the term “antibody” includes complete immunoglobulins, antigen binding fragments of immunoglobulins, as well as antigen binding proteins that comprise antigen binding domains of immunoglobulins. Antigen binding fragments of immunoglobulins include, for example, Fab, Fab′, F(ab′)₂, scFv and dAbs. Modified antibody formats have been developed which retain binding specificity, but have other characteristics that may be desirable, including for example, bispecificity, multivalence (more than two binding sites), and compact size (e.g., binding domains alone). Single chain antibodies lack some or all of the constant domains of the whole antibodies from which they are derived. Therefore, they can overcome some of the problems associated with the use of whole antibodies. For example, single-chain antibodies tend to be free of certain undesired interactions between heavy-chain constant regions and other biological molecules. Additionally, single-chain antibodies are considerably smaller than whole antibodies and can have greater permeability than whole antibodies, allowing single-chain antibodies to localize and bind to target antigen-binding sites more efficiently. Furthermore, the relatively small size of single-chain antibodies makes them less likely to provoke an unwanted immune response in a recipient than whole antibodies.

Multiple single chain antibodies, each single chain having one VH and one VL domain covalently linked by a first peptide linker, can be covalently linked by at least one or more peptide linker to form multivalent single chain antibodies, which can be monospecific or multispecific. Each chain of a multivalent single chain antibody includes a variable light chain fragment and a variable heavy chain fragment, and is linked by a peptide linker to at least one other chain. The peptide linker is composed of at least fifteen amino acid residues. The maximum number of linker amino acid residues is approximately one hundred. Two single chain antibodies can be combined to form a diabody, also known as a bivalent dimer. Diabodies have two chains and two binding sites, and can be monospecific or bispecific. Each chain of the diabody includes a VH domain connected to a VL domain. The domains are connected with linkers that are short enough to prevent pairing between domains on the same chain, thus driving the pairing between complementary domains on different chains to recreate the two antigen-binding sites. Three single chain antibodies can be combined to form triabodies, also known as trivalent trimers. Triabodies are constructed with the amino acid terminus of a VL or VH domain directly fused to the carboxyl terminus of a VL or VH domain, i.e., without any linker sequence. The triabody has three Fv heads with the polypeptides arranged in a cyclic, head-to-tail fashion. A possible conformation of the triabody is planar with the three binding sites located in a plane at an angle of 120 degrees from one another. Triabodies can be monospecific, bispecific or trispecific. Thus, antibodies useful in the methods described herein include, but are not limited to, naturally occurring antibodies, bivalent fragments such as (Fab′)₂, monovalent fragments such as Fab, single chain antibodies, single chain Fv (scFv), single domain antibodies, multivalent single chain antibodies, diabodies, triabodies, and the like that bind specifically with an antigen. While both polyclonal and monoclonal antibodies can be used in the methods described herein, it is preferred that a monoclonal antibody is used where conditions require increased specificity for a particular protein. Antibodies can be raised against a putative β-cell surface marker by methods known to those skilled in the art. Such methods are described in detail, for example, in Harlow et al., 1988 in: Antibodies, A Laboratory Manual, Cold Spring Harbor, N.Y. Those skilled in the art will appreciate how to conjugate such antibodies to other molecules of interest according to routine methods.

In some embodiments, the contacting occurs in vitro. In some embodiments, the contacting occurs ex vivo. In some embodiments, the contacting occurs in vivo. In some embodiments, a molecule of interest can be delivered to a β-cell or a population of β-cells in vivo by administering the composition to an individual in need of the molecule of interest.

In some embodiments, the molecule of interest is a therapeutic agent that has a beneficial effect on β-cells. Examples of such therapeutic agents include, but are not limited to, Metformin or other Biguanides, DPP4 inhibitors, Sulfonylureas or Metiglitinides, SGLT2 inhibitors, Glucokinase activators, Thiazolidinediones, PPARdelta agonists, non-activating PPARgamma modulators, Glp-1 analogs, GIP analogs, Glp-1-receptor agonists, combined Glp-1/GIP receptor agonists, FGF21, agonistic FGFR monoclonal antibodies, Oxyntomodulin analogs, IAPP analogs, Leptin or Leptin analogs, Adiponectin or Adiponectin analogs, Insulin or Insulin analogs, proton pump inhibitors or gastrin receptor agonists, Reg family proteins/Reg family protein derived peptides or alpha-glucosidase inhibitors.

In some embodiments, the therapeutic agent is capable of modulating a β-cell associated disorder. In some embodiments, the β-cell associated disorder is a disorder associated with aberrant insulin production or responsiveness or aberrant blood glucose levels. Disorders include, but are not limited to, diabetes (e.g., Type I or Type II), gestational diabetes, prediabetes, obesity, hyperglycemia, glucose intolerance, insulin resistance, hyperinsulinemia, metabolic syndrome, or syndrome X. The term “diabetes” refers to a disease of a mammalian subject, and includes Type 1 NIDDM-transient, Type 1 IDDM, Type 2 IDDM-transient, Type 2 NIDDM, or in another embodiment, MODY.

In some aspects, the present invention provides a method of identifying a candidate agent that modulates differentiation of β-cells. An exemplary method of identifying a candidate agent that modulates differentiation of β-cells, comprises: (a) contacting a cell, population of cells, cell line or cell culture with a test agent; and monitoring the cell, population of cells, cell line, or cell culture for expression of one or more β-cell specific transcription factors in the presence of the test agent, wherein the β-cell specific transcription factors are selected from the group consisting of ASCL2, NR0B1, SIX4, CHD7, TOX, OLIG1, TSHZ3, DACH1, TSNAX, DACH2, MYT1L, PEG3, ZNF10, NDN, ZNF395, ETV5, ZNF540, HOPX, RXRG and ZNF672; and (b) identifying the test agent as a candidate agent that modulates differentiation of β-cells if the cell, population of cells, cell line, or cell culture expresses one or more of the β-cell specific transcription factors in the presence of the test agent.

In some aspects, the present invention provides a composition for differentiating a precursor cell to a pancreatic β-cell, comprising one or more β-cell specific transcription factors selected from the group consisting of ASCL2, NR0B1, SIX4, CHD7, TOX, OLIG1, TSHZ3, DACH1, TSNAX, DACH2, MYT1L, PEG3, ZNF10, NDN, ZNF395, ETV5, ZNF540, HOPX, RXRG and ZNF672.

In some aspects, the present invention provides a method for differentiating a precursor cell to a pancreatic β-cell. An exemplary method for differentiating a precursor cell to a pancreatic β-cell comprises causing the precursor cell to upregulate expression of one or more β-cell specific transcription factors selected from the group consisting of ASCL2, NR0B1, SIX4, CHD7, TOX, OLIG1, TSHZ3, DACH1, TSNAX, DACH2, MYT1L, PEG3, ZNF10, NDN, ZNF395, ETV5, ZNF540, HOPX, RXRG and ZNF672. The precursor cell can be any cell that is capable of differentiating into a pancreatic β-cell (e.g., iPSCs, hESCs, pancreatic progenitor cells, etc.). The present invention contemplates causing the precursor cell to upregulate expression of the one or more β-cell specific transcription factors. In some embodiments, the precursor cell can be transfected with a vector comprising a nucleic acid encoding the one or more β-cell specific transcription factors. In some embodiments, causing the precursor cell to upregulate expression of the one or more β-cell specific transcription factors comprises contacting the precursor cells with a small molecule that causes the cell to upregulate expression of the one or more β-cell specific transcription factors. In some embodiments, causing the cell to upregulate expression of the one or more β-cell specific transcription factors comprises contacting the precursor cell with an exogenous β-cell specific transcription factors. In some embodiments, causing the cell to upregulate expression of the one or more β-cell specific transcription factors comprises delivering an mRNA encoding the one or more β-cell specific transcription factors. In some embodiments, causing the cell to upregulate expression of the one or more β-cell specific transcription factors comprising contacting the precursor cell with an agent that modulates a signaling pathway that causes the one or more β-cell specific transcription factors to be expressed in the cell. Other suitable methods will be apparent to the skilled artisan.

The invention further provides packaged products and kits, including pluripotent cells or pancreatic progenitor cells that are capable of differentiating into pancreatic β-cells, cell lines, cell cultures, populations and compositions, including, as well as cells, cultures, populations, and compositions enriched or selected for any developmental, maturation or differentiation stage, packaged into suitable packaging material. In some embodiments, a packaged product or kit includes pluripotent cells or pancreatic progenitor cells that are capable of differentiating into pancreatic β-cells, cell lines, cultures, populations or compositions, or a mixed population thereof.

In some aspects, a packaged product or kit includes a label, such as a list of the contents of the package, or instructions for using the kit e.g., instructions for culturing, expanding (increasing cell numbers), proliferating, differentiating, maintaining, or delivery, administering, implanting or transplanting in vivo, or screening for a compound or agent that modulates a function or activity of β-cells thereof.

In certain embodiments, a packaged product or kit includes a container, such as a sealed pouch or shipping container, or an article of manufacture, for culturing, expanding (increasing cell numbers), proliferating, differentiating, maintaining, or preserving pluripotent cells or pancreatic progenitor cells that are capable of differentiating into pancreatic β-cells, such as a tissue culture dish, tube, flask, roller bottle or plate (e.g., a single multi-well plate or dish such as an 8, 16, 32, 64, 96, 384 and 1536 multi-well plate or dish).

In some embodiments, a packaged product or kit includes an article of manufacture, for example, an article of manufacture for delivering, administering or transplanting or implanting mature β-cells or in vitro-differentiated insulin-positive β-like cells into a subject locally, regionally or systemically.

The term “packaging material” refers to a physical structure housing the product or components of the kit. The packaging material can maintain the components sterilely, and can be made of material commonly used for such purposes (e.g., paper, corrugated fiber, glass, plastic, foil, ampules, etc.). A label or packaging insert can be included, listing contents or appropriate written instructions, for example, practicing a method of the invention.

A packaged product or kit can therefore include instructions for practicing any of the methods of the invention described herein. For example, pluripotent cells or pancreatic progenitor cells that are capable of differentiating into pancreatic β-cells can be included in a tissue culture dish, tube, flask, roller bottle or plate (e.g., a single multi-well plate or dish such as an 8, 16, 32, 64, 96, 384 and 1536 multi-well plate or dish) together with instructions for culturing, expanding (increasing cell numbers), proliferating, differentiating, maintaining, preserving or screening. In some embodiments, pluripotent cells or pancreatic progenitor cells that are capable of differentiating into pancreatic β-cells can be included in a container, pack, or dispenser together with instructions for delivery, administration, implantation or transplantation into a subject in need thereof.

Instructions may be on “printed matter,” e.g., on paper or cardboard within the kit, on a label affixed to the package, kit or packaging material, or attached to a tissue culture dish, tube, flask, roller bottle, plate (e.g., a single multi-well plate or dish such as an 8, 16, 32, 64, 96, 384 and 1536 multi-well plate or dish) or vial containing a component (e.g., pluripotent cells or pancreatic progenitor cells that are capable of differentiating into pancreatic β-cells) of the kit. Instructions may comprise voice or video tape and additionally be included on a computer readable medium, such as a disk (floppy diskette or hard disk), optical CD such as CD- or DVD-ROM/RAM, magnetic tape, electrical storage media such as RAM and ROM and hybrids of these such as magnetic/optical storage media.

Invention kits can additionally include cell growth medium, buffering agent, a preservative, or a cell stabilizing agent. Each component of the kit can be enclosed within an individual container or in a mixture and all of the various containers can be within single or multiple packages.

Mature β-cells or in vitro-differentiated insulin-positive β-like cells, as well as cell lines, populations, cultures and compositions comprising the mature β-cells or in vitro-differentiated insulin-positive β-like cells can be included in or employed in a pharmaceutical formulation. In some embodiments, a composition comprising a molecule of interest described herein and an antibody that binds to a putative β-cell surface marker can be included or employed in a pharmaceutical formulation.

Pharmaceutical formulations include “pharmaceutically acceptable” and “physiologically acceptable” carriers, diluents or excipients. The terms “pharmaceutically acceptable” and “physiologically acceptable” mean that the formulation is compatible with pharmaceutical administration. Such pharmaceutical formulations are useful for treatment of, or administration or delivery to, or transplanting or implanting into, a subject in vivo or ex vivo.

Pharmaceutical formulations can be made to be compatible with a particular local, regional or systemic administration or delivery route. Thus, pharmaceutical formulations include carriers, diluents, or excipients suitable for administration by particular routes. Specific non-limiting examples of routes of administration for compositions of the invention are parenteral, e.g., intravenous, intrarterial, intradermal, intramuscular, subcutaneous, intra-pleural, injection, transdermal (topical), transmucosal, intra-cranial, intra-spinal, intra-ocular, rectal, oral (alimentary), mucosal administration, and any other formulation suitable for a method or administration protocol.

One skilled in the art readily appreciates that the present invention is well adapted to carry out the objects and obtain the ends and advantages mentioned, as well as those inherent therein. The details of the description and the examples herein are representative of certain embodiments, are exemplary, and are not intended as limitations on the scope of the invention. Modifications therein and other uses will occur to those skilled in the art. These modifications are encompassed within the spirit of the invention. It will be readily apparent to a person skilled in the art that varying substitutions and modifications may be made to the invention disclosed herein without departing from the scope and spirit of the invention.

The articles “a” and “an” as used herein in the specification and in the claims, unless clearly indicated to the contrary, should be understood to include the plural referents. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention also includes embodiments in which more than one, or all of the group members are present in, employed in, or otherwise relevant to a given product or process. Furthermore, it is to be understood that the invention provides all variations, combinations, and permutations in which one or more limitations, elements, clauses, descriptive terms, etc., from one or more of the listed claims is introduced into another claim dependent on the same base claim (or, as relevant, any other claim) unless otherwise indicated or unless it would be evident to one of ordinary skill in the art that a contradiction or inconsistency would arise. It is contemplated that all embodiments described herein are applicable to all different aspects of the invention where appropriate. It is also contemplated that any of the embodiments or aspects can be freely combined with one or more other such embodiments or aspects whenever appropriate. Where elements are presented as lists, e.g., in Markush group or similar format, it is to be understood that each subgroup of the elements is also disclosed, and any element(s) can be removed from the group. It should be understood that, in general, where the invention, or aspects of the invention, is/are referred to as comprising particular elements, features, etc., certain embodiments of the invention or aspects of the invention consist, or consist essentially of, such elements, features, etc. For purposes of simplicity those embodiments have not in every case been specifically set forth in so many words herein. It should also be understood that any embodiment or aspect of the invention can be explicitly excluded from the claims, regardless of whether the specific exclusion is recited in the specification. For example, any one or more active agents, additives, ingredients, optional agents, types of organism, disorders, subjects, or combinations thereof, can be excluded.

Where the claims or description relate to a composition of matter, it is to be understood that methods of making or using the composition of matter according to any of the methods disclosed herein, and methods of using the composition of matter for any of the purposes disclosed herein are aspects of the invention, unless otherwise indicated or unless it would be evident to one of ordinary skill in the art that a contradiction or inconsistency would arise. Where the claims or description relate to a method, e.g., it is to be understood that methods of making compositions useful for performing the method, and products produced according to the method, are aspects of the invention, unless otherwise indicated or unless it would be evident to one of ordinary skill in the art that a contradiction or inconsistency would arise.

Where ranges are given herein, the invention includes embodiments in which the endpoints are included, embodiments in which both endpoints are excluded, and embodiments in which one endpoint is included and the other is excluded. It should be assumed that both endpoints are included unless indicated otherwise. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise. It is also understood that where a series of numerical values is stated herein, the invention includes embodiments that relate analogously to any intervening value or range defined by any two values in the series, and that the lowest value may be taken as a minimum and the greatest value may be taken as a maximum. Numerical values, as used herein, include values expressed as percentages. For any embodiment of the invention in which a numerical value is prefaced by “about” or “approximately”, the invention includes an embodiment in which the exact value is recited. For any embodiment of the invention in which a numerical value is not prefaced by “about” or “approximately”, the invention includes an embodiment in which the value is prefaced by “about” or “approximately”. “Approximately” or “about” generally includes numbers that fall within a range of 1% or in some embodiments within a range of 5% of a number or in some embodiments within a range of 10% of a number in either direction (greater than or less than the number) unless otherwise stated or otherwise evident from the context (except where such number would impermissibly exceed 100% of a possible value). It should be understood that, unless clearly indicated to the contrary, in any methods claimed herein that include more than one act, the order of the acts of the method is not necessarily limited to the order in which the acts of the method are recited, but the invention includes embodiments in which the order is so limited. It should also be understood that unless otherwise indicated or evident from the context, any product or composition described herein may be considered “isolated”.

### EXAMPLES

### Example 1: The Mature β-Cell Transcriptome and Differentially Expressed Genes, Signaling Pathways, and Biological Processes in Mature and Immature β-Cells

### Materials and Methods

### Human Islet Isolation

Fresh human pancreatic islets from non-diabetic donors were obtained through the National Disease Research Interchange (NDRI). The use of human tissue was approved by the Harvard University Committee on the Use of Human Subjects in Research. Clinical information on islet donors is listed in Table 5. Islets were collected with cold ischemia time less than 12 hours and shipped overnight in CMRL 1066 media (Invitrogen), 1% human serum albumin, no phenopthalein. The NDRI reported islet purity, as determined through dithizone (diphenylthiocarbazone) staining, and cell viability, as determined by trypan blue exclusion.

Upon arrival, islets were washed with PBS and cultured in CMRL-1066 (modified for islet cell culture by Cellgro/Mediatech) with 5% fetal bovine serum (FBS), penicillin (100 U/ml), streptomycin (100 μg/ml), and 5.5 mM D-glucose. Islets were gently dispersed by incubation with Accutase at 37° C. for 10 minutes and occasional trituration with a P1000 micropipettor. Undispersed cell aggregates were removed with a 40 μm strainer (BD Falcon).

Cells were seeded onto plates coated with Matrigel (BD Biosciences) and cultured for one to seven days before analysis.

TABLE 5
Human islet donor clinical information and islet quality.

| | Donor 1 | Donor 2 | Donor 3 |
|---|---|---|---|
| Sex | Male | Male | Female |
| Age (years) | 27 | 19 | 24 |
| Race | Caucasian | Caucasian | Caucasian |
| BMI (kg/m2) | 30.3 | 29.2 | 22.7 |
| Blood type | O | A | B |
| Diabetes | no | no | no |
| Chemo/Rad | no | no | nd* |
| Cause of death | head trauma | trauma | head trauma |
| Islet purity | 60-70% | 90% | 90% |
| Cell viability | >90% | 91% | 90% |

BMI, body mass index.
Chemo/rad, exposure to chemotherapy or radiation therapy.
nd*, no data, but medical history indicated no cancer.

hESC Culture and Directed Differentiation

Mouse embryonic fibroblast-conditioned media (MEF-CM) was created by incubating media on a confluent mitomycin C-treated layer of MEFs. The media consisted of DMEM/F12 supplemented with 20% KnockOut Serum Replacement, 2 mM Glutamax, 1.1 mM β-mercaptoethanol, 1 mM nonessential amino acids, and 1×penicillin/streptomycin (all from GIBCO). Recombinant human basic fibroblast growth factor (bFGF; Invitrogen) was added to the media at a concentration of 20 ng/ml immediately prior to use. Human embryonic stem cells (hESCs; H1, NIH designation WA01) were grown in MEF-CM/bFGF and passaged at the ratio of 1:6-1:20 every 4-7 days using TrypLE Express (a trypsin replacement; Invitrogen) onto tissue culture treated dishes coated with a 1:30 dilution of growth factor reduced Matrigel (BD Biosciences). At each passage, hESCs were treated with 10 μM of the Rho-associated kinase inhibitor Y27632 for 24 hours (Calbiochem). To induce differentiation, hESCs were expanded for two to three days following a passage until 60-70% confluency. The cells were cultured in ‘basal medium,’ consisting of MCDB 131 (Invitrogen) supplemented with 2% fatty acid-free bovine serum albumin (Proliant/Lampire) and exposed to culture conditions as follows and outlined in FIG. 1A.

In stage 1 (induction of definitive endoderm), cells were exposed to 100 ng/mL activin A (AA; R&D) and 20 ng/mL Wnt3A (R&D) for one day only. For three additional days, cells were exposed to 100 ng/mL AA only. In stage 2 (induction of primitive foregut), for two days, cells were treated with 50 ng/mL FGF7 (Peprotech). In stage 3 (induction of early pancreatic progenitors), for four days, cells were exposed to 20 ng/mL AA, 50 ng/mL FGF7, 250 nM SANT-1 (Sigma), 2 μM retinoic acid (Sigma), and 100 ng/mL recombinant human noggin (R&D). From stage 3 and forward, the basal media also contained a 1:200 dilution of insulin-transferrin-selenium (ITS-X; Invitrogen). In stage 4 (induction of late pancreatic progenitors), for three days, cells were exposed to 250 nM SANT-1, 100 ng/mL noggin, and 100 nM phorbol 12,13-dibutyrate (EMD Biosciences). In stage 5 (induction of endocrine progenitors), for three days, cells were incubated with 100 ng/mL noggin and 1 μM ALK5 inhibitor II (Axxora). In stage 6 (differentiation into endocrine hormone-expressing cells), cells were kept in basal media supplemented with ITS-X for three to seven days until analysis.

FIG. 1B provides an expanded version of the differentiation protocol depicted in FIG. 1A. In particular, hPSCs were routinely cultured on hESC-certified matrigel (BD Biosciences) in mTeSR medium (Stemcell Technologies). Cells were passaged at the ratio of 1:6-1:20 every 4-7 days using TrypLE Express (Invitrogen). Two different basal medias were used during differentiation. Basal media 1 (BM-1) contained MCDB-131 (Invitrogen) supplemented to 10 mM glucose, 1×GlutaMAX (Gibco, Life Technologies), 2.35 g/l NaHCO₃ and 0.1% reagent grade BSA (Proliant). Basal media 2 (BM-2) contained MCDB-131 (Invitrogen) supplemented to 8 mM glucose, 1×GlutaMAX (Gibco, Life Technologies), 2.93 g/l NaHCO₃, 2% reagent grade BSA (Proliant), 1:200 ITS-X (Invitrogen) and 44 mg/l Vitamin C.

To initiate differentiation the cells were dissociated using TrypLE Express to single cells and seeded at 150,000 cell/cm2 onto 1:30 dilution of growth factor reduced matrigel (BD Biosciences) in DMEM/F12 in mTeSR media with 10 uM Y27632 (StemGent). Two days following seeding the differentiation was started. Day 1 (stage 1.1) cells were exposed to BM-1 supplemented with and 3 uM CHIR-99021 (Stemgent)+100 ng/ml rhActivinA (R&D Systems). Day 2-3 (stage 1.2): BM-1+100 ng/ml rhActivinA (R&D Systems). Day 4-5 (stage 2): BM-1+50 ng/ml FGF7 (Peprotech) (stage 2). Day 6-9 (stage 3) BM-2+50 ng/ml FGF7 (Peprotech)+2 μM RA (Sigma)+0.25 μM SANT-1 (Sigma)+20 ng/ml rhActivinA (R&D Systems)+100 ng/ml rhNoggin (R&D Systems). Day 10-12 (stage 4) BM-2+100 ng/ml rhNoggin (R&D Systems)+0.25 μM SANT-1 (Sigma)+100 nM PDBu (EMD Biosciences) (stage 4). Day 13-15 (stage 5) BM-2+100 ng/ml rhNoggin (R&D Systems)+1 μM Alk5 inhibitor (Axxora). Day 15 onwards cells were kept in BM-2 media awaiting analysis.

### Immunohistochemistry

Dispersed cells were washed with PBS and fixed by immersion in 4% paraformaldehyde (PFA) at room temperature for 20 minutes. Intact islets were fixed overnight at 4° C. to allow thorough penetration of fixative. Culture plates were blocked by incubation at room temperature for 1 h with 10% donkey serum (Jackson Immunoresearch) in PBS and 0.1% Triton-X (Sigma). Cells were incubated with primary and then secondary antibodies in blocking solution each for an hour at room temperature while gently rocking (specific antibodies and concentrations listed in Table 6 and Table 7). After each stain, cells were washed by incubation with PBS/0.1% Triton-X for 5 min while rocking at room temperature. Nuclei were visualized by brief incubation with Hoescht 33342 (Invitrogen; 1:5000 in PBS). Images in each fluorescent channel were taken on an Olympus IX51 Microscope.

TABLE 6
Primary antibodies used in study.

| | | | | Dilution | Dilution |
|---|---|---|---|---|---|
| | | | | for | for |
| | | Manufac- | Catalog | IHC/ | West- |
| Antigen | Species | turer | # | FACS | erns |
| Insulin | guinea | Dako | A0564 | 1:1000 | |
| | pig | | | | |
| C-peptide | rabbit | Cell | 4593 | 1:500 | |
| | | Signaling | | | |
| Glucagon | mouse | Abcam | ab82270 | 1:500 | |
| Chromogranin A | rabbit | Abcam | ab15160 | 1:500 | |
| Somatostatin | goat | Santa Cruz | sc-7819 | 1:1000 | |
| Nkx6.1 | mouse | DSHB | F55A12 | 1:100 | |
| MafA | rabbit | Abcam | ab-26405 | 1:1000 | |
| Pdx1 | goat | R&D | AF2419 | 1:500 | 1:300 |
| Glucokinase | rabbit | Santa Cruz | sc-130765 | | 1:200 |
| Ran | mouse | BD | 610341 | | 1:1000 |
| | | Biosciences | | | |

IHC, immunohistochemistry.
FACS, fluorescence activated cell sorting.
DSHB, Developmental Studies Hybridoma Bank.

TABLE 7
Secondary antibodies used in study.

| | | | | Dilution | Dilution |
|---|---|---|---|---|---|
| | | | | for | for |
| Antigen | Species/Conjugate | Manufacturer | Catalog # | IHC/FACS | Westerns |
| Guinea pig IgG | goat/AF647 | Invitrogen | A21450 | 1:500 | |
| Guinea pig IgG | donkey/AF594 | Invitrogen | A11076 | 1:500 | |
| Guinea pig IgG | donkey/DL649 | Jackson IR | 706-496-148 | 1:500 | |
| Mouse IgG | donkey/AF488 | Invitrogen | A21202 | 1:500 | |
| Rabbit IgG | donkey/AF488 | Invitrogen | A21206 | 1:500 | |
| Rabbit IgG | goat/AF405 | Invitrogen | A31556 | 1:500 | |
| CD142 | mouse/R-PE | BD Biosciences | 550312 | 1:10 | |
| CD56 | mouse/APC | BD Biosciences | 555518 | 1:10 | |
| CD200 | mouse/APC | R&D | MAB27241 | 1:10 | |
| CD56 | mouse/H V450 | BD Biosciences | 560361 | 1:20 | |
| Mouse IgG | sheep/HRP | GE Healthcare | NXA931 | | 1:5000 |
| Goat IgG | rabbit/HRP | Invitrogen | 811620 | | 1:5000 |
| Rabbit IgG | donkey/HRP | GE Healthcare | NA934 | | 1:5000 |

AF, Alexa Fluor.
DL, DyLight.
H, BD Horizon.
PE, phycoerythrin.
APC, allophycocyanin.
HRP, horseradish peroxidase.
IR, ImmunoResearch Laboratories.

### Glucose-Stimulated Insulin Secretion Assay

In 96-well format, approximately 1000 islet cells amidst 5×105 hESCs (for cell-cell contact and attachment) were plated or, alternatively, approximately 5×105 hESC-derived S6 cells were plated and allowed to attach overnight. Cells were then washed three times in PBS.

Cells were incubated for 1 hour in Kreb's buffer with no glucose (128 mM NaCl, 5 mM KCl, 2.7 mM CaCl2, 1.2 mM MgCl2, 1 mM Na2HPO4, 1.2 mM KH2PO4, 5 mM NaHCO3, 10 mM HEPES, 0.1% BSA) at 37° C., 5% CO2, and then incubated for 60 min in Kreb's buffer with 2.8 mM (low) glucose, followed by 60 min in Kreb's buffer with 16.7 mM (high) glucose, or 30 mM KCl in the same conditions. Supernatant fractions after each exposure to glucose were collected and stored at −80° C. until analysis. Insulin concentrations were measured using the Mouse Ultrasensitive Insulin ELISA kit (Alpco, 80-INSMSU-E01), which cross-reacts >100% with human insulin. Concentrations were calculated from cubic spline interpolation of a standard curve and normalized to input cell number.

### Quantitative RT-PCR

Complementary DNA (cDNA) was made from 4 ng unamplified total RNA with random hexamer priming using the High Capacity cDNA Reverse Transcription with RNase Inhibitor kit (Applied Biosystems). One-fourth of the volume of cDNA was used for each TaqMan quantitative reverse transcription polymerase chain reaction (qRT-PCR). Reactions contained transcript-specific TaqMan probes (Applied Biosystems, Table 8) and Fast Universal PCR Master Mix with no AmpErase UNG (Applied Biosystems). Reactions were run on an Applied Biosystems 7900HT Fast Real-Time PCR System with default settings.

Detection thresholds were automatically computed by SDS 2.3 software (Applied Biosystems). Threshold data were analyzed in DataAssist 3.0 (Applied Biosystems) using the Comparative Ct (ΔΔCt) relative quantitation method, using β-actin as the endogenous

control.

TABLE 8
TaqMan gene expression assays.

| Gene (SYMBOL) | Probe name |
|---|---|
| β-Actin (ACTB) | Hs99999903_m1 |
| Insulin (INS) | Hs00355773_m1 |
| Glucagon (GCG) | Hs00174967_m1 |
| Somatostatin (SST) | Hs00356144_m1 |

### Global Gene Expression Analysis—Microarray

Using the Illumina TotalPrep RNA Amplification kit (Ambion), double-stranded

cDNA was generated following reverse transcription from 100 ng of total RNA. In vitro transcription overnight with biotin-labeled nucleotides created amplified mRNA (cRNA), which was concentrated by vacuum centrifugation at 30° C. 750 ng cRNA per sample was then hybridized to Human HT-12 Expression BeadChips (Illumina) using the Whole-Genome Expression Direct Hybridization kit (Illumina). Finally, chips were scanned on the Illumina Beadstation 500. The chip annotation manifest was version 4, revision 1. For differential expression analysis and the generation of gene lists for functional annotation and pathway analysis, microarray data were processed in GenomeStudio (Illumina, V2011.1). Raw data were adjusted by background subtraction and rank-invariant normalization. Before calculating fold change, an offset of 20 was added to all probe set means to eliminate negative signals. The p-values for differences between mean signals were calculated in GenomeStudio by t-test and corrected for multiple hypotheses testing by the Benjamini-Hochberg method in combination with the Illumina custom false discovery rate (FDR) model.

### Global Gene Expression Analysis—RNA-Seq

Isolated RNA was obtained from 2 biological replicates of HUES8-derived INS⁺ cells and human adult β cell, as well as one replicate of live and processed stage 6 cells. Samples were poly-A purified and converted to cDNA libraries using the Illumina TruSeq protocol, and prepared into Illumina libraries using the Beckman Coulter Genomics SPRI-works system using custom adapters. Ent 3′ barcodes were added during PCR enrichment and the resulting fragments were evaluated using Agilent BioAnalyzer 2100. Samples were multiplexed 2-per-lane for sequencing using the Illumina HiSeq 2000 platform with paired-end read lengths of 80 nt, resulting in 68M to 112M paired reads per sample, and an average biological fragment length of 168-179 nt. Reads were aligned to the human genome (GRCh37/hg19) using TopHat (version 2.0.4) (Trapnell et al. Nature Protocols. 2012; 7:562-578; Trapnell et al. Nature Biotechnology. 2010; 28:511-515) guided by Ensembl gene annotations. RNA-seq enrichment in annotated Ensemble transcripts was determined by Cuffdiff (version 2.0.2) (Trapnell et al. Nature Protocols. 2012; 7:562-578; Trapnell et al. Nature Biotechnology. 2010; 28:511-515) which performed a maximum likelihood estimate of transcript abundance measured in fragments per kilobase of exon per million fragments mapped (FPKM). Statistically significant transcript differential expression was calculated by Cuffdiff using the default negative binomial model, along with associated p-values.

### RNA-Seq Transcript Integrity Analysis

To identify any RNA fragment length bias introduced by the protocol, TopHat aligned RNA-seq read counts from before and after were analyzed at Ensembl annotated exon locations (GRCh37/hg19 version 68) (Flicek et al. Nucleic acids research. 2012; 40:90). Per-bp RNA-seq read coverage was normalized by total mean log counts and summed over all exonic regions. Transcripts were binned by exonic length and average per-transcript coverage was adjusted for length (FIG. 10). In transcripts longer than 2.5 knt (% of genome annotations), the processed sample 3′ bias was greater than the live sample by roughly 2% (AUC differential).

### Data Preprocessing

For clustering analysis, principal component analysis, gene set enrichment analysis, and all heatmap visualization, the microarray data were examined using GenePattern (Broad Institute; version 3.3.3). Raw data were first converted into .gct expression files. Background signal, calculated as the mean of the negative control signal values, was subtracted from all gene probe values. Expression data were then normalized by the quantile method. The data were filtered by first removing rows (probes) in which all columns (samples) had average signals less than 20 and then equating all negative signals to 0.1. For differential expression analysis and the generation of gene lists for functional annotation and pathway analysis, microarray data were processed in GenomeStudio (Illumina, V2011.1). As above, raw data were adjusted by background subtraction and quantile normalization. Before calculating fold change, an offset of 20 was added to all probe set means to eliminate negative signals. The p values for differences between mean signals were calculated in GenomeStudio by t-test and corrected for multiple hypotheses testing by the Benjamini-Hochberg method incombination with the Illumina custom false discovery rate (FDR) model.

### Microarray Expression Clustering

Gene level microarray expression values were generated by GenomeStudio, using rank invariant normalization with background subtraction, and analyzed using the R package (Suzuki and Shimodaira. Bioinformatics. 2006; 22:1540-1542). Per-sample and per-condition (averaging gene levels) hierarchical clustering was performed based on Pearson correlation and dendogram visualizations were drawn. pvclust's multiscale bootstrap resampling was used (with 500 k iterations) to estimate “approximately unbiased” (AU) p-values indicating the significance of each subcluster choice in the hierarchy given the underlying data.

### DAVID Gene Ontology

The representation of gene ontology (GO) biological process terms within the lists of differentially expressed genes was assessed using the Database for Annotation, Visualization, and Integrated Discovery (DAVID; http://david.abcc.ncifcrf.gov) Bioinformatics Resource. The p-values were corrected for multiple hypotheses testing by the Benjamini-Hochberg FDR procedure and expressed as a q-value. Terms were ranked by q-value.

### Hierarchical Clustering and Principal Component Analysis

In GenePattern, we performed unsupervised hierarchical clustering, a common approach to group samples by similar expression profiles and to find the innate sample structure within the data (Eisen et al., 1998). The data were centered by mean subtraction and normalized within each row. To place gene expression induction and repression on the same linear scale, log 2 transformation was performed. Hierarchical clustering was performed using Pearson's correlation coefficient and pairwise centroid-linkage as the similarity measure and clustering algorithm, respectively. Results were visualized in dendrograms, with distances representing differences in centroid correlation.

To validate groups discovered by hierarchical clustering and verify the main variables driving variation in the data, in GenePattern, we performed principal components analysis (PCA), a mathematical transformation procedure that reduces the high-dimensional, noisy gene expression data to low-dimensional components that represent major, invariant expression patterns (Raychaudhuri et al., 2000). Preprocessed data, but not log-transformed, centered, nor row-normalized, were used to cluster samples according to PCA.

### Differential Expression Analysis

Differential expression compared to a reference sample group was performed in GenomeStudio to generate t-test-based adjusted p-values for differences in expression. Selection of differentially expressed genes was based on bio-weight analysis (Rosenfeld et al., 2004). Bio-weight is the product of the log 2 fold change in means and the negative log 10 of pvalue, thereby providing a smooth transition in data sets between these two parameters. Bioweight accounts for significance defined both by small t-test p-values as well as large fold changes. Especially for smaller sample sets, bio-weight tends to produce a higher truepositive discovery rate compared to traditionally used fold change-based or p-value based analysis alone (Rosenfeld et al., 2004). Thus, with one metric, users can identify differentially expressed genes that may be of both biological and statistical significance.

Here, we used an absolute value bio-weight cutoff of 2 to determine differentially expressed probe sets. This metric is equivalent to a two-fold change at p=0.01 and a 2.9-fold change at p=0.05. Differentially expressed probes lacking an ENTREZ gene ID were removed from further analysis (n=8).

To display heatmaps of differentially expressed genes, unique probe identifiers from the preceding analyses were exported to GenePattern to generate a .gct file containing only that subset of probes. Rows were reordered by hierarchical clustering on Pearson correlation or K-means clustering on Euclidean distance and then visualized with row-normalized coloring.

### Functional Annotation and Pathway Analysis

To assess the biological patterns of differential gene expression, we measured the representation of gene ontology (GO) terms (association of gene products with their related biological processes and molecular functions) within the lists of differentially expressed genes using the Database for Annotation, Visualization, and Integrated Discovery (DAVID; http://david.abcc.ncifcrf.gov) Bioinformatics Resource. This allowed us to examine whether any GO term was over- or under-represented as compared with what is expected occur by chance by a Fisher's exact test with the EASE (Expression Analysis Systematic Explorer) modification. The p-values were corrected for multiple hypotheses testing by the Benjamini-Hochberg FDR procedure and expressed as a q-value.

Overlap statistics were calculated between the differentially expressed gene list and the category GOTERM_BP_FAT, an agglomeration of all GO biological process terms in DAVID that has removed the broadest, most overlapping categories. Terms were ranked by q-value. To further define non-overlapping categories, we used the DAVID functional annotation clustering tool, which groups terms that share a significant subset of the same genes (e.g. “cell cycle” and “cell cycle regulation”). For annotation clustering, only terms with a nominal EASE score (p-value) less than 0.05 were included, and were grouped under the “low classification stringency” setting. The most enriched term (lowest p-value) from each cluster, which usually but not always was the term containing the most genes represented, were displayed in pie charts with section size determined by the number of genes.

Overrepresentation of biological pathways (specifically, signaling networks and targets of transcription factors) in the list of differentially expressed genes were visualized in Ingenuity Pathway Analysis (IPA), which makes use of a proprietary, knowledge-based database of gene sets. As before, differential expression was defined by bio-weight and pvalues are derived from the probability of observing the overlap between the differential gene list and the pathway gene list by chance alone.

### Generation of Lists of Transcription Factors and Surface Markers

From the lists of differentially expressed genes generated by microarray, gene products were filtered by GO terms within GenomeStudio. To determine a list of transcription factors, genes were selected based on GO terms matching any of the following identifiers (GOIDs) and definitions: 3700 (“sequence-specific DNA binding transcription factor activity”), 30528 (“transcription regulator activity”), 43565 (“sequence-specific DNA binding”), or the combination of 6355 (“regulation of transcription, DNA-dependent”) and 3677 (“DNA binding”).

To determine a list of candidate surface markers in silico, differentially expressed genes were filtered based on the GO cellular component terms 5887 (“integral to plasma membrane”), or the combination of 16021 (“integral to membrane”) and 5886 (“plasma membrane”). From this list, transmembrane topology was evaluated using Phobius (http://phobius.sbc.su.se), a hidden Markov model-based prediction program (Käll et al., 2004). Proteins with fewer than 6 consecutive extracellular amino acids or no transmembrane domains were excluded.

### Gene Set Enrichment Analysis

Gene set enrichment analysis (GSEA) was used in GenePattern to assess enrichment of defined pathways between sample groups using a rank-based metric across the whole dataset rather than using bio-weight cutoffs to define a subset of differentially expressed genes (Subramanian et al., 2005). This computational method is more sensitive to small concordant changes that occur broadly across a defined pathway. GSEA was performed with most default settings, including mean centering, weighted scoring, and using only the maximum expression value of the multiple probes mapping to the same gene. The metric for ranking was signal-to-noise ratio, defined as the difference in means divided by the sum of standard deviations. Gene sets were randomly permuted for 1000 iterations to determine the p-value of discovering enriched sets by chance. The gene set database used was canonical pathways from Reactome (http://www.reactome.org) or custom-made β-cell signature gene sets. Publically available gene sets with fewer than 15 members or more than 500 members represented in the data set were excluded because enrichment scores can be hard to interpret beyond in these ranges. A stringent FDR cutoff of q≤0.05 was used to determine statistical significance.

### Statistical Weighted Voting

All 1403 features of the β/non-β signature gene sets were used to generate a class predictor model (Golub et al., 1999). Each feature (probe) was assigned a signal-to-noise statistic that reflects how well a probe correlates with class distinction, defined as S=(μ0−μ1)/(ρ0+ρ!), where 0 and 1 are the classes β-cell and non-β islet cell), μ is the class mean signal, and ρ is the class standard deviation. Half-way decision boundaries were defined as Bx=(μ0+μ1)/2 for each probe x. To predict the class of a sample, each probe casted a vote of win or lose based on the deviation from the decision boundary and weighted by the signal-to-noise ratio:

Vx=Sx(Gx−Bx).

A vote went to class 0 if sign(VxSx)=1 and to class 1 if sign(VxSx)=−1. The prediction confidence index, reflecting margin of victory, was defined as (Vwin−Vlose)/(Vwin+Vlose), where Vwin and Vlose are the sum of votes for or against a class, respectively.

### Results

### Establishment of a Beta-Cell Molecular Signature

Having established the general robustness of gene expression data obtained from cells purified by modified IC-FACS, we proceeded to investigate the transcriptional signature of β-cells. We identified 1403 probe sets (3.0% of probes on the array) differentially expressed between biological triplicates of β-cells and non-β islet cells (i.e. islets depleted of β-cells) by a t-test-based metric (|bio-weight|>2, where bio-weight=−log 10 p-value×log 2 fold-change). These included 514 probes (455 genes; FIG. 12) with expression specific to β-cells, and 889 probes (760 genes; FIG. 13) with expression specific to non-β islet cells.

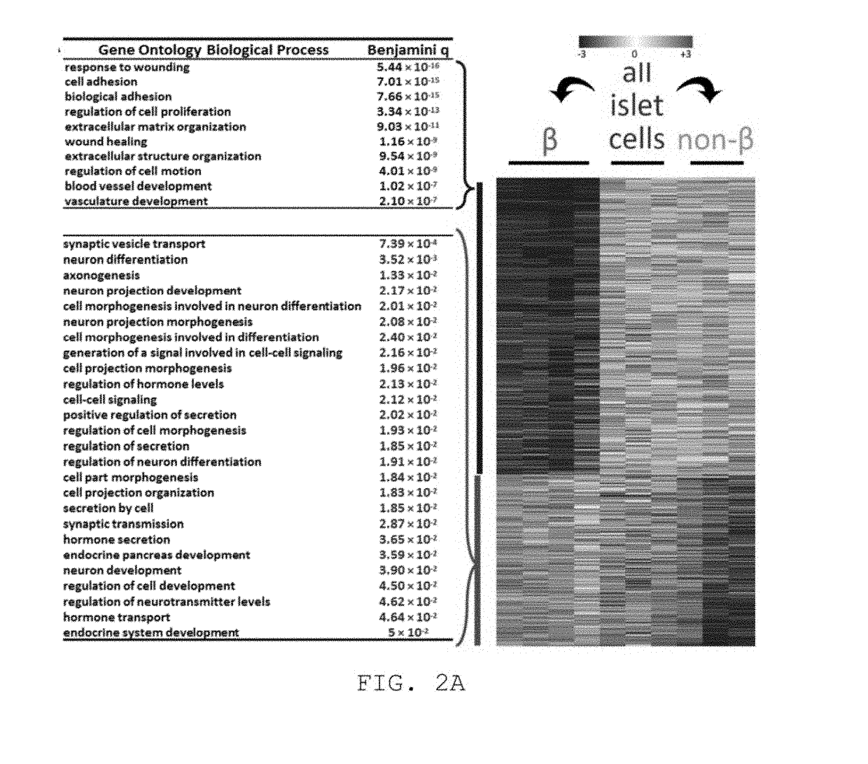

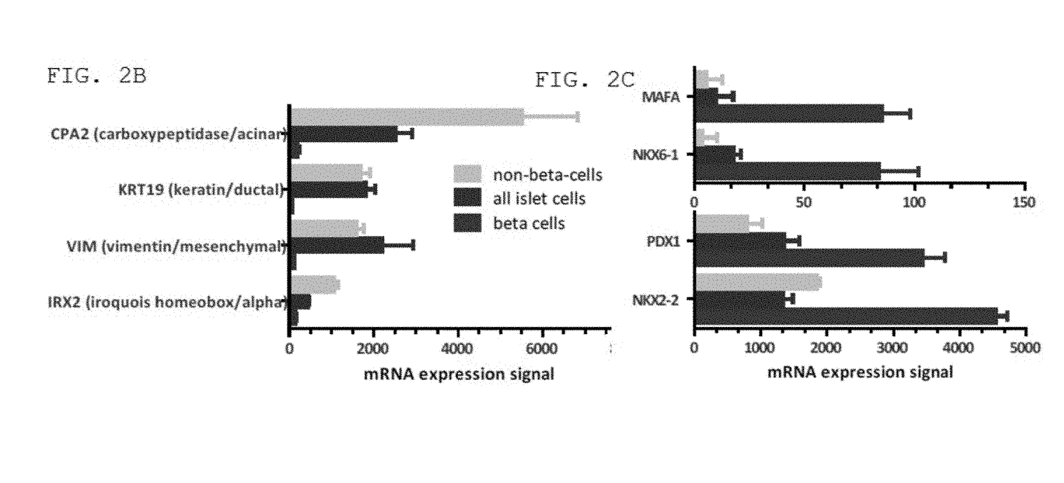

We examined the gene ontology (GO) biological process annotations for each gene and assessed whether certain terms appeared more often than would be expected by chance (FIG. 2A). From the genes enriched in non-β islet cells, the most significantly overrepresented annotations included ones related to adhesion, extracellular matrix, wound response, and vascular development (Benjamini-Hochberg FDR-corrected q<10-6). These terms are consistent with the presence of connective tissue within the islet cell mixture, including mesenchyme, endothelial cells, and blood cells. From the genes enriched in β-cells, significantly overrepresented annotations included ones related to neuronal differentiation, morphogenesis, and secretion, indicating a concordance between neuronal regulatory pathways and pancreatic endocrine function. Other significantly overrepresented terms included those related to regulation of hormone secretion and endocrine pancreas development (q<0.05). Together, these observations demonstrate that the lists of differentially expressed genes in β-cells relative to other cells present in the pancreas are generally consistent with expected biological functions.

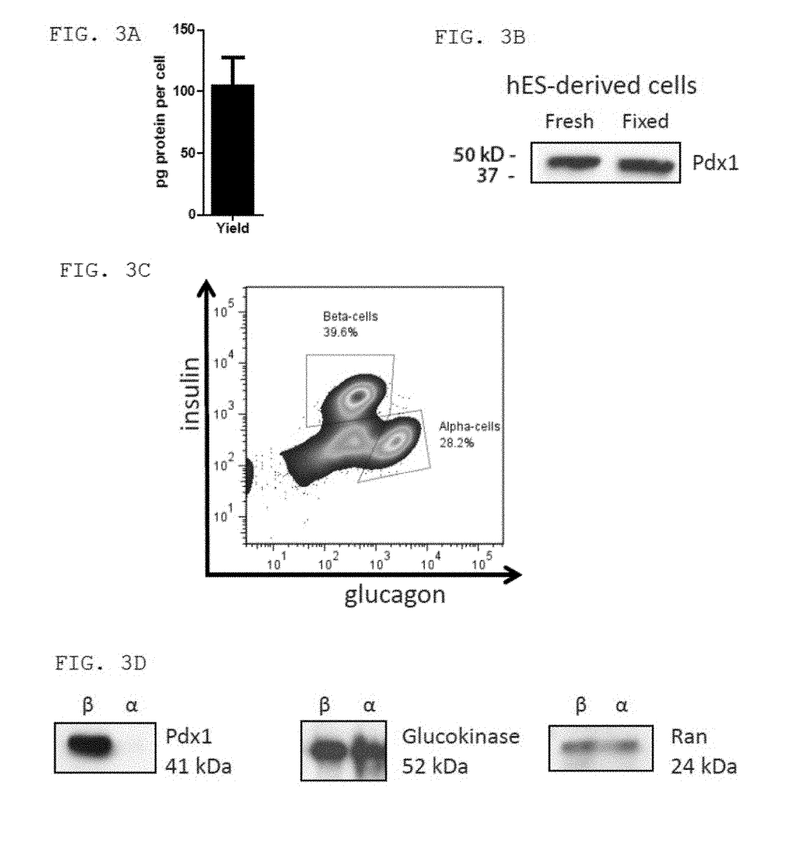

To further confirm the proper sorting of different cell types, we examined the expression of specific, established markers of various pancreatic cell types within the list of differentially expressed genes. The non-β islet samples showed significantly higher expression of acinar enzymes (e.g. carboxypeptidase, carbonic anhydrase, trypsins, chymotrypsinogens, elastase, pancreatic lipases), ductal markers (keratin 19, mucin 1, HNF1β), mesenchymal proteins (vimentin), endothelial markers (VCAM1), and the α-cellassociated transcriptional regulator IRX2 (FIG. 2B). The α-cell transcription factor ARX did not show a significant difference on our array, though we have not confirmed this finding by qRT-PCR. Failure of this marker to reach statistical significance may be attributed in part to the relatively small number of α-cells in the non-β sorted fraction (<30%). In contrast, many known β-cell-specific markers were expressed significantly higher in the purified β-cell population. These included many transcription factors (see below), as well as insulin biosynthesis machinery (e.g. PCSK1, which cleaves proinsulin) and signaling receptors (e.g. ABCC8, the sulfonyl urea receptor and potassium channel that is the target of some diabetes drugs; and GCGR, the glucagon receptor). At the protein level, we confirmed the β-cell-specific expression of PDX1 and the non-differential expression of glucokinase (GCK), a critical molecule for glucose sensing in β-cells but also ubiquitously expressed as part of the glycolysis pathway (FIG. 3). These data demonstrate the validity of our experimental approach through their concurrence with previous knowledge of the different pancreatic lineages. Many differentially expressed genes, however, were identified that had no known roles specifically in cell types of the pancreas. We have therefore defined a comprehensive molecular signature that distinguishes human β-cells from other pancreatic cells.

Because cell identity is largely determined by factors that control the expression of a large number of genes involved in cellular functions, we investigated differentially expressed regulators of transcription. Within the β-cell-specific signature gene list, 32 genes were transcriptional regulators that interact selectively with DNA (Table 9). This list includes well-established β-cell transcription factors like PDX1, MAFA, NKX6-1 (FIG. 2C); known pancreatic development regulators not well-characterized in human cells like NKX2-2 and MNX1; factors with only some evidence of relevance to β-cell biology, like CLOCK, DACH1, MYT1L, and MLXIPL; and many regulators not previously described at all in pancreatic islets. Thus, we have identified transcriptional regulators that selectively express in human β-cells.

TABLE 9
Transcription factors specific to human β-cells.

ASCL2\hat{ }
CHD7***
CLOCK**
DACH1***
DACH2***
DDIT3*
ETV5\hat{ }
HOPX\hat{ }
INSM1**
MAFA*
MLXIPL**
MNX1/HLXB9*
MYT1L***
NDN***
NKX2-2*
NKX6-1*
NR0B1\hat{ }
NR3C1**
OLIG1\hat{ }
PDX1*
PEG3***
PKNOX1**
PLAGL1**
RXRG***
SIX4***
TOX\hat{ }
TSHZ3\hat{ }
TSNAX\hat{ }
ZNF10\hat{ }
ZNF395\hat{ }
ZNF540\hat{ }
ZNF672\hat{ }
Scheme is based on number of publications listed in the Type 1 Diabetes database associated with a given gene, as a index for whether the gene has known roles in β-cell biology:
*n ≥ 10;
**1 < n < 10;
***n = 1;
\hat{ }n = 0.

A secondary goal of this study was to identify β-cell surface markers. Though these molecules are unlikely to determine cell identity in any active sense, surface markers are necessary for functional and developmental assays that require isolation of live β-cells.

Analysis of GO cellular component annotations and topology prediction identified integral plasma membrane proteins with extracellular domains within the β-cell signature gene list (Table 10). Though validation of these markers was beyond the scope of this thesis, this in silico study identified several expected molecules, including ABCC8 and GCGR.

Notably, FXYD2, a regulating subunit of the Na+-K+-ATPase, was recently identified as a β-cell-selective biomarker, though flow cytometric analyses have not yet been reported (Flamez et al., 2010). Thus, this list provides a starting point for identifying novel β-cellspecific surface markers with sorting potential.

TABLE 10
Potential surface markers of human β-cells.

| | | | | | | |
|---|---|---|---|---|---|---|
| ABCA3 | CD79B | FXYD2 | KCNB2 | NLGN1 | PTPRU | SLC6A9 |
| ABCC8 | CD8A | GCGR | KCNF1 | NPR2 | ROBO1 | SORL1 |
| ABCG1 | CDH2 | GPR120 | KCNG3 | NRCAM | RTN4 | SVOP |
| ACSL1 | CDH22 | GPR19 | KCNH2 | PCDHA1 | SEMA5A | TGFBR3 |
| ATP1B2 | CHRNA5 | GRIA2 | KCNMA1 | PCDHA3 | SERP2 | TRPM2 |
| CACNA1H | CYB561 | KCNH1 | KCNQ2 | PIGU | SLC17A6 | TRPM5 |
| CADM1 | EFNB3 | IGSF11 | MADD | PLXNA2 | SLC43A2 | TSPAN13 |
| CASR | FFAR1 | IL17RB | NEO1 | PRRG2 | SLC6A6 | UNC5A |

Transcriptional Profiling of hESC-Derived Insulin+ Cells

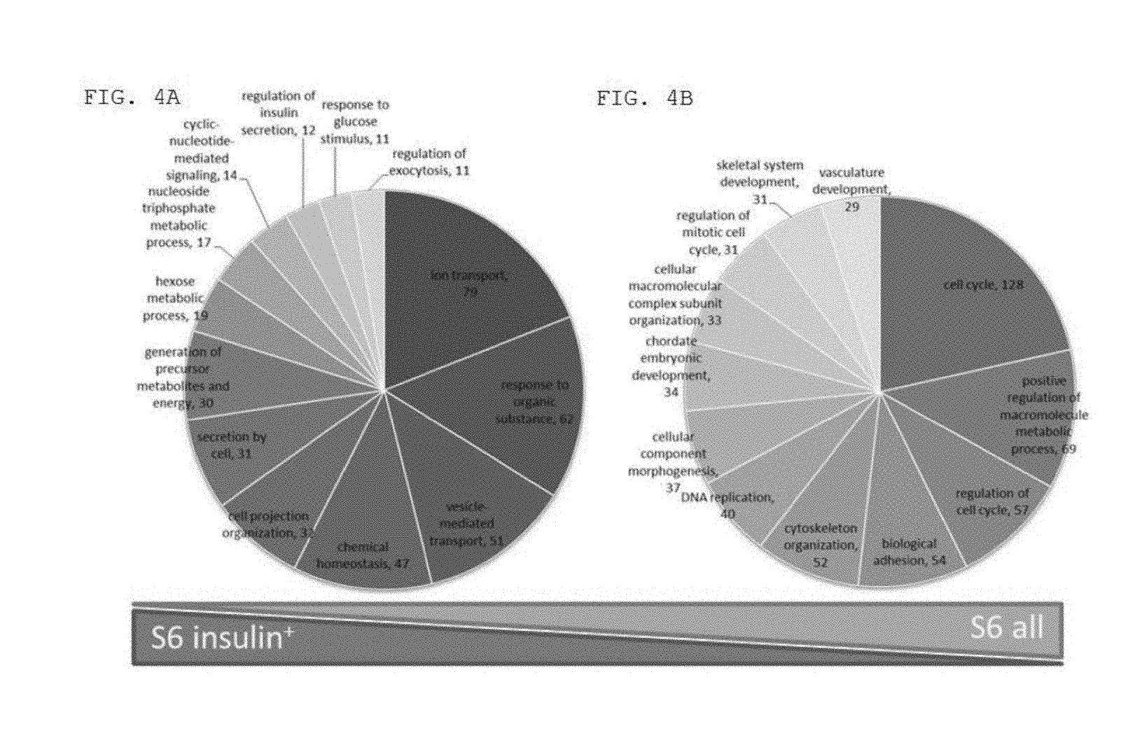

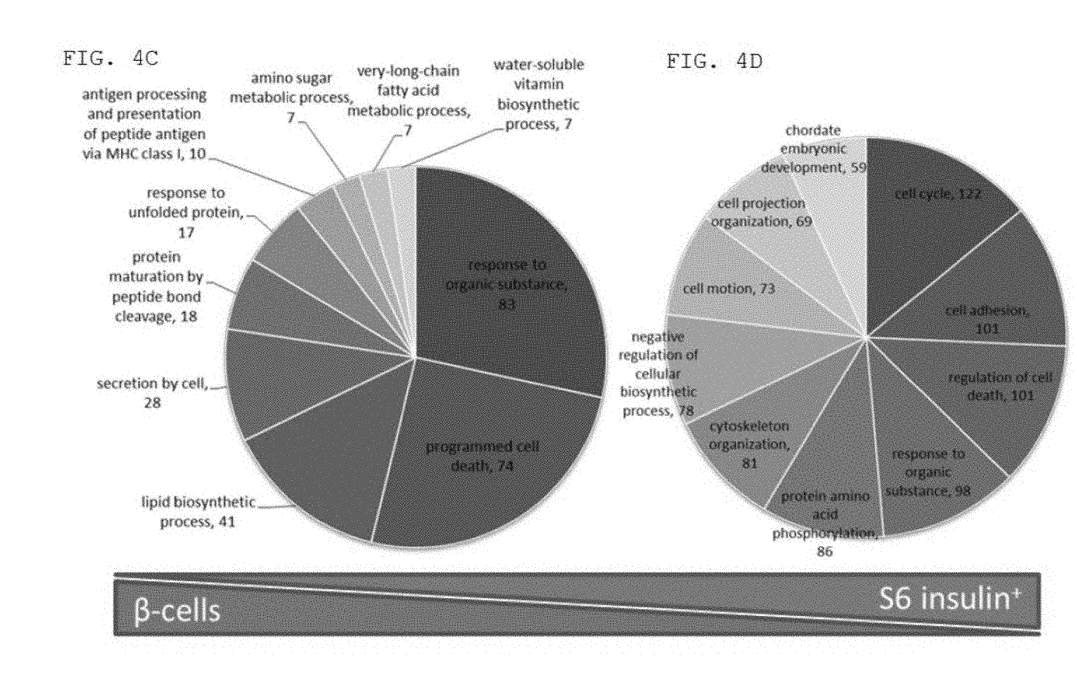

We next performed transcriptional profiling to characterize hESC-derived insulin+ cells. Global analysis of S6 insulin+ cells compared to unsorted S6 cells revealed that 1222 probes (2.6%) had significantly increased signal in insulin+ cells and 1104 (2.3%) had significantly decreased signal. Clustering analysis of GO biological process terms revealed that insulin+ cells had higher expression in genes associated with ion transport, response to organic substances, cell secretion and regulation of insulin secretion, vesicle-mediated transport, and glucose metabolism, among other terms (FIG. 4A). These relations suggest that insulin+ S6 cells have more potential for endocrine functionality than the bulk S6 population. In contrast, unsorted S6 cells were enriched for genes involved in cell cycle regulation, adhesion, DNA replication, morphogenesis, embryonic development, skeletal development, and vascular development (FIG. 4B). These associations are suggestive of proliferating progenitor cells in the process of differentiation into diverse lineages.

Global analysis of hESC-derived S6 insulin+ cells compared to β-cells identified 2326 probes (4.9%) with significantly increased expression in S6 insulin+ cells and 1869 (4.0%) with significantly decreased expression. Compared to S6 insulin+ cells, β-cells had higher expression in genes that participate in lipid and fatty acid metabolism, amino sugar metabolism, cellular secretion, and protein maturation by peptide bond cleavage (FIG. 4C). These processes are consistent with a mature endocrine phenotype that responds to nutrient levels and secretes insulin. In contrast, compared to β-cells, S6 insulin+ cells had higher expression of genes that participate in the cell cycle, adhesion, cell motion, embryonic development, and negative regulation of cellular biosynethic processes (FIG. 4D).

These processes are suggestive of a relatively immature phenotype in which cells are not terminally differentiated and are devoting more resources to expansion than to biosynthesis.

In both β-cells and S6 insulin+ cells, “response to organic substance” appeared as an enriched term, suggesting that this process is dysregulated in S6 insulin+ cells but GO term analysis is unable to dissect apart components of this process and assign directionality.

### Candidate Endocrine Gene Analysis

To scrutinize more specific components of endocrine specification, we examined expression patterns of 73 genes thought to be involved in endocrine pancreas development or function (FIG. 5A). We derived this approach from a previous report characterizing hESC-derived cells (Basford et al., 2011) and refined the list of genes based on the literature on rodent and human islet developmental biology. First, we compared unsorted S6 cells and S6 insulin+ cells to assess the degree of their commitment to the endocrine lineage. Consistent with our findings that insulin+ cells express higher insulin, glucagon, and somatostatin than the bulk S6 population (not shown), we found here that insulin+ cells express significantly higher levels of endocrine markers of the pancreas, stomach, and intestine, including CHGB (chromogranin B), GAST (gastrin), GHRL (ghrelin), PPY (pancreatic polypeptide Y), PYY (peptide YY), and IAPP (islet amyloid polypeptide). Of all the genes examined, 40 were differentially expressed between unsorted and insulin+ fractions, with all but three of those having higher expression levels in the insulin+ fraction (FIG. 5B).

Within that list, many transcription factors that control endocrine cell identity were more highly expressed in the S6 insulin+ than the unsorted fraction. In contrast, the transcriptional regulators HES1, NOTCH1, and SOX9, which are involved in earlier patterning of gut endoderm, showed higher expression in the unsorted S6 fraction. Insulin+ cells expressed most genes involved in endocrine function. These included many components involved in exocytosis (RIMS2, SNAP25, STX1A, SYP, SYT7, and STXBP1) and KATP and Ca2+ ion channels critical for islet secretory function (including genes that encode Kir6.2, SUR1, CaV2.1, CaV1.2). Additionally, GCK, which functions as a glucose sensor and metabolic regulator, was expressed more highly in insulin+ cells. FFAR1, a fatty acid receptor that modulates β-cell metabolism, and SLC30A8, a zinc transporter implicated in insulin granule maturation, were also elevated. Taken together, these expression patterns strongly suggest that compared to the bulk S6 population, the insulin+ fraction more closely resemble endocrine lineage cells, albeit heterogeneous or polyhormonal.

Next, we compared S6 insulin+ cells to sorted β-cells to evaluate the correspondence between hESC-derived cells and mature ones. Of the genes examined, 30 were differentially expressed (FIG. 5C). Compared to S6 insulin+ cells, β-cells expressed low levels of GAST, GHRL, and PYY, but high levels of IAPP and CHGB, consistent with the phenotype of mature β-cells. The early endoderm transcription factors HES1 and SOX9 were equally expressed in β-cells and S6 insulin+ cells, but NOTCH1 was upregulated in S6 insulin+ cells. A cluster of transcription factors that participate in early endocrine lineage specification were expressed at similar levels (NEUROG3, NEUROD1, MAFB, and PAX6), though others (ISL1, PROX1, and FOXA3) were found at higher levels in S6 insulin+ cells.

Of the genes involved in both early pancreas development and mature islet function, PDX1 levels were similar, but several were upregulated in S6 insulin+ cells (FOXA1, HNF1β, SOX4, and PBX1). Notably, β-cells had higher levels of the known β-cell-specific factors MAFA, NKX6-1, and MNX1, whereas S6 insulin+ expressed significantly more of the α-cellspecific factors ARX and IRX2, raising the hypothesis that S6 insulin+ cells express a transcriptional program gravitating towards α-cell fate. In total, 15 of 36 transcription factors examined were differentially expressed (both up and downregulation) in S6 insulin+ cells compared to mature β-cells, indicating that large differences in their identities exist.

These differences were also reflected in genes controlling β-cell function. S6 insulin+ cells expressed significantly less of the signaling factors GCGR and the estrogen receptor (ESR1). These cells also exhibited lower levels of a cluster of proteins involved in insulin biosynthesis, processing, and granule exocytosis (PCSK1 and 2, SLC30A8, STXBP1, and VAMP2). However, S6 insulin+ cells had similar levels of GCK and SLC2A1, the primary glucose transporter in β-cells, and lower levels of G6PC2, which is proposed to counteract GCK. Interestingly, S6 insulin+ cells expressed similar or higher levels of all the Ca2+ and K+ ion channels examined than β-cells did. These data suggest that, at least on the mRNA level, a deficiency of transporters and glycolytic genes cannot explain the lack of stimulus-secretion coupling in S6 insulin+ cells, while a deficiency in insulin processing might. On the whole, this approach of examining a list of candidate genes has revealed the dysregulation of a large number of transcription factors and functional elements thought to be important in β-cells.

### Human β Cell Maturation

Study of human fetal development has been hampered by the absence of reporters and cell surface markers to permit sorting of specific cell types derived ex vivo, as well as the scarcity of human fetal material for study. Researchers have thus relied on studies in model organisms, primarily the mouse, as the basis for understanding human development and optimizing directed differentiation. Recently, our laboratory identified differential gene expression patterns that distinguish fetal β cells from adult β cells in the mouse (Blum et al. Nature biotechnology. 2012; 30:261-264). To uncover markers of human β cell maturation, we performed a purification and transcriptome-wide molecular characterization of human fetal and adult β cells.

Human pancreata at 15-16 weeks gestational age were used, since β cells at this stage are immature and glucose non-responsive (Hayek and Beattie. J. Clinical Endocrinology and Metabolism. 1997; 82:2471-2476; Beattie et al. Cell Transplantation. 1994; 3:421-426). Adult human cadaveric islets and fetal pancreata were dispersed, stained for insulin and FACS sorted. RNA was isolated and analyzed by Illumina microarrays (FIG. 6A).

Human β cells maturation between gestational week 16 and adulthood is characterized by gene expression changes in 643 genes of which 39 were transcription factors (p<0.05, fold change >3) (FIG. 6B). UCN3, which we identified previously as a marker of mouse β cell maturation was expressed 1.1× fold higher in sorted human adult β cells over fetal β cells, indicating that UCN3 expression does not significantly change between human week 16 and adult β cells (FIG. 6C). Collectively, the differentially expressed genes presented in this study could be used as genetic markers of human β cell maturation. Gene Ontology analysis of all differentially expressed genes (p<0.05) suggests that several metabolic and secretory biological processes are significantly enriched in adult β cells over fetal β cells (FIG. 6D).

Together, this data provides the first transcriptome-wide molecular characterization of human fetal and adult β cells. This analysis of genes differentially expressed between fetal and adult β cells points to significant differences between mouse and human β cell maturation. Further analysis of β cells at multiple time-points during human and mouse development is necessary to elucidate these species divergence.

Transcriptional Differences Between hES-Derived INS⁺ Cells and Human Adult β Cells

The goal of hPSC directed differentiation is to generate functional human β cells. We therefore compared gene expression by microarray and RNA-seq between hPSC-INS⁺ cells and adult human β cells. We first focused our analysis on 152 endocrine lineage genes. Expression of 26 of the 152 genes was significantly different between adult β cells and hPSC-INS⁺ cells based on microarray analysis (>3 fold, p<0.05). RNA-seq analysis of two samples of human adult INS⁺ cells and two samples of HUES8-INS⁺ cells confirmed 24/26 genes as differentially expressed >3 fold (FIG. 7A). Using immunofluorescent staining, we confirmed the lack of expression of NKX6-1 and relatively heterogeneous expression of PDX1 and MAFA in hPSC-INS⁺ cells (FIG. 7B). PDX1, MAFA and NKX6-1 are robustly expressed in normal human β cells and in vivo matured hPSC-derived INS⁺ cells (Rezania et al. Diabetes. 2012; 61(8):2016-29).

Many of the incorrectly expressed transcription factors have known roles in endocrine subtype specification: PDX1, NKX6-1, MNX1, FOXA1, ARX, PAX4 and IRX2. Several misexpressed genes from the list are involved in glucose stimulated insulin secretion (GSIS) MAFA, PAX4, GHRL, CHRB, PROX1, KCNK1, KCNK3. Expression of genes sets involved in sterol metabolism/biosynthesis indicates the presence of liver-enriched genetic programs in hPSC-INS⁺ cells.

Since human fetal β cells are also immature and non-functional with respect to accurate GSIS, we wondered which of the genes were also misexpressed in human fetal β cells. Interestingly, the endocrine subtype specification genes were not misexpressed in human fetal β cells while the GSIS genes were (FIG. 7A). This suggests two possibly distinct challenges to produce functional human β cells from hPSC-INS⁺ cells: 1) β cell lineage commitment and 2) functional maturation. Our observations support the hypothesis that hPSC-INS⁺ cells resemble human fetal cells that are not (yet) committed to the β cell lineage. The modulation of these genes along with others with the same pattern of expression may be critical for the acquisition of mature glucose response.

We next expanded the analysis to the whole genome. Of the 755 genes that were differentially expressed (p<0.05, >3 fold) between hPSC-INS⁺ cells and human β cells, 583 were confirmed by RNA-Seq (>3 fold). We present a list of all differentially expressed transcription factors as they are of particular interest for they role in modulating cell fates (FIG. 7C). Genes identified here can be used as markers for differentiation into human β cells or may be manipulated to direct the conversion of hPSC-INS⁺ cells into human functional β cells. Additionally, gene ontology analysis of all differentially expressed genes (p<0.05 by microarray) identified 22 statistically enriched (q<0.05) biological processes in hPSC-INS⁺ cells (Table 11 below). Among the ten most differentially expressed processes (FIG. 7D) three involve cholesterol/sterol biosynthesis or metabolism indicating that hPSC-INS⁺ cells additionally express non-pancreatic, liver-specific genetic pathways.

TABLE 11
GO Biological Pathways enriched in hPSC-INS+ cells

| | Gene Ontology Biological Process | Benjamini q value |
|---|---|---|
| Enriched | cytoskeleton organization | 0.0014108 |
| in hPSC-INS⁺ | sterol biosynthetic process | 0.0014891 |
| cells | cholesterol biosynthetic process | 0.0018188 |
| | cell morphogenesis | 0.0018485 |
| | cellular component morphogenesis | 0.0045675 |
| | actin filament-based process | 0.0150359 |
| | actin cytoskeleton organization | 0.0167663 |
| | sterol metabolic process | 0.0174035 |
| | cell projection organization | 0.0179661 |
| | neuron projection development | 0.0185401 |
| | cholesterol metabolic process | 0.0187551 |
| | neuron development | 0.0199759 |
| | cell part morphogenesis | 0.0201410 |
| | neuron projection morphogenesis | 0.0213534 |
| | cell projection morphogenesis | 0.0228888 |
| | lipid transport | 0.0264480 |
| | lipid localization | 0.0286305 |
| | isoprenoid biosynthetic process | 0.0295110 |
| | neuron differentiation | 0.0301086 |
| | lipoprotein particle clearance | 0.0440558 |
| | response to organic substance | 0.0444088 |
| | steroid biosynthetic process | 0.0456816 |

### Gene Set and Pathway Analysis

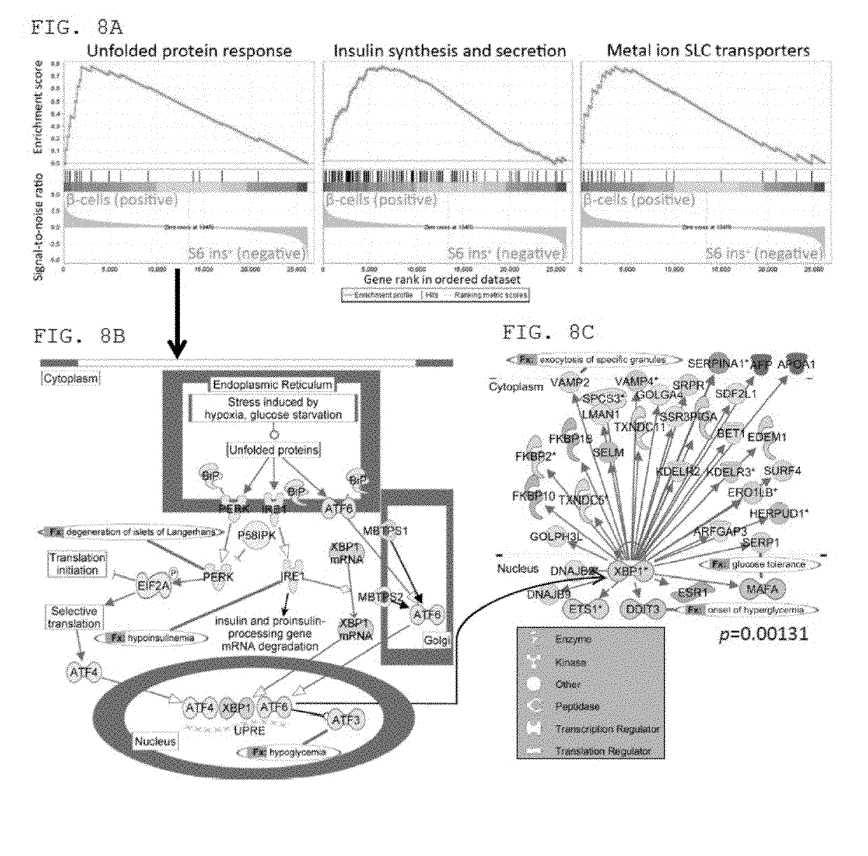

We wished to take a less subjectively biased and more systems-based approach in identifying dysregulated elements, so we next performed gene set enrichment analysis (GSEA) using canonical pathways from Reactome. This method allows the comparison of sample groups across a large number of manually curated gene lists that have known biological relationships, in a way that is more sensitive than traditional differential expression analysis to small, but concordant, changes that occur broadly across a defined pathway (Subramanian et al., 2005). GSEA identified only three Reactome gene sets that were significantly enriched in β-cells compared to S6 insulin+ cells after adjusting for multiple hypotheses testing (FDR q<0.05; FIG. 8A). One was “insulin synthesis and secretion” (q=0.031), confirming our previous findings that on the transcriptional level, S6 insulin+ cells have defective insulin processing machinery. Genes in the leading edge (most enriched) included ones tested in our candidate approach (e.g. PCSK1 and 2, VAMP2, STXBP1, etc.) and others less specific, but nevertheless important, for β-cell function (e.g. signal peptidases, protein localization signal receptors, and ribosomal proteins). A second enriched pathway was “metal ion SLC transporters” (q=0.027). Leading edge analysis revealed that SLC30A8 was the most enriched member in β-cells, confirming our previous candidate-based findings, within a list of 11 transporters, most of them zinc carriers. Zinc is important in insulin granule maturation (Chimienti et al., 2004; Lemaire et al., 2009), so this result suggests that an inability to produce zinc flux may contribute to the dysfunction of S6 insulin+ cells.

Interestingly, the most enriched pathway in β-cells compared to S6 insulin+ cells was the “unfolded protein response” (UPR; q<0.001). We confirmed this association using Ingenuity Pathway Analysis (IPA), which relies on a different algorithm and proprietary, but similar gene lists (FIG. 8B). Members of this pathway, including PERK (EIF2AK3) and IRE1α, have been implicated in mature β-cell function through animal and human genetics studies. In particular, dysregulation of the UPR pathway can disturb proinsulin synthesis, intracellular insulin trafficking, and expression of β-cell-related genes (see Chapter 4.2 for discussion). In fact, IPA revealed that a transcription factor downstream in the UPR pathway, XBP1, is expressed significantly lower in S6 insulin+ cells and regulates or interacts with many of the other genes downregulated in S6 insulin+ cells, including the exocytosis proteins VAMP2 and VAMP4, and the transcription factors MAFA, ESR1, and DDIT3 (FIG. 8C). The overlap between the list of genes with depressed expression in S6 insulin+ cells and the list of genes directly downstream of XBP1 was the most statistically significant of any transcription factor-based network in IPA (p=0.00131). These data strongly indicate that the UPR pathway is dysregulated in S6 insulin+ cells and may account for part of the broader functional defects observed.

Design and interpretation of the preceding candidate gene evaluations and knowledge-based pathway analyses are necessarily limited and biased by the available experimental evidence, which are largely derived from animal or cell culture models. In the case of transcription factors, genes can have vaguely defined effects on cell identity that vary across developmental stages and species. Our evaluation of transcription factors derived from the literature on pancreatic development and function revealed many genes that were overexpressed and many that were underexpressed in S6 insulin+ cells compared to β-cells.

The implications of these varied differences on mature cell type specification remained unclear. Hence, we hypothesized that the examination of human β-cell-specific genes could clarify how closely S6 insulin+ cells resemble β-cells as opposed to other pancreatic cells.

### Beta-Cell Molecular Signature Expression

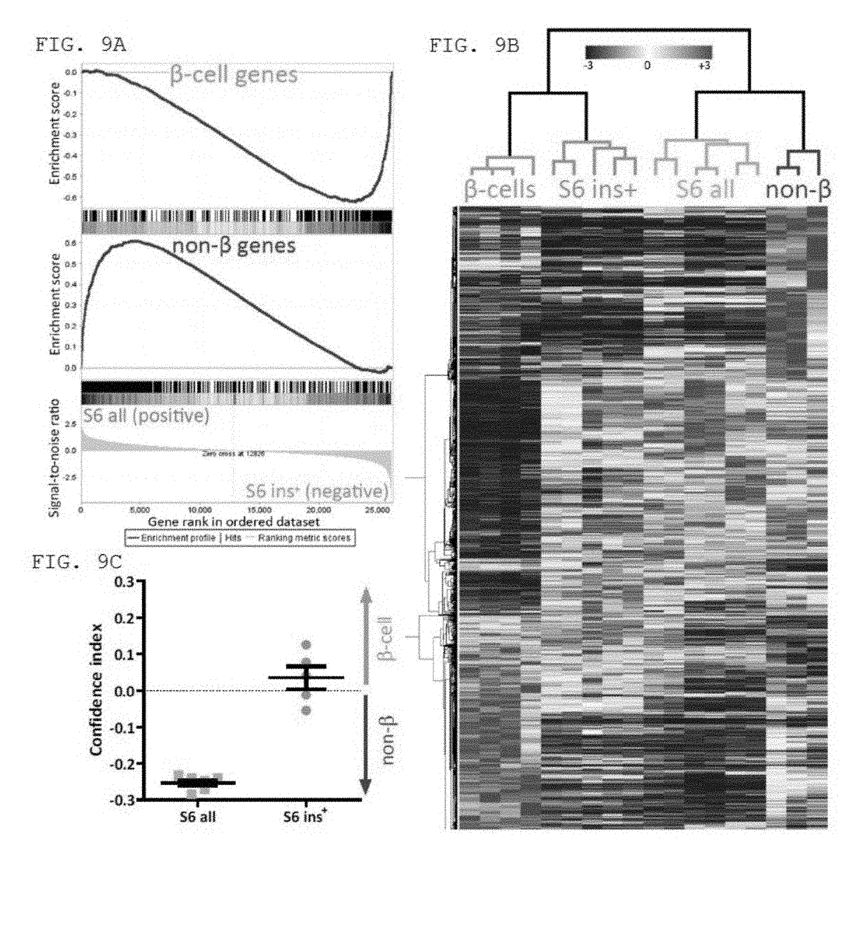

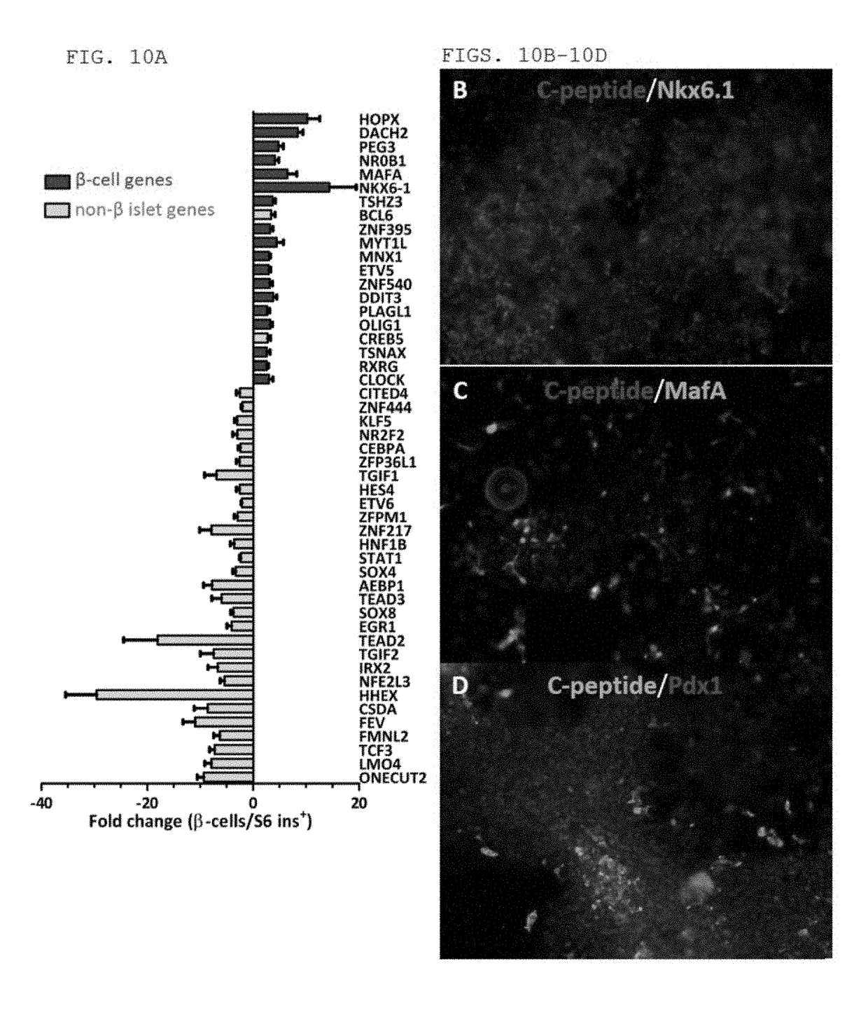

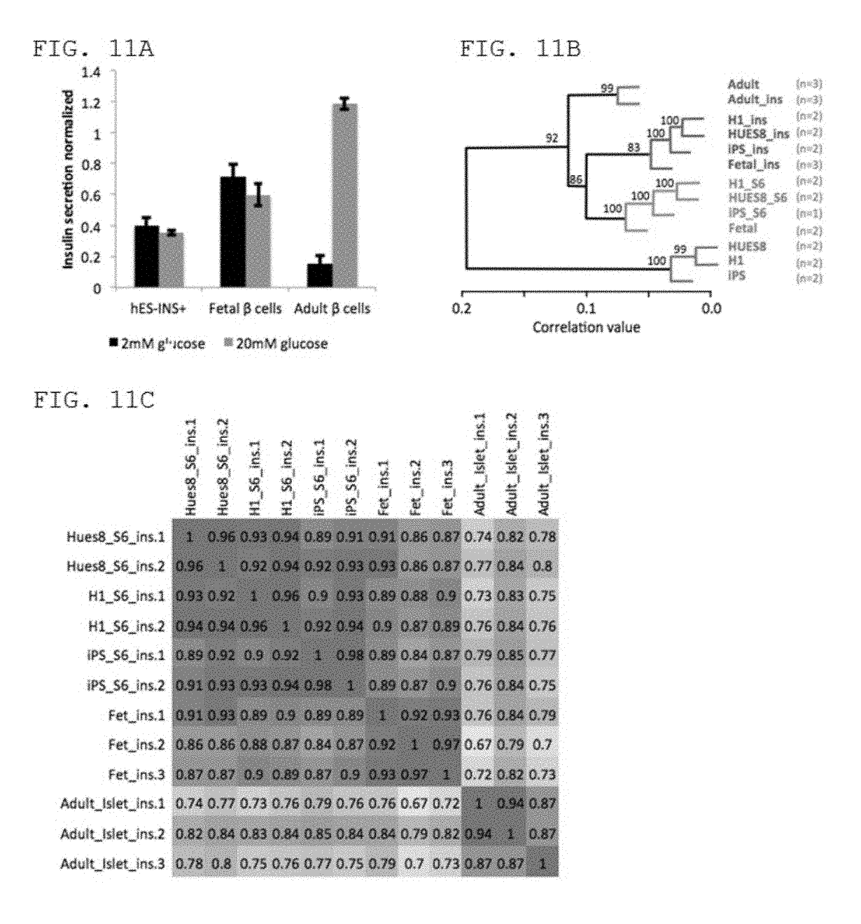

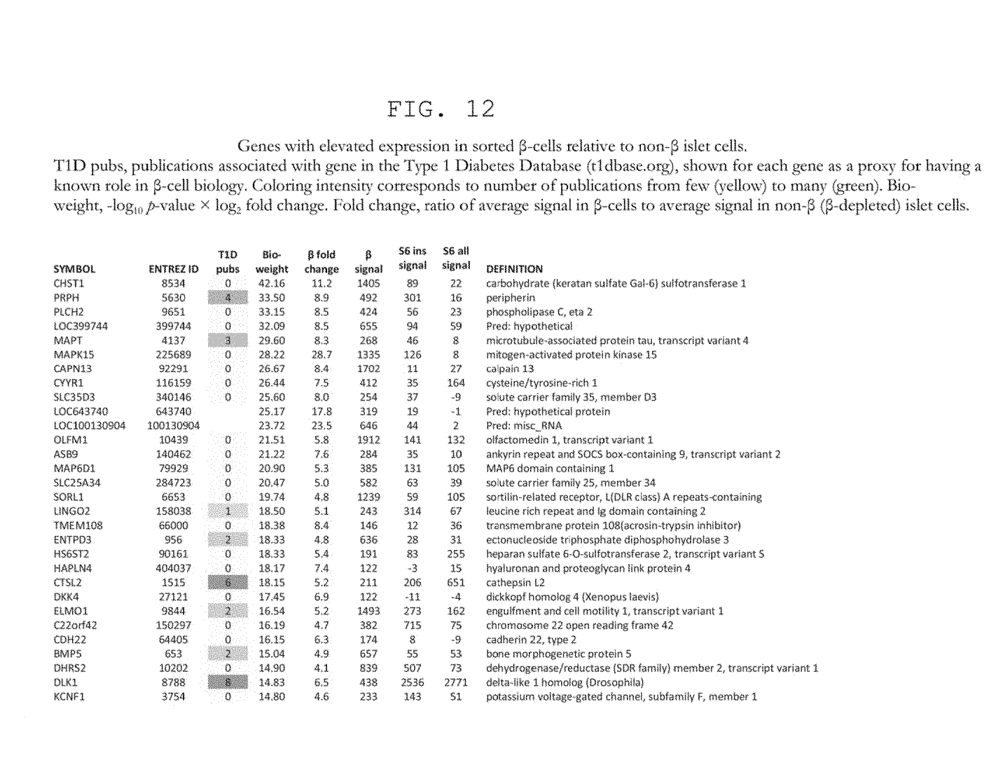

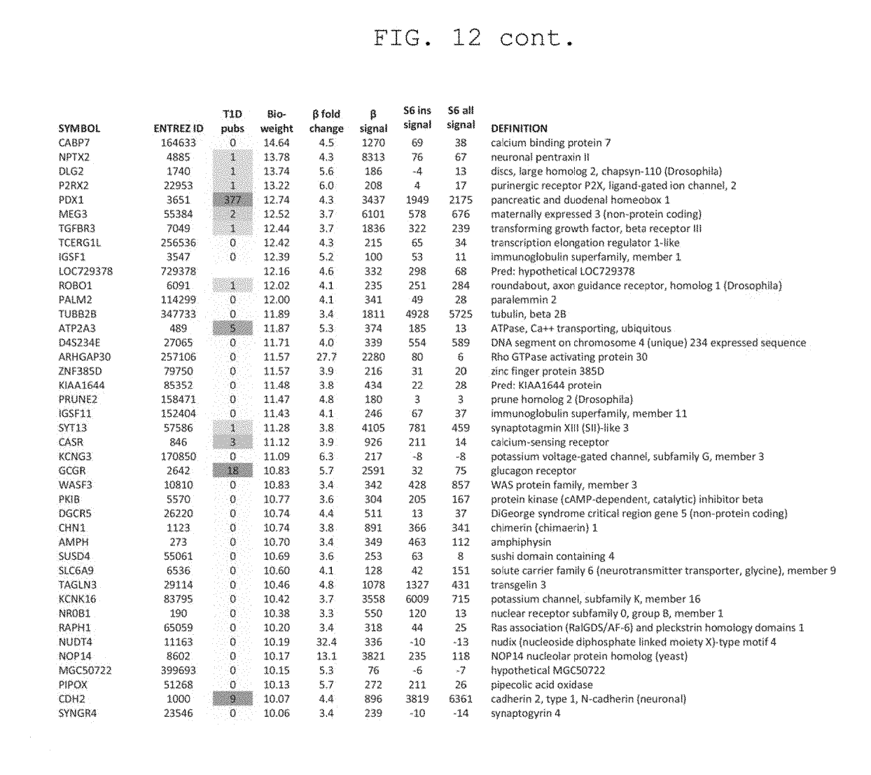

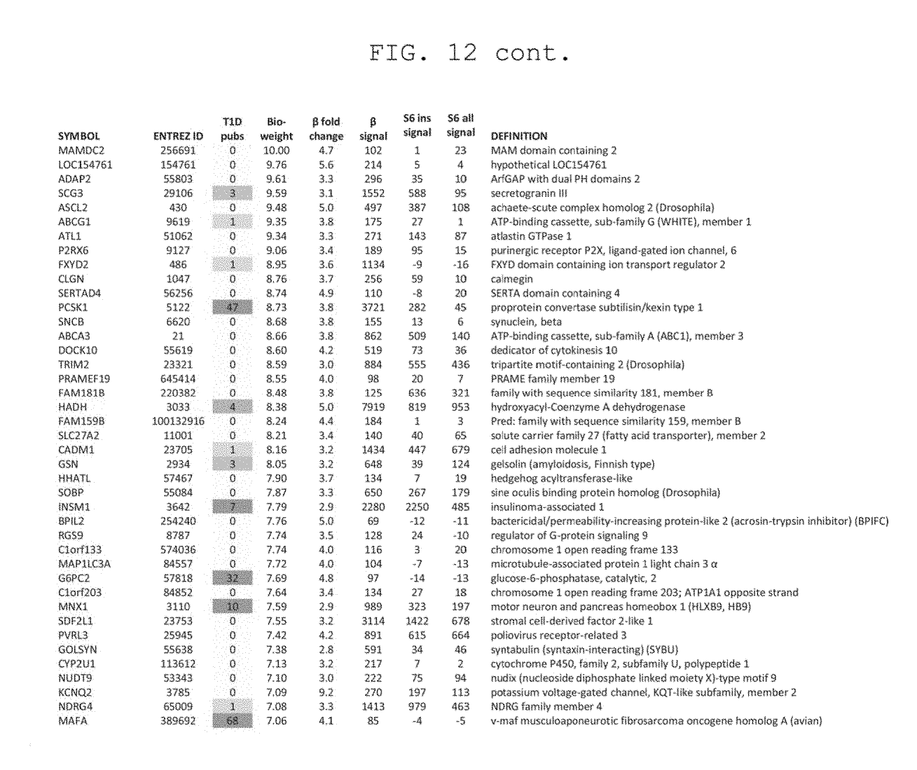

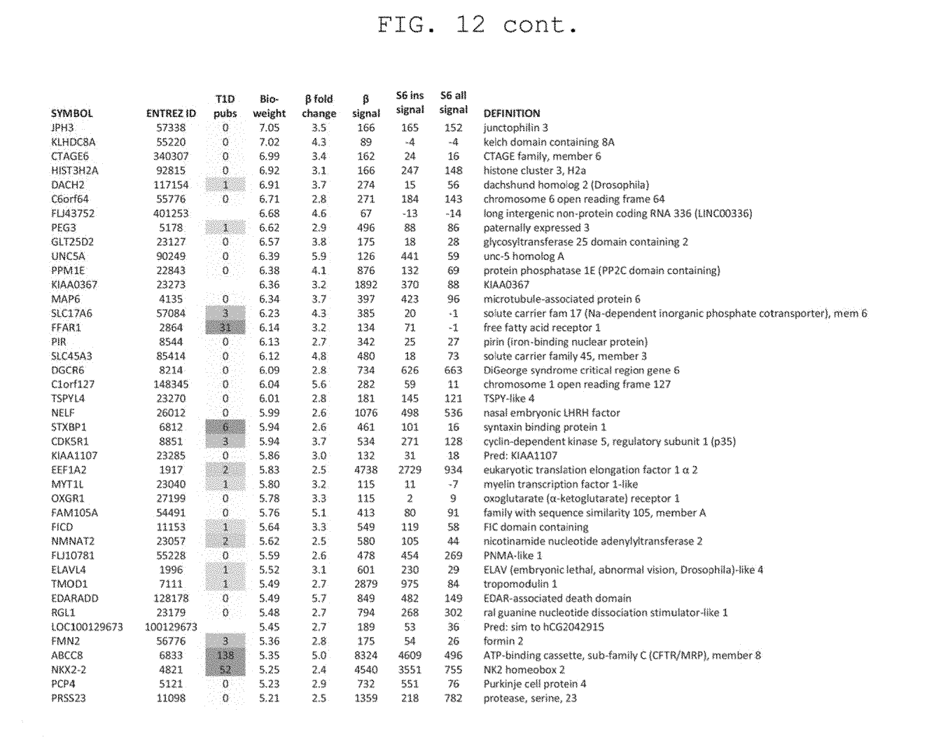

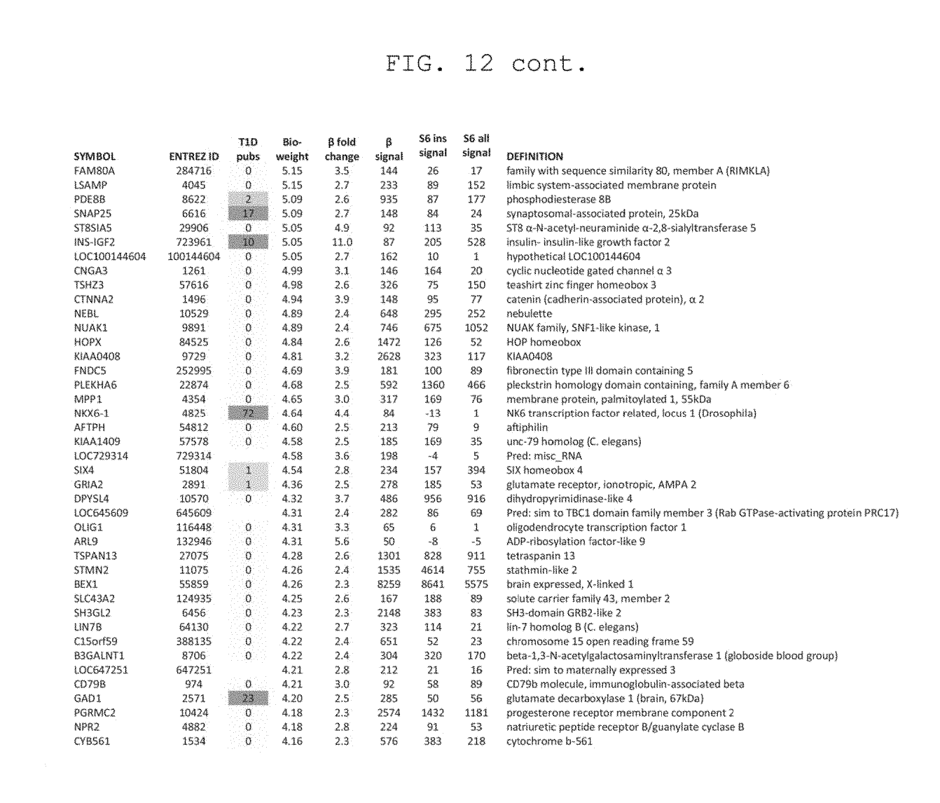

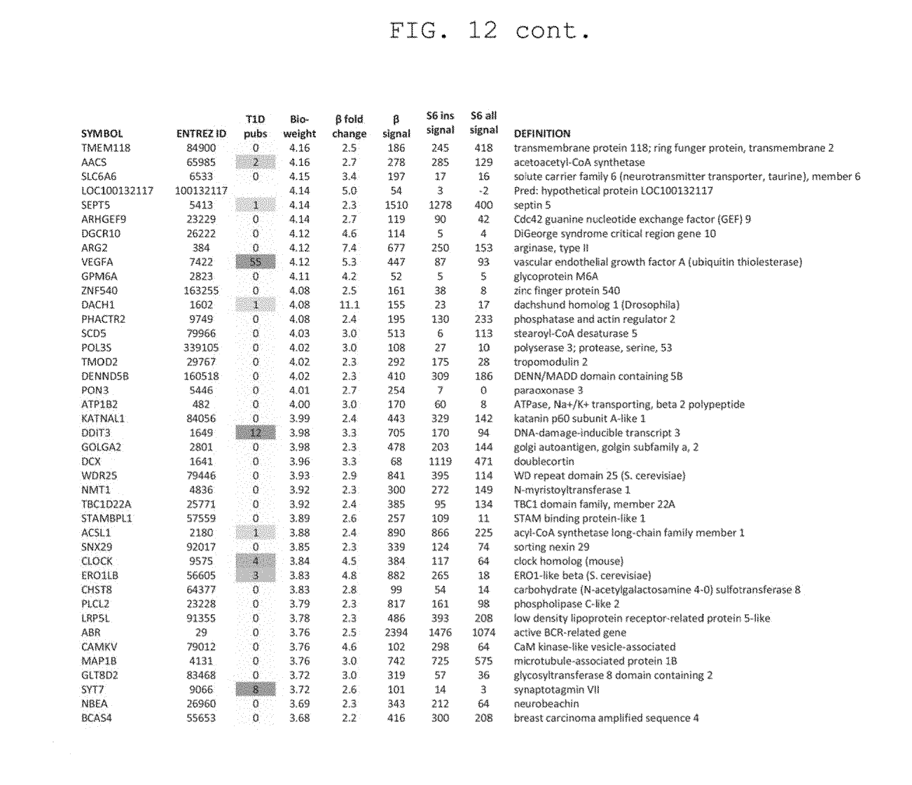

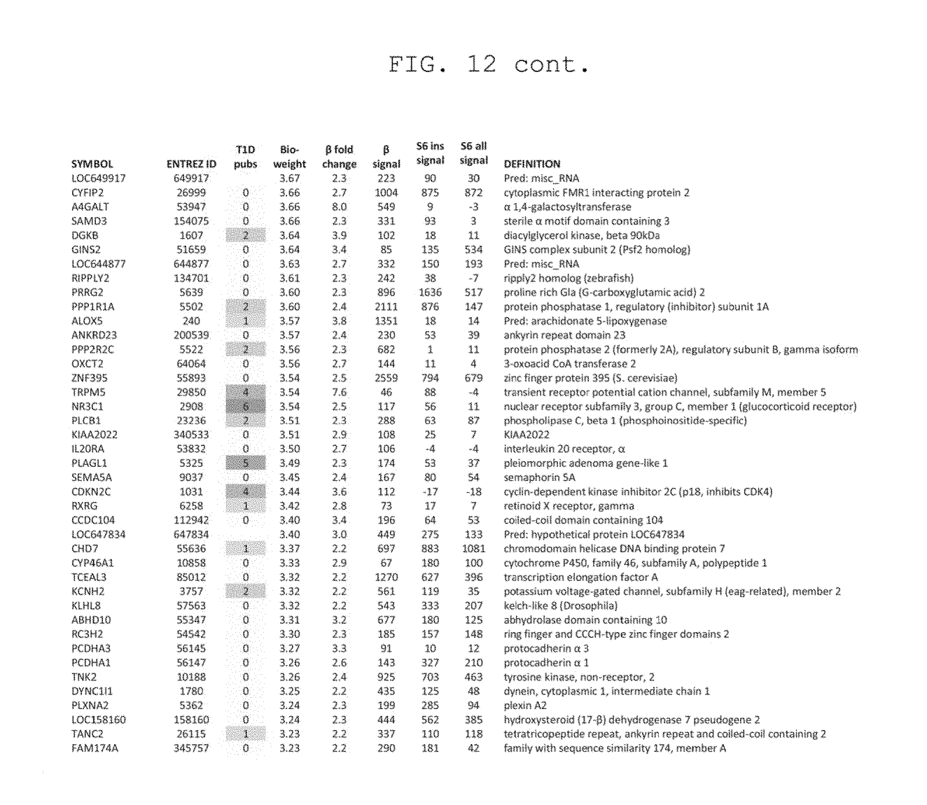

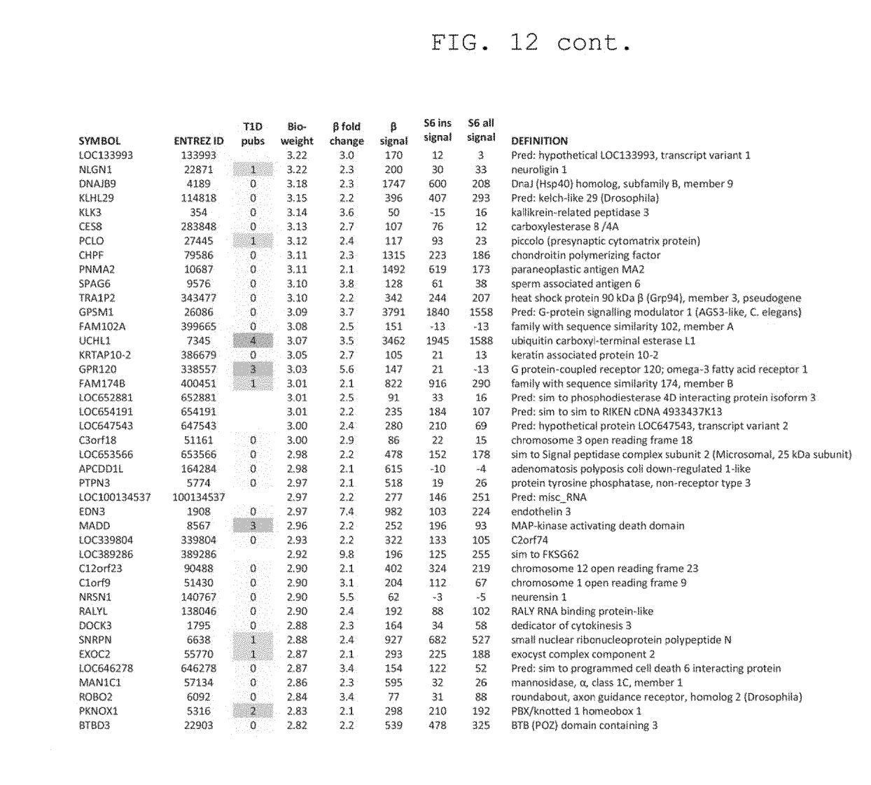

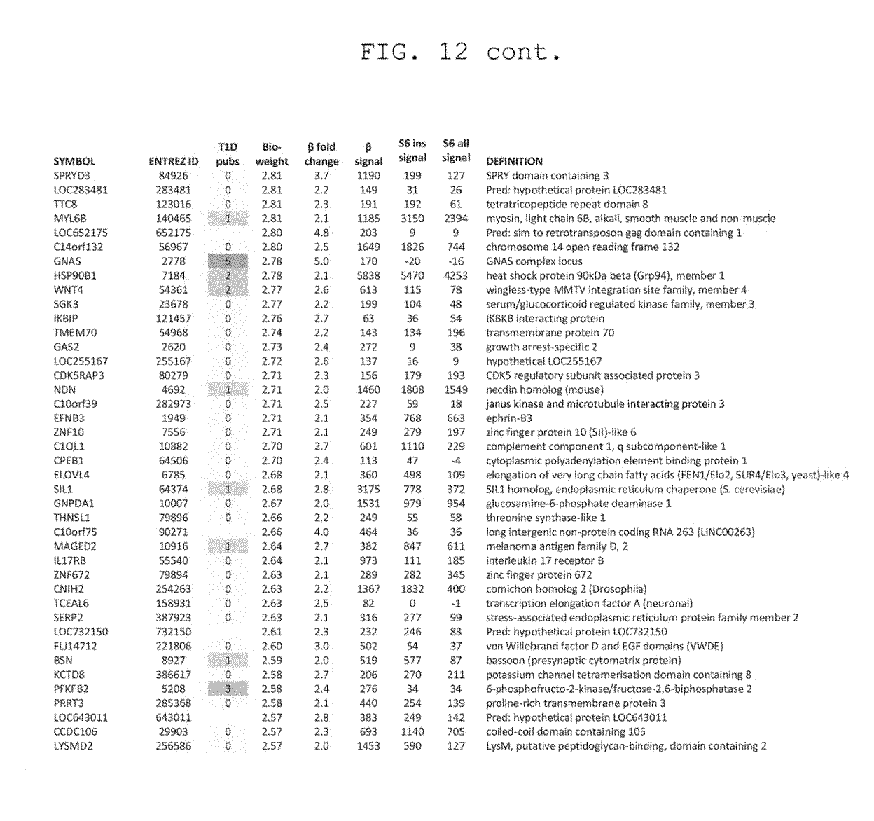

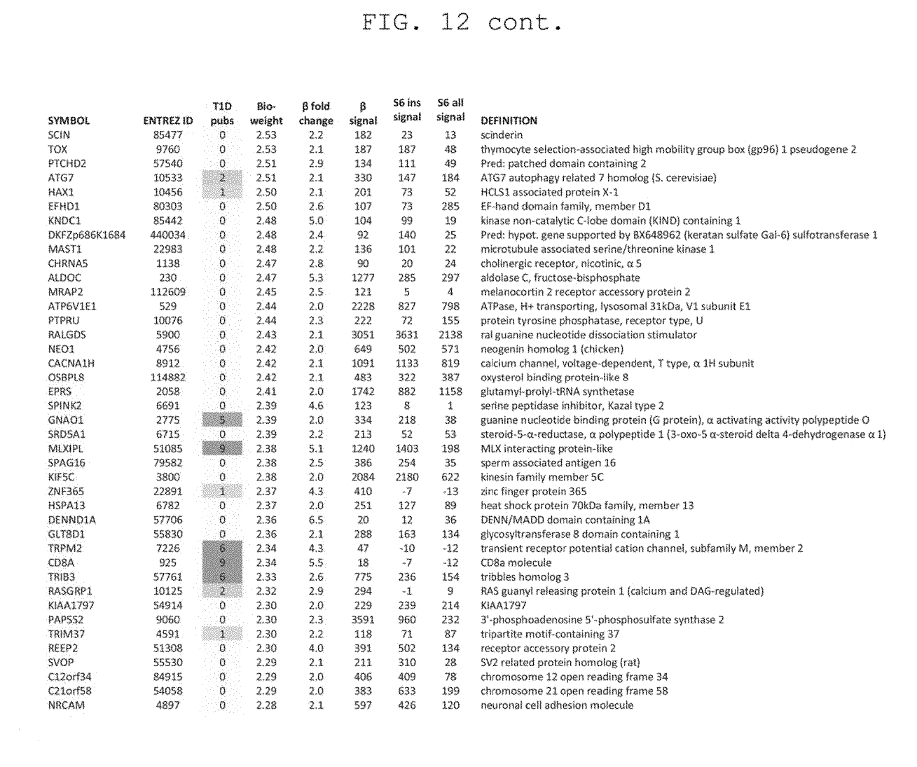

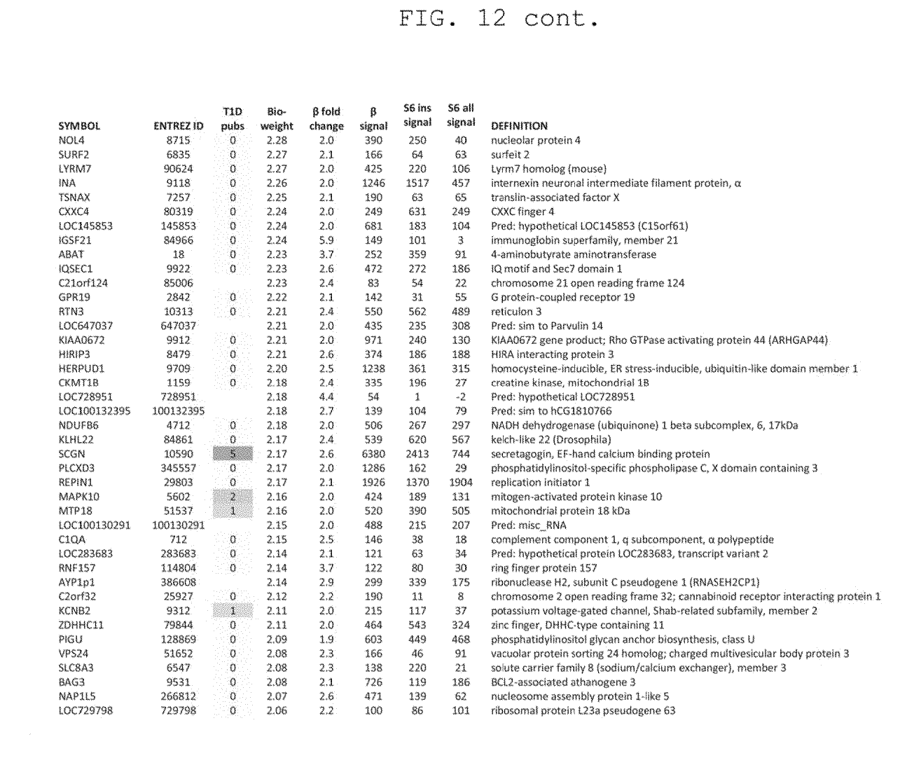

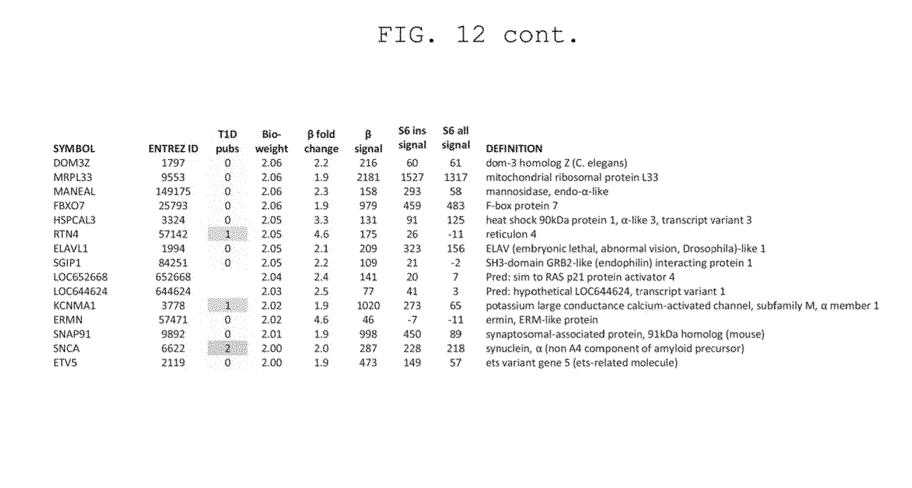

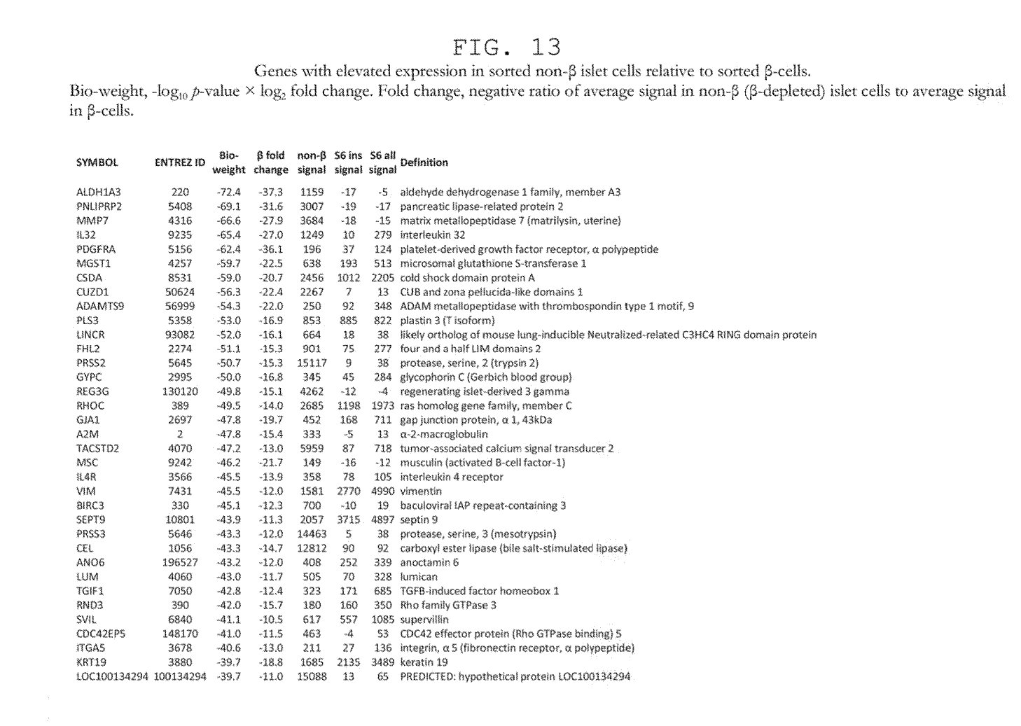

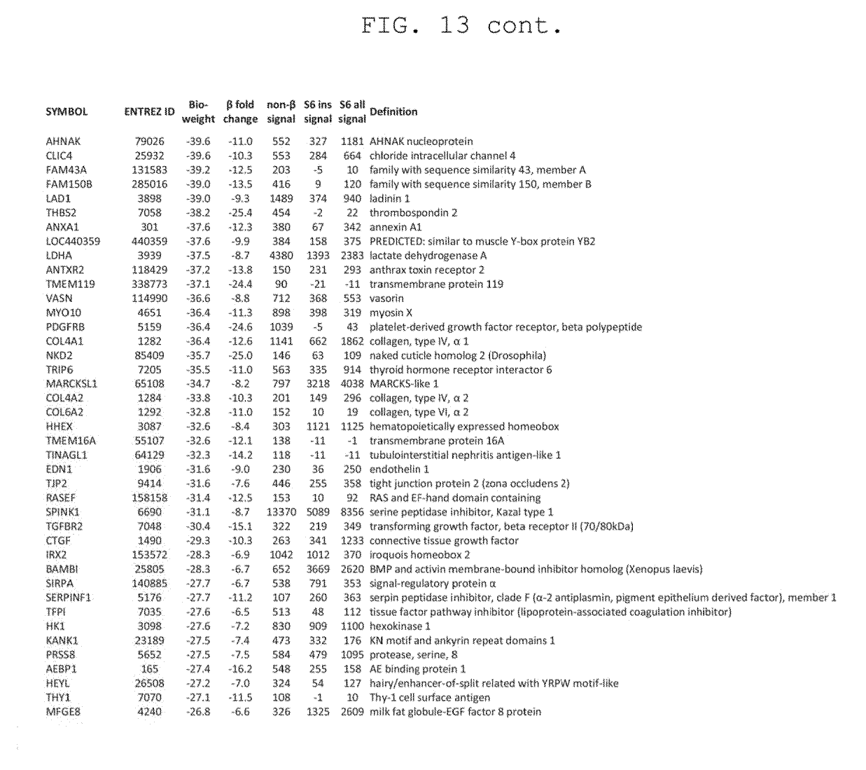

Towards this end, we asked whether the β-cell molecular signature previously generated could inform the characterization of hESC-derived differentiation products. We assessed the expression of the 1403 probes in the signature (up and downregulated in β-cells relative to non-β islet cells) in unsorted S6 cells and the insulin+ fraction. First, we defined the 458 genes preferentially expressed in β-cells as the “β-cell gene set” and the 775 genes preferentially expressed in β-depleted islet cells as the “non-β gene set.” GSEA using these two gene sets and a rank-based metric revealed that, in comparison to S6 insulin+ cells, unsorted S6 cells were significantly enriched for non-β genes (p<0.001; FIG. 9A). Conversely, in comparison to unsorted S6 cells, S6 insulin+ cells were significantly enriched for β-cell genes (p<0.001). These data simply indicate that the insulin+ fraction is more β-cell-like while the bulk S6 population is more akin to a mixed population of pancreatic tissue exclusive of β-cells.

Hierarchical clustering based on row-normalized signature probes led to a similar conclusion. S6 insulin+ cells clustered more closely with β-cells while unsorted S6 cells clustered more closely with non-β islet cells (FIG. 9B). Inspection of the heatmap and dendrogram, however, revealed that many differences within each branch existed. In fact, 717 of 1403 probes were significantly different between β-cells and S6 insulin+ cells (|bioweight|>2; mean signals listed in FIG. 12 and FIG. 13). To investigate further the absolute degree of similarity between β-cells and S6 insulin+ cells (as opposed to relative difference based on comparison to unsorted S6 cells), we used a statistical weighted voting model using the 1403 probe signature (Golub et al., 1999). Each probe votes for classification as either β-cell or non-β cell depending on whether the expression level in the sample of interest is closer to the mean of levels in β-cells or non-β cells. Further, the votes are weighted by how well the probe is correlated with the difference between β-cell and non-β cell samples. Our model classified unsorted S6 cells as non-β pancreatic cells with modest confidence, but S6 insulin+ cells could not be confidently classified as β-cells (FIG. 9C). These data indicate that while S6 cells as a whole are marginally reminiscent of a mixed population of pancreatic cells, the cells in the insulin+ fraction do not significantly resemble mature β-cells in particular.

To hone in on the salient differences, we examined the transcription factors belonging to the β-cell/non-β signature set that were differentially expressed between S6 insulin+ cells and β-cells (|bio-weight|>2). Of the 20 factors having higher expression levels in β-cells than S6 insulin+ cells, 18 are normally β-cell-selective in the pancreas (FIG. 10A). Conversely, of the 29 transcription factors having higher expression levels in S6 insulin+ cells, all are normally expressed more in non-β islet cells than in β-cells (FIG. 10A). This remarkable degree of category concordance suggests that S6 insulin+ cells are systematically expressing transcription factors that define non-β cell identity while underexpressing transcription factors that define β-cell identity.

The list of misexpressed transcription factors included known and novel genes in islet biology. Among the β-cell-specific factors examined via our candidate approach were NKX6-1, MAFA, and MNX1. NKX6-1 mRNA was expressed 14-fold higher in β-cells than S6 insulin+ cells, a difference we confirmed here by immunohistochemistry: almost no coexpression of c-peptide (the by-product of de novo insulin synthesis) and Nkx6.1 protein was detected in S6 culture (FIG. 10B). The many Nkx6.1+ cells present were likely less differentiated pancreatic progenitors, as Pdx1 was also observed in a large number of S6 cells (FIG. 10D). MAFA mRNA was expressed 6.5-fold higher in β-cells than in S6 insulin+ cells; as expected, upon staining, we observed that only a minority of c-peptide+ cells expressed detectable MafA protein. In contrast, PDX1 mRNA levels were not significantly different between β-cells and S6 insulin+ cells and accordingly, Pdx1 protein was highly expressed in all c-peptide+ cells (FIG. 10D). Among the differentially expressed non-β-specific factors identified both in our candidate approach and the signature gene list informed approach were IRX2, HNF1β, and SOX4. Taken together, the data imply that β-cell signature genes may serve as informative, specific markers for monitoring in vitro differentiation. This focused list of transcription factors elucidates key differences between S6 insulin+ cells and β-cells and provides potential targets for improving directed differentiation.

### Discussion

### Beta-Cell Transcriptome

We generated a gene expression atlas specific to β-cells that correlated with a subset of findings by Grompe and colleagues, who used a surface marker FACS approach that partially purified β-cells with δ-cells (Dorrell et al., 2011). Out of the 43 genes that the prior study listed with >20-fold elevated expression in putative sorted β-cells relative to sorted α-cells, we found significant enrichment in 15 when comparing β-cells to mixed non-β islet cells: ASB9, CAPN11, CDH22, CLIC6, CNGA3, DCX, DGKB, DLK1, GLT25D2, HADH, IGSF11, PLCH2, PRPH, SCD5, SGIP1, SLC17A6, and SOD3. Among these are genes with known human β-cell functions. HADH encodes an enzyme involved in mitochondrial fatty acid oxidation and regulation of insulin secretion (Hardy et al., 2007), inactivating mutations in which cause congenital hyperinsulinism (Clayton et al., 2001).

Others are suspected human β-cell factors. The locus of DGKB, which encodes a kinase involved in glycerolipid metabolism, is associated with type 2 diabetes (Dupuis et al., 2010) and impaired glucose-stimulated insulin secretion (Boesgaard et al., 2010). In addition, the imprinted DLK1 region is associated with paternally inherited risk of T1DM (Wallace et al., 2010). Due to the design of our study, we can now confidently assign the expression of these genes to the β-cells within the adult human pancreatic islet.

A notable finding of this study is that adult human β-cells express only a small set of unique transcription factors. We identified 31 such genes that have varying levels of existing evidence for a role in β-cell biology. Most reassuringly, gene targeting experiments in mice have revealed critical, now canonical, roles for MAFA (Matsuoka et al., 2004; Zhao et al., 2005), MNX1/HLXB9 (Harrison et al., 1999; Li et al., 1999), NKX2-2 (Sussel et al., 1998), NKX6-1 (Sander et al., 2000), and PDX1 (Jonsson et al., 1994) in β-cell specification, maturation, and function. Human genetics have verified some of these findings. Most notably, various mutations in PDX1 cause maturity onset diabetes of the young type IV (MODY4), pancreatic agenesis (Stoffers et al., 1997), and susceptibility to T2DM (Hani et al., 1999; Macfarlane et al., 1999). Polymorphisms in human MAFA are associated with T1DM susceptibility (Noso et al., 2010). These data corroborate our transcription factor expression profiles that robustly show for the first time the β-cell-specificity of these factors in humans.

A handful of other transcription factors identified have plausible roles in β-cells and merit discussion. First, in the case of CLOCK, conditional ablation of this circadian oscillator resulted in diabetes in mice due to detective stimulus-secretion coupling in β-cells (Marcheva et al., 2010). Two circadian rhythm regulators (MTNR1B, CRY2) have been linked to fasting glucose levels in human genome-wide association studies (Dupuis et al., 2010). Though these two genes were not identified as β-cell-specific in our study, they do provide evidence for a role of this CLOCK-related pathway in glucose homeostasis. Second, in the case of DACH1, deletion in mice blocked the perinatal burst of differentiated β-cell expansion, resulting in lower β-cell mass in the adult (Kalousova et al., 2010). This gene and its homologues appear to play a conserved role from zebrafish to mice in regulating the proliferation of pancreatic endocrine cells. Third, the expression of HOPX in sorted β-cells was previously observed and confirmed on the protein level (Dorrell et al., 2011). Though its function in the pancreas is unclear, there is evidence that in the developing heart and lung, HOPX functions downstream of NKX2-1 and NKX2-5, respectively (Chen et al., 2002; Yin et al., 2006). Since human β-cells also express NK-family homeobox genes, HOPX may play a role in endocrine lineage specification. Fourth, MLXIPL (ChREBP) regulates glycolytic and lipogenic gene expression in the mouse β-cell line MIN6 in a glucose-dependent manner (Wang and Wollheim, 2002; Da Silva Xavier et al., 2006). Inactivation or overexpression of the transcription factor have also been shown to modulate levels of PDX1, MAFA, GCK, and INS in MIN6 cells (da Silva Xavier et al., 2010). MLXIPL may therefore be an important regulator of β-cell gene expression that responds to elevated glucose concentrations. Fifth is MYTL1, which has not been previously described in normal adult or embryonic pancreas. When its paralog, MYT1, was knocked out in the developing mouse pancreas, the mutant animals exhibited mildly attenuated glucose-stimulated insulin secretion and, interestingly, abnormal polyhormonal cells within the adult islets (Wang et al., 2007). In these knockouts, MYTL1 was upregulated and, the authors speculated, reduced the full effects of MYT1 deletion via a redundant, compensatory mechanism. These data point to a potential ability for MYT1L to regulate β-cell function or differentiation.

Finally, PEG3 is an imprinted gene whose product inhibits Wnt signaling in human cells by binding to the signal transduction protein β-catenin and promoting its degradation (Jiang et al., 2010). Exposure to Wnt3a (a Wnt family member expressed in human islets) or forced expression of activated β-catenin causes the proliferation of differentiated mouse β-cells in culture and in vivo (Rulifson et al., 2007). In humans, hypomethylation of PEG3 and some other imprinted loci, caused by the mutation of an upstream transcription factor, is associated with transient neonatal diabetes mellitus (Mackay et al., 2008). These data suggest a role for PEG3 in promoting or maintaining β-cell mass.

Collectively, however, these gene expression observations provide plausible heuristics for finding reprogramming agents that enforce β-cell identity. We further speculate that a combination of human genetics studies and our functional genomics approach will provide focused insight on the unique roles of genes in human β-cell biology.

Immature Phenotype of hESC-Derived Insulin+ Cells

Glucose-stimulated insulin secretion in hESC-derived cells remains elusive. We found that S6 insulin+ cells secreted insulin above baseline only under direct membrane depolarization. On a per cell basis, KCl-induced insulin release occured at lower levels in S6 cells than in islets. Consistent with this functional finding, we detected lower expression of many genes involved in insulin processing and granule exocytosis (e.g. PCSK1 and 2, SLC30A8/ZnT8, VAMP2, and STXBP1). The identification of SLC30A8 as a dysregulated element was corroborated by pathway analysis and stands out because ample evidence points to its importance in humans. A polymorphism within the human SLC30A8 gene is associated with indices of β-cell function (Dupuis et al., 2010) and susceptibility to T2DM, a genome-wide significant finding that has been well-replicated (Saxena et al., 2007; Scott et al., 2007; Sladek et al., 2007; Zeggini et al., 2007).

The plainest hypotheses for explaining the lack of glucose responsiveness, regardless of total insulin content, are deficiencies in glucose sensing and metabolism, or in ion channels required for triggering secretion. Unfortunately, we found few remarkable deficits in the glucose transporter Glut1, glycolytic machinery, and KATP and Ca2+ ion channel expression, consistent with a previous report on hESC-derived cells (Basford et al., 2011). That prior report did demonstrate reduced levels of functional ion channels despite normal mRNA levels, but electrophysiological analysis of the insulin+ cells from our protocol remains a future goal. In a search for more satisfying hypotheses that would provide targets for the improvement of directed differentiation, we then examined transcription factors and members of signaling pathways.

The deficiency of the glucagon receptor (GCGR) in hESC-derived insulin+ cells is noteworthy. In mice, overexpression of GCGR specifically in β-cells resulted in enhanced β-cell function and mass (Gelling et al., 2009). In humans, different genetic variants in GCGR are associated with type 2 diabetes (Hager et al., 1995) and, in the case of one homozygous missense mutation, hyperglucagonemia due to α-cell hyperplasia (Zhou et al., 2009). In the latter instance, the patient's α-cells also expressed pancreatic polypeptide, which is normally produced by PP cells of the pancreatic islets, suggesting that, in a background of GCGR deficiency, these glucagon-expressing cells have an immature phenotype. Though the limited pancreas biopsy from this patient did not contain glucagon+/insulin+ cells, such polyhormonal cells were readily observed in adult GCGR knockout mice (Vuguin et al., 2006). Additionally, there were a larger proportion of α-cells and δ-cells in these animals, and the α-cells expressed the β-cell markers SLC2A2/GLUT2 and PDX1, while the β-cells exhibited lower than normal levels of insulin, GLUT2, MAFA, PDX1, and PCSK1. This immature phenotype is strikingly similar to that of insulin+ cells produced in vitro. In this study, we found that hESC-derived insulin+ cells frequently co-expressed other endocrine hormones, expressed α-cell transcription factors ARX and IRX2, and had lower levels of MAFA, GLUT2, and PCSK1 compared to mature β-cells. These results suggest that activating glucagon signaling could aid the maturation of hESC-derived insulin+ cells.

This study additionally found markedly decreased levels of XBP1, PERK, eIF2α, and other members of the unfolded protein response (UPR) signaling pathways in hESC-derived insulin+ cells compared to adult human β-cells. Normally, pancreatic β-cell survival and function depend on the UPR pathways coordinated by PERK and eIF2α, on one branch, or by IRE1α and XBP1, on the other (FIG. 6B). With respect to the first branch, in humans, mutations in the kinase PERK underlie Wolcott-Rallison syndrome, which is characterized by permanent neonatal or early infancy insulin-dependent diabetes (Delépine et al., 2000). Evidence from mouse models indicates that PERK is required for fetal β-cell differentiation and, in the adult organism, PERK regulates proinsulin trafficking and quality control in response to metabolic demands (Zhang et al., 2006; Gupta et al., 2010). PERK phosphorylates eIF2α, which attenuates mRNA translation in β-cells. When the eIF2α phosphorylation site was inducibly mutated in mouse β-cells, the animals experienced severe diabetes within a few weeks due to unregulated proinsulin translation, defective protein trafficking, and reduced expression of the β-cell genes PDX1, MAFA, GLUT2, IAPP, and insulin (Back et al., 2009).

With respect to the second branch, very recent evidence shows that a feedback loop involving IRE1a and its splicing product XBP1 is necessary for optimal insulin secretion and glucose homeostasis (Lee et al., 2011). β-cell-specific ablation of the transcription factor XBP1 in mice markedly reduced the number of insulin granules in β-cells, impaired the processing of proinsulin, attenuated glucose-stimulated insulin secretion, and inhibited β-cell proliferation. In addition to the effects on protein processing, deficiency of XBP1 caused hyperactivation of its upstream activator, IRE1α, which can degrade a set of mRNAs encoding insulin, PCSK1, and PCSK2 (Lipson et al., 2006, 2008; Lee et al., 2011).

Collectively, these data point to the UPR signaling pathway as an exciting target for enhancing insulin biosynthesis in hESC-derived cells. Further targets were suggested in our examination of β-cell-specific transcription factors in hESC-derived insulin+ cells. In particular, MAFA, NKX6-1, and MNX1 were among those underexpressed in S6 insulin+ cells compared to mature β-cells. These and others with hypothesized roles in β-cells (see above), such as HOPX, MYT1L, CLOCK, and DACH1, together form a high priority list of factors to promote β-cell identity following the immature endocrine cell stages of directed differentiation. In addition, factors not preferentially expressed in β-cells, including ARX, IRX2, and HNF1β, represent potential targets for the inhibition of polyhormonal fates.

### REFERENCES

Abeyta, M. J., Clark, A. T., Rodriguez, R. T., Bodnar, M. S., Pera, R. A. R., Firpo, M. T., 2004. Unique gene expression signatures of independently-derived human embryonic stem cell lines. Hum. Mol. Genet. 13, 601-608.
Adewumi, O., Aflatoonian, B., Ahrlund-Richter, L., Amit, M., Andrews, P. W., et al., 2007. Characterization of human embryonic stem cell lines by the International Stem Cell Initiative. Nat. Biotechnol. 25, 803-816.
Afrikanova, I., Yebra, M., Simpkinson, M., Xu, Y., Hayek, A., Montgomery, A., 2011. Inhibitors of Src and Focal Adhesion Kinase Promote Endocrine Specification. Journal of Biological Chemistry 286, 36042-36052.
Allegrucci, C., Young, L. E., 2007. Differences between human embryonic stem cell lines. Hum. Reprod. Update 13, 103-120.
Amit, M., Carpenter, M. K., Inokuma, M. S., Chiu, C. P., Harris, C. P., Waknitz, M. A., Itskovitz-Eldor, J., Thomson, J. A., 2000. Clonally derived human embryonic stem cell lines maintain pluripotency and proliferative potential for prolonged periods of culture. Dev. Biol. 227, 271-278.
Andrews, P. W., Damjanov, I., Simon, D., Banting, G. S., Carlin, C., Dracopoli, N. C., Føgh, J., 1984. Pluripotent embryonal carcinoma clones derived from the human teratocarcinoma cell line Tera-2. Differentiation in vivo and in vitro. Lab. Invest. 50, 147-162.
Assady, S., Maor, G., Amit, M., Itskovitz-Eldor, J., Skorecki, K. L., Tzukerman, M., 2001. Insulin production by human embryonic stem cells. Diabetes 50, 1691-1697. Back, S. H., Scheuner, D., Han, J., Song, B., Ribick, M., Wang, J., Gildersleeve, R. D., Pennathur, S., Kaufman, R. J., 2009. Translation attenuation through eIF2alpha phosphorylation prevents oxidative stress and maintains the differentiated state in beta cells. Cell Metab. 10, 13-26.
Baharvand, H., Jafary, H., Massumi, M., Ashtiani, S. K., 2006. Generation of insulinsecreting cells from human embryonic stem cells. Dev. Growth Differ. 48 , 323-332.
Banting, F., 1925. Frederick G. Banting—Nobel Lecture [WWW Document]. The Nobel Prize in Physiology or Medicine 1923. URL http.//www.nobelprize.org/nobel_prizes/medicine/laureates/1923/bantinglecture.
Basford, C. L., Prentice, K. J., Hardy, A. B., Sarangi, F., Micallef, S. J., Li, X., Guo, Q., Elefanty, A. G., Stanley, E. G., Keller, G., Allister, E. M., Nostro, M. C., Wheeler, M. B., 2011. The functional and molecular characterisation of human embryonic stem cell-derived insulin-positive cells compared with adult pancreatic beta cells. Diabetologia. Bertuzzi, F., Verzaro, R., Provenzano, V., Ricordi, C., 2007. Brittle type 1 diabetes mellitus. Curr. Med. Chem. 14, 1739-1744.
Bhattacharya, B., Miura, T., Brandenberger, R., Mejido, J., Luo, Y., Yang, A. X., Joshi, B. H., Ginis, I., Thies, R. S., Amit, M., Lyons, I., Condie, B. G., Itskovitz-Eldor, J., Rao, M. S., Puri, R. K., 2004. Gene expression in human embryonic stem cell lines: unique molecular signature. Blood 103, 2956-2964.
Bocian-Sobkowska, J., Zabel, M., Wozniak, W., Surdyk-Zasada, J., 1999. Polyhormonal aspect of the endocrine cells of the human fetal pancreas. Histochem. Cell Biol. 112, 147-153.
Boesgaard, T. W., Grarup, N., Jorgensen, T., Borch-Johnsen, K., Hansen, T., Pedersen, O., 2010. Variants at DGKB/TMEM195, ADRA2A, GLIS3 and C2CD4B loci are associated with reduced glucose-stimulated beta cell function in middle-aged Danish people. Diabetologia 53, 1647-1655.
Bongso, A., Fong, C. Y., Ng, S. C., Ratnam, S., 1994. Isolation and culture of inner cell mass cells from human blastocysts. Hum. Reprod. 9, 2110-2117.
Borowiak, M., 2010. The new generation of beta-cells: replication, stem cell differentiation, and the role of small molecules. Rev Diabet Stud 7, 93-104.
Borowiak, M., Maehr, R., Chen, S., Chen, A. E., Tang, W., Fox, J. L., Schreiber, S. L., Melton, D. A., 2009. Small Molecules Efficiently Direct Endodermal Differentiation of Mouse and Human Embryonic Stem Cells. Cell Stem Cell 4, 348-358.
Brissova, M., Fowler, M. J., Nicholson, W. E., Chu, A., Hirshberg, B., Harlan, D. M., Powers, A. C., 2005. Assessment of Human Pancreatic Islet Architecture and Composition by Laser Scanning Confocal Microscopy. J Histochem Cytochem 53, 1087-1097.
Butler, A. E., Janson, J., Bonner-Weir, S., Ritzel, R., Rizza, R. A., Butler, P. C., 2003. Beta-cell deficit and increased beta-cell apoptosis in humans with type 2 diabetes. Diabetes 52, 102-110.
Byrne, J. A., Pedersen, D. A., Clepper, L. L., Nelson, M., Sanger, W. G., Gokhale, S., Wolf,
D. P., Mitalipov, S. M., 2007. Producing primate embryonic stem cells by somatic cell nuclear transfer. Nature 450, 497-502.
Cabrera, O., 2006. The unique cytoarchitecture of human pancreatic islets has implications for islet cell function. Proceedings of the National Academy of Sciences 103, 2334-2339.
Cai, J., Yu, C., Liu, Y., Chen, S., Guo, Y., Yong, J., Lu, W., Ding, M., Deng, H., 2010.
Generation of homogeneous PDX1(+) pancreatic progenitors from human ES cell-derived endoderm cells. J Mol Cell Biol 2, 50-60.
Chen, F., Kook, H., Milewski, R., Gitler, A. D., Lu, M. M., Li, J., Nazarian, R., Schnepp,
R., Jen, K., Biben, C., Runke, G., Mackay, J. P., Novotny, J., Schwartz, R. J., Harvey, R. P., Mullins, M. C., Epstein, J. A., 2002. Hop is an unusual homeobox gene that modulates cardiac development. Cell 110, 713-723.
Chen, S., Borowiak, M., Fox, J. L., Maehr, R., Osafune, K., Davidow, L., Lam, K., Peng,
L. F., Schreiber, S. L., Rubin, L. L., Melton, D., 2009. A small molecule that directs differentiation of human ESCs into the pancreatic lineage. Nat Chem Biol 5, 258-265.
Chimienti, F., Devergnas, S., Favier, A., Seve, M., 2004. Identification and cloning of a beta-cell-specific zinc transporter, ZnT-8, localized into insulin secretory granules. Diabetes 53, 2330-2337.
Clayton, P. T., Eaton, S., Aynsley-Green, A., Edginton, M., Hussain, K., Krywawych, S.,
Datta, V., Malingre, H. E., Berger, R., van den Berg, I. E., 2001. Hyperinsulinism in short-chain L-3-hydroxyacyl-CoA dehydrogenase deficiency reveals the importance of beta-oxidation in insulin secretion. J. Clin. Invest. 108, 457-465.
Clément-Ziza, M., Munnich, A., Lyonnet, S., Jaubert, F., Besmond, C., 2008. Stabilization of RNA during laser capture microdissection by performing experiments under argon atmosphere or using ethanol as a solvent in staining solutions. RNA 14, 2698-2704.
Cohen, D. E., Melton, D., 2011. Turning straw into gold: directing cell fate for regenerative medicine. Nat Rev Genet 12, 243-252.
Cowan, C. A., Klimanskaya, I., McMahon, J., Atienza, J., Witmyer, J., Zucker, J. P., Wang, S., Morton, C. C., McMahon, A. P., Powers, D., Melton, D. A., 2004.
Derivation of embryonic stem-cell lines from human blastocysts. N. Engl. J. Med. 350, 1353-1356.
Cox, M. L., Schray, C. L., Luster, C. N., Stewart, Z. S., Korytko, P. J., et al., 2006. Assessment of fixatives, fixation, and tissue processing on morphology and RNA integrity. Exp Mol Pathol 80, 86-91.
D'Amour, K. A., Agulnick, A. D., Eliazer, S., Kelly, O. G., Kroon, E., Baetge, E. E., 2005.
Efficient differentiation of human embryonic stem cells to definitive endoderm. Nat. Biotechnol. 23, 1534-1541.
D'Amour, K. A., Bang, A. G., Eliazer, S., Kelly, O. G., Agulnick, A. D., Smart, N. G., Moorman, M. A., Kroon, E., Carpenter, M. K., Baetge, E. E., 2006. Production of pancreatic hormone-expressing endocrine cells from human embryonic stem cells. Nat Biotech 24, 1392-1401.
Daneman, D., 2006. Type 1 diabetes. Lancet 367, 847-858.
Delépine, M., Nicolino, M., Barrett, T., Golamaully, M., Lathrop, G. M., Julier, C., 2000.
EIF2AK3, encoding translation initiation factor 2-alpha kinase 3, is mutated in patients with Wolcott-Rallison syndrome. Nat. Genet. 25, 406-409.
Dorrell, C., Abraham, S. L., Lanxon-Cookson, K. M., Canaday, P. S., Streeter, P. R.,
Grompe, M., 2008. Isolation of major pancreatic cell types and long-term cultureinitiating cells using novel human surface markers. Stem Cell Res 1, 183-194.
Dorrell, C., Schug, J., Lin, C. F., Canaday, P. S., Fox, A. J., Smirnova, O., Bonnah, R., Streeter, P. R., Stoeckert, C. J., Kaestner, K. H., Grompe, M., 2011. Transcriptomes of the major human pancreatic cell types. Diabetologia 54, 2832-2844.
Dupuis, J., Langenberg, C., Prokopenko, I., Saxena, R., Soranzo, N., et al., 2010. New genetic loci implicated in fasting glucose homeostasis and their impact on type 2 diabetes risk. Nat. Genet. 42, 105-116.
Eisen, M. B., Spellman, P. T., Brown, P. O., Botstein, D., 1998. Cluster Analysis and Display of Genome-Wide Expression Patterns. Proc Natl Acad Sci USA 95 , 14863-14868.
Eshpeter, A., Jiang, J., Au, M., Rajotte, R. V., Lu, K., Lebkowski, J. S., Majumdar, A. S.,
Korbutt, G. S., 2008. In vivo characterization of transplanted human embryonic stem cell-derived pancreatic endocrine islet cells. Cell Prolif. 41, 843-858.
Evans, M. J., Kaufman, M. H., 1981. Establishment in culture of pluripotential cells from mouse embryos. Nature 292, 154-156.
Flamez, D., Roland, I., Berton, A., Kutlu, B., Dufrane, D., Beckers, M. C., De Waele, E.,
Rooman, I., Bouwens, L., Clark, A., Lonneux, M., Jamar, J. F., Goldman, S., Maréchal, D., Goodman, N., Gianello, P., Van Huffel, C., Salmon, I., Eizirik, D. L., 2010. A genomic-based approach identifies FXYD domain containing ion transport regulator 2 (FXYD2) gammaa as a pancreatic beta cell-specific biomarker. Diabetologia 53, 1372-1383.
French, A. J., Adams, C. A., Anderson, L. S., Kitchen, J. R., Hughes, M. R., Wood, S. H., 2008. Development of human cloned blastocysts following somatic cell nuclear transfer with adult fibroblasts. Stem Cells 26, 485-493.
Fujikawa, T., Oh, S.-H., Pi, L., Hatch, H. M., Shupe, T., Petersen, B. E., 2005. Teratoma
formation leads to failure of treatment for type I diabetes using embryonic stem cell-derived insulin-producing cells. Am. J. Pathol. 166, 1781-1791.
Furth, M. E., Atala, A., 2009. Stem cell sources to treat diabetes. Journal of Cellular Biochemistry 106, 507-511.
Gao, R., Ustinov, J., Korsgren, O., Otonkoski, T., 2005. In vitro neogenesis of human islets reflects the plasticity of differentiated human pancreatic cells. Diabetologia 48, 2296-2304.
Gelling, R. W., Vuguin, P. M., Du, X. Q., Cui, L., Romer, J., Pederson, R. A., Leiser, M.,
Sorensen, H., Holst, J. J., Fledelius, C., Johansen, P. B., Fleischer, N., McIntosh, C. H. S., Nishimura, E., Charron, M. J., 2009. Pancreatic B-Cell Overexpression of the Glucagon Receptor Gene Results in Enhanced B-Cell Function and Mass. Am J Physiol Endocrinol Metab 297, E695-E707.
Gibly, R. F., Graham, J. G., Luo, X., Lowe, W. L., Hering, B. J., Shea, L. D., 2011. Advancing islet transplantation: from engraftment to the immune response. Diabetologia 54, 2494-2505.
Goldwaite, C., 2006. Are Stem Cells the Next Frontier for Diabetes Treatment?, in: Regenerative Medicine. Department of Health and Human Services.
Golub, T. R., Slonim, D. K., Tamayo, P., Huard, C., Gaasenbeek, M., Mesirov, J. P., Coller, H., Loh, M. L., Downing, J. R., Caligiuri, M. A., Bloomfield, C. D., Lander, E. S., 1999. Molecular classification of cancer: class discovery and class prediction by gene expression monitoring. Science 286, 531-537.
Gradwohl, G., Dierich, A., LeMeur, M., Guillemot, F., 2000. neurogenin3 is required for
the development of the four endocrine cell lineages of the pancreas. Proc Natl Acad Sci USA 97, 1607-1611.
Gu, G., Dubauskaite, J., Melton, D. A., 2002. Direct evidence for the pancreatic lineage:
NGN3+ cells are islet progenitors and are distinct from duct progenitors. Development 129, 2447-2457.
Gunton, J. E., Kulkarni, R. N., Yim, S., Okada, T., Hawthorne, W. J., Tseng, Y.-H., Roberson, R. S., Ricordi, C., O'Connell, P. J., Gonzalez, F. J., Kahn, C. R., 2005. Loss of ARNT/HIF1beta mediates altered gene expression and pancreatic-islet dysfunction in human type 2 diabetes. Cell 122, 337-349.
Gupta, S., McGrath, B., Cavener, D. R., 2010. PERK (EIF2AK3) regulates proinsulin trafficking and quality control in the secretory pathway. Diabetes 59, 1937-1947. Hager, J., Hansen, L., Vaisse, C., Vionnet, N., Philippi, A., Poller, W., Velho, G.,
Carcassi, C., Contu, L., Julier, C., 1995. A missense mutation in the glucagon receptor gene is associated with non-insulin-dependent diabetes mellitus. Nat. Genet. 9, 299-304.
Hani, E. H., Stoffers, D. A., Chèvre, J. C., Durand, E., Stanojevic, V., Dina, C., Habener,
J. F., Froguel, P., 1999. Defective mutations in the insulin promoter factor-1 (IPF-1) gene in late-onset type 2 diabetes mellitus. J. Clin. Invest. 104, R41-48. Hansson, M., Tonning, A., Frandsen, U., Petri, A., Rajagopal, J., Englund, M. C. O., Heller, R. S., Håkansson, J., Fleckner, J., Sköld, H. N., Melton, D., Semb, H., Serup, P., 2004. Artifactual insulin release from differentiated embryonic stem cells. Diabetes 53, 2603-2609.
Hardy, O. T., Hohmeier, H. E., Becker, T. C., Manduchi, E., Doliba, N. M., Gupta, R. K., White, P., Stoeckert, C. J., Jr, Matschinsky, F. M., Newgard, C. B., Kaestner, K. H., 2007. Functional genomics of the beta-cell: short-chain 3-hydroxyacyl-coenzyme A dehydrogenase regulates insulin secretion independent of K+ currents. Mol. Endocrinol. 21, 765-773.
Harrison, K. A., Thaler, J., Pfaff, S. L., Gu, H., Kehrl, J. H., 1999. Pancreas dorsal lobe agenesis and abnormal islets of Langerhans in Hlxb9-deficient mice. Nat. Genet. 23, 71-75.
Hering, B., Bellin, M., Barton, F., 2010. Induction immunosuppression with T-cell depleting antibodies facilitates long-term insulin independence after islet allotransplantation in type 1 diabetes. Am J Transplant 10, 141-142.
Hering, B. J., Kandaswamy, R., Ansite, J. D., Eckman, P. M., Nakano, M., Sawada, T., Matsumoto, I., Ihm, S.-H., Zhang, H.-J., Parkey, J., Hunter, D. W., Sutherland, D. E. R., 2005. Single-donor, marginal-dose islet transplantation in patients with type 1 diabetes. JAMA 293, 830-835.
Holemans, K., Aerts, L., Van Assche, F. A., 2003. Lifetime consequences of abnormal fetal pancreatic development. J Physiol 547, 11-20.
Jacopin/Science Photo Library, n.d. Human blastocyst.
Jenny, M., Uhl, C., Roche, C., Duluc, I., Guillermin, V., Guillemot, F., Jensen, J., Kedinger, M., Gradwohl, G., 2002. Neurogenin3 is differentially required for endocrine cell fate specification in the intestinal and gastric epithelium. EMBO J. 21, 6338-6347.
Jiang, J., Au, M., Lu, K., Eshpeter, A., Korbutt, G., Fisk, G., Majumdar, A. S., 2007. Generation of insulin-producing islet-like clusters from human embryonic stem cells. Stem Cells 25, 1940-1953.
Jiang, W., Shi, Y., Zhao, D., Chen, S., Yong, J., Zhang, J., Qing, T., Sun, X., Zhang, P.,
Ding, M., Li, D., Deng, H., 2007. In vitro derivation of functional insulinproducing cells from human embryonic stem cells. Cell Res 17, 333-344.
Jiang, X., Yu, Y., Yang, H. W., Agar, N. Y. R., Frado, L., Johnson, M. D., 2010. The imprinted gene PEG3 inhibits Wnt signaling and regulates glioma growth. J. Biol. Chem. 285, 8472-8480.
Johannesson, M., Ståhlberg, A., Ameri, J., Sand, F. W., Norrman, K., Semb, H., 2009. FGF4 and retinoic acid direct differentiation of hESCs into PDX1-expressing foregut endoderm in a time- and concentration-dependent manner. PLoS ONE 4, e4794.
Jonsson, J., Carlsson, L., Edlund, T., Edlund, H., 1994. Insulin-promoter-factor 1 is required for pancreas development in mice. Nature 371, 606-609.
Käll, L., Krogh, A., Sonnhammer, E. L. L., 2004. A combined transmembrane topology and signal peptide prediction method. J. Mol. Biol. 338, 1027-1036.
Kalousova, A., Mavropoulos, A., Adams, B. A., Nekrep, N., Li, Z., Krauss, S., Stainier,
D. Y., German, M. S., 2010. Dachshund homologues play a conserved role in islet cell development. Dev. Biol. 348, 143-152.
Katsuta, H., Akashi, T., Katsuta, R., Nagaya, M., Kim, D., Arinobu, Y., Hara, M., Bonner-Weir, S., Sharma, A. J., Akashi, K., Weir, G. C., 2010. Single pancreatic beta-cells co-express multiple islet hormone genes in mice. Diabetologia 53, 128-138.
Kaufman, D. S., Hanson, E. T., Lewis, R. L., Auerbach, R., Thomson, J. A., 2001. Hematopoietic colony-forming cells derived from human embryonic stem cells. Proc. Natl. Acad. Sci. U.S.A. 98, 10716-10721.
Kelly, O.G., Chan, M. Y., Martinson, L. A., Kadoya, K., Ostertag, T. M., Ross, K. G., Richardson, M., Carpenter, M. K., D'Amour, K. A., Kroon, E., Moorman, M., Baetge, E. E., Bang, A. G., 2011. Cell-surface markers for the isolation of pancreatic cell types derived from human embryonic stem cells. Nat. Biotechnol. 29, 750-U114.
Khardori, R., 2011. Type 1 Diabetes Mellitus—Medscape Reference [WWW Document].
Medscape Reference. URL http://emedicine.medscape.com/article/117739-overview#aw2aab6b2b5
Kleinsmith, L. J., Pierce, G. B., Jr, 1964. Multipotentiality of single embryonal carcinoma cells. Cancer Res. 24, 1544-1551.
Korsgren, O., Nilsson, B., Berne, C., Felldin, M., Foss, A., Kallen, R., Lundgren, T., Salmela, K., Tibell, A., Tufveson, G., 2005. Current status of clinical islet transplantation. Transplantation 79, 1289-1293.
De Krijger, R. R., Aanstoot, H. J., Kranenburg, G., Reinhard, M., Visser, W. J., Bruining,
G. J., 1992. The midgestational human fetal pancreas contains cells coexpressing islet hormones. Dev. Biol. 153, 368-375.
Kroon, E., Martinson, L. A., Kadoya, K., Bang, A. G., Kelly, O. G., Eliazer, S., Young, H.,
Richardson, M., Smart, N. G., Cunningham, J., Agulnick, A. D., D'Amour, K. A., Carpenter, M. K., Baetge, E. E., 2008. Pancreatic endoderm derived from human embryonic stem cells generates glucose-responsive insulin-secreting cells in vivo. Nat Biotech 26, 443-452.
Kutlu, B., Burdick, D., Baxter, D., Rasschaert, J., Flamez, D., Eizirik, D. L., Welsh, N.,
Goodman, N., Hood, L., 2009. Detailed transcriptome atlas of the pancreatic beta cell. BMC Med Genomics 2, 3.
Lacoste, A., Berenshteyn, F., Brivanlou, A. H., 2009. An efficient and reversible transposable system for gene delivery and lineage-specific differentiation in human embryonic stem cells. Cell Stem Cell 5, 332-342.
Lamba, D. A., Karl, M. O., Ware, C. B., Reh, T. A., 2006. Efficient generation of retinal progenitor cells from human embryonic stem cells. Proc. Natl. Acad. Sci. U.S.A. 103, 12769-12774.
Lee, A.-H., Heidtman, K., Hotamisligil, G. S., Glimcher, L. H., 2011. Dual and opposing roles of the unfolded protein response regulated by IRE1alpha and XBP1 in proinsulin processing and insulin secretion. Proc. Natl. Acad. Sci. U.S.A. 108 , 8885-8890.
Lee, C. S., Perreault, N., Brestelli, J. E., Kaestner, K. H., 2002. Neurogenin 3 is essential for the proper specification of gastric enteroendocrine cells and the maintenance of gastric epithelial cell identity. Genes & Development 16, 1488-1497.
Lemaire, K., Ravier, M. A., Schraenen, A., Creemers, J. W. M., Van de Plas, R., Granvik,
M., Van Lommel, L., Waelkens, E., Chimienti, F., Rutter, G. A., Gilon, P., Veld, P. A. i., Schuit, F. C., 2009. Insulin crystallization depends on zinc transporter ZnT8 expression, but is not required for normal glucose homeostasis in mice. Proceedings of the National Academy of Sciences 106, 14872-14877.
Lerou, P. H., Yabuuchi, A., Huo, H., Takeuchi, A., Shea, J., Cimini, T., Ince, T. A., Ginsburg, E., Racowsky, C., Daley, G. Q., 2008. Human embryonic stem cell derivation from poor-quality embryos. Nat. Biotechnol. 26, 212-214.
Li, H., Arber, S., Jessell, T. M., Edlund, H., 1999. Selective agenesis of the dorsal pancreas in mice lacking homeobox gene Hlxb9. Nat. Genet. 23, 67-70.
Lipson, K. L., Fonseca, S. G., Ishigaki, S., Nguyen, L. X., Foss, E., Bortell, R., Rossini, A. A., Urano, F., 2006. Regulation of insulin biosynthesis in pancreatic beta cells by an endoplasmic reticulum-resident protein kinase IRE1. Cell Metab. 4, 245-254.
Lipson, K. L., Ghosh, R., Urano, F., 2008. The role of IRE1alpha in the degradation of insulin mRNA in pancreatic beta-cells. PLoS ONE 3, e1648.
Löser, P., Schirm, J., Guhr, A., Wobus, A. M., Kurtz, A., 2010. Human embryonic stem cell lines and their use in international research. Stem Cells 28, 240-246. Lukinius, A., Ericsson, J. L., Grimelius, L., Korsgren, O., 1992. Ultrastructural studies of the ontogeny of fetal human and porcine endocrine pancreas, with special reference to colocalization of the four major islet hormones. Dev. Biol. 153, 376-385.
Lumelsky, N., Blondel, O., Laeng, P., Velasco, I., Ravin, R., McKay, R., 2001. Differentiation of embryonic stem cells to insulin-secreting structures similar to pancreatic islets. Science 292, 1389-1394.
Lyttle, B. M., Li, J., Krishnamurthy, M., Fellows, F., Wheeler, M. B., Goodyer, C. G., Wang, R., 2008. Transcription factor expression in the developing human fetal endocrine pancreas. Diabetologia 51, 1169-1180.
Macfarlane, W. M., Frayling, T. M., Ellard, S., Evans, J. C., Allen, L. I., Bulman, M. P., Ayres, S., Shepherd, M., Clark, P., Millward, A., Demaine, A., Wilkin, T., Docherty, K., Hattersley, A. T., 1999. Missense mutations in the insulin promoter factor-1 gene predispose to type 2 diabetes. J. Clin. Invest. 104, R33-39.
Mackay, D. J. G., Callaway, J. L. A., Marks, S. M., White, H. E., Acerini, C. L., Boonen, S. E., Dayanikli, P., Firth, H. V., Goodship, J. A., Haemers, A. P., Hahnemann, J. M. D., Kordonouri, O., Masoud, A. F., Oestergaard, E., Storr, J., Ellard, S., Hattersley, A. T., Robinson, D. O., Temple, I. K., 2008. Hypomethylation of multiple imprinted loci in individuals with transient neonatal diabetes is associated with mutations in ZFP57. Nature Genetics 40, 949.
Maffei, A., Liu, Z., Witkowski, P., Moschella, F., Del Pozzo, G., Liu, E., Herold, K., Winchester, R. J., Hardy, M. A., Harris, P. E., 2004. Identification of tissuerestricted transcripts in human islets. Endocrinology 145, 4513-4521.
Marcheva, B., Ramsey, K. M., Buhr, E. D., Kobayashi, Y., Su, H., Ko, C. H., Ivanova, G.,
Omura, C., Mo, S., Vitaterna, M. H., Lopez, J. P., Philipson, L. H., Bradfield, C. A., Crosby, S. D., JeBailey, L., Wang, X., Takahashi, J. S., Bass, J., 2010. Disruption of the clock components CLOCK and BMAL1 leads to hypoinsulinaemia and diabetes. Nature 466, 627-631.
Marselli, L., Thorne, J., Ahn, Y.-B., Omer, A., Sgroi, D. C., Libermann, T., Otu, H. H., Sharma, A., Bonner-Weir, S., Weir, G. C., 2007. Gene Expression of Purified β-Cell Tissue Obtained from Human Pancreas with Laser Capture Microdissection. Journal of Clinical Endocrinology & Metabolism 93, 1046-1053.
Martin, G. R., 1981. Isolation of a pluripotent cell line from early mouse embryos cultured in medium conditioned by teratocarcinoma stem cells. Proc. Natl. Acad. Sci. U.S.A. 78, 7634-7638.
Matsui, Y., Toksoz, D., Nishikawa, S., Nishikawa, S.-I., Williams, D., Zsebo, K., Hogan,
B. L. M., 1991. Effect of Steel factor and leukaemia inhibitory factor on murine primordial germ cells in culture. Nature 353, 750-752.
Matsuoka, T., Artner, I., Henderson, E., Means, A., Sander, M., Stein, R., 2004. The MafA transcription factor appears to be responsible for tissue-specific expression of insulin. Proc. Natl. Acad. Sci. U.S.A. 101, 2930-2933.
Matveyenko, A. V., Georgia, S., Bhushan, A., Butler, P. C., 2010. Inconsistent formation and nonfunction of insulin-positive cells from pancreatic endoderm derived from human embryonic stem cells in athymic nude rats. American Journal of Physiology—Endocrinology And Metabolism 299, E713-E720.
McLean, A. B., D'Amour, K. A., Jones, K. L., Krishnamoorthy, M., Kulik, M. J., Reynolds,
D. M., Sheppard, A. M., Liu, H., Xu, Y., Baetge, E. E., Dalton, S., 2007. Activin a efficiently specifies definitive endoderm from human embryonic stem cells only when phosphatidylinositol 3-kinase signaling is suppressed. Stem Cells 25, 29-38.
Medeiros, F., Rigl, C. T., Anderson, G. G., Becker, S. H., Halling, K. C., 2007. Tissue handling for genome-wide expression analysis: A review of the issues, evidence, and opportunities. Arch Pathol Lab Med 131, 1805-1816.
Meissner, A., Jaenisch, R., 2006. Generation of nuclear transfer-derived pluripotent ES cells from cloned Cdx2-deficient blastocysts. Nature 439, 212-215.
Micallef, S. J., Li, X., Schiesser, J. V., Hirst, C. E., Yu, Q. C., Lim, S. M., Nostro, M. C., Elliott, D. A., Sarangi, F., Harrison, L. C., Keller, G., Elefanty, A. G., Stanley, E. G., 2011. INS (GFP/w) human embryonic stem cells facilitate isolation of in vitro derived insulin-producing cells. Diabetologia.
Michael, M., Wainwright, S., Williams, C., Farsides, B., Cribb, A., 2010. From Core Set to Assemblage: On the Dynamics of Exclusion and Inclusion in the Failure to Derive Beta Cells from Stem Cells. Science Studies 20, 5-25.
Moore, K., Persaud, T. V. N., 1998. The Developing Human: Clinically Oriented Embryology, 6th ed. Saunders, Philadelphia.
Mummery, C., Ward-van Oostwaard, D., Doevendans, P., Spijker, R., van den Brink, S.,
Hassink, R., van der Heyden, M., Opthof, T., Pera, M., de la Riviere, A. B., Passier, R., Tertoolen, L., 2003. Differentiation of human embryonic stem cells to cardiomyocytes: role of coculture with visceral endoderm-like cells. Circulation 107, 2733-2740.
National Diabetes Information Clearinghouse, 2011. Causes of Diabetes [WWW Document]. URL http://diabetes.niddk.nih.gov/dm/pubs/causes/
Ng, E. S., Davis, R. P., Azzola, L., Stanley, E. G., Elefanty, A. G., 2005. Forced aggregation of defined numbers of human embryonic stem cells into embryoid bodies fosters robust, reproducible hematopoietic differentiation. Blood 106 , 1601-1603.
Noggle, S., Fung, H.-L., Gore, A., Martinez, H., Satriani, K. C., Prosser, R., Oum, K., Paull, D., Druckenmiller, S., Freeby, M., Greenberg, E., Zhang, K., Goland, R., Sauer, M. V., Leibel, R. L., Egli, D., 2011. Human oocytes reprogram somatic cells to a pluripotent state. Nature 478, 70-75.
Noso, S., Kataoka, K., Kawabata, Y., Babaya, N., Hiromine, Y., Yamaji, K., Fujisawa,
T., Aramata, S., Kudo, T., Takahashi, S., Ikegami, H., 2010. Insulin transactivator MafA regulates intrathymic expression of insulin and affects susceptibility to type 1 diabetes. Diabetes 59, 2579-2587.
Nostro, M. C., Sarangi, F., Ogawa, S., Holtzinger, A., Corneo, B., Li, X., Micallef, S. J.,
Park, I.-H., Basford, C., Wheeler, M. B., Daley, G. Q., Elefanty, A. G., Stanley, E. G., Keller, G., 2011. Stage-specific signaling through TGFβ family members and WNT regulates patterning and pancreatic specification of human pluripotent stem cells. Development 138, 861-871.
Oliver-Krasinski, J. M., Stoffers, D. A., 2008. On the Origin of the B Cell. Genes Dev. 22, 1998-2021.
Osafune, K., Caron, L., Borowiak, M., Martinez, R. J., Fitz-Gerald, C. S., Sato, Y., Cowan, C. A., Chien, K. R., Melton, D. A., 2008. Marked differences in differentiation propensity among human embryonic stem cell lines. Nat Biotech 26, 313-315.
Pan, Y., Ouyang, Z., Wong, W. H., Baker, J. C., 2011. A New FACS Approach Isolates hESC Derived Endoderm Using Transcription Factors. PLoS ONE 6, e17536.
Pang, Z. P., Yang, N., Vierbuchen, T., Ostermeier, A., Fuentes, D. R., Yang, T. Q., Citri,
A., Sebastiano, V., Marro, S., SUdhof, T. C., Wernig, M., 2011. Induction of human neuronal cells by defined transcription factors. Nature.
Park, I.-H., Zhao, R., West, J. A., Yabuuchi, A., Huo, H., Ince, T. A., Lerou, P. H., Lensch,
M. W., Daley, G. Q., 2008. Reprogramming of human somatic cells to pluripotency with defined factors. Nature 451, 141-146.
Pechhold, S., Stouffer, M., Walker, G., Martel, R., Seligmann, B., Hang, Y., Stein, R., Harlan, D. M., Pechhold, K., 2009. Transcriptional analysis of intracytoplasmically stained, FACS-purified cells by high-throughput, quantitative nuclease protection. Nat. Biotechnol. 27, 1038-1042.
Pera, M. F., Cooper, S., Mills, J., Parrington, J. M., 1989. Isolation and characterization of a multipotent clone of human embryonal carcinoma cells. Differentiation 42, 10-23.
Perrier, A. L., Tabar, V., Barberi, T., Rubio, M. E., Bruses, J., Topf, N., Harrison, N. L., Studer, L., 2004. Derivation of midbrain dopamine neurons from human embryonic stem cells. Proc. Natl. Acad. Sci. U.S.A. 101, 12543-12548.
Pinney, S. E., Oliver-Krasinski, J., Ernst, L., Hughes, N., Patel, P., Stoffers, D. A., Russo,
P., De León, D. D., 2011. Neonatal diabetes and congenital malabsorptive diarrhea attributable to a novel mutation in the human neurogenin-3 gene coding sequence. J. Clin. Endocrinol. Metab. 96, 1960-1965.
Rajagopal, J., Anderson, W. J., Kume, S., Martinez, O. I., Melton, D. A., 2003. Insulin Staining of ES Cell Progeny from Insulin Uptake. Science 299, 363.
Raychaudhuri, S., Stuart, J. M., Altman, R. B., 2000. Principal components analysis to summarize microarray experiments: application to sporulation time series. Pac. Symp. Biocomput. 455-466.
Resnick, J. L., Bixler, L. S., Cheng, L., Donovan, P. J., 1992. Long-term proliferation of mouse primordial germ cells in culture. Nature 359, 550-551.
Reubinoff, B. E., Pera, M. F., Fong, C. Y., Trounson, A., Bongso, A., 2000. Embryonic stem cell lines from human blastocysts: somatic differentiation in vitro. Nat. Biotechnol. 18, 399-404.
Rezania, A., Riedel, M. J., Wideman, R. D., Karanu, F., Ao, Z., Warnock, G. L., Kieffer,
T. J., 2011. Production of functional glucagon-secreting α-cells from human embryonic stem cells. Diabetes 60, 239-247.
Riedel, M. J., Asadi, A., Wang, R., Ao, Z., Warnock, G. L., Kieffer, T. J., 2011. Immunohistochemical characterisation of cells co-producing insulin and glucagon in the developing human pancreas. Diabetologia 55, 372-381.
Roozemond, R. C., 1976. Ultramicrochemical determination of nucleic acids in individual cells using the Zeiss UMSP-I microspectrophotometer. Application to isolated rat hepatocytes of different ploidy classes. Histochem. J. 8, 625-638.
Rosenfeld, S., Wang, T., Kim, Y., Milner, J., 2004. Numerical Deconvolution of cDNA
Microarray Signal: Simulation Study. Ann NY Acad Sci 1020, 110-123.
Rubio-Cabezas, O., Codner, E., Jensen, J., Hodgson, M., Serrup, P., Ellard, S., Hattersley, A., 2009. Neurogenin 3 deficiency is a novel cause of permanent neonatal diabetes and severe congenital diarrhea. Horm Res 72, 61-62.
Rulifson, I. C., Karnik, S. K., Heiser, P. W., ten Berge, D., Chen, H., Gu, X., Taketo, M. M., Nusse, R., Hebrok, M., Kim, S. K., 2007. Wnt signaling regulates pancreatic beta cell proliferation. Proc. Natl. Acad. Sci. U.S.A. 104, 6247-6252.
Sambrook, J., Russell, D., 2001. Molecular Cloning: A Laboratory Manual, 3rd ed. Cold
Spring Harbor Labortory Press, Cold Spring Harbor, N. Y.
Sander, M., Sussel, L., Conners, J., Scheel, D., Kalamaras, J., Dela Cruz, F., Schwitzgebel, V., Hayes-Jordan, A., German, M., 2000. Homeobox gene Nkx6.1 lies downstream of Nkx2.2 in the major pathway of beta-cell formation in the pancreas. Development 127, 5533-5540.
Saxena, R., Voight, B. F., Lyssenko, V., Burtt, N. P., de Bakker, P. I. W., et al., 2007.
Genome-wide association analysis identifies loci for type 2 diabetes and triglyceride levels. Science 316, 1331-1336.
Schonhoff, S. E., Giel-Moloney, M., Leiter, A. B., 2004. Neurogenin 3-expressing progenitor cells in the gastrointestinal tract differentiate into both endocrine and non-endocrine cell types. Dev. Biol. 270, 443-454.
Schroeder, A., Mueller, O., Stocker, S., Salowsky, R., Leiber, M., Gassmann, M., Lightfoot, S., Menzel, W., Granzow, M., Ragg, T., 2006. The RIN: an RNA integrity number for assigning integrity values to RNA measurements. BMC Mol. Biol. 7, 3.
Scott, L. J., Mohlke, K. L., Bonnycastle, L. L., Willer, C. J., Li, Y., Duren, W. L., Erdos, M. R., Stringham, H. M., Chines, P. S., Jackson, A. U., Prokunina-Olsson, L., Ding, C.-J., Swift, A. J., Narisu, N., Hu, T., Pruim, R., Xiao, R., Li, X.-Y., Conneely, K. N., Riebow, N. L., Sprau, A. G., Tong, M., White, P. P., Hetrick, K. N., Barnhart, M. W., Bark, C. W., Goldstein, J. L., Watkins, L., Xiang, F., Saramies, J., Buchanan, T. A., Watanabe, R. M., Valle, T. T., Kinnunen, L., Abecasis, G. R., Pugh, E. W., Doheny, K. F., Bergman, R. N., Tuomilehto, J., Collins, F. S., Boehnke, M., 2007. A genome-wide association study of type 2 diabetes in Finns detects multiple susceptibility variants. Science 316, 1341-1345.
Segev, H., Fishman, B., Ziskind, A., Shulman, M., Itskovitz-Eldor, J., 2004. Differentiation of human embryonic stem cells into insulin-producing clusters. Stem Cells 22, 265-274.
Shamblott, M. J., Axelman, J., Wang, S., Bugg, E. M., Littlefield, J. W., Donovan, P. J., Blumenthal, P. D., Huggins, G. R., Gearhart, J. D., 1998. Derivation of pluripotent stem cells from cultured human primordial germ cells. Proceedings of the National Academy of Sciences 95, 13726-13731.
Shapiro, A. M., Lakey, J. R., Ryan, E. A., Korbutt, G. S., Toth, E., Warnock, G. L., Kneteman, N. M., Rajotte, R. V., 2000. Islet transplantation in seven patients with type 1 diabetes mellitus using a glucocorticoid-free immunosuppressive regimen. N. Engl. J. Med. 343, 230-238.
Shapiro, A. M. J., 2011. Strategies toward single-donor islets of Langerhans transplantation. Curr Opin Organ Transplant 16, 627-631.
Shapiro, A. M. J., Ricordi, C., Hering, B. J., Auchincloss, H., Lindblad, R., Robertson, R. P., Secchi, A., Brendel, M. D., Berney, T., Brennan, D. C., Cagliero, E., Alejandro, R., Ryan, E. A., DiMercurio, B., Morel, P., Polonsky, K. S., Reems, J.-A., Bretzel, R. G., Bertuzzi, F., Froud, T., Kandaswamy, R., Sutherland, D. E. R., Eisenbarth, G., Segal, M., Preiksaitis, J., Korbutt, G. S., Barton, F. B., Viviano, L., Seyfert-Margolis, V., Bluestone, J., Lakey, J. R. T., 2006. International trial of the Edmonton protocol for islet transplantation. N. Engl. J. Med. 355, 1318-1330.
Da Silva Xavier, G., Rutter, G. A., Diraison, F., Andreolas, C., Leclerc, I., 2006. ChREBP
Binding to Fatty Acid Synthase and L-Type Pyruvate Kinase Genes Is Stimulated by Glucose in Pancreatic B-Cells. J. Lipid Res. 47, 2482-2491.
da Silva Xavier, G., Sun, G., Qian, Q., Rutter, G. A., Leclerc, I., 2010. ChREBP regulates
Pdx-1 and other glucose-sensitive genes in pancreatic β-cells. Biochem. Biophys. Res. Commun. 402, 252-257. 127
Sladek, R., Rocheleau, G., Rung, J., Dina, C., Shen, L., Serre, D., Boutin, P., Vincent, D.,
Belisle, A., Hadjadj, S., Balkau, B., Heude, B., Charpentier, G., Hudson, T. J., Montpetit, A., Pshezhetsky, A. V., Prentki, M., Posner, B. I., Balding, D. J., Meyre, D., Polychronakos, C., Froguel, P., 2007. A genome-wide association study identifies novel risk loci for type 2 diabetes. Nature 445, 881-885.
Spits, C., Mateizel, I., Geens, M., Mertzanidou, A., Staessen, C., Vandeskelde, Y., Van der Elst, J., Liebaers, I., Sermon, K., 2008. Recurrent chromosomal abnormalities in human embryonic stem cells. Nat. Biotechnol. 26, 1361-1363.
Stoffers, D. A., Zinkin, N. T., Stanojevic, V., Clarke, W. L., Habener, J. F., 1997. Pancreatic agenesis attributable to a single nucleotide deletion in the human IPF1 gene coding sequence. Nat. Genet. 15, 106-110.
Subramanian, A., Tamayo, P., Mootha, V. K., Mukherjee, S., Ebert, B. L., Gillette, M. A.,
Paulovich, A., Pomeroy, S. L., Golub, T. R., Lander, E. S., Mesirov, J. P., 2005. Gene set enrichment analysis: A knowledge-based approach for interpreting genome-wide expression profiles. Proc Natl Acad Sci USA 102, 15545-15550. Sun, N., Longaker, M. T., Wu, J. C., 2010. Human iPS cell-based therapy: Considerations before clinical applications. Cell Cycle 9, 880-885.
Sussel, L., Kalamaras, J., Hartigan-O'Connor, D. J., Meneses, J. J., Pedersen, R. A., Rubenstein, J. L., German, M. S., 1998. Mice lacking the homeodomain transcription factor Nkx2.2 have diabetes due to arrested differentiation of pancreatic beta cells. Development 125, 2213-2221.
Szabat, M., Luciani, D. S., Piret, J. M., Johnson, J. D., 2009. Maturation of adult beta-cells
revealed using a Pdx1/insulin dual-reporter lentivirus. Endocrinology 150, 1627-1635.
Takahashi, K., Tanabe, K., Ohnuki, M., Narita, M., Ichisaka, T., Tomoda, K., Yamanaka,
S., 2007. Induction of pluripotent stem cells from adult human fibroblasts by defined factors. Cell 131, 861-872.
Takahashi, K., Yamanaka, S., 2006. Induction of pluripotent stem cells from mouse embryonic and adult fibroblast cultures by defined factors. Cell 126, 663-676. Tan, P., 2004. Experimental Validation of Microarray Data.
Thomson, J. A., Itskovitz-Eldor, J., Shapiro, S. S., Waknitz, M. A., Swiergiel, J. J., Marshall, V. S., Jones, J. M., 1998. Embryonic stem cell lines derived from human blastocysts. Science 282, 1145-1147.
Vuguin, P. M., Kedees, M. H., Cui, L., Guz, Y., Gelling, R. W., Nejathaim, M., Charron,
M. J., Teitelman, G., 2006. Ablation of the Glucagon Receptor Gene Increases Fetal Lethality and Produces Alterations in Islet Development and Maturation. Endocrinology 147, 3995-4006.
Wallace, C., Smyth, D. J., Maisuria-Armer, M., Walker, N. M., Todd, J. A., Clayton, D. G., 2010. The imprinted DLK1-MEG3 gene region on chromosome 14q32.2 alters susceptibility to type 1 diabetes. Nat. Genet. 42, 68-71. 128
Wang, H., Wollheim, C. B., 2002. ChREBP Rather Than USF2 Regulates Glucose Stimulation of Endogenous L-Pyruvate Kinase Expression in Insulin-Secreting Cells. J. Biol. Chem. 277, 32746-32752.
Wang, J., Cortina, G., Wu, S. V., Tran, R., Cho, J.-H., Tsai, M.-J., Bailey, T. J., Jamrich,
M., Ament, M. E., Treem, W. R., Hill, I. D., Vargas, J. H., Gershman, G., Farmer, D. G., Reyen, L., Martin, M. G., 2006. Mutant Neurogenin-3 in Congenital Malabsorptive Diarrhea. N Engl J Med 355, 270-280.
Wang, P., Rodriguez, R. T., Wang, J., Ghodasara, A., Kim, S. K., 2011. Targeting SOX17 in Human Embryonic Stem Cells Creates Unique Strategies for Isolating and Analyzing Developing Endoderm. Cell Stem Cell 8, 335-346.
Wang, S., Zhang, J., Zhao, A., Hipkens, S., Magnuson, M. A., Gu, G., 2007. Loss of Myt1 function partially compromises endocrine islet cell differentiation and pancreatic physiological function in the mouse. Mech. Dev. 124, 898-910.
Xu, X., Browning, V. L., Odorico, J. S., 2011. Activin, BMP and FGF pathways cooperate to promote endoderm and pancreatic lineage cell differentiation from human embryonic stem cells. Mechanisms of Development 128, 412-427.
Yang, L., Soonpaa, M. H., Adler, E. D., Roepke, T. K., Kattman, S. J., Kennedy, M., Henckaerts, E., Bonham, K., Abbott, G. W., Linden, R. M., Field, L. J., Keller, G. M., 2008. Human cardiovascular progenitor cells develop from a KDR+ embryonic-stem-cell-derived population. Nature 453, 524-528.
Yin, Z., Gonzales, L., Kolla, V., Rath, N., Zhang, Y., Lu, M. M., Kimura, S., Ballard, P. L., Beers, M. F., Epstein, J. A., Morrisey, E. E., 2006. Hop functions downstream of Nkx2.1 and GATA6 to mediate HDAC-dependent negative regulation of pulmonary gene expression. Am. J. Physiol. Lung Cell Mol. Physiol. 291, L191-199.
Yu, J., Vodyanik, M. A., Smuga-Otto, K., Antosiewicz-Bourget, J., Frane, J. L., Tian, S.,
Nie, J., Jonsdottir, G. A., Ruotti, V., Stewart, R., Slukvin, I. I., Thomson, J. A., 2007. Induced pluripotent stem cell lines derived from human somatic cells. Science 318, 1917-1920.
Zeggini, E., Weedon, M. N., Lindgren, C. M., Frayling, T. M., Elliott, K. S., Lango, H., Timpson, N. J., Perry, J. R. B., Rayner, N. W., Freathy, R. M., Barrett, J. C., Shields, B., Morris, A. P., Ellard, S., Groves, C. J., Harries, L. W., Marchini, J. L., Owen, K. R., Knight, B., Cardon, L. R., Walker, M., Hitman, G. A., Morris, A. D., Doney, A. S. F., McCarthy, M. I., Hattersley, A. T., 2007. Replication of genome-wide association signals in UK samples reveals risk loci for type 2 diabetes. Science 316, 1336-1341.
Zhang, D., Jiang, W., Liu, M., Sui, X., Yin, X., Chen, S., Shi, Y., Deng, H., 2009. Highly efficient differentiation of human ES cells and iPS cells into mature pancreatic insulin-producing cells. Cell Res 19, 429-438.
Zhang, W., Feng, D., Li, Y., Iida, K., McGrath, B., Cavener, D. R., 2006. PERK EIF2AK3 control of pancreatic beta cell differentiation and proliferation is required for postnatal glucose homeostasis. Cell Metab. 4, 491-497. 129
Zhao, L., Guo, M., Matsuoka, T.-A., Hagman, D. K., Parazzoli, S. D., Poitout, V., Stein, R., 2005. The islet beta cell-enriched MafA activator is a key regulator of insulin gene transcription. J. Biol. Chem. 280, 11887-11894.
Zhou, C., Dhall, D., Nissen, N. N., Chen, C.-R., Yu, R., 2009. Homozygous P86S Mutation of the Human Glucagon Receptor Is Associated With Hyperglucagonemia, α Cell Hyperplasia, and Islet Cell Tumor. Pancreas 38, 941-946.
Zhou, Q., Law, A. C., Rajagopal, J., Anderson, W. J., Gray, P. A., Melton, D. A., 2007. A multipotent progenitor domain guides pancreatic organogenesis. Dev Cell 13 , 103-114.

1. A method comprising:
(a) differentiating in vitro a population of cells comprising stem cells and/or pancreatic progenitor cells to arrive at a population of differentiated cells that express ESR1 and G6PC2 genes, wherein the differentiating comprises contacting the population of cells with at least one of Activin A, a fibroblast growth factor, a sonic hedgehog pathway antagonist, a Rho-associated kinase inhibitor, a protein kinase C agonist, and a TFG-β signaling inhibitor; and
(b) measuring expression of ESR1 and G6PC2 genes in the population of differentiated cells, wherein detection of expression indicates the presence of one or more β-cells capable of glucose-stimulated insulin secretion (GSIS).

2. A method comprising:
(a) measuring expression of ESR1 and G6PC2 genes in a population of cells to identify at least one cell expressing ESR1 and G6PC2, wherein said measuring is conducted with at least one binding assay; and
(b) administering the population of cells to a subject upon detection of expression of the ESR1 and G6PC2 genes.

3-13. (canceled)

14. The method of claim 1, wherein the stem cells are selected from the group consisting of human embryonic stem cells (hESCs), induced pluripotent stem cells (iPSCs), and combinations thereof.

15.-19. (canceled)

20. The method of claim 1, wherein the one or more β-cells comprises at least one mature β-cell.

21-24. (canceled)

25. The method of claim 1, further comprising sorting the one or more β-cells to separate immature and mature β-cells.

26. The method of claim 25, wherein sorting the one or more β-cells to separate the immature and mature β-cells comprises conducting fluorescence-activated cell sorting (FACS).

27. (canceled)

28. The method of claim 25, further comprising quantifying the sorted β-cells.

29. The method of claim 25 further comprising preserving the sorted β-cells.

30. The method of claim 1, further comprising conducting a GSIS assay on the one or more β-cells.

31-32. (canceled)

33. The method of claim 1, wherein the one or more β-cells are derived from human cells selected from the group consisting of human embryonic stem cells, reprogrammed human somatic cells, and induced human pluripotent stem cells.

34-45. (canceled)

46. The method of claim 1, wherein the sonic hedgehog pathway antagonist is SANT-1.

47. The method of claim 1, wherein the TGF-β signaling inhibitor is Alk5i.

48. The method of claim 1, wherein the fibroblast growth factor is keratinocyte growth factor (KGF).

49. The method of claim 1, wherein the Rho-associated kinase inhibitor is Y27632.

50. The method of claim 1, wherein the protein kinase C agonist is PDBU.

51. The method of claim 1, wherein said measuring is conducted with at least one binding assay.

52. The method of claim 1, further comprising administering the population of differentiated cells to a subject.

53. The method of claim 52, further comprising enriching for the one or more β-cells prior to said administering.

54. The method of claim 2, wherein the population of cells is obtained from an in vitro source.

55. The method of claim 2, wherein the population of cells is obtained from an in vivo source.

56. The method of claim 2, wherein the at least one cell expressing ESR1 and G6PC2 is a β-cell.

57. The method of claim 56, further comprising identifying the at least one cell expressing ESR1 and G6PC2 as the β-cell.

58. The method of claim 56, further comprising enriching the β-cell prior to said administering the population of cells to a subject.

59. The method of claim 56, wherein the β-cell is a mature β-cell.

60. The method of claim 59, further comprising sorting the population of cells to separate the mature β-cell from one or more immature β-cells.

61. The method of claim 56, further comprising conducting a GSIS assay on the β-cell.

62. The method of claim 2, wherein the population of cells is derived from human cells selected from the group consisting of human embryonic stem cells, reprogrammed human somatic cells, and induced human pluripotent stem cells.
